(19) United States
(12) Patent Application Publication
(10) Pub. No.: US 2024/0140997 A1
(43) Pub. Date: May 2, 2024

(54) IMMUNOGENIC COMPOSITIONS COMPRISING MYCOBACTERIUM TUBERCULOSIS POLYPEPTIDES AND FUSIONS THEREOF

(71) Applicant: ACCESS TO ADVANCED HEALTH INSTITUTE, Seattle, WA (US)

(72) Inventors: Steven G. REED, Bellevue, WA (US); Rhea N. COLER, Seattle, WA (US); Gregory C. IRETON, Seattle, WA (US); Sylvie BERTHOLET, Gaithersburg, MD (US)

(21) Appl. No.: 18/407,367

(22) Filed: Jan. 8, 2024

Related U.S. Application Data
(63) Continuation of application No. 17/367,812, filed on Jul. 6, 2021, now Pat. No. 11,897,922.
Application No. 17/367,812 is a division of application No. 15/815,512, filed on Nov. 16, 2017, now Pat. No. 11,091,521.
Application No. 15/815,512 is a division of application No. 13/791,511, filed on Mar. 8, 2013, now Pat. No. 9,822,152.
Application No. 13/791,511 is a continuation of application No. 12/594,806, filed on Jan. 6, 2010, now Pat. No. 8,486,414.

(51) Int. Cl. C07K 14/35; A61K 39/04; C07K 14/285; G01N 33/569; G01N 33/68

(52) U.S. Cl. CPC C07K 14/35; A61K 39/04; C07K 14/285; G01N 33/5695; G01N 33/68; A61K 2039/55561

(57) ABSTRACT

The present invention relates to compositions and fusion proteins containing at least two Mycobacterium sp. antigens, and polynucleotides encoding such compositions and fusion proteins. The invention also relates to methods for their use in the treatment, prevention and/or diagnosis of tuberculosis infections.

### CROSS-REFERENCE TO RELATED APPLICATION(S)

This non-provisional application is a division of U.S. patent application Ser. No. 17/367,812, filed on Jul. 6, 2021, which is a division of U.S. patent application Ser. No. 15/815,512, filed on Nov. 16, 2017, now issued as U.S. Pat. No. 11,091,521, which is a divisional of U.S. patent application Ser. No. 13/791,511, filed on Mar. 8, 2013, now issued as U.S. Pat. No. 9,822,152, which is a continuation of U.S. patent application Ser. No. 12/594,806, filed on Jan. 6, 2010, now issued as U.S. Pat. No. 8,486,414, which is a 371 national phase entry of international patent number PCT/US2008/059500, filed Apr. 4, 2008 which claims the benefit and priority of U.S. provisional application Ser. No. 60/910,169, filed on Apr. 4, 2007, all of which are hereby incorporated by reference herein in their entirety, including all references and appendices cited therein, for all purposes, as if fully set forth herein.

### STATEMENT OF GOVERNMENT INTEREST

This invention was made in part with government support under Grant Nos. AI-025038 and AI-067251 awarded by the National Institutes of Health. The government has certain rights in this invention.

### STATEMENT REGARDING SEQUENCE LISTING

The content of the Sequence Listing XML of the sequence listing named “5-US-4_SequenceListingXML.xml” which is 470,512 bytes in size was created on Jan. 5, 2024, and electronically submitted is incorporated herein by reference in its entirety.

### BACKGROUND

### Technical Field

The present invention relates generally to compositions comprising antigenic and/or immunogenic combinations of Mycobacterium tuberculosis antigens and their use in the diagnosis, treatment, and prevention of tuberculosis.

### Description of the Related Art

Tuberculosis is a chronic infectious disease caused by infection with Mycobacterium tuberculosis and other Mycobacterium species. It is a major disease in developing countries, as well as an increasing problem in developed areas of the world, with several million new cases each year. Although infection may be asymptomatic for a considerable period of time, the disease is most commonly manifested as an acute inflammation of the lungs, resulting in fever and a nonproductive cough. If untreated, serious complications and death typically result.

Although tuberculosis can generally be controlled using extended antibiotic therapy, such treatment is not sufficient to prevent the spread of the disease. Infected individuals may be asymptomatic, but contagious, for some time. In addition, although compliance with the treatment regimen is critical, patient behavior is difficult to monitor. Some patients do not complete the course of treatment, which can lead to ineffective treatment and the development of drug resistance.

In order to control the spread of tuberculosis, effective vaccination and accurate early diagnosis of the disease are critical. Currently, vaccination with live bacteria is the most widely used method for inducing protective immunity. The most common Mycobacterium employed for this purpose is Bacillus Calmette-Guerin (BCG), an avirulent strain of Mycobacterium bovis. However, the safety and efficacy of BCG is a source of controversy and some countries, such as the United States, do not vaccinate the general public with this agent.

Diagnosis of tuberculosis is commonly achieved using a skin test, which involves intradermal exposure to tuberculin PPD (protein-purified derivative). Antigen-specific T cell responses result in measurable induration at the injection site by 48-72 hours after injection, which indicates exposure to mycobacterial antigens. Sensitivity and specificity have, however, been problematic, and individuals vaccinated with BCG cannot be distinguished from infected individuals.

Accordingly, there is a need for improved reagents and methods for diagnosing, preventing and treating tuberculosis. The present invention fulfills these needs and offers other related advantages.

### BRIEF SUMMARY

The present invention relates generally to compositions comprising at least two heterologous antigens, fusion polypeptides comprising the antigens and polynucleotides encoding the antigens, where the antigens are from a Mycobacterium species, particularly Mycobacterium tuberculosis. The present invention also relates methods of using the polypeptides and polynucleotides of the invention in the diagnosis, treatment and prevention of Mycobacterium infection. The antigens of the invention, when employed in combination and/or as fusion polypeptides or polynucleotides as described herein, offer improved and unexpected levels of immunogenicity, resulting in decrease in lung bacterial burden, and thus are particularly useful in the context of vaccine development.

For example, in one aspect of the invention, there are provided compositions comprising an immunostimulant and a combination of two or more Mycobacterium tuberculosis antigens, or immunogenic fragments thereof, wherein the antigens are selected from the group consisting of Rv0164 (SEQ ID NO: 1), Rv0496 (SEQ ID NO: 6), Rv2608 (SEQ ID NO: 26), Rv3020 (SEQ ID NO: 36), Rv3478 (SEQ ID NO: 41), Rv3619 (SEQ ID NO: 46), Rv3620 (SEQ ID NO: 51), RV1738 (SEQ ID NO: 11), Rv1813 (SEQ ID NO: 16), Rv3810 (SEQ ID NO: 56), Rv2389 (SEQ ID NO: 21), Rv2866 (SEQ ID NO: 31), Rv3876 (SEQ ID NO: 61), Rv0054 (SEQ ID NO: 100), Rv0410 (SEQ ID NO: 106), Rv0655 (SEQ ID NO: 112), Rv0831 (SEQ ID NO: 115), Rv1009 (SEQ ID NO: 118), Rv1099 (SEQ ID NO: 121), Rv1240 (SEQ ID NO: 124), Rv1288 (SEQ ID NO: 127), Rv1410 (SEQ ID NO: 130), Rv1569 (SEQ ID NO: 133), Rv1789 (SEQ ID NO: 136), Rv1818 (SEQ ID NO: 139), Rv1860 (SEQ ID NO: 142), Rv1886 (SEQ ID NO: 145), Rv1908 (SEQ ID NO: 148), Rv2220 (SEQ ID NO: 154), Rv2032 (SEQ ID NO: 151), Rv2623 (SEQ ID NO: 160), Rv2875 (SEQ ID NO: 163), Rv3044 (SEQ ID NO: 166), Rv3310 (SEQ ID NO: 169), Rv3881 (SEQ ID NO: 178), Rv0577 (SEQ ID NO: 184), Rv1626 (SEQ ID NO: 187), Rv0733 (SEQ ID NO: 190), Rv2520 (SEQ ID NO: 193), Rv1253 (SEQ ID NO: 196), Rv1980 (SEQ ID NO: 199), Rv3628 (SEQ ID NO: 202) Rv1884 (SEQ ID NO: 205), Rv3872 (SEQ ID NO: 208), Rv3873 (SEQ ID NO: 211), Rv1511 (SEQ ID NO: 214) and Rv3875 (SEQ ID NO: 292) and antigens having at least 80%, 90% or 95% identity to any of the foregoing sequences.

In certain embodiments, the combination of two or more antigens is selected from the group consisting of:
(a) a combination comprising Rv1813 (SEQ ID NO: 16); Rv3620 (SEQ ID NO: 51) and Rv2608 (SEQ ID NO: 26);
(b) a combination comprising Rv2608 (SEQ ID NO: 26) and Rv3619 (SEQ ID NO: 46); and
(c) a combination comprising Rv3478 (SEQ ID NO: 41) and Rv3619 (SEQ ID NO: 46).

In a particular embodiment, the composition of (a) above, comprising Rv2608 (SEQ ID NO: 26), Rv1813 (SEQ ID NO: 16) and Rv3620 (SEQ ID NO: 51), further comprises one or more antigens selected from the group consisting of: Rv1886 (SEQ ID NO: 145), Rv2389 (SEQ ID NO: 21), Rv3478 (SEQ ID NO: 41), Rv2875 (SEQ ID NO: 163), Rv2220 (SEQ ID NO: 154), Rv0733 (SEQ ID NO: 190), Rv0577 (SEQ ID NO: 184), Rv3044 (SEQ ID NO: 166), Rv1626 (SEQ ID NO: 187), Rv3619 (SEQ ID NO: 46) and Rv3020 (SEQ ID NO: 36).

In a more particular embodiment, the composition comprises Rv1813 (SEQ ID NO: 16); Rv3620 (SEQ ID NO: 51), Rv2608 (SEQ ID NO: 26) and Rv2389 (SEQ ID NO: 21).

In related particular embodiment, the composition comprises Rv2608 (SEQ ID NO: 26); Rv1813 (SEQ ID NO: 16), Rv3620 (SEQ ID NO: 51) and Rv3619 (SEQ ID NO: 46).

In certain other embodiments of the invention, the composition of (b) above, comprising Rv2608 (SEQ ID NO: 26) and Rv3619 (SEQ ID NO: 46), further comprises one or more antigens selected from the group consisting of: Rv1886 (SEQ ID NO: 145), Rv2389 (SEQ ID NO: 21), Rv1813 (SEQ ID NO: 16), Rv2875 (SEQ ID NO: 163), Rv2220 (SEQ ID NO: 154), Rv0733 (SEQ ID NO: 190), Rv0577 (SEQ ID NO: 184), Rv3044 (SEQ ID NO: 166), Rv1626 (SEQ ID NO: 187), Rv3620 (SEQ ID NO: 51), Rv3478 (SEQ ID NO: 41), and Rv3020 (SEQ ID NO: 36).

In a particular embodiment, the composition comprises Rv2608 (SEQ ID NO: 26), Rv3619 (SEQ ID NO: 46), and Rv1886 (SEQ ID NO: 145).

In another particular embodiment, the composition further comprises one or more antigens selected from the group consisting of: Rv2389 (SEQ ID NO: 21), Rv1813 (SEQ ID NO: 16), Rv2875 (SEQ ID NO: 163), Rv2220 (SEQ ID NO: 154), Rv0733 (SEQ ID NO: 190), Rv0577 (SEQ ID NO: 184), Rv3044 (SEQ ID NO: 166), Rv1626 (SEQ ID NO: 187), Rv3620 (SEQ ID NO: 51) and Rv3020 (SEQ ID NO: 36).

In a more particular embodiment, the composition comprises Rv2608 (SEQ ID NO: 26), Rv3619 (SEQ ID NO: 46), Rv1813 (SEQ ID NO: 16) and Rv3620 (SEQ ID NO: 51).

In certain other embodiments of the invention, the composition of (c) above, comprising Rv3478 (SEQ ID NO: 41) and Rv3619 (SEQ ID NO: 46), further comprises one or more antigens selected from the group consisting of: Rv1886 (SEQ ID NO: 145), Rv2389 (SEQ ID NO: 21), Rv1813 (SEQ ID NO: 16), Rv2875 (SEQ ID NO: 163), Rv2220 (SEQ ID NO: 154), Rv0733 (SEQ ID NO: 190), Rv0577 (SEQ ID NO: 184), Rv3044 (SEQ ID NO: 166), Rv1626 (SEQ ID NO: 187), Rv3620 (SEQ ID NO: 51), Rv2608 (SEQ ID NO: 26), and Rv3020 (SEQ ID NO: 36).

In a particular embodiment, the composition comprises Rv3478 (SEQ ID NO: 41), Rv3619 (SEQ ID NO: 46) and Rv1886 (SEQ ID NO: 145).

In another embodiment, the combination further comprises one or more antigens selected from the group consisting of: Rv2389 (SEQ ID NO: 21), Rv1813 (SEQ ID NO: 16), Rv2875 (SEQ ID NO: 163), Rv2220 (SEQ ID NO: 154), Rv0733 (SEQ ID NO: 190), Rv0577 (SEQ ID NO: 184), Rv3044 (SEQ ID NO: 166), Rv1626 (SEQ ID NO: 187) and Rv3020 (SEQ ID NO: 36).

The combination of two or more antigens described herein can include a combination of two or more separate recombinant antigens, or antigenic/immunogenic fragments thereof. Alternatively, the two or more antigens, or antigenic/immunogenic fragments thereof, may be covalently linked in the form of a fusion polypeptide.

According to another aspect of the invention, there are provided isolated fusion polypeptides comprising a combination of two or more covalently linked Mycobacterium tuberculosis antigens, or immunogenic fragments thereof, wherein the antigens are selected from the group consisting of Rv0164 (SEQ ID NO: 1), Rv0496 (SEQ ID NO: 6), Rv2608 (SEQ ID NO: 26), Rv3020 (SEQ ID NO: 36), Rv3478 (SEQ ID NO: 41), Rv3619 (SEQ ID NO: 46), Rv3620 (SEQ ID NO: 51), RV1738 (SEQ ID NO: 11), Rv1813 (SEQ ID NO: 16), Rv3810 (SEQ ID NO: 56), Rv2389 (SEQ ID NO: 21), Rv2866 (SEQ ID NO: 31), Rv3876 (SEQ ID NO: 61), Rv0054 (SEQ ID NO: 100), Rv0410 (SEQ ID NO: 106), Rv0655 (SEQ ID NO: 112), Rv0831 (SEQ ID NO: 115), Rv1009 (SEQ ID NO: 118), Rv1099 (SEQ ID NO: 121), Rv1240 (SEQ ID NO: 124), Rv1288 (SEQ ID NO: 127), Rv1410 (SEQ ID NO: 130), Rv1569 (SEQ ID NO: 133), Rv1789 (SEQ ID NO: 136), Rv1818 (SEQ ID NO: 139), Rv1860 (SEQ ID NO: 142), Rv1886 (SEQ ID NO: 145), Rv1908 (SEQ ID NO: 148), Rv2220 (SEQ ID NO: 154), Rv2032 (SEQ ID NO: 151), Rv2623 (SEQ ID NO: 160), Rv2875 (SEQ ID NO: 163), Rv3044 (SEQ ID NO: 166), Rv3310 (SEQ ID NO: 169), Rv3881 (SEQ ID NO: 178), Rv0577 (SEQ ID NO: 184), Rv1626 (SEQ ID NO: 187), Rv0733 (SEQ ID NO: 190), Rv2520 (SEQ ID NO: 193), Rv1253 (SEQ ID NO: 196), Rv1980 (SEQ ID NO: 199), Rv3628 (SEQ ID NO: 202) Rv1884 (SEQ ID NO: 205), Rv3872 (SEQ ID NO: 208), Rv3873 (SEQ ID NO: 211), Rv1511 (SEQ ID NO: 214), and Rv3875 (SEQ ID NO: 292) and antigens having at least 80%, 90% or 95% identity to any of the foregoing sequences.

In certain embodiments, the fusion polypeptide comprises a combination of covalently linked antigens selected from the group consisting of:
(a) a combination comprising Rv1813 (SEQ ID NO: 16); Rv3620 (SEQ ID NO: 51) and Rv2608 (SEQ ID NO: 26);
(b) a combination comprising Rv2608 (SEQ ID NO: 26) and Rv3619 (SEQ ID NO: 46); and
(c) a combination comprising Rv3478 (SEQ ID NO: 41) and Rv3619 (SEQ ID NO: 46).

In a particular embodiment, the fusion polypeptide of (a) above, comprising Rv2608 (SEQ ID NO: 26), Rv1813 (SEQ ID NO: 16) and Rv3620 (SEQ ID NO: 51), further comprises one or more antigens selected from the group consisting of: Rv1886 (SEQ ID NO: 145), Rv2389 (SEQ ID NO: 21), Rv1813 (SEQ ID NO: 16), Rv2875 (SEQ ID NO: 163), Rv2220 (SEQ ID NO:154), Rv0733 (SEQ ID NO: 190), Rv0577 (SEQ ID NO: 184), Rv3044 (SEQ ID NO: 166), Rv1626 (SEQ ID NO: 187), Rv3619 (SEQ ID NO: 46), Rv3478 (SEQ ID NO: 41) and Rv3020 (SEQ ID NO: 36).

In a more particular embodiment, the fusion polypeptide comprises Rv1813 (SEQ ID NO: 16); Rv3620 (SEQ ID NO: 51); Rv2608 (SEQ ID NO: 26) and Rv2389 (SEQ ID NO: 21).

In a related particular embodiment, the fusion polypeptide comprises Rv1813 (SEQ ID NO: 16); Rv3620 (SEQ ID NO: 51); Rv2608 (SEQ ID NO: 26) and Rv3619 (SEQ ID NO: 46).

In certain other embodiments of the invention, the fusion polypeptide of (b) above, comprising Rv2608 (SEQ ID NO: 26) and Rv3619 (SEQ ID NO: 46), further comprises one or more antigens selected from the group consisting of: Rv1886 (SEQ ID NO: 145), Rv2389 (SEQ ID NO: 21), Rv1813 (SEQ ID NO: 16), Rv2875 (SEQ ID NO: 163), Rv2220 (SEQ ID NO: 154), Rv0733 (SEQ ID NO: 190), Rv0577 (SEQ ID NO: 184), Rv3044 (SEQ ID NO: 166), Rv1626 (SEQ ID NO: 187), Rv3620 (SEQ ID NO: 51), Rv3478 (SEQ NO: 41), and Rv3020 (SEQ ID NO: 36).

In a particular embodiment, the fusion polypeptide comprises Rv2608 (SEQ ID NO: 26), Rv1813 (SEQ NO: 16), Rv3619 (SEQ NO: 46), and Rv1886 (SEQ NO: 145). In another particular embodiment, the fusion polypeptide further comprises one or more antigens selected from the group consisting of: Rv2389 (SEQ ID NO: 21), Rv1813 (SEQ ID NO: 16), Rv2875 (SEQ ID NO: 163), Rv2220 (SEQ ID NO: 154), Rv0733 (SEQ NO: 190), Rv0577 (SEQ ID NO: 184), Rv3044 (SEQ ID NO: 166), Rv1626 (SEQ NO: 187), Rv3620 (SEQ ID NO: 51) and Rv3020 (SEQ ID NO: 36).

In a more particular embodiment, the fusion polypeptide comprises Rv2608 (SEQ ID NO: 26), Rv3619 (SEQ ID NO: 46), Rv1813 (SEQ NO: 16) and Rv3620 (SEQ NO: 51).

In certain other embodiments of the invention, the fusion polypeptide of (c) above, comprising Rv3478 (SEQ NO: 41) and Rv3619 (SEQ ID NO: 46), further comprises one or more antigens selected from the group consisting of: Rv1886 (SEQ ID NO: 145), Rv2389 (SEQ ID NO: 21), Rv1813 (SEQ ID NO: 16), Rv2875 (SEQ NO: 163), Rv2220 (SEQ NO: 154), Rv0733 (SEQ ID NO: 190), Rv0577 (SEQ ID NO: 184), Rv3044 (SEQ ID NO: 166), Rv1626 (SEQ ID NO: 187), Rv3620 (SEQ ID NO: 51), Rv2608 (SEQID NO: 26), and Rv3020 (SEQ ID NO: 36).

In a particular embodiment, the fusion polypeptide comprises Rv3478 (SEQ ID NO: 41), Rv3619 (SEQ ID NO: 46) and Rv1886 (SEQ ID NO: 145).

In another embodiment, the fusion polypeptide further comprises one or more antigens selected from the group consisting of: Rv2389 (SEQ ID NO: 21), Rv1813 (SEQ ID NO: 16), Rv2875 (SEQ ID NO: 163), Rv2220 (SEQ NO: 154), Rv0733 (SEQ NO: 190), Rv0577 (SEQ ID NO: 184), Rv3044 (SEQ NO: 166), Rv1626 (SEQ ID NO: 187) and Rv3020 (SEQ ID NO: 36).

In certain particular embodiments, fusion polypeptides are provided which comprise an amino acid sequence selected from the group consisting of: ID83 (SEQ NO: 91), ID94 (SEQ ID NO: 95), ID93 (SEQ ID NO: 226), ID91 (SEQ ID NO: 236), ID71 (SEQ ID NO: 245), ID114 (SEQ NO: 251), ID125 (SEQ ID NO: 257).

According to another aspect of the invention, there are provided isolated polynucleotides encoding any of the antigens and/or fusion polypeptides described herein.

It will be understood that, in many embodiments, the compositions, polypeptides and polynucleotides of the invention are preferably formulated in combination with one or more immunostimulants in order to improve the immune response elicited by the antigens described herein. Numerous immunostimulant and adjuvant systems are known and available in the art and can be used in the context of the present invention, illustrative examples of which include AS-2, ENHANZYN™, MPL™, 3D-MPL™, IFA, QS21, CWS, TDM, AGPs, CpG-containing oligonucleotides, Toll-like receptor agonists (e.g., TLR9 agonists, TLR7 agonists, TLR7/8 agonists, TLR5 agonists, TLR4 agonists, TLR2 agonists, TLR3 agonists, etc.), LelF, saponins, saponin mimetics, and biological and synthetic lipid A, imiquimod, gardiquimod, resiquimod, polyI:C, flagellin, or a combination thereof.

The fusion polynucleotides, fusion polypeptides, or compositions of the invention have been found to be highly antigenic. Therefore, according to another aspect of the invention, there are provided vaccines and related methods for stimulating a protective immune response in a subject by administering an effective amount of a composition as described herein. Isolated or purified polynucleotides may be used to produce recombinant fusion polypeptide antigens in vitro, which are then administered as a vaccine. Alternatively, the polynucleotides may be administered directly to a subject as a DNA-based vaccine to cause antigen expression in the subject, and the subsequent induction of an anti Mycobacterium tuberculosis immune response.

In addition, the compositions, fusion polypeptides and polynucleotides are useful as diagnostic tools in patients that may have been infected with Mycobacterium. For example, the compositions, fusion polypeptides, and polynucleotides of the invention may be used in in vitro and in vivo assays for detecting humoral antibodies or cell-mediated immunity against Mycobacterium tuberculosis for diagnosis of infection, monitoring of disease progression and/or test-of-cure evaluation. In one embodiment, there are provided diagnostic kits for detecting Mycobacterium tuberculosis infection in a biological sample, comprising (a) a polypeptide comprising at least an immunogenic portion of an antigen or fusion polypeptide described herein, (b) a detection reagent.

In another embodiment, methods are provided for detecting the presence of Mycobacterium tuberculosis infection in a biological sample, comprising (a) contacting a biological sample with a monoclonal antibody that binds to an antigen or fusion polypeptide described herein; and (b) detecting in the biological sample the presence of Mycobacterium tuberculosis proteins that bind to the monoclonal antibody.

In yet another embodiment, methods are provided for detecting Mycobacterium tuberculosis infection in a biological sample, comprising (a) contacting the biological sample with an antigen combination or fusion polypeptide as described herein and (b) detecting in the biological sample the presence of antibodies and/or T-cells that bind thereto.

In a particular embodiment, methods are provided for detecting Mycobacterium tuberculosis infection in a biological sample, comprising (a) contacting the biological sample with a combination of two or more antigens selected from the group consisting of Rv0164 (SEQ ID NO: 1), Rv0496 (SEQ ID NO: 6), Rv2608 (SEQ ID NO: 26), Rv3020 (SEQ ID NO: 36), Rv3478 (SEQ ID NO: 41), Rv3619 (SEQ ID NO: 46), Rv3620 (SEQ ID NO: 51), RV1738 (SEQ ID NO: 11), Rv1813 (SEQ ID NO: 16), Rv3810 (SEQ ID NO: 56), Rv2389 (SEQ ID NO: 21), Rv2866 (SEQ ID NO: 31), Rv3876 (SEQ ID NO: 61), Rv0054 (SEQ ID NO: 100), Rv0410 (SEQ ID NO: 106), Rv0655 (SEQ ID NO: 112), Rv0831 (SEQ ID NO: 115), Rv1009 (SEQ ID NO: 118), Rv1099 (SEQ ID NO: 121), Rv1240 (SEQ ID NO: 124), Rv1288 (SEQ ID NO: 127), Rv1410 (SEQ ID NO: 130), Rv1569 (SEQ ID NO: 133), Rv1789 (SEQ ID NO: 136), Rv1818 (SEQ ID NO: 139), Rv1860 (SEQ ID NO: 142), Rv1886 (SEQ ID NO: 145), Rv1908 (SEQ ID NO: 148), Rv2220 (SEQ ID NO: 154), Rv2032 (SEQ ID NO: 151), Rv2623 (SEQ ID NO: 160), Rv2875 (SEQ ID NO: 163), Rv3044 (SEQ ID NO: 166), Rv3310 (SEQ ID NO: 169), and Rv3881 (SEQ ID NO: 178), Rv0577 (SEQ ID NO: 184), Rv1626 (SEQ ID NO: 187), Rv0733 (SEQ ID NO: 190), Rv2520 (SEQ ID NO: 193), Rv1253 (SEQ ID NO: 196), Rv1980 (SEQ ID NO: 199), Rv3628 (SEQ ID NO: 202) Rv1884 (SEQ ID NO: 205), Rv3872 (SEQ ID NO: 208), Rv3873 (SEQ ID NO: 211), Rv1511 (SEQ ID NO: 214) and Rv3875 (SEQ ID NO: 292), or immunogenic portions thereof; and (b) detecting in the biological sample the presence of antibodies and/or T-cells that bind thereto.

In a particular embodiment, a method for detecting Mycobacterium tuberculosis infection in a biological sample comprises: contacting the biological sample with a fusion polypeptide selected from the group consisting of: DID85 (SEQ ID NO: 265); DID92 (SEQ ID NO: 273); DID108 (SEQ ID NO: 283) and DID93 (SEQ ID NO: 291); and detecting in the biological sample the presence of antibodies and/or T-cells that bind thereto.

In another particular embodiment, the invention provides diagnostic kits for detecting Mycobacterium tuberculosis infection in a biological sample, comprising: (a) a combination of two or more antigens selected from the group consisting of Rv0164 (SEQ ID NO: 1), Rv0496 (SEQ ID NO: 6), Rv2608 (SEQ ID NO: 26), Rv3020 (SEQ ID NO: 36), Rv3478 (SEQ ID NO: 41), Rv3619 (SEQ ID NO: 46), Rv3620 (SEQ ID NO: 51), RV1738 (SEQ ID NO: 11), Rv1813 (SEQ ID NO: 16), Rv3810 (SEQ ID NO: 56), Rv2389 (SEQ ID NO: 21), Rv2866 (SEQ ID NO: 31), Rv3876 (SEQ ID NO: 61), Rv0054 (SEQ ID NO: 100), Rv0410 (SEQ ID NO: 106), Rv0655 (SEQ ID NO: 112), Rv0831 (SEQ ID NO: 115), Rv1009 (SEQ ID NO: 118), Rv1099 (SEQ ID NO: 121), Rv1240 (SEQ ID NO: 124), Rv1288 (SEQ ID NO: 127), Rv1410 (SEQ ID NO: 130), Rv1569 (SEQ ID NO: 133), Rv1789 (SEQ ID NO: 136), Rv1818 (SEQ ID NO: 139), Rv1860 (SEQ ID NO: 142), Rv1886 (SEQ ID NO: 145), Rv1908 (SEQ ID NO: 148), Rv2220 (SEQ ID NO: 154), Rv2032 (SEQ ID NO: 151), Rv2623 (SEQ ID NO: 160), Rv2875 (SEQ ID NO: 163), Rv3044 (SEQ ID NO: 166), Rv3310 (SEQ ID NO: 169), and Rv3881 (SEQ ID NO: 178), Rv0577 (SEQ ID NO: 184), Rv1626 (SEQ ID NO: 187), Rv0733 (SEQ ID NO: 190), Rv2520 (SEQ ID NO: 193), Rv1253 (SEQ ID NO: 196), Rv1980 (SEQ ID NO: 199), Rv3628 (SEQ ID NO: 202) Rv1884 (SEQ ID NO: 205), Rv3872 (SEQ ID NO:208), Rv3873 (SEQ ID NO: 211), Rv1511 (SEQ ID NO: 214) and Rv3875 (SEQ ID NO: 292), or immunogenic portions thereof; and (b) a detection reagent.

In a particular embodiment, a kit of the present invention for detecting Mycobacterium tuberculosis infection in a biological sample comprises: a fusion polypeptide selected from the group consisting of: DID85 (SEQ ID NO: 265), DID92 (SEQ ID NO: 273), DID108 (SEQ ID NO: 283) and DID93 (SEQ ID NO: 291), and a detection reagent.

### BRIEF DESCRIPTION OF THE SEVERAL VIEWS OF THE DRAWINGS

FIG. 1 shows the levels of IFN-y released by antigen stimulated human PBMC. PPD⁻ and PPD⁺ PBMC were incubated for 72 h in media, 10 μm/ml PHA, 10 μg/ml Mtb lysate, 50 μg/ml of the Mtb recombinant proteins. Mean (Mean_{Ag}−Mean_{media})±SEM are shown for PPD⁺ (n=18) and PPD⁻ (n=7) PBMC.

FIGS. 2A and 2B show the levels of TNF+ splenocytes upon in vitro antigen stimulation with different Mtb recombinant proteins. Splenocytes from mice infected with a low dose of virulent M. tuberculosis H37Rv were collected 4 wks (FIG. 2A) and 12 wks (FIG. 2B) post infection (p.i.) and tested for antigen specific TNF cytokine responses by ELISPOT. The splenocytes were incubated for 48 h in media, 10 μg/ml Mtb lysate, or 10 μg/ml of the Mtb recombinant proteins. The data shown is the mean±SD (n=2) in a representative experiment.

FIGS. 3A-3D shows protection against M. tuberculosis infection and antigen specific immune responses.

FIG. 3A shows Log 10 CFU in the lung of immunized mice after an aerosol challenge with M. tuberculosis. Lungs from mice (n=7) immunized with CpG, 3 various Mtb Rv antigens, or a combination thereof were collected 4 wks after an aerosol challenge with 50-100 Mtb bacilli. CFU were counted after 2 wks of in vitro growth on agar plate. The data shown is the mean±SEM of a representative experiment.

FIG. 3B shows serum IgG2c antibody endpoint titers. Sera from mice (n=3-6) immunized with CpG, 3 various Mtb Rv antigens, or a combination thereof were collected 1 week after the 3^{rd} immunization and tested for antigen specific IgG2c antibodies by ELISA. The sera from CpG groups were tested against all Rv antigens, while the other sera were tested against the Rv antigen used for immunization. The data shown is the mean±SD of a representative experiment.

FIG. 3C shows IFN-y released by antigen stimulated splenocytes. Splenocytes from mice immunized with CpG, 3 various Mtb Rv antigens, or a combination thereof were collected 3 weeks after the 3^{rd} immunization and tested for antigen specific IFN-y cytokine responses by ELISA. The splenocytes were incubated for 72 h in media, or 10 μg/ml of the Rv antigens used for the immunization. The data shown is the mean±SD (n=3) in a representative experiment.

FIG. 3D shows relative frequencies of TNF⁺ splenocytes in response to antigen specific stimulation. Splenocytes from mice immunized with CpG, 3 various Mtb Rv antigens, or a combination thereof were collected 3 weeks after the 3^{rd} immunization and tested for antigen specific TNF cytokine responses by ELISPOT. The splenocytes were incubated for 48 h in media, or 10 μg/ml of the Rv antigens used for the immunization. The data shown is the mean±SD (n=3) in a representative experiment

FIGS. 4A and 4B show the immunogenicity of ID83 and ID93 fusion proteins with GLA-SE in C57BL/6 mice.

FIG. 4A shows antigen specific serum IgG1 and IgG2c antibody endpoint titers. Sera from mice (n=3-6) immunized with saline, ID83, or ID93 fusion protein in GLA-SE adjuvant formulations were collected 1 week after the 3^{rd} immunization and tested for ID83 and ID93 specific IgG1 and IgG2c antibodies by ELISA. The data shown is the mean±SD in a representative experiment.

FIG. 4B shows levels of IFN-y released by antigen stimulated splenocytes. Splenocytes from mice immunized with ID83 or ID93 in GLA-SE adjuvant formulation were collected 3 weeks after the 3^{rd} immunization and tested for antigen specific IFN-y cytokine responses by ELISA. The splenocytes were incubated for 72 h in media, 3 μg/ml ConA, or 10 μg/ml of ID83 or ID93 fusion proteins. The data shown is the mean±SD (n=3) in a representative experiment.

FIGS. 5A and 5B show the immunogenicity of ID83 with different adjuvant formulations in C578L/6 mice.

FIG. 5A shows antigen specific serum IgG1 and IgG2c antibody endpoint titers. Sera from mice (n=3-6) immunized with saline, or ID83 fusion protein with different adjuvant formulations were collected 1 week after the 3^{rd} immunization and tested for ID83 specific IgG1 and IgG2c antibodies by ELISA. The data shown is the mean±SD in a representative experiment.

FIG. 5B shows levels of IFN-y released by antigen stimulated splenocytes. Splenocytes from mice immunized with saline or ID83 with different adjuvant formulation were collected 3 weeks after the 3^{rd} immunization and tested for antigen specific IFN-y cytokine responses by ELISA. The splenocytes were incubated for 72 h in media, 3 μg/ml ConA, or 10 μg/ml of ID83 fusion proteins. The data shown is the mean±SD (n=3) in a representative experiment.

FIG. 6 shows the survival after infection with Mtb of guinea pigs immunized with ID83 fusion protein with GLA/CpG-SE. Guinea pigs were immunized with 1 dose of BCG, or 3 doses of ID83 with GLA/CpG-SE adjuvant and challenged with a low dose aerosol of M. tuberculosis H37Rv 4 wks after the last boost. Survival was monitored for 200 days until ¾ of the animal in the placebo group (saline) died.

FIGS. 7A and 7B show Ad5-ID83-specific immune responses and protection against an M. tuberculosis challenge.

FIG. 7A shows relative frequencies of IFN-y+ splenocytes in response to antigen specific stimulation. Splenocytes from mice immunized with saline, or 5×10⁹ Ad5-ID83 viral particles were collected 3 weeks after the 3^{rd} immunization and tested for antigen specific IFN-y cytokine responses by ELISPOT. The splenocytes were incubated for 48 h in media, or 10 μg/ml ID83 fusion protein. The data shown is the mean±SD (n=3) in a representative experiment.

FIG. 7B shows Log 10 CFU in the lung of immunized mice after an aerosol challenge with M. tuberculosis. Lungs from mice (n=7) immunized with saline, or 5×10⁹ Ad5-ID83 viral particles were collected 4 wks after an aerosol challenge with 50-100 Mtb bacilli. CFU were counted after 2 wks of in vitro growth on agar plate. The data shown is the mean±SEM of a representative experiment.

FIG. 8 shows the survival of M. tuberculosis-infected SWR mice (n=8) treated with a combination of antibiotics (Rx; rifampin+ioniazide for 60 days)+immunotherapy (three injections of a mixture containing Rv2608, Rv1813, and Rv3620 with GLA-SE), antibiotics alone (Rx; rifampin+ioniazide for 60 days) or left untreated (saline). The results demonstrate that the combination of drugs+immunotherapy extends the survival of mice infected with M. tuberculosis.

FIG. 9 shows the results of ELISA experiments in which a panel of sputum positive, Tb confirmed serum samples (n=80-92) and a panel of Tb negative, healthy control serum (n=40-46) were analyzed for reactivity with selected Tb antigens. The results demonstrate that 100% positive responses can be obtained by employing different antigen combinations.

### BRIEF DESCRIPTION OF SEQUENCE IDENTIFIERS

SEQ ID NO: 1 represents the predicted amino acid sequence for Mtb Rv0164.

SEQ ID NO: 2 represents the sequence of a PCR amplified nucleic sequence encoding Mtb Rv0164.

SEQ ID NO: 3 represents the amino acid sequence of a recombinant Mtb Rv0164, including His tag.

SEQ ID NOs: 4 and 5 represent primers used to amplify Mtb Rv0164.

SEQ ID NO: 6 represents the predicted amino acid sequence for Mtb Rv0496.

SEQ ID NO: 7 represents the sequence of a PCR amplified nucleic sequence encoding Mtb Rv0496.

SEQ ID NO: 8 represents the amino acid sequence of a recombinant Mtb Rv0496, including His tag.

SEQ ID NOs: 9 and 10 represent primers used to amplify Mtb Rv0496.

SEQ ID NO: 11 represents the predicted amino acid sequence for Mtb Rv1738.

SEQ ID NO: 12 represents the sequence of a PCR amplified nucleic sequence encoding Mtb Rv1738.

SEQ ID NO: 13 represents the amino acid sequence of a recombinant Mtb Rv1738, including His tag.

SEQ ID NOs: 14 and 15 represent primers used to amplify Mtb Rv1738.

SEQ ID NO: 16 represents the predicted amino acid sequence for Mtb Rv1813.

SEQ ID NO: 17 represents the sequence of a PCR amplified nucleic sequence encoding Mtb Rv1813.

SEQ ID NO: 18 represents the amino acid sequence of a recombinant Mtb Rv1813, including His tag.

SEQ ID NOs: 19 and 20 represent primers used to amplify Mtb Rv1813.

SEQ ID NO: 21 represents the predicted amino acid sequence for Mtb Rv2389.

SEQ ID NO: 22 represents the sequence of a PCR amplified nucleic sequence encoding Mtb Rv2389.

SEQ ID NO: 23 represents the amino acid sequence of a recombinant Mtb Rv2389, including His tag.

SEQ ID NOs: 24 and 25 represent primers used to amplify MtbRv2389.

SEQ ID NO: 26 represents the predicted amino acid sequence for Mtb Rv2608.

SEQ ID NO: 27 represents the sequence of a PCR amplified nucleic sequence encoding Mtb Rv2608.

SEQ ID NO: 28 represents the amino acid sequence of a recombinant Mtb Rv2608, including His tag.

SEQ ID NOs: 29 and 30 represent primers used to amplify Mtb Rv2608.

SEQ ID NO: 31 represents the predicted amino acid sequence for Mtb Rv2866.

SEQ ID NO: 32 and 33 represent primers used to amplify Mtb Rv2866.

SEQ ID NO: 34 represents the sequence of a PCR amplified nucleic sequence encoding Mtb Rv2866.

SEQ ID NO: 35 represents the amino acid sequence of a recombinant Mtb Rv2866, including His tag.

SEQ ID NO: 36 represents the predicted amino acid sequence for Mtb Rv3020.

SEQ ID NO: 37 represents the sequence of a PCR amplified nucleic sequence encoding Mtb Rv3020.

SEQ ID NO: 38 represents the amino acid sequence of a recombinant Mtb Rv3020, including His tag.

SEQ ID NOs: 39 and 40 represent primers used to amplify Mtb Rv3020.

SEQ ID NO: 41 represents the predicted amino acid sequence for Mtb Rv3478.

SEQ ID NO: 42 represents the sequence of a PCR amplified nucleic sequence encoding Mtb Rv3478.

SEQ ID NO: 43 represents the amino acid sequence of a recombinant Mtb Rv3478, including His tag.

SEQ ID NOs: 44 and 45 represent primers used to amplify Mtb Rv3478.

SEQ ID NO: 46 represents the predicted amino acid sequence for Mtb Rv3619.

SEQ ID NO: 47 represents the sequence of a PCR amplified nucleic sequence encoding Mtb Rv3619.

SEQ ID NO: 48 represents the amino acid sequence of a recombinant Mtb Rv3619, including His tag.

SEQ ID NOs: 49 and 50 represent primers used to amplify MtbRv3619. Mtb Rv3620.

SEQ ID NO: 51 represents the predicted amino acid sequence for

SEQ ID NO: 52 represents the sequence of a PCR amplified nucleic sequence encoding Mtb Rv3620.

SEQ ID NO: 53 represents the amino acid sequence of a recombinant Mtb Rv3620, including His tag.

SEQ ID NOs: 54 and 55 represent primers used to amplify Mtb Rv3620.

SEQ ID NO: 56 represents the predicted amino acid sequence for Mtb Rv3810.

SEQ ID NO: 57 represents the sequence of a PCR amplified nucleic sequence encoding Mtb Rv3810.

SEQ ID NO: 58 represents the amino acid sequence of a recombinant Mtb Rv3810, including His tag.

SEQ ID NOs: 59 and 60 represent primers used to amplify Mtb Rv3810.

SEQ ID NO: 61 represents the predicted amino acid sequence for Mtb Rv3876.

SEQ ID NO: 62 represents the sequence of a PCR amplified nucleic sequence encoding Mtb Rv3876.

SEQ ID NO: 63 represents the amino acid sequence of a recombinant Mtb Rv3876, including His tag.

SEQ ID NOs: 64 and 65 represent primers used to amplify Mtb Rv3876.

SEQ ID NO: 66 represents a polynucleotide sequence encoding the fusion polypeptide Mtb36f. 1.

SEQ ID NO: 67 represents the amino acid sequence of the recombinant Mtb fusion polypeptide Mtb36f 1, including His tag.

SEQ ID NOs: 68-71 represent primers used in the amplification and cloning of Mtb36f. 1.

SEQ ID NO: 72 represents a polynucleotide sequence encoding the fusion polypeptide ID58.

SEQ ID NOs: 73-78 represent primers used in the amplification and cloning of ID58.

SEQ ID NO: 79 represents the amino acid sequence of the recombinant Mtb fusion polypeptide ID58, including His tag.

SEQ ID NO: 80 represents a polynucleotide sequence encoding the fusion polypeptide ID69.

SEQ ID NOs: 81-82 represent primers used in the amplification and cloning of ID69.

SEQ ID NO: 83 represents the amino acid sequence of the recombinant Mtb fusion polypeptide ID69, including His tag.

SEQ ID NO: 84 represents a polynucleotide sequence encoding the fusion polypeptide ID83.

SEQ ID NOs: 85-90 represent primers used in the amplification and cloning of ID83.

SEQ ID NO: 91 represents the amino acid sequence of the recombinant Mtb fusion polypeptide ID83, including His tag.

SEQ ID NO: 92 represents a polynucleotide sequence encoding the fusion polypeptide ID94.

SEQ ID NOs: 93-94 represent primers used in the amplification and cloning of ID94.

SEQ ID NO: 95 represents the amino acid sequence of the recombinant Mtb fusion polypeptide ID94, including His tag.

SEQ ID NO: 96 represents a polynucleotide sequence encoding the fusion polypeptide ID95.

SEQ ID NO: 97 represents the amino acid sequence of the recombinant Mtb fusion polypeptide ID95, including His tag.

SEQ ID NO: 98 represents a polynucleotide sequence encoding the fusion polypeptide ID120.

SEQ ID NO: 99 represents the amino acid sequence of the recombinant Mtb fusion polypeptide ID120, including His tag.

SEQ ID NO: 100 represents the predicted amino acid sequence for Rv0054.

SEQ ID NO: 101 represents the sequence of a PCR amplified nucleic sequence encoding Rv0054.

SEQ ID NO: 102 represents the amino acid sequence of a recombinant Rv0054, including His tag.

SEQ ID NO: 103 represents the predicted amino acid sequence for Rv0164.

SEQ ID NO: 104 represents the sequence of a PCR amplified nucleic sequence encoding Rv0164.

SEQ ID NO: 105 represents the amino acid sequence of a recombinant Rv0164, including His tag.

SEQ ID NO: 106 represents the predicted amino acid sequence for Rv0410.

SEQ ID NO: 107 represents the sequence of a PCR amplified nucleic sequence encoding Rv0410.

SEQ ID NO: 108 represents the amino acid sequence of a recombinant Rv0410, including His tag.

SEQ ID NO: 109 represents the predicted amino acid sequence for Rv0496.

SEQ ID NO: 110 represents the sequence of a PCR amplified nucleic sequence encoding Rv0496.

SEQ ID NO: 111 represents the amino acid sequence of a recombinant Rv0496, including His tag.

SEQ ID NO: 112 represents the predicted amino acid sequence for Rv0655.

SEQ ID NO: 113 represents the sequence of a PCR amplified nucleic sequence encoding Rv0655.

SEQ ID NO: 114 represents the amino acid sequence of a recombinant Rv0655, including His tag.

SEQ ID NO: 115 represents the predicted amino acid sequence for Rv0831.

SEQ ID NO: 116 represents the sequence of a PCR amplified nucleic sequence encoding Rv0831.

SEQ ID NO: 117 represents the amino acid sequence of a recombinant Rv0831, including His tag.

SEQ ID NO: 118 represents the predicted amino acid sequence for Rv1009.

SEQ ID NO: 119 represents the sequence of a PCR amplified nucleic sequence encoding Rv1009.

SEQ ID NO: 120 represents the amino acid sequence of a recombinant Rv1009, including His tag.

SEQ ID NO: 121 represents the predicted amino acid sequence for Rv1099.

SEQ ID NO: 122 represents the sequence of a PCR amplified nucleic sequence encoding Rv1099.

SEQ ID NO: 123 represents the amino acid sequence of a recombinant Rv1099, including His tag.

SEQ ID NO: 124 represents the predicted amino acid sequence for Rv1240.

SEQ ID NO: 125 represents the sequence of a PCR amplified nucleic sequence encoding Rv1240.

SEQ ID NO: 126 represents the amino acid sequence of a recombinant Rv1240, including His tag.

SEQ ID NO: 127 represents the predicted amino acid sequence for Rv1288.

SEQ ID NO: 128 represents the sequence of a PCR amplified nucleic sequence encoding Rv1288.

SEQ ID NO: 129 represents the amino acid sequence of a recombinant Rv1288, including His tag.

SEQ ID NO: 130 represents the predicted amino acid sequence for Rv1410.

SEQ ID NO: 131 represents the sequence of a PCR amplified nucleic sequence encoding Rv1410.

SEQ ID NO: 132 represents the amino acid sequence of a recombinant Rv1410, including His tag.

SEQ ID NO: 133 represents the predicted amino acid sequence for Rv1569.

SEQ ID NO: 134 represents the sequence of a PCR amplified nucleic sequence encoding Rv1569.

SEQ ID NO: 135 represents the amino acid sequence of a recombinant Rv1569, including His tag.

SEQ ID NO: 136 represents the predicted amino acid sequence for Rv1789.

SEQ ID NO: 137 represents the sequence of a PCR amplified nucleic sequence encoding Rv1789.

SEQ ID NO: 138 represents the amino acid sequence of a recombinant Rv1789, including His tag.

SEQ ID NO: 139 represents the predicted amino acid sequence for Rv1818.

SEQ ID NO: 140 represents the sequence of a PCR amplified nucleic sequence encoding Rv1818.

SEQ ID NO: 141 represents the amino acid sequence of a recombinant Rv1818, including His tag.

SEQ ID NO: 142 represents the predicted amino acid sequence for Rv1860.

SEQ ID NO: 143 represents the sequence of a PCR amplified nucleic sequence encoding Rv1860.

SEQ ID NO: 144 represents the amino acid sequence of a recombinant Rv1860, including His tag.

SEQ ID NO: 145 represents the predicted amino acid sequence for Rv1886.

SEQ ID NO: 146 represents the sequence of a PCR amplified nucleic sequence encoding Rv1886.

SEQ ID NO: 147 represents the amino acid sequence of a recombinant Rv1886, including His tag.

SEQ ID NO: 148 represents the predicted amino acid sequence for Rv1908.

SEQ ID NO: 149 represents the sequence of a PCR amplified nucleic sequence encoding Rv1908.

SEQ ID NO: 150 represents the amino acid sequence of a recombinant Rv1908, including His tag.

SEQ ID NO: 151 represents the predicted amino acid sequence for Rv2032.

SEQ ID NO: 152 represents the sequence of a PCR amplified nucleic sequence encoding Rv2032.

SEQ ID NO: 153 represents the amino acid sequence of recombinant Rv2032, including His tag.

SEQ ID NO: 154 represents the predicted amino acid sequence for Rv2220.

SEQ ID NO: 155 represents the sequence of a PCR amplified nucleic sequence encoding Rv2220.

SEQ ID NO: 156 represents the amino acid sequence of a recombinant Rv2220, including His tag.

SEQ ID NO: 157 represents the predicted amino acid sequence for Rv2608.

SEQ ID NO: 158 represents the sequence of a PCR amplified nucleic sequence encoding Rv2608.

SEQ ID NO: 159 represents the amino acid sequence of a recombinant Rv2608, including His tag.

SEQ ID NO: 160 represents the predicted amino acid sequence for Rv2623.

SEQ ID NO: 161 represents the sequence of a PCR amplified nucleic sequence encoding Rv2623.

SEQ ID NO: 162 represents the amino acid sequence of a recombinant Rv2623, including His tag.

SEQ ID NO: 163 represents the predicted amino acid sequence for Rv2875.

SEQ ID NO: 164 represents the sequence of a PCR amplified nucleic sequence encoding Rv2875.

SEQ ID NO: 165 represents the amino acid sequence of a recombinant Rv2875, including His tag.

SEQ ID NO: 166 represents the predicted amino acid sequence for Rv3044.

SEQ ID NO: 167 represents the sequence of a PCR amplified nucleic sequence encoding Rv3044.

SEQ ID NO: 168 represents the amino acid sequence of a recombinant Rv3004, including His tag.

SEQ ID NO: 169 represents the predicted amino acid sequence for Rv3310.

SEQ ID NO: 170 represents the sequence of a PCR amplified nucleic sequence encoding Rv3310.

SEQ ID NO: 171 represents the amino acid sequence of a recombinant Rv3310, including His tag.

SEQ ID NO: 172 represents the predicted amino acid sequence for Rv3619.

SEQ ID NO: 173 represents the sequence of a PCR amplified nucleic sequence encoding Rv3619.

SEQ ID NO: 174 represents the amino acid sequence of a recombinant Rv3619, including His tag.

SEQ ID NO: 175 represents the predicted amino acid sequence for Rv3810.

SEQ ID NO: 176 represents the sequence of a PCR amplified nucleic sequence encoding Rv3810.

SEQ ID NO: 177 represents the amino acid sequence of a recombinant Rv3810, including His tag.

SEQ ID NO: 178 represents the predicted amino acid sequence for Rv3881.

SEQ ID NO: 179 represents the sequence of a PCR amplified nucleic sequence encoding Rv3881.

SEQ ID NO: 180 represents the amino acid sequence of a recombinant Rv3881, including His tag.

SEQ ID NO: 181 represents the predicted amino acid sequence for Rv0455.

SEQ ID NO: 182 represents the sequence of a PCR amplified nucleic sequence encoding Rv0455.

SEQ ID NO: 183 represents the amino acid sequence of a recombinant Rv0455, including His tag.

SEQ ID NO: 184 represents the predicted amino acid sequence for Rv0577.

SEQ ID NO: 185 represents the sequence of a PCR amplified nucleic sequence encoding Rv0577.

SEQ ID NO: 186 represents the amino acid sequence of a recombinant Rv0577, including His tag.

SEQ ID NO: 187 represents the predicted amino acid sequence for Rv1626.

SEQ ID NO: 188 represents the sequence of a PCR amplified nucleic sequence encoding Rv1626.

SEQ ID NO: 189 represents the amino acid sequence of a recombinant Rv1626, including His tag.

SEQ ID NO: 190 represents the predicted amino acid sequence for Rv0733.

SEQ ID NO: 191 represents the sequence of a PCR amplified nucleic sequence encoding Rv0733.

SEQ ID NO: 192 represents the amino acid sequence of a recombinant Rv0733, including His tag.

SEQ ID NO: 193 represents the predicted amino acid sequence for Rv2520.

SEQ ID NO: 194 represents the sequence of a PCR amplified nucleic sequence encoding Rv2520.

SEQ ID NO: 195 represents the amino acid sequence of a recombinant Rv2520, including His tag.

SEQ ID NO: 196 represents the predicted amino acid sequence for Rv1253.

SEQ ID NO: 197 represents the sequence of a PCR amplified nucleic sequence encoding Rv1253.

SEQ ID NO: 198 represents the amino acid sequence of a recombinant Rv1253, including His tag.

SEQ ID NO: 199 represents the predicted amino acid sequence for Rv1980.

SEQ ID NO: 200 represents the sequence of a PCR amplified nucleic sequence encoding Rv1980.

SEQ ID NO: 201 represents the amino acid sequence of a recombinant Rv1980, including His tag.

SEQ ID NO: 202 represents the predicted amino acid sequence for Rv3628.

SEQ ID NO: 203 represents the sequence of a PCR amplified nucleic sequence encoding Rv3628.

SEQ ID NO: 204 represents the amino acid sequence of a recombinant Rv3628, including His tag.

SEQ ID NO: 205 represents the predicted amino acid sequence for Rv1884.

SEQ ID NO: 206 represents the sequence of a PCR amplified nucleic sequence encoding Rv1884.

SEQ ID NO: 207 represents the amino acid sequence of a recombinant Rv1884, including His tag.

SEQ ID NO: 208 represents the predicted amino acid sequence for Rv3872.

SEQ ID NO: 209 represents the sequence of a PCR amplified nucleic sequence encoding Rv3872.

SEQ ID NO: 210 represents the amino acid sequence of a recombinant Rv3872, including His tag.

SEQ ID NO: 211 represents the predicted amino acid sequence for Rv3873.

SEQ ID NO: 212 represents the sequence of a PCR amplified nucleic sequence encoding Rv3873.

SEQ ID NO: 213 represents the amino acid sequence of a 10 recombinant Rv3873, including His tag.

SEQ ID NO: 214 represents the predicted amino acid sequence for Rv1511.

SEQ ID NO: 215 represents the sequence of a PCR amplified nucleic sequence encoding Rv1511.

SEQ ID NO: 216 represents the amino acid sequence of a recombinant Rv1511, including His tag.

SEQ ID NO: 217 represents a polynucleotide sequence encoding the fusion polypeptide ID93.

SEQ ID NOs: 218-225 represent primers used in the amplification and cloning of ID93.

SEQ ID NO: 226 represents the amino acid sequence of the recombinant Mtb fusion polypeptide ID93, including His tag.

SEQ ID NO: 227 represents a polynucleotide sequence encoding the fusion polypeptide ID91.

SEQ ID NOs: 228-235 represent primers used in the amplification and cloning of ID91.

SEQ ID NO: 236 represents the amino acid sequence of the recombinant Mtb fusion polypeptide ID91, including His tag.

SEQ ID NO: 237 represents a polynucleotide sequence encoding the fusion polypeptide ID71.

SEQ ID NOs: 238-244 represent primers used in the amplification and cloning of ID71.

SEQ ID NO: 245 represents the amino acid sequence of the recombinant Mtb fusion polypeptide ID71, including His tag.

SEQ ID NO: 246 represents a polynucleotide sequence encoding the fusion polypeptide ID114.

SEQ ID NOs: 247-250 represent primers used in the amplification and cloning of ID114.

SEQ ID NO: 251 represents the amino acid sequence of the recombinant Mtb fusion polypeptide ID114, including His tag.

SEQ ID NO: 252 represents a polynucleotide sequence encoding the fusion polypeptide ID125.

SEQ ID NOs: 253-256 represent primers used in the amplification and cloning of ID125.

SEQ ID NO: 257 represents the amino acid sequence of the recombinant Mtb fusion polypeptide ID125, including His tag.

SEQ ID NO: 258 represents a polynucleotide sequence encoding the fusion polypeptide DID85.

SEQ ID NOs: 259-264 represent primers used in the amplification and cloning of DID85.

SEQ ID NO: 265 represents the amino acid sequence of the recombinant Mtb fusion polypeptide DID85, including His tag.

SEQ ID NO: 266 represents a polynucleotide sequence encoding the fusion polypeptide DID92.

SEQ ID NOs: 267-272 represent primers used in the amplification and cloning of DID92.

SEQ ID NO: 273 represents the amino acid sequence of the recombinant Mtb fusion polypeptide DID92, including His tag.

SEQ ID NO: 274 represents a polynucleotide sequence encoding the fusion polypeptide DID108.

SEQ ID NOs: 275-282 represent primers used in the amplification and cloning of DID108.

SEQ ID NO: 283 represents the amino acid sequence of the recombinant Mtb fusion polypeptide DID108, including His tag.

SEQ ID NO: 284 represents a polynucleotide sequence encoding the fusion polypeptide DID93.

SEQ ID NOs: 285-290 represent primers used in the amplification and cloning of DID93.

SEQ ID NO: 291 represents the amino acid sequence of the recombinant Mtb fusion polypeptide DID93, including His tag.

SEQ ID NO: 292 represents the predicted amino acid sequence for Rv3875.

SEQ ID NO: 293 represents the sequence of a PCR amplified nucleic sequence encoding Rv3875.

SEQ ID NO: 294 represents the amino acid sequence of a recombinant Rv3875, including His tag.

SEQ ID NOs: 295-296 represent primers used in the amplification and cloning of Rv0577.

SEQ ID NOs: 297-298 represent primers used in the amplification and cloning of Rv1626.

SEQ ID NOs: 299-300 represent primers used in the amplification and cloning of Ry0733.

SEQ ID NOs: 301-302 represent primers used in the amplification and cloning of Rv2520.

SEQ ID NOs: 303-304 represent primers used in the amplification and cloning of Rv1253.

SEQ ID NOs: 305-306 represent primers used in the amplification and cloning of Rv1980.

SEQ ID NOs: 307-308 represent primers used in the amplification and cloning of Rv3628.

SEQ ID NOs: 309-310 represent primers used in the amplification and cloning of Rv1844.

SEQ ID NOs: 311-312 represent primers used in the amplification and cloning of Ry3872.

SEQ ID NOs: 313-314 represent primers used in the amplification and cloning of Ry3873.

SEQ ID NOs: 315-316 represent primers used in the amplification and cloning of Ry1511.

SEQ ID NOs: 317-318 represent primers used in the amplification and cloning of Rv3875.

### DETAILED DESCRIPTION

The present invention relates to highly antigenic/immunogenic compositions comprising Mycobacterium antigens. The compositions of the present invention generally comprise at least two heterologous polypeptides of a Mycobacterium species of the tuberculosis complex. A Mycobacterium species of the tuberculosis complex includes those species traditionally considered as causing the disease tuberculosis, as well as Mycobacterium environmental and opportunistic species that cause tuberculosis and lung disease in immune compromised patients, such as patients with AIDS, e.g., Mycobacterium tuberculosis (Mtb), Mycobacterium bovis, or Mycobacterium africanum, BCG, Mycobacterium avium, Mycobacterium intracel/ulare, Mycobacterium celatum, Mycobacterium genavense, Mycobacterium haemophilum, Mycobacterium kansasii, Mycobacterium simiae, Mycobacterium vaccae, Mycobacterium fortuitum, and Mycobacterium scrofulaceum (see, e.g., Harrison's Principles of Internal Medicine, volume 1, pp. 1004-1014 and 1019-1020. In a preferred embodiment, the Mycobacterium species to be prevented, treated or diagnosed according to the invention is Mycobacterium tuberculosis (Mtb). The sequences of antigens from Mycobacterium species are readily available. For example, Mycobacterium tuberculosis sequences can be found

in Cole et al., Nature 393:537 (1998) and can be found at websites such as those maintained by the Wellcome Trust Sanger Institute and Institut Pasteur.

### A. Mycobacterium Antigens and Fusions Thereof

The present invention, in one aspect, provides isolated Mycobacterium polypeptides, as described herein, including fusion polypeptides, and compositions containing same. Generally, a polypeptide of the invention will be an isolated polypeptide and may be a fragment (e.g., an antigenic/immunogenic portion) from an amino acid sequence disclosed herein, or may comprise an entire amino acid sequence disclosed herein. Polypeptides of the invention, antigenic/immunogenic fragments thereof, and other variants may be prepared using conventional recombinant and/or synthetic techniques.

In certain preferred embodiments, the polypeptides of the invention are antigenic/immunogenic, i.e., they react detectably within an immunoassay (such as an ELISA or T cell stimulation assay) with antisera and/or T cells from an infected subject. Screening for immunogenic activity can be performed using techniques well known to the skilled artisan. For example, such screens can be performed using methods such as those described in Harlow and Lane, Antibodies: A Laboratory Manual, Cold Spring Harbor Laboratory, 1988. In one illustrative example, a polypeptide may be immobilized on a solid support and contacted with patient sera to allow binding of antibodies within the sera to the immobilized polypeptide. Unbound sera may then be removed and bound antibodies detected using, for example, ¹²⁵I-labeled Protein A.

As would be recognized by the skilled artisan, immunogenic portions of the polypeptides disclosed herein are also encompassed by the present invention. An “immunogenic portion,” as used herein, is a fragment of an immunogenic polypeptide of the invention that itself is immunologically reactive (i.e., specifically binds) with the B-cells and/or T cell surface antigen receptors that recognize the polypeptide. Immunogenic portions may generally be identified using well known techniques, such as those summarized in Paul, Fundamental Immunology, 3rd ed., 243-247 (Raven Press, 1993) and references cited therein. Such techniques include screening polypeptides for the ability to react with antigen-specific antibodies, antisera and/or T cell lines or clones. As used herein, antisera and antibodies are “antigen-specific” if they specifically bind to an antigen (i.e., they react with the protein in an immunoassay, and do not react detectably with unrelated proteins). Such antisera and antibodies may be prepared as described herein, and using well-known techniques.

In a particular embodiment, an antigenic/immunogenic portion of a polypeptide of the present invention is a portion that reacts with antisera and/or T cells at a level that is not substantially less than the reactivity of the full-length polypeptide (e.g., in an ELISA and/or T cell reactivity assay). Preferably, the level of immunogenic activity of the antigenic/immunogenic portion is at least about 50%, preferably at least about 70% and most preferably greater than about 90% of the immunogenicity for the full-length polypeptide. In some instances, preferred immunogenic portions will be identified that have a level of immunogenic activity greater than that of the corresponding full-length polypeptide, e.g., having greater than about 100% or 150% or more immunogenic activity.

A polypeptide composition of the invention may also comprise one or more polypeptides that are immunologically reactive with T cells and/or antibodies generated against a polypeptide of the invention, particularly a polypeptide having an amino acid sequence disclosed herein, or to an immunogenic fragment or variant thereof.

In another embodiment of the invention, polypeptides are provided that comprise one or more polypeptides that are capable of eliciting T cells and/or antibodies that are immunologically reactive with one or more polypeptides described herein, or one or more polypeptides encoded by contiguous polynucleotide sequences contained in the polynucleotide sequences disclosed herein, or immunogenic fragments or variants thereof, or to one or more polynucleotide sequences which hybridize to one or more of these sequences under conditions of moderate to high stringency.

The present invention also provides polypeptide fragments, including antigenic/immunogenic fragments, comprising at least about 5, 10, 15, 20, 25, 50, or 100 contiguous amino acids, or more, including all intermediate lengths, of a polypeptide composition set forth herein, or those encoded by a polynucleotide sequence set forth herein.

In another aspect, the present invention provides variants of the polypeptide compositions described herein. Polypeptide variants generally encompassed by the present invention will typically exhibit at least about 70%, 75%, 80%, 85%, 90%, 91%, 92%, 93%, 94%, 95%, 96%, 97%, 98%, or 99% or more identity (determined as described below), along its length, to a polypeptide sequence set forth herein.

A polypeptide “variant,” as the term is used herein, is a polypeptide that typically differs from a polypeptide specifically disclosed herein in one or more substitutions, deletions, additions and/or insertions. Such variants may be naturally occurring or may be synthetically generated, for example, by modifying one or more of the above polypeptide sequences of the invention and evaluating their immunogenic activity as described herein using any of a number of techniques well known in the art.

For example, certain illustrative variants of the polypeptides of the invention include those in which one or more portions, such as an N-terminal leader sequence or transmembrane domain, have been removed. Other illustrative variants include variants in which a small portion (e.g., about 1-30 amino acids) has been removed from the N- and/or C-terminal of a mature protein.

In many instances, a variant will contain conservative substitutions. A “conservative substitution” is one in which an amino acid is substituted for another amino acid that has similar properties, such that one skilled in the art of peptide chemistry would expect the secondary structure and hydropathic nature of the polypeptide to be substantially unchanged. As described above, modifications may be made in the structure of the polynucleotides and polypeptides of the present invention and still obtain a functional molecule that encodes a variant or derivative polypeptide with desirable characteristics, e.g., with immunogenic characteristics. When it is desired to alter the amino acid sequence of a polypeptide to create an equivalent, or even an improved, immunogenic variant or portion of a polypeptide of the invention, one skilled in the art will typically change one or more of the codons of the encoding DNA sequence according to Table 1.

For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid sequence substitutions can be made in a protein sequence, and, of course, its underlying DNA coding sequence, and nevertheless obtain a protein with like properties. It is thus contemplated that various changes may be made in the peptide sequences of the disclosed compositions, or corresponding DNA sequences which encode said peptides without appreciable loss of their biological utility or activity.

TABLE 1

| Amino Acids | | | Codons | | | | | |
|---|---|---|---|---|---|---|---|---|
| Alanine | Ala | A | GCA | GCC | GCG | GCU | | |
| Cysteine | Cys | C | UGC | UGU | | | | |
| Aspartic acid | Asp | D | GAC | GAU | | | | |
| Glutemic acid | Glu | E | GAA | GAG | | | | |
| Phenylalanine | Phe | F | UUC | UUU | | | | |
| Glycine | Gly | G | GGA | GGC | GGG | GGU | | |
| Histidine | His | H | CAC | CAU | | | | |
| Isoleucine | Ile | I | AUA | AUG | AUU | | | |
| Lysine | Lys | K | AAA | AAG | | | | |
| Leucine | Leu | L | UUA | UUG | CUA | CUC | CUG | CUU |
| Methionine | Met | M | AUG | | | | | |
| Asparagine | Asn | N | AAC | AAU | | | | |
| Proline | Pro | P | CCA | CCC | CCG | CCU | | |
| Glutamine | Gln | Q | CAA | CAG | | | | |
| Arginine | Arg | R | AGA | AGG | CGA | CGC | COG | CGU |
| Serine | Ser | S | AGC | AGU | UCA | UCC | UCG | UCU |
| Threonine | Thr | T | ACA | ACC | ACC | ACU | | |
| Valine | Val | V | GUA | GUC | GUG | GUU | | |
| Tryptophan | Trp | W | UGG | | | | | |
| Tyrosine | Tyr | Y | UAC | UAU | | | | |

In making such changes, the hydropathic index of amino acids may be considered. The importance of the hydropathic amino acid index in conferring interactive biologic function on a protein is generally understood in the art (Kyte and Doolittle, 1982, incorporated herein by reference). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index on the basis of its hydrophobicity and charge characteristics (Kyte and Doolittle, 1982). These values are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is known in the art that certain amino acids may be substituted by other amino acids having a similar hydropathic index or score and still result in a protein with similar biological activity, i.e. still obtain a biological functionally equivalent protein. In making such changes, the substitution of amino acids whose hydropathic indices are within ±2 is preferred, those within ±1 are particularly preferred, and those within ±0.5 are even more particularly preferred. It is also understood in the art that the substitution of like amino acids can be made effectively on the basis of hydrophilicity.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions are generally therefore based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include: arginine and lysine; glutamate and aspartate; serine and threonine; glutamine and asparagine; and valine, leucine and isoleucine.

In addition, any polynucleotide may be further modified to increase stability in vivo. Possible modifications include, but are not limited to, the addition of flanking sequences at the 5′ and/or 3′ ends; the use of phosphorothioate or 2′ O-methyl rather than phosphodiesterase linkages in the backbone; and/or the inclusion of nontraditional bases such as inosine, queosine and wybutosine, as well as acetyl- methyl-, thio- and other modified forms of adenine, cytidine, guanine, thymine and uridine.

Amino acid substitutions may further be made on the basis of similarity in polarity, charge, solubility, hydrophobicity, hydrophilicity and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine and valine; glycine and alanine; asparagine and glutamine; and serine, threonine, phenylalanine and tyrosine. Other groups of amino acids that may represent conservative changes include: (1) ala, pro, gly, glu, asp, gin, asn, ser, thr; (2) cys, ser, tyr, thr; (3) val, ile, leu, met, ala, phe; (4) lys, arg, his; and (5) phe, tyr, trp, his. A variant may also, or alternatively, contain nonconservative changes. In a preferred embodiment, variant polypeptides differ from a native sequence by substitution, deletion or addition of five amino acids or fewer. Variants may also (or alternatively) be modified by, for example, the deletion or addition of amino acids that have minimal influence on the immunogenicity, secondary structure and hydropathic nature of the polypeptide.

As noted above, polypeptides may comprise a signal (or leader) sequence at the N-terminal end of the protein, which co-translationally or post-translationally directs transfer of the protein. The polypeptide may also be conjugated to a linker or other sequence for ease of synthesis, purification or identification of the polypeptide (e.g., poly-His), or to enhance binding of the polypeptide to a solid support. For example, a polypeptide may be conjugated to an immunoglobulin Fc region.

When comparing polypeptide sequences, two sequences are said to be “identical” if the sequence of amino acids in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, WI), using default parameters. This program embodies several alignment schemes described in the following references: Dayhoff, M. O. (1978) A model of evolutionary change in proteins—Matrices for detecting distant relationships. In Dayhoff, M. O. (ed.) Atlas of Protein Sequence and Structure, National Biomedical Research Foundation, Washington DC Vol. 5, Suppl. 3, pp. 345-358; Hein J. (1990) Unified Approach to Alignment and Phylogenes pp. 626-645 Methods in Enzymology vol. 183, Academic Press, Inc., San Diego, CA; Higgins, D. G. and Sharp, P. M. (1989) CABIOS 5:151-153; Myers, E. W. and Muller W. (1988) CABIOS 4:11-17; Robinson, E. D. (1971) Comb. Theor 11:105; Santou, N. Nes, M. (1987) Mol. Biol. Evol. 4:406-425; Sneath, P. H. A. and Sokal, R. R. (1973) Numerical Taxonomy—the Principles and Practice of Numerical Taxonomy, Freeman Press, San Francisco, CA; Wilbur, W. J. and Lipman, D. J. (1983) Proc. Nat'l Acad., Sci. USA 80:726-730.

Alternatively, optimal alignment of sequences for comparison may be conducted by the local identity algorithm of Smith and Waterman (1981) Add. APL. Math 2:482, by the identity alignment algorithm of Needleman and Wunsch (1970) J. Mol. Biol. 48:443, by the search for similarity methods of Pearson and Lipman (1988) Proc. Nat'l Acad. Sci. USA 85: 2444, by computerized implementations of these algorithms (GAP, BESTFIT, BLAST, FASTA, and TFASTA in the Wisconsin Genetics Software Package, Genetics Computer Group (GCG), 575 Science Dr., Madison, WI), or by inspection.

One preferred example of algorithms that are suitable for determining percent sequence identity and sequence similarity are the BLAST and BLAST 2.0 algorithms, which are described in Altschul et al. (1977) Nucl. Acids Res. 25:3389-3402 and Altschul et al. (1990) J. Mol. Biol. 215:403-410, respectively. BLAST and BLAST 2.0 can be used, for example with the parameters described herein, to determine percent sequence identity for the polynucleotides and polypeptides of the invention. Software for performing BLAST analyses is publicly available through the National Center for Biotechnology Information. For amino acid sequences, a scoring matrix can be used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment.

In one preferred approach, the “percentage of sequence identity” is determined by comparing two optimally aligned sequences over a window of comparison of at least 20 positions, wherein the portion of the polypeptide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical amino acid residue occurs in both sequences to yield the number of matched positions, dividing the number of matched positions by the total number of positions in the reference sequence (i.e., the window size) and multiplying the results by 100 to yield the percentage of sequence identity.

In certain preferred embodiments of the invention, there are provided Mycobacterium tuberculosis fusion polypeptides, and polynucleotides encoding fusion polypeptides. Fusion polypeptide and fusion proteins refer to a polypeptide having at least two heterologous Mycobacterium sp. polypeptides, such as Mycobacterium tuberculosis polypeptides, covalently linked, either directly or via an amino acid linker. The polypeptides forming the fusion protein are typically linked C-terminus to N-terminus, although they can also be linked C-terminus to C-terminus, N-terminus to N-terminus, or N-terminus to C-terminus. The polypeptides of the fusion protein can be in any order. Fusion polypeptides or fusion proteins can also include conservatively modified variants, polymorphic variants, alleles, mutants, subsequences, interspecies homologs, and immunogenic fragments of the antigens that make up the fusion protein. Mycobacterium tuberculosis antigens are described in Cole et al., Nature 393:537 (1998), which discloses the entire Mycobacterium tuberculosis genome. Antigens from other Mycobacterium species that correspond to Mycobacterium tuberculosis antigens can be identified, e.g., using sequence comparison algorithms, as described herein, or other methods known to those of skill in the art, e.g., hybridization assays and antibody binding assays.

The fusion polypeptides of the invention generally comprise at least two antigenic polypeptides as described herein, and may further comprise other unrelated sequences, such as a sequence that assists in providing T helper epitopes (an immunological fusion partner), preferably T helper epitopes recognized by humans, or that assists in expressing the protein (an expression enhancer) at higher yields than the native recombinant protein. Certain preferred fusion partners are both immunological and expression enhancing fusion partners. Other fusion partners may be selected so as to increase the solubility of the protein or to enable the protein to be targeted to desired intracellular compartments. Still further fusion partners include affinity tags, which facilitate purification of the protein.

Fusion proteins may generally be prepared using standard techniques. Preferably, a fusion protein is expressed as a recombinant protein. For example, DNA sequences encoding the polypeptide components of a desired fusion may be assembled separately, and ligated into an appropriate expression vector. The 3′ end of the DNA sequence encoding one polypeptide component is ligated, with or without a peptide linker, to the 5′ end of a DNA sequence encoding the second polypeptide component so that the reading frames of the sequences are in phase. This permits translation into a single fusion protein that retains the biological activity of both component polypeptides.

A peptide linker sequence may be employed to separate the first and second polypeptide components by a distance sufficient to ensure that each polypeptide folds into its secondary and tertiary structures, if desired. Such a peptide linker sequence is incorporated into the fusion protein using standard techniques well known in the art. Certain peptide linker sequences may be chosen based on the following factors: (1) their ability to adopt a flexible extended conformation; (2) their inability to adopt a secondary structure that could interact with functional epitopes on the first and second polypeptides; and (3) the lack of hydrophobic or charged residues that might react with the polypeptide functional epitopes. Preferred peptide linker sequences contain Gly, Asn and Ser residues. Other near neutral amino acids, such as Thr and Ala may also be used in the linker sequence. Amino acid sequences which may be usefully employed as linkers include those disclosed in Maratea et al., Gene 40:39 46 (1985); Murphy et al., Proc. Natl. Acad. Sci. USA 83:8258 8262 (1986); U.S. Pat. Nos. 4,935,233 and 4,751,180. The linker sequence may generally be from 1 to about 50 amino acids in length. Linker sequences are not required when the first and second polypeptides have non-essential N-terminal amino acid regions that can be used to separate the functional domains and prevent steric interference.

The ligated DNA sequences are operably linked to suitable transcriptional or translational regulatory elements. The regulatory elements responsible for expression of DNA are located only 5′ to the DNA sequence encoding the first polypeptides. Similarly, stop codons required to end translation and transcription termination signals are only present 3′ to the DNA sequence encoding the second polypeptide.

Within preferred embodiments, an immunological fusion partner for use in a fusion polypeptide of the invention is derived from protein D, a surface protein of the gram-negative bacterium Haemophilus influenza B (WO 91/18926). Preferably, a protein D derivative comprises approximately the first third of the protein (e.g., the first N-terminal 100 110 amino acids), and a protein D derivative may be lipidated. Within certain preferred embodiments, the first 109 residues of a lipoprotein D fusion partner is included on the N-terminus to provide the polypeptide with additional exogenous T cell epitopes and to increase the expression level in E. coli (thus functioning as an expression enhancer). The lipid tail ensures optimal presentation of the antigen to antigen presenting cells. Other fusion partners include the non-structural protein from influenzae virus, NS1 (hemaglutinin). Typically, the N-terminal 81 amino acids are used, although different fragments that include T-helper epitopes may be used.

In another embodiment, an immunological fusion partner comprises an amino acid sequence derived from the protein known as LYTA, or a portion thereof (preferably a C-terminal portion). LYTA is derived from Streptococcus pneumoniae, which synthesizes an N-acetyl-L-alanine amidase known as amidase LYTA (encoded by the LytA gene; Gene 43:265-292 (1986)). LYTA is an autolysin that specifically degrades certain bonds in the peptidoglycan backbone. The C-terminal domain of the LYTA protein is responsible for the affinity to the choline or to some choline analogues such as DEAE. This property has been exploited for the development of E. coli C-LYTA expressing plasmids useful for expression of fusion proteins. Purification of hybrid proteins containing the C-LYTA fragment at the amino terminus has been described (see Biotechnology 10:795-798 (1992)). Within a preferred embodiment, a repeat portion of LYTA may be incorporated into a fusion protein. A repeat portion is found in the C-terminal region starting at residue 178. A particularly preferred repeat portion incorporates residues 188-305.

In general, polypeptides and fusion polypeptides (as well as their encoding polynucleotides) are isolated. An “isolated” polypeptide or polynucleotide is one that is removed from its original environment. For example, a naturally-occurring protein is isolated if it is separated from some or all of the coexisting materials in the natural system. Preferably, such polypeptides are at least about 90% pure, more preferably at least about 95% pure and most preferably at least about 99% pure. A polynucleotide is considered to be isolated if, for example, it is cloned into a vector that is not a part of the natural environment.

### B. Polynucleotide Compositions

The present invention also provides isolated polynucleotides, particularly those encoding the fusion polypeptides of the invention, as well as compositions comprising such polynucleotides. As used herein, the terms “DNA” and “polynucleotide” and “nucleic acid” refer to a DNA molecule that has been isolated free of total genomic DNA of a particular species. Therefore, a DNA segment encoding a polypeptide refers to a DNA segment that contains one or more coding sequences yet is substantially isolated away from, or purified free from, total genomic DNA of the species from which the DNA segment is obtained. Included within the terms “DNA segment” and “polynucleotide” are DNA segments and smaller fragments of such segments, and also recombinant vectors, including, for example, plasmids, cosmids, phagemids, phage, viruses, and the like.

As will be understood by those skilled in the art, the polynucleotide sequences of this invention can include genomic sequences, extra-genomic and plasmid-encoded sequences and smaller engineered gene segments that express, or may be adapted to express, proteins, polypeptides, peptides and the like. Such segments may be naturally isolated, or modified synthetically by the hand of man.

As will be recognized by the skilled artisan, polynucleotides may be single-stranded (coding or antisense) or double-stranded, and may be DNA (genomic, cDNA or synthetic) or RNA molecules. Additional coding or non-coding sequences may, but need not, be present within a polynucleotide of the present invention, and a polynucleotide may, but need not, be linked to other molecules and/or support materials.

Polynucleotides may comprise a native sequence (i.e., an endogenous sequence that encodes a Mycobacterium antigen or a portion thereof) or may comprise a variant, or a biological or antigenic functional equivalent of such a sequence. Polynucleotide variants may contain one or more substitutions, additions, deletions and/or insertions, as further described below, preferably such that the immunogenicity of the encoded polypeptide is not diminished, relative to the native protein. The effect on the immunogenicity of the encoded polypeptide may generally be assessed as described herein. The term “variants” also encompasses homologous genes of xenogenic origin.

In additional embodiments, the present invention provides isolated polynucleotides comprising various lengths of contiguous stretches of sequence identical to or complementary to one or more of the sequences disclosed herein. For example, polynucleotides are provided by this invention that comprise at least about 15, 20, 30, 40, 50, 75, 100, 150, 200, 300, 400, 500 or 1000 or more contiguous nucleotides of one or more of the sequences disclosed herein as well as all intermediate lengths there between. It will be readily understood that “intermediate lengths”, in this context, means any length between the quoted values, such as 16, 17, 18, 19, etc.; 21, 22, 23, etc.; 30, 31, 32, etc.; 50, 51, 52, 53, etc.; 100, 101, 102, 103, etc.; 150, 151, 152, 153, etc.; including all integers through 200 500; 500 1,000, and the like.

The polynucleotides of the present invention, or fragments thereof, regardless of the length of the coding sequence itself, may be combined with other DNA sequences, such as promoters, polyadenylation signals, additional restriction enzyme sites, multiple cloning sites, other coding segments, and the like, such that their overall length may vary considerably. It is therefore contemplated that a polynucleotide fragment of almost any length may be employed, with the total length preferably being limited by the ease of preparation and use in the intended recombinant DNA protocol.

Moreover, it will be appreciated by those of ordinary skill in the art that, as a result of the degeneracy of the genetic code, there are many nucleotide sequences that encode a polypeptide as described herein. Some of these polynucleotides bear minimal homology to the nucleotide sequence of any native gene. Nonetheless, polynucleotides that vary due to differences in codon usage are specifically contemplated by the present invention, for example polynucleotides that are optimized for human and/or primate codon selection. Further, alleles of the genes comprising the polynucleotide sequences provided herein are within the scope of the present invention. Alleles are endogenous genes that are altered as a result of one or more mutations, such as deletions, additions and/or substitutions of nucleotides. The resulting mRNA and protein may, but need not, have an altered structure or function. Alleles may be identified using standard techniques (such as hybridization, amplification and/or database sequence comparison).

Mycobacterium polynucleotides and fusions thereof may be prepared, manipulated and/or expressed using any of a variety of well established techniques known and available in the art.

For example, polynucleotide sequences or fragments thereof which encode polypeptides of the invention, or fusion proteins or functional equivalents thereof, may be used in recombinant DNA molecules to direct expression of a polypeptide in appropriate host cells. Due to the inherent degeneracy of the genetic code, other DNA sequences that encode substantially the same or a functionally equivalent amino acid sequence may be produced and these sequences may be used to clone and express a given polypeptide.

As will be understood by those of skill in the art, it may be advantageous in some instances to produce polypeptide-encoding nucleotide sequences possessing non-naturally occurring codons. For example, codons preferred by a particular prokaryotic or eukaryotic host can be selected to increase the rate of protein expression or to produce a recombinant RNA transcript having desirable properties, such as a half-life which is longer than that of a transcript generated from the naturally occurring sequence.

Moreover, the polynucleotide sequences of the present invention can be engineered using methods generally known in the art in order to alter polypeptide encoding sequences for a variety of reasons, including but not limited to, alterations which modify the cloning, processing, expression and/or immunogenicity of the gene product.

In order to express a desired polypeptide, a nucleotide sequence encoding the polypeptide, or a functional equivalent, may be inserted into appropriate expression vector, i.e., a vector which contains the necessary elements for the transcription and translation of the inserted coding sequence. Methods which are well known to those skilled in the art may be used to construct expression vectors containing sequences encoding a polypeptide of interest and appropriate transcriptional and translational control elements. These methods include in vitro recombinant DNA techniques, synthetic techniques, and in vivo genetic recombination. Such techniques are described in Sambrook et al., Molecular Cloning, A Laboratory Manual (1989), and Ausubel et al., Current Protocols in Molecular Biology (1989).

A variety of expression vector/host systems are known and may be utilized to contain and express polynucleotide sequences. These include, but are not limited to, microorganisms such as bacteria transformed with recombinant bacteriophage, plasmid, or cosmid DNA expression vectors; yeast transformed with yeast expression vectors; insect cell systems infected with virus expression vectors (e.g., baculovirus); plant cell systems transformed with virus expression vectors (e.g., cauliflower mosaic virus, CaMV; tobacco mosaic virus, TMV) or with bacterial expression vectors (e.g., Ti or pBR322 plasmids); or animal cell systems.

The “control elements” or “regulatory sequences” present in an expression vector are those non-translated regions of the vector—enhancers, promoters, 5′ and 3′ untranslated regions—which interact with host cellular proteins to carry out transcription and translation. Such elements may vary in their strength and specificity. Depending on the vector system and host utilized, any number of suitable transcription and translation elements, including constitutive and inducible promoters, may be used. For example, when cloning in bacterial systems, inducible promoters such as the hybrid lacZ promoter of the PBLUESCRIPT phagemid (Stratagene, La Jolla, Calif.) or PSPORT1 plasmid (Gibco BRL, Gaithersburg, Md.) and the like may be used. In mammalian cell systems, promoters from mammalian genes or from mammalian viruses are generally preferred. If it is necessary to generate a cell line that contains multiple copies of the sequence encoding a polypeptide, vectors based on SV40 or EBV may be advantageously used with an appropriate selectable marker.

In bacterial systems, a number of expression vectors may be selected depending upon the use intended for the expressed polypeptide. For example, when large quantities are needed, vectors which direct high level expression of fusion proteins that are readily purified may be used. Such vectors include, but are not limited to, the multifunctional E. coli cloning and expression vectors such as BLUESCRIPT (Stratagene), in which the sequence encoding the polypeptide of interest may be ligated into the vector in frame with sequences for the amino-terminal Met and the subsequent 7 residues of □-galactosidase so that a hybrid protein is produced; pIN vectors (Van Heeke & Schuster, J. Biol. Chem. 264:5503 5509 (1989)); and the like. pGEX Vectors (Promega, Madison, Wis.) may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. Proteins made in such systems may be designed to include heparin, thrombin, or factor XA protease cleavage sites so that the cloned polypeptide of interest can be released from the GST moiety at will.

In the yeast, Saccharomyces cerevisiae, a number of vectors containing constitutive or inducible promoters such as alpha factor, alcohol oxidase, and PGH may be used. For reviews, see Ausubel et al. (supra) and Grant et al., Methods Enzymol. 153:516-544 (1987).

In cases where plant expression vectors are used, the expression of sequences encoding polypeptides may be driven by any of a number of promoters. For example, viral promoters such as the 35S and 19S promoters of CaMV may be used alone or in combination with the omega leader sequence from TMV (Takamatsu, EMBO J. 6:307-311 (1987)). Alternatively, plant promoters such as the small subunit of RUBISCO or heat shock promoters may be used (Coruzzi et al., EMBO J. 3:1671-1680 (1984); Broglie et al., Science 224:838-843 (1984); and Winter et al., Results Probl. Cell Differ. 17:85-105 (1991)). These constructs can be introduced into plant cells by direct DNA transformation or pathogen-mediated transfection. Such techniques are described in a number of generally available reviews (see, e.g., Hobbs in McGraw Hill, Yearbook of Science and Technology, pp. 191-196 (1992)).

An insect system may also be used to express a polypeptide of interest. For example, in one such system, Autographa californica nuclear polyhedrosis virus (AcNPV) is used as a vector to express foreign genes in Spodoptera frugiperda cells or in Trichoplusia larvae. The sequences encoding the polypeptide may be cloned into a non-essential region of the virus, such as the polyhedrin gene, and placed under control of the polyhedrin promoter. Successful insertion of the polypeptide-encoding sequence will render the polyhedrin gene inactive and produce recombinant virus lacking coat protein. The recombinant viruses may then be used to infect, for example, S. frugiperda cells or Trichoplusia larvae in which the polypeptide of interest may be expressed (Engelhard et al., Proc. Natl. Acad. Sci. U.S.A. 91:3224-3227 (1994)).

In mammalian host cells, a number of viral-based expression systems are generally available. For example, in cases where an adenovirus is used as an expression vector, sequences encoding a polypeptide of interest may be ligated into an adenovirus transcription/translation complex consisting of the late promoter and tripartite leader sequence. Insertion in a non-essential E1 or E3 region of the viral genome may be used to obtain a viable virus which is capable of expressing the polypeptide in infected host cells (Logan & Shenk, Proc. Natl. Acad. Sci. U.S.A. 81:3655-3659 (1984)). In addition, transcription enhancers, such as the Rous sarcoma virus (RSV) enhancer, may be used to increase expression in mammalian host cells.

Specific initiation signals may also be used to achieve more efficient translation of sequences encoding a polypeptide of interest. Such signals include the ATG initiation codon and adjacent sequences. In cases where sequences encoding the polypeptide, its initiation codon, and upstream sequences are inserted into the appropriate expression vector, no additional transcriptional or translational control signals may be needed. However, in cases where only coding sequence, or a portion thereof, is inserted, exogenous translational control signals including the ATG initiation codon should be provided. Furthermore, the initiation codon should be in the correct reading frame to ensure translation of the entire insert. Exogenous translational elements and initiation codons may be of various origins, both natural and synthetic. The efficiency of expression may be enhanced by the inclusion of enhancers which are appropriate for the particular cell system which is used, such as those described in the literature (Scharf. et al., Results Probl. Cell Differ. 20:125-162 (1994)).

In addition, a host cell strain may be chosen for its ability to modulate the expression of the inserted sequences or to process the expressed protein in the desired fashion. Such modifications of the polypeptide include, but are not limited to, acetylation, carboxylation, glycosylation, phosphorylation, lipidation, and acylation. Post-translational processing which cleaves a “prepro” form of the protein may also be used to facilitate correct insertion, folding and/or function. Different host cells such as CHO, HeLa, MDCK, HEK293, and W138, which have specific cellular machinery and characteristic mechanisms for such post-translational activities, may be chosen to ensure the correct modification and processing of the foreign protein.

For long-term, high-yield production of recombinant proteins, stable expression is generally preferred. For example, cell lines which stably express a polynucleotide of interest may be transformed using expression vectors which may contain viral origins of replication and/or endogenous expression elements and a selectable marker gene on the same or on a separate vector. Following the introduction of the vector, cells may be allowed to grow for 1-2 days in an enriched media before they are switched to selective media. The purpose of the selectable marker is to confer resistance to selection, and its presence allows growth and recovery of cells which successfully express the introduced sequences. Resistant clones of stably transformed cells may be proliferated using tissue culture techniques appropriate to the cell type.

Any number of selection systems may be used to recover transformed cell lines. These include, but are not limited to, the herpes simplex virus thymidine kinase (Wigler et al., Cell 11:223-232 (1977)) and adenine phosphoribosyltransferase (Lowy et al., Cell 22:817-823 (1990)) genes which can be employed in tk- or aprt-cells, respectively. Also, antimetabolite, antibiotic or herbicide resistance can be used as the basis for selection; for example, dhfr which confers resistance to methotrexate (Wigler et al., Proc. Natl. Acad. Sci. U.S.A. 77:3567-70 (1980)); npt, which confers resistance to the aminoglycosides, neomycin and G-418 (Colbere-Garapin et al., J. Mol. Biol. 150:1-14 (1981)); and als or pat, which confer resistance to chlorsulfuron and phosphinotricin acetyltransferase, respectively (Murry, supra). Additional selectable genes have been described, for example, trpB, which allows cells to utilize indole in place of tryptophan, or hisD, which allows cells to utilize histinol in place of histidine (Hartman & Mulligan, Proc. Natl. Acad. ScL U.S.A. 85:8047-51 (1988)). The use of visible markers has gained popularity with such markers as anthocyanins, β-glucuronidase and its substrate GUS, and luciferase and its substrate luciferin, being widely used not only to identify transformants, but also to quantify the amount of transient or stable protein expression attributable to a specific vector system (Rhodes et al., Methods Mol. Biol. 55:121-131 (1995)).

A variety of protocols for detecting and measuring the expression of polynucleotide-encoded products, using either polyclonal or monoclonal antibodies specific for the product are known in the art. Examples include enzyme-linked immunosorbent assay (ELISA), radioimmunoassay (RIA), and fluorescence activated cell sorting (FACS). These and other assays are described, among other places, in Hampton et al., Serological Methods, a Laboratory Manual (1990) and Maddox et al., J. Exp. Med. 158:1211-1216 (1983).

A wide variety of labels and conjugation techniques are known by those skilled in the art and may be used in various nucleic acid and amino acid assays. Means for producing labeled hybridization or PCR probes for detecting sequences related to polynucleotides include oligolabeling, nick translation, end-labeling or PCR amplification using a labeled nucleotide. Alternatively, the sequences, or any portions thereof may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits. Suitable reporter molecules or labels, which may be used include radionuclides, enzymes, fluorescent, chemiluminescent, or chromogenic agents as well as substrates, cofactors, inhibitors, magnetic particles, and the like.

Host cells transformed with a polynucleotide sequence of interest may be cultured under conditions suitable for the expression and recovery of the protein from cell culture. The protein produced by a recombinant cell may be secreted or contained intracellularly depending on the sequence and/or the vector used. As will be understood by those of skill in the art, expression vectors containing polynucleotides of the invention may be designed to contain signal sequences which direct secretion of the encoded polypeptide through a prokaryotic or eukaryotic cell membrane. Other recombinant constructions may be used to join sequences encoding a polypeptide of interest to nucleotide sequence encoding a polypeptide domain which will facilitate purification of soluble proteins.

In addition to recombinant production methods, polypeptides of the invention, and fragments thereof, may be produced by direct peptide synthesis using solid-phase techniques (Merrifield, J. Am. Chem. Soc. 85:2149-2154 (1963)). Protein synthesis may be performed using manual techniques or by automation. Automated synthesis may be achieved, for example, using Applied Biosystems 431A Peptide Synthesizer (Perkin Elmer). Alternatively, various fragments may be chemically synthesized separately and combined using chemical methods to produce the full length molecule.

### C. Pharmaceutical and Vaccine Compositions

In another aspect, the present invention concerns formulations of one or more of the polynucleotide, polypeptide or other compositions disclosed herein in pharmaceutically-acceptable or physiologically-acceptable solutions for administration to a cell or an animal, either alone, or in combination with one or more other modalities of therapy. Such pharmaceutical compositions are particularly preferred for use as vaccines when formulated with a suitable immunostimulant/adjuvant system. The compositions are also suitable for use in a diagnostic context.

It will also be understood that, if desired, the compositions of the invention may be administered in combination with other agents as well, such as, e.g., other proteins or polypeptides or various pharmaceutically-active agents. There is virtually no limit to other components that may also be included, provided that the additional agents do not cause a significant adverse effect upon the objectives according to the invention.

In certain preferred embodiments the compositions of the invention are used as vaccines and are formulated in combination with one or more immunostimulants. An immunostimulant may be any substance that enhances or potentiates an immune response (antibody and/or cell-mediated) to an exogenous antigen. Examples of immunostimulants include adjuvants, biodegradable microspheres (e.g., polylactic galactide) and liposomes (into which the compound is incorporated; see, e.g., Fullerton, U.S. Pat. No. 4,235,877). Vaccine preparation is generally described in, for example, Powell & Newman, eds., Vaccine Design (the subunit and adjuvant approach) (1995).

Any of a variety of immunostimulants may be employed in the vaccines of this invention. For example, an adjuvant may be included. Many adjuvants contain a substance designed to protect the antigen from rapid catabolism, such as aluminum hydroxide or mineral oil, and a stimulator of immune responses, such as lipid A (natural or synthetic), Bortadella pertussis or Mycobacterium species or Mycobacterium derived proteins. Suitable adjuvants are commercially available as, for example, Freund's Incomplete Adjuvant and Complete Adjuvant (Difco Laboratories, Detroit, Mich.); Merck Adjuvant 65 (Merck and Company, Inc., Rahway, N.J.); AS-2 and derivatives thereof (SmithKline Beecham, Philadelphia, Pa.); CWS, TDM, Leif, aluminum salts such as aluminum hydroxide gel (alum) or aluminum phosphate; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quil A. Cytokines, such as GM-CSF or interleukin-2, -7, or -12, may also be used as adjuvants.

In certain preferred embodiments, the adjuvant used in the present invention is a glucopyranosyl lipid A (GLA) adjuvant, as described in pending U.S. patent application Ser. No. 11/862,122, the disclosure of which is incorporated herein by reference in its entirety. For example, certain GLA compounds of interest are represented by the following formula:

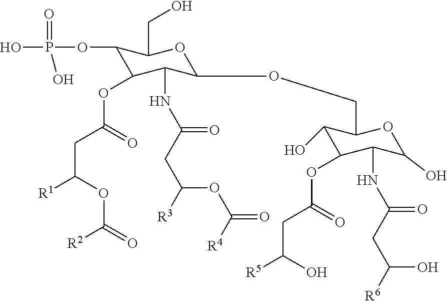

where: R¹, R³, R⁵ and R⁶ are C₁₁-C₂₀ alkyl; and R² and R⁴ are C₁₂-C₂₀ alkyl. In a more particular embodiment, R¹, R², R³, R⁴, R⁵ and R⁶ are C₁₄.

Other illustrative adjuvants useful in the context of the invention include Toll-like receptor agonists, such as TLR7 agonists, TLR7/8 agonists, and the like. Still other illustrative adjuvants include imiquimod (IMQ), gardiquimod (GDQ), resiquimod (RSQ), and related compounds.

Certain preferred vaccines employ adjuvant systems designed to induce an immune response predominantly of the Th1 type. High levels of Th1-type cytokines (e.g., IFN-γ, TNF, IL-2 and IL-12) tend to favor the induction of cell mediated immune responses to an administered antigen. In contrast, high levels of Th2-type cytokines (e.g., IL-4, IL-5, IL-6 and IL-10) tend to favor the induction of humoral immune responses. Following application of a vaccine as provided herein, a patient will support an immune response that includes Th1- and Th2-type responses. Within a preferred embodiment, in which a response is predominantly Th1-type, the level of Th1-type cytokines will increase to a greater extent than the level of Th2-type cytokines. The levels of these cytokines may be readily assessed using standard assays. For a review of the families of cytokines, see Mossman & Coffman, Ann. Rev. Immunol. 7:145-173 (1989).

Certain adjuvants for use in eliciting a predominantly Th1-type response include, for example, a combination of monophosphoryl lipid A, preferably 3-de-O-acylated monophosphoryl lipid A (3D-MPL™), together with an aluminum salt (U.S. Pat. Nos. 4,436,727; 4,877,611; 4,866,034; and 4,912,094). CpG-containing oligonucleotides (in which the CpG dinucleotide is unmethylated) also induce a predominantly Th1 response. Such oligonucleotides are well known and are described, for example, in WO 96/02555, WO 99/33488 and U.S. Pat. Nos. 6,008,200 and 5,856,462. Immunostimulatory DNA sequences are also described, for example, by Sato et al., Science 273:352 (1996). Another illustrative adjuvant comprises a saponin, such as Quil A, or derivatives thereof, including QS21 and QS7 (Aquila Biopharmaceuticals Inc., Framingham, Mass.); Escin; Digitonin; or Gypsophila or Chenopodium quinoa saponins. Other illustrative formulations include more than one saponin in the adjuvant combinations of the present invention, for example combinations of at least two of the following group comprising QS21, QS7, Quil A, escin, or digitonin.

In a particular embodiment, the adjuvant system includes the combination of a monophosphoryl lipid A and a saponin derivative, such as the combination of QS21 and 3D-MPL™ adjuvant, as described in WO 94/00153, or a less reactogenic composition where the QS21 is quenched with cholesterol, as described in WO 96/33739. Other formulations comprise an oil-in-water emulsion and tocopherol. Another adjuvant formulation employing QS21, 3D-MPL™ adjuvant and tocopherol in an oil-in-water emulsion is described in WO 95/17210.

Another enhanced adjuvant system involves the combination of a CpG-containing oligonucleotide and a saponin derivative as disclosed in WO 00/09159.

Other illustrative adjuvants include Montanide ISA 720 (Seppic, France), SAF (Novartis, Calif., United States), ISCOMS (CSL), MF-59 (Chiron), the SBAS series of adjuvants (e.g., SBAS-2, AS2′, AS2,″ SBAS-4, or SBAS6, available from GlaxoSmithKline, Rixensart, Belgium), Detox, RC-529 (GlaxoSmithKline, Hamilton, Mont.) and other aminoalkyl glucosaminide 4-phosphates (AGPs), such as those described in pending U.S. patent application Ser. Nos. 08/853,826 and 09/074,720, the disclosures of which are incorporated herein by reference in their entireties, and polyoxyethylene ether adjuvants such as those described in WO 99/52549A1.

Compositions of the invention may also, or alternatively, comprise T cells specific for a Mycobacterium antigen. Such cells may generally be prepared in vitro or ex vivo, using standard procedures. For example, T cells may be isolated from bone marrow, peripheral blood, or a fraction of bone marrow or peripheral blood of a patient. Alternatively, T cells may be derived from related or unrelated humans, non-human mammals, cell lines or cultures.

T cells may be stimulated with a polypeptide of the invention, polynucleotide encoding such a polypeptide, and/or an antigen presenting cell (APC) that expresses such a polypeptide. Such stimulation is performed under conditions and for a time sufficient to permit the generation of T cells that are specific for the polypeptide. Preferably, the polypeptide or polynucleotide is present within a delivery vehicle, such as a microsphere, to facilitate the generation of specific T cells.

T cells are considered to be specific for a polypeptide of the invention if the T cells specifically proliferate, secrete cytokines or kill target cells coated with the polypeptide or expressing a gene encoding the polypeptide. T cell specificity may be evaluated using any of a variety of standard techniques. For example, within a chromium release assay or proliferation assay, a stimulation index of more than two fold increase in lysis and/or proliferation, compared to negative controls, indicates T cell specificity. Such assays may be performed, for example, as described in Chen et al., Cancer Res. 54:1065-1070 (1994)). Alternatively, detection of the proliferation of T cells may be accomplished by a variety of known techniques. For example, T cell proliferation can be detected by measuring an increased rate of DNA synthesis (e.g., by pulse-labeling cultures of T cells with tritiated thymidine and measuring the amount of tritiated thymidine incorporated into DNA). Contact with a polypeptide of the invention (100 ng/ml-100 μg/ml, preferably 200 ng/ml-25 μg/ml) for 3-7 days should result in at least a two fold increase in proliferation of the T cells. Contact as described above for 2-3 hours should result in activation of the T cells, as measured using standard cytokine assays in which a two fold increase in the level of cytokine release (e.g., TNF or IFN-γ) is indicative of T cell activation (see Coligan et al., Current Protocols in Immunology, vol. 1 (1998)). T cells that have been activated in response to a polypeptide, polynucleotide or polypeptide-expressing APC may be CD4+ and/or CD8+. Protein-specific T cells may be expanded using standard techniques. Within preferred embodiments, the T cells are derived from a patient, a related donor or an unrelated donor, and are administered to the patient following stimulation and expansion.

In the pharmaceutical compositions of the invention, formulation of pharmaceutically-acceptable excipients and carrier solutions is well-known to those of skill in the art, as is the development of suitable dosing and treatment regimens for using the particular compositions described herein in a variety of treatment regimens, including e.g., oral, parenteral, intravenous, intranasal, intradermal, subcutaneous, and intramuscular administration and formulation.

In certain applications, the pharmaceutical compositions disclosed herein may be delivered via oral administration to a subject. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

In certain circumstances it will be desirable to deliver the pharmaceutical compositions disclosed herein parenterally, intravenously, intramuscularly, or even intraperitoneally as described, for example, in U.S. Pat. Nos. 5,543,158; 5,641,515 and 5,399,363 (each specifically incorporated herein by reference in its entirety). Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be facilitated by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion (see, e.g., Remington: The Science and Practice of Pharmacy, 20th Edition. Baltimore, MD: Lippincott Williams & Wilkins, 2000). Some variation in dosage will necessarily occur depending on the condition of the subject being treated. The person responsible for administration will, in any event, determine the appropriate dose for the individual subject. Moreover, for human administration, preparations should meet sterility, pyrogenicity, and the general safety and purity standards as required by FDA Office of Biologics standards.

Sterile injectable solutions are prepared by incorporating the active compounds in the required amount in the appropriate solvent with the various other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The compositions disclosed herein may be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically-acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared. The preparation can also be emulsified.

In certain embodiments, the pharmaceutical compositions may be delivered by intranasal sprays, inhalation, and/or other aerosol delivery vehicles. Methods for delivering genes, polynucleotides, and peptide compositions directly to the lungs via nasal aerosol sprays has been described e.g., in U.S. Pat. Nos. 5,756,353 and 5,804,212 (each specifically incorporated herein by reference in its entirety). Likewise, the delivery of drugs using intranasal microparticle resins (Takenaga et al., 1998) and lysophosphatidyl-glycerol compounds (U.S. Pat. No. 5,725,871, specifically incorporated herein by reference in its entirety) are also well-known in the pharmaceutical arts. Likewise, transmucosal drug delivery in the form of a polytetrafluoroetheylene support matrix is described in U.S. Pat. No. 5,780,045 (specifically incorporated herein by reference in its entirety).

In certain embodiments, the delivery may occur by use of liposomes, nanocapsules, microparticles, microspheres, lipid particles, vesicles, and the like, for the introduction of the compositions of the present invention into suitable host cells. In particular, the compositions of the present invention may be formulated for delivery either encapsulated in a lipid particle, a liposome, a vesicle, a nanosphere, a nanoparticle or the like. The formulation and use of such delivery vehicles can be carried out using known and conventional techniques.

### D. Diagnostic Methods and Kits

As noted above, the compositions, fusion polypeptides and polynucleotides are also useful as diagnostic reagents for detecting and/or monitoring Mycobacterium tuberculosis infection in a patient. For example, the compositions, fusion polypeptides, and polynucleotides of the invention may be used in in vitro and in vivo assays for detecting humoral antibodies or cell-mediated immunity against Mycobacterium tuberculosis for diagnosis of infection, monitoring of disease progression or test-of-cure evaluation.

Therefore, in certain embodiments, the invention provides improved diagnostic antigens for differentially diagnosing Mycobacterium tuberculosis infection based on serological examination, wherein the Mycobacterium antigens used in the diagnosis are selected from the group consisting of Rv0164 (SEQ ID NO: 1), Rv0496 (SEQ ID NO: 6), Rv2608 (SEQ ID NO: 26), Rv3020 (SEQ ID NO: 36), Rv3478 (SEQ ID NO: 41), Rv3619 (SEQ ID NO: 46), Rv3620 (SEQ ID NO: 51), RV1738 (SEQ ID NO: 11), Rv1813 (SEQ ID NO: 16), Rv3810 (SEQ ID NO: 56), Rv2389 (SEQ ID NO: 21), Rv2866 (SEQ ID NO: 31), Rv3876 (SEQ ID NO: 61), Rv0054 (SEQ ID NO: 100), Rv0410 (SEQ ID NO: 106), Rv0655 (SEQ ID NO: 112), Rv0831 (SEQ ID NO: 115), Rv1009 (SEQ ID NO: 118), Rv1099 (SEQ ID NO: 121), Rv1240 (SEQ ID NO: 124), Rv1288 (SEQ ID NO: 127), Rv1410 (SEQ ID NO: 130), Rv1569 (SEQ ID NO: 133), Rv1789 (SEQ ID NO: 136), Rv1818 (SEQ ID NO: 139), Rv1860 (SEQ ID NO: 142), Rv1886 (SEQ ID NO: 145), Rv1908 (SEQ ID NO: 148), Rv2220 (SEQ ID NO: 154), Rv2032 (SEQ ID NO: 151), Rv2623 (SEQ ID NO: 160), Rv2875 (SEQ ID NO: 163), Rv3044 (SEQ ID NO: 166), Rv3310 (SEQ ID NO: 169), and Rv3881 (SEQ ID NO: 178), Rv0577 (SEQ ID NO: 184), Rv1626 (SEQ ID NO: 187), Rv0733 (SEQ ID NO: 190), Rv2520 (SEQ ID NO: 193), Rv1253 (SEQ ID NO: 196), Rv1980 (SEQ ID NO: 199), Rv3628 (SEQ ID NO: 202) Rv1884 (SEQ ID NO: 205), Rv3872 (SEQ ID NO: 208), Rv3873 (SEQ ID NO: 211), Rv1511 (SEQ ID NO: 214) and Rv3875 (SEQ ID NO: 292), or immunogenic portions or variants thereof, in any combination thereof mixed as separate antigens, or in fusion gene constructs. As demonstrated herein, combinations of the disclosed diagnostic antigens offer improved sensitivity in serological diagnostic testing.

The diagnostic methods and kits preferably employ a combination of two or more antigens as described herein. In certain embodiments, it will be preferred to use a multiple antigens as described herein, e.g., three or more, four or more, five or more, six or more, etc., in a diagnostic method of the invention. The antigens may be used in essentially any assay format desired, e.g., as individual antigens assayed separately, as multiple antigens assays simultaneously, as antigens immobilized on a solid support such as an array, or the like.

In a particular embodiment, the diagnostic antigens used in the methods herein are selected from the group consisting of Rv0164 (SEQ ID NO: 1), Rv0496 (SEQ ID NO: 6), Rv2608 (SEQ ID NO: 26), Rv3020 (SEQ ID NO: 36), Rv3478 (SEQ ID NO: 41), Rv3619 (SEQ ID NO: 46), Rv3620 (SEQ ID NO: 51), RV1738 (SEQ ID NO: 11), Rv1813 (SEQ ID NO: 16), Rv3810 (SEQ ID NO: 56), Rv2389 (SEQ ID NO: 21), Rv2866 (SEQ ID NO: 31), Rv3876 (SEQ ID NO: 61), Rv0054 (SEQ ID NO: 100), Rv0410 (SEQ ID NO: 106), Rv0655 (SEQ ID NO: 112), Rv0831 (SEQ ID NO: 115), Rv1009 (SEQ ID NO: 118), Rv1099 (SEQ ID NO: 121), Rv1240 (SEQ ID NO: 124), Rv1288 (SEQ ID NO: 127), Rv1410 (SEQ ID NO: 130), Rv1569 (SEQ ID NO: 133), Rv1789 (SEQ ID NO: 136), Rv1818 (SEQ ID NO: 139), Rv1860 (SEQ ID NO: 142), Rv1886 (SEQ ID NO: 145), Rv1908 (SEQ ID NO: 148), Rv2220 (SEQ ID NO: 154), Rv2032 (SEQ ID NO: 151), Rv2623 (SEQ ID NO: 160), Rv2875 (SEQ ID NO: 163), Rv3044 (SEQ ID NO: 166), Rv3310 (SEQ ID NO: 169), and Rv3881 (SEQ ID NO: 178), Rv0577 (SEQ ID NO: 184), Rv1626 (SEQ ID NO: 187), Rv0733 (SEQ ID NO: 190), Rv2520 (SEQ ID NO: 193), Rv1253 (SEQ ID NO: 196), Rv1980 (SEQ ID NO: 199), Rv3628 (SEQ ID NO: 202) Rv1884 (SEQ ID NO: 205), Rv3872 (SEQ ID NO: 208), Rv3873 (SEQ ID NO: 211), Rv1511 (SEQ ID NO: 214) and Rv3875 (SEQ ID NO: 292), or immunogenic portions or variants thereof, in any combination thereof mixed as separate antigens, or in fusion gene constructs.

In one embodiment, there are provided diagnostic kits for detecting Mycobacterium tuberculosis infection in a biological sample, comprising (a) a polypeptide comprising at least an immunogenic portion of an antigen or fusion polypeptide described herein, and (b) a detection reagent.

In another embodiment, there are provided diagnostic kits for detecting Mycobacterium tuberculosis infection in a biological sample, comprising (a) an antibody or antigen binding fragment thereof that is specific for a polypeptide comprising at least an immunogenic portion of an antigen or fusion polypeptide described herein, and (b) a detection reagent.

In another embodiment, methods are provided for detecting the presence of Mycobacterium tuberculosis infection in a biological sample, comprising (a) contacting a biological sample with a monoclonal antibody that binds to an antigen or fusion polypeptide described herein; and (b) detecting in the biological sample the presence of Mycobacterium tuberculosis proteins that bind to the monoclonal antibody.

In yet another embodiment, methods are provided for detecting Mycobacterium tuberculosis infection in a biological sample, comprising (a) contacting the biological sample with an antigen combination or fusion polypeptide as described herein and (b) detecting in the biological sample the presence of antibodies and/or T-cells that bind thereto.

There are a variety of assay formats known to those of ordinary skill in the art for using purified antigen or fusion polypeptide to detect antibodies in a sample. See, e.g., Harlow and Lane, Antibodies: A Laboratory Manual, Cold Spring Harbor Laboratory, 1988. In one embodiment, the assay involves the use of polypeptide immobilized on a solid support to bind to and remove the antibody from the sample. The bound antibody may then be detected using a detection reagent that binds to the antibody/peptide complex and contains a detectable reporter group. Suitable detection reagents include antibodies that bind to the antibody/polypeptide complex and free polypeptide labeled with a reporter group (e.g., in a semi-competitive assay). Alternatively, a competitive assay may be utilized, in which an antibody that binds to the polypeptide is labeled with a reporter group and allowed to bind to the immobilized antigen after incubation of the antigen with the sample. The extent to which components of the sample inhibit the binding of the labeled antibody to the polypeptide is indicative of the reactivity of the sample with the immobilized polypeptide.

The solid support may be any solid material known to those of ordinary skill in the art to which the antigen may be attached. For example, the solid support may be a test well in a microtiter plate or a nitrocellulose or other suitable membrane. Alternatively, the support may be a bead or disc, such as glass, fiberglass, latex or a plastic material such as polystyrene or polyvinylchloride. The support may also be a magnetic particle or a fiber optic sensor, such as those disclosed, for example, in U.S. Pat. No. 5,359,681.

The polypeptide may be bound to the solid support using any of a variety of techniques known and available in the art. The term “bound” refers to both noncovalent association, such as adsorption, and covalent attachment (which may be a direct linkage between the antigen and functional groups on the support or may be a linkage by way of a cross-linking agent). Binding by adsorption to a well in a microtiter plate or to a membrane is preferred. In such cases, adsorption may be achieved by contacting the polypeptide, in a suitable buffer, with the solid support for a suitable amount of time.

In certain embodiments, the diagnostic assay employed is an enzyme linked immunosorbent assay (ELISA). This assay may be performed by first contacting a polypeptide antigen that has been immobilized on a solid support, commonly the well of a microtiter plate, with the sample, such that antibodies to the polypeptide within the sample are allowed to bind to the immobilized polypeptide. Unbound sample is then removed from the immobilized polypeptide and a detection reagent capable of binding to the immobilized antibody-polypeptide complex is added. The amount of detection reagent that remains bound to the solid support is then determined using a method appropriate for the specific detection reagent.

Once the polypeptide is immobilized on the support, the remaining protein binding sites on the support are typically blocked. Any suitable blocking agent known to those of ordinary skill in the art, such as bovine serum albumin or Tween 20™ (Sigma Chemical Co., St. Louis, MO). The immobilized polypeptide is then incubated with the sample, and antibody (if present in the sample) is allowed to bind to the antigen. The sample may be diluted with a suitable diluent, such as phosphate-buffered saline (PBS) prior to incubation. In general, an appropriate contact time (i.e., incubation time) is that period of time that is sufficient to detect the presence of antibody to Mycobacterium tuberculosis within an infected sample. Preferably, the contact time is sufficient to achieve a level of binding that is at least 95% of that achieved at equilibrium between bound and unbound antibody. Those of ordinary skill in the art will recognize that the time necessary to achieve equilibrium may be readily determined by assaying the level of binding that occurs over a period of time. At room temperature, an incubation time of about 30 minutes is generally sufficient.

Unbound sample may then be removed by washing the solid support with an appropriate buffer, such as PBS containing 0.1% Tween 20™. Detection reagent may then be added to the solid support. An appropriate detection reagent is any compound that binds to the immobilized antibody-polypeptide complex and that can be detected by any of a variety of means known to those in the art. The detection reagent generally contains a binding agent (such as, for example, Protein A, Protein G, immunoglobulin, lectin or free antigen) conjugated to a reporter group. Illustrative reporter groups include enzymes (such as horseradish peroxidase), substrates, cofactors, inhibitors, dyes, radionuclides, luminescent groups, fluorescent groups and biotin. The conjugation of binding agent to reporter group may be achieved using standard methods known to those of ordinary skill in the art.

The detection reagent is then incubated with the immobilized antibody-polypeptide complex for an amount of time sufficient to detect the bound antibody. An appropriate amount of time may generally be determined from the manufacturer's instructions or by assaying the level of binding that occurs over a period of time. Unbound detection reagent is then removed and bound detection reagent is detected using the reporter group. The method employed for detecting the reporter group depends upon the nature of the reporter group. For radioactive groups, scintillation counting or autoradiographic methods are generally appropriate. Spectroscopic methods may be used to detect dyes, luminescent groups and fluorescent groups. Biotin may be detected using avidin, coupled to a different reporter group (commonly a radioactive or fluorescent group or an enzyme). Enzyme reporter groups may generally be detected by the addition of substrate (generally for a specific period of time), followed by spectroscopic or other analysis of the reaction products.

To determine the presence or absence of Mycobacterium tuberculosis antibodies in a sample, the signal detected from the reporter group that remains bound to the solid support is generally compared to a signal that corresponds to a predetermined cut-off value. This cut-off value is preferably the average mean signal obtained when the immobilized antigen is incubated with samples from an uninfected patient. In general, a sample generating a signal that is three standard deviations above the mean is considered positive for Mycobacterium tuberculosis antibodies and Mycobacterium tuberculosis infection. In another embodiment, the cut-off value is determined using a Receiver Operator Curve, according to the method of Sackett et al., Clinical Epidemiology: A Basic Science for Clinical Medicine, p. 106-7 (Little Brown and Co., 1985). Briefly, in this embodiment, the cut-off value may be determined from a plot of pairs of true positive rates (i.e., sensitivity) and false positive rates (100%-specificity) that correspond to each possible cut-off value for the diagnostic test result. The cut-off value on the plot that is the closest to the upper left-hand corner (i.e., the value that encloses the largest area) is the most accurate cut-off value, and a sample generating a signal that is higher than the cut-off value determined by this method may be considered positive. Alternatively, the cut-off value may be shifted to the left along the plot, to minimize the false positive rate, or to the right, to minimize the false negative rate. In general, a sample generating a signal that is higher than the cut-off value determined by this method is considered positive for Mycobacterium tuberculosis infection.

In another embodiment, a diagnostic assay may be performed in a flow-through or strip test format, wherein the antigen or fusion polypeptide is immobilized on a membrane such as nitrocellulose. In the flow-through test, antibodies within the sample bind to the immobilized polypeptide as the sample passes through the membrane. A detection reagent (e.g., protein A-colloidal gold) then binds to the antibody-polypeptide complex as the solution containing the detection reagent flows through the membrane. The detection of bound detection reagent may then be performed as described above. In the strip test format, one end of the membrane to which polypeptide is bound is immersed in a solution containing the sample. The sample migrates along the membrane through a region containing detection reagent and to the area of immobilized polypeptide. Concentration of detection reagent at the polypeptide indicates the presence of Mycobacterium tuberculosis antibodies in the sample. Such tests can typically be performed with a very small amount (e.g., one drop) of patient serum or blood.

In yet another embodiment, methods are provided for detecting Mycobacterium tuberculosis in a biological sample using antibodies (which may be polyclonal or monoclonal) and/or T-cells specific for one or more antigens, fusion polypeptides and/or immunogenic portions of the invention.

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims. The following examples are provided by way of illustration only and not by way of limitation. Those of skill in the art will readily recognize a variety of noncritical parameters that could be changed or modified to yield essentially similar results.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, are incorporated herein by reference, in their entirety. Aspects of the embodiments can be modified, if necessary to employ concepts of the various patents, applications and publications to provide yet further embodiments.

### EXAMPLES

### Example 1

### Cloning and Expression of Recombinant Rv0164

Using H37Rv genomic DNA as template, Rv0164 was PCR amplified using the primers set forth in SEQ ID NOs: 4 and 5, below:

Primer 5′-Rv0164-5his-NdeI:
(SEQ ID NO: 4)
TAGGATCCCATATGACGGCAATCTCGTGCTCAC
Primer 3′-Rv0164-3HindIII:
(SEQ ID NO: 5)
TAGAATTCAAGCTTTTAGCTGGCCGCCAGCTGCTC

The following amplification conditions were used: 94° C. 0.5 min., 55° C. 0.5 min., 68° C. 1 min for 30 cycles to give the product set forth in SEQ ID NO: 2. The PCR product was digested with NdeI/HindIII and cloned into pET 28a. Plasmid containing the Rv0164 gene was transformed into expression host and Rosetta2 pLysS. Cultures were grown in shake flask at 37° C. in 2× YT media supplemented with 34 mg/L Chloramphenicol, 35 mg/L Kanamycin to an OD600=0.5-0.6 and induced with 1 mM IPTG for 3-4 hrs. The cell paste was pelleted at 10000× g and stored at −20° C. After lysis of a 1 L induction by sonication and clarification of the supernatant, the Rv0164 protein remained in the insoluble fraction. This fraction was then washed 2× in 1% CHAPS detergent, 20 mM Tris HCl pH 8.0, and then solublized in 8M Urea. Purification was achieved using 2 rounds of Ni-NTA affinity chromatography (Qiagen) under denaturing conditions with and the Rv0164 protein was eluted using 300 mM Imidazole. After SDS-PAGE analysis, fractions containing the purified protein were dialyzed against 10 mM Tris pH 8.0. Protein concentration was determined by Bradford Assay and residual endotoxin levels were determined by the Llimulus Amoebcyte Assay. The amino acid sequence of the recombinant protein is set forth in SEQ ID NO: 3.

### Example 2

### Cloning and Expression of Recombinant Rv0496

Using H37Rv genomic DNA as template, Rv0496 was PCR amplified using the following primers:

5′-Rv0496-5his-NdeI
(SEQ ID NO: 9)
TAGGATCCCATATGGTCGATGCCCACCGCGGC
3′-Rv0496-3HindIII
(SEQ ID NO: 10)
TAGAATTCAAGCTTTCATGGTTTGCTGCCTCTCGA

Amplification was performed under the following conditions: 94° C. 0.5 min., 55° C. 0.5 min., 68° C. 2 min for 30 cycles, to give the product set forth in SEQ ID NO: 7. The PCR product was digested with NdeI/HindIII and cloned into pET28a. Rv0496 was transformed into expression hosts and Rosetta2 plysS. After lysis of a 1 L induction, it went into the inclusion body. Ni-NTA was performed twice under denaturing conditions, then dialyzed against 10 mM Tris pH 10. The amino acid sequence of the recombinant protein is set forth in SEQ ID NO: 8.

### Example 3

### Cloning and Expression of Recombinant Rv1738

Using H37Rv genomic DNA as template, Rv1738 was PCR amplified using the following primers:

5′-Rv1738-5his-NdeI
(SEQ ID NO: 14)
CAATTACATATGCATCACCATCACCATCACATGTGCGGCGAC
CAGTCGGAT
3′-Rv1738-3EcoRI
(SEQ ID NO: 15)
CAATTAGAATTCTCAATACAACAATCGCGCCGG

Amplification was performed using the following conditions: 95° C. 1 min., 58° C. 1 min., 72° C. 1 min for 35 cycles, to give the product set forth as SEQ ID NO: 12. The PCR product was digested with NdeI/EcoRI and cloned into pET 17b. Rv1738 was transformed into expression hosts BL-21plysE and plysS. After lysis of a 1 L induction, protein remained in the soluble supernatant. Ni-NTA was performed under denaturing conditions, then dialyzed against 10 mM Tris pH 8.0. The amino acid sequence of the recombinant protein is set forth in SEQ ID NO: 13.

### Example 4

### Cloning and Expression of Recombinant Rv1813

Using H37Rv genomic DNA as template, Rv1813 was PCR amplified using the following primers:

5′-Rv1813-5his33-NdeI-
(SEQ ID NO: 19)
CAATTACATATGCATCACCATCACCATCACCATCTCGCCAAC
GGtTTCGATG
3′-Rv1813-3EcoRI-
(SEQ ID NO: 20)
CAATTAGAATTCTTAGTTGCACGCCCAGTTGAC

The amplification was performed using the following conditions 95° C. 1 min., 58° C. 1 min., 72° C. 1 min for 35 cycles, to give the product set forth in SEQ ID NO: 17. The PCR product was digested with NdeI/EcoRI and cloned into pET 17b. Rv1813 was transformed into expression hosts BL-21plysE and Rosetta plysS. After lysis of a 1 L induction, protein went into the inclusion body. Ni-NTA was performed under denaturing conditions, then dialyzed against 10 mM Tris pH 8.0. The amino acid sequence of the recombinant protein is set forth in SEQ ID NO: 18.

### Example 5

### Cloning and Expression of Recombinant Rv2389 (RPF-D)

Using H37Rv genomic DNA as template, Rv2389 was PCR amplified using the following primers:

5′-Rv2389-5his50-NdeI-
(SEQ ID NO: 24)
CAATTACATATGCATCACCATCACCATCACGACGACATCGATT
GGGACGCC
3′-Rv2389-3EcoRI-
(SEQ ID NO: 25)
CAATTAGAATTCTCAATCGTCCCTGCTCCCCGA

Amplification was performed under the following conditions: 95° C. 1 min., 58° C. 1 min., 72° C. 1 min for 35 cycles, to give the product set forth in SEQ ID NO: 22. The PCR product was digested with NdeI/EcoRI and cloned into pET 17b (pET construct begins at aa49). Rv2389 was transformed into expression hosts BL-21plysE and Rosetta plysS. After lysis of a 1 L induction, protein remained in the soluble fraction. Ni-NTA was performed under denaturing conditions, then dialyzed against 10 mM Tris pH 8.0. The amino acid sequence of the recombinant protein is set forth in SEQ ID NO: 23.

### Example 6

### Cloning and Expression of Recombinant Rv2608

Using H37Rv genomic DNA as template, Rv2608 was PCR amplified using the following primers:

5′-Rv2608-5-NdeI-
(SEQ ID NO: 29)
TAGGATCCCATATGAATTTCGCCGTTTTGCCG
3′-Rv2608-3-HindIII-
(SEQ ID NO: 30)
TAGAATTCAAGCTTTTAGAAAAGTCGGGGTAGCGCC

Amplification was performed using the following conditions 94° C. 0.5 min., 55° C. 0.5 min., 68° C. 2 min for 30 cycles, to give the product set forth in SEQ ID NO: 27. The gel purified PCR product was digested with NdeI/HindIII and cloned into the expression vector pET28a (Clonetech) (pET construct begins at amino acid 1). Rv2608 was transformed into expression hosts and Rosetta2 pLysS. Cultures were grown in shake flask at 37° C. in 2× YT media supplemented with 34 mg/L Chloramphenicol, 35 mg/L Kanamycin to an OD600=0.5-0.6 and induced with 1 mM IPTG for 3-4 hrs. The cell paste was pelleted at 10000× g and stored at −20° C. After lysis of a 1 L induction by sonication and clarification of the supernatant, the Rv2608 protein remained in the insoluble fraction. This fraction was then washed 2× in 1% CHAPS detergent, 10 mM Tris HCl pH 8.0, and then solublized in 8M Urea. Purification was performed using Ni-NTA affinity chromatography (Qiagen) 2× under denaturing conditions with and the Rv2608 protein was eluted using 300 mM Imidazole. After SDS-PAGE analysis, fractions containing the purified protein were dialyzed against 10 mM Tris pH 8.0. Protein concentration was determined by BCA assay and residual endotoxin levels were determined by the Llimulus Amoebcyte Assay. The amino acid sequence of the recombinant protein is set forth in SEQ ID NO: 28.

### Example 7

### Cloning and Expression of Recombinant Rv2866

Rv2866 was amplified from genomic template by PCR, using the following primers:

5′-Rv2866-5NdeI-
(SEQ ID NO: 32)
CAATTACATATGCCTTCCACCGTGCCCTTCACC
3′-Rv2866-3HindIII-
(SEQ ID NO: 33)
CAATTAAAGCTTCTATCGGCGGTAGATGTCCGCGCG.

The following amplification conditions were used: 94° C. for 0.5 min., 66° C. for 0.50 min., 68° C. for 1.50 min., 35 cycles), to give the product set forth in SEQ ID NO: 34. Product was digested with NdeI/HindIll and cloned into pET28.a vector. Rv2866 was expressed by host strain BL-21 plysS. The pellet and supernatant were bound with Ni resin under denaturing conditions. Dialysis was performed in 20 mM Tris pH 6. The amino acid sequence of the recombinant protein is set forth in SEQ ID NO: 35.

### Example 8

### Cloning and Expression of Recombinant Rv3020

Using H37 genomic DNA as template, Rv3020 was PCR amplified using the following primers:

5′-Rv3020-5his-NdeI-
(SEQ ID NO: 39)
TAGGATCCCATATGAGTTTGTTGGATGCCCATAT
3′-Rv3020-3HindIII-
(SEQ ID NO: 40)
TAGAATTCAAGCTTTTAAAACCCGGTGTAGCTGGAC

The following amplification conditions were employed: 94° C. 0.5 min., 55° C. 0.5 min., 68° C. 1 min. for 30 cycles, yielding the product set forth in SEQ ID NO: 37. The PCR product was digested with NdeI/HindIII and cloned into pET 28a. Plasmid containing the Rv3020 gene was transformed into expression host and Rosetta2 pLysS. Cultures were grown in shake flask at 37° C. in 2× YT media supplemented with 34 mg/L Chloramphenicol, 35 mg/L Kanamycin to an OD600=0.5-0.6 and induced with 1 mM IPTG for 3-4 hrs. The cell paste was pelleted at 10000× g and stored at −20° C. After lysis of a 1 L induction by sonication and clarification of the supernatant, the Rv3020 protein remained in the insoluble fraction. This fraction was then washed 2× in 1% CHAPS detergent, 20 mM Tris HCl pH 8.0, and then solublized in 8M Urea. Purification was performed using Ni-NTA affinity chromatography (Qiagen) under denaturing conditions with and the Rv3020 protein was eluted using 250 mM Imidazole. After SDS-PAGE analysis, fractions containing the purified protein were dialyzed against 10 mM Tris pH 8.0. Protein concentration was determined by Bradford Assay and residual endotoxin levels were determined by the Llimulus Amoebcyte Assay. The amino acid sequence of the recombinant protein is set forth in SEQ ID NO: 38.

### Example 9

### Cloning and Expression of Recombinant Rv3478

Using H37Rv genomic DNA as template, Rv3478 was amplified using the following primers:

5′-Rv3478-5his-NdeI-
(SEQ ID NO: 44)
TAGGATCCCATATGGTGGATTTCGGGGCGTTAC
3′-Rv3478-3HindIII-
(SEQ ID NO: 45)
TAGAATTCAAGCTTCTATCCGGCGGCCGGTGTGCG

Rv3478 was amplified using polymerase chain reaction (PCR) with the following conditions 94° C. 0.5 min., 55° C. 0.5 min., 68° C. 2 min. for 30 cycles. The gel purified PCR product (SEQ ID NO: 42) was digested with NdeI/HindIII and cloned into the expression vector pET28a (Clonetech). Rv3478 was transformed into expression hosts and Rosetta2 pLysS. Cultures were grown in shake flask at 37° C. in 2× YT media supplemented with 34 mg/L Chloramphenicol, 35 mg/L Kanamycin to an OD600=0.5-0.6 and induced with 1 mM IPTG for 3-4 hrs. The cell paste was pelleted at 10000×g and stored at −20° C. After lysis of a 1 L induction by sonication and clarification of the supernatant, the Rv3478 protein remained in the insoluble fraction. This fraction was then washed 2× in 1% CHAPS detergent, 10 mM Tris HCl pH 8.0, and then solublized in 8M Urea. Purification was done using Ni-NTA affinity chromatography (Qiagen) 2× under denaturing conditions with and the Rv3478 protein was eluted using 300 mM Imidazole. After SDS-PAGE analysis, fractions containing the purified protein were dialyzed against 10 mM Tris pH 8.0. Protein concentration was determined by BCA assay and residual endotoxin levels were determined by the Llimulus Amoebcyte Assay. The amino acid sequence of the recombinant protein is set forth in SEQ ID NO: 43.

### Example 10

### Cloning and Expression of Recombinant Rv3619

Using H37Rv genomic DNA as template, Rv3619 was amplified using the following primers.

5′-Rv3619-5his-NdeI-
(SEQ ID NO: 49)
TAGGATCCCATATGACCAT
CAACTATCAATTCG
3′-Rv3619-3HindIII-
(SEQ ID NO: 50)
TAGAATTCAAGCTTTTAGG
CCCAGCTGGAGCCGAC

Rv3619 was amplified using polymerase chain reaction (PCR) with the following conditions 94° C. 0.5 min., 55° C. 0.5 min., 68° C. 1 min. for 30 cycles. The gel purified PCR product (SEQ ID NO: 47) was digested with NdeI/HindIII and cloned into the expression vector pET28a (Clonetech).

Rv3619 was transformed into expression hosts and Rosetta2 pLysS. Cultures were grown in shake flask at 37° C. in 2× YT media supplemented with 34 mg/L Chloramphenicol, 35 mg/L Kanamycin to an OD600=0.5-0.6 and induced with 1 mM IPTG for 3-4 hrs. The cell paste was pelleted at 10000× g and stored at −20° C. After lysis of a 1 L induction by sonication and clarification of the supernatant, the Rv3619 protein remained in the insoluble fraction. This fraction was then washed 2× in 1% CHAPS detergent, 10 mM Tris HCl pH 8.0, and then solublized in 8M Urea. Purification was performed using Ni-NTA affinity chromatography (Qiagen) under denaturing conditions with and the Rv3619 protein was eluted using 300 mM Imidazole. After SDS-PAGE analysis, fractions containing the purified protein were dialyzed against 10 mM Tris pH 8.0. Protein concentration was determined by Bradford Assay and residual endotoxin levels were determined by the Llimulus Amoebcyte Assay. The amino acid sequence of the recombinant protein is set forth in SEQ ID NO: 48.

### Example 11

### Cloning and Expression of Recombinant Rv3620

Using H37Rv genomic DNA as template, Rv3620 was PCR amplified using the following primers:

5′-Rv3620-5his-NdeI-
(SEQ ID NO: 54)
TAGGATCCCATATGACCT
CGCGTTTTATGACG
3′-Rv3620-3HindIII-
(SEQ ID NO: 55)
TAGAATTCAAGCTTTCAGC
TGCTGAGGATCTGCTG

Rv3620 was PCR amplified with conditions 94° C. 0.5 min., 55° C. 0.5 min., 68° C. 1 min. for 30 cycles. The PCR product (SEQ ID NO: 52) was digested with NdeI/HindIII and cloned into pET28a. Rv3620 was transformed into expression host Rosetta2 plysS. After lysis of a 1 L induction, protein went into the inclusion body. Ni-NTA was performed under denaturing conditions, then purified antigen dialyzed against 20 mM Tris pH 8.0, 50 mM NaCl. The amino acid sequence of the recombinant protein is set forth in SEQ ID NO: 53.

### Example 12

### Cloning and Expression of Recombinant Rv3810

Using H37Rv genomic DNA as template, Rv3810 was PCR amplified using the following primers:

5′-Rv3810-5his23-NdeI-
(SEQ ID NO: 59)
CAATTACATATGCATCACCATCA
CCATCACAGTCCTTGTGCATATT
TTCTTGTC
3′-Rv3810-3XhoI-
(SEQ ID NO: 60)
CAATTACTCGAGTT
AGGCGACCGGCACGGTGATTGG

Rv3810 was PCR amplified with conditions 95° C. 1 min., 58° C. 1 min., 72° C. 1.5 min. for 35 cycles. The PCR product (SEQ ID NO: 57) was digested with NdeI/Xhol and cloned into pET 17b (pET construct begins at amino acid 23). Rv3810 was transformed into expression hosts BL-21plysE and Rosetta plysS. After lysis of a 1 L induction, protein went into the inclusion body. Ni-NTA was performed under denaturing conditions, then dialyzed against 10 mM Tris pH 8.0. The amino acid sequence of the recombinant protein is set forth in SEQ ID NO: 58.

### Example 13

### Cloning and Expression of Recombinant Rv3876

Rv3876 was PCR amplified from genomic DNA using the following amplification primers:

Rv3876F-Nde-5′:
(SEQ ID NO: 64)
GATCCCATGGGCATATGGCGGCCGACTACGAC
Rv3876R-EcorRI-3′:
(SEQ ID NO: 65)
GTCAGAATTCTCAACGACGTCCAGCCCT

Amplification was performed using the following conditions: 94° C. 30 sec., 55° C. 30 sec., 72° C. 2 min. for 30 cycles. The PCR product was ligated into the shuttle vector pGemT. Positive clones were identified on LB agar-x-gal plates by blue/white selection. The Rv3876 gene product was digested with NdeI/EcoRI and cloned into pET 28a. Rv3876c was transformed into expression host BL-21 (DE3)plysS. After lysis of a 1 L induction, protein remained in the insoluble fraction. Ni-NTA was performed under denaturing conditions, then dialyzed against 20 mM Tris pH 8.0. The amino acid sequence of the recombinant protein is set forth in SEQ ID NO: 63.

### Example 14

### Cloning and Expression of Recombinant Fusion Protein MTB36F.1

The following primers were used in the construction of fusion construct Mtb36f.1:

5′-Rv2389-5NdeI50-
(SEQ ID NO: 68)
CAATTACATATGGACGAC
ATCGATTGGGACGCC
3′-Rv2389-3SacIgo-
(SEQ ID NO: 69)
CAATTAGAGCTCATCGTCC
CTGCTCCCCGAACA
5′-Rv3810-5SacI23-
(SEQ ID NO: 70)
CAATTAGAGCTCAGTCCT
TGTG]CATATTTTCTTG
3′-Rv3810-3HindIII-KpnI-
(SEQ ID NO. 71)
CAATTAAAGCTTTTAGGTACCGG
CGACCGGCACGGTGATTGG

Using previously cloned plasmid DNA of Rv2389 and Rv3810, the Mtb36f.1 components were PCR amplified using the following conditions: 94° C. 30 sec., 58° C. 30 sec., 68° C. 1 min. for 35 cycles. The 5′ Rv2389 PCR product as digested with NdeI/SacI and cloned into pET 28a. The 3′ Rv3810 PCR product was digested with SacI/HindIII and cloned into the Rv2389 containing pET 28a construct. Mtb36f.1 (SEQ ID NO: 66) was transformed into expression host BL-21(DE3)plysS. After lysis of a 1 L induction, protein remained in the soluble fraction. Ni-NTA was performed under native conditions, then dialyzed against 20 mM Tris pH 8.0. The amino acid sequence of the recombinant fusion protein is set forth in SEQ ID NO: 67.

### Example 15

### Cloning and Expression of Recombinant Fusion Protein ID58

The following primers were used in for cloning the fusion construct ID58, which comprises fusion partners derived from Mtb Rv1813, Rv3620 and Rv0496

5′: Rv1813mat-5NdeI-KpnI
(SEQ ID NO: 73)
CAATTACATATGGGTACCCATCT
CGCCAACGGTTCGATG
3′: Rv1813mat-3SacIgo
(SEQ ID NO: 74)
CAATTAGAGCTCGTTGCACGCC
CAGTTGACGAT
5′: Rv3620-5SacI
(SEQ ID NO: 75)
CAATTAGAGCTCATGACCTCGC
GTTTTATGACG
3′: Rv3620-3SaIIgo
(SEQ ID NO: 76)
CAATTAGTCGACGCTGCTGAGGA
TCTGCTGGGA
5′: Rv0496-5SaII
(SEQ ID NO: 77)
CAATTAGTCGACATGGTCGATGC
CCACCGCGGC
3′: Rv0496-3ScaI-HindIII
(SEQ ID NO: 78)
CAATTAAAGCTTTTAAGTACTTG
GTTTGCTGCCTCTCGATCG

Rv1813 and Rv3620 were PCR amplified from genomic template DNA (94° C. for 0.5 min., 58° C. for 0.5 min., 58° C. for 1:5 min.; 35 cycles). Rv1813 was digested with NdeI/SacI then cloned into pET28.a vector. Rv3620 was digested with SacI/SaII then ligated into the Rv1813pET construct. Rv0496 was amplified from plasmid template by PCR (94° C. for 0:30; 60° C. for 0:30; 68° C. for 1:30; 35 cycles). Product was digested with SaII/HindIII and cloned into pET28.a-Rv1813-3620 vector. ID58-pET28.a had some point mutations so site directed mutagenesis was used to insert the correct nucleic acids. The ID58 fusion construct has a polynucleotide sequence set forth in SEQ ID NO: 72, encoding the fusion protein set forth in SEQ ID NO: 79. ID58 was expressed in host BL-21plysS 2XYT growth media, 37° C.). Induction was with 1 mM IPTG at OD 0.471 and cells were harvested at OD 1.36. Cell pellet was suspended in lysis buffer (20 mM Tris pH8, 100 mM NaCl, 2 mM PMSF) and froze. ID58 forms an inclusion body and was processed the same as ID83. Fractions from the flow through bind were dialyzed in 20 mM Tris pH 8.5.

### Example 16

### Cloning and Expression of Recombinant Fusion Protein ID69

The following primers were used in for cloning the fusion construct ID69, which comprises fusion partners derived from Rv2389, Rv1813, Rv3620 and Rv0496:

5′: Rv2389mat-5NdeI
SEQ ID NO: 81)
CAATTACATATGGACGACATCGATTGGGACGC
3′: Rv2389mat-3KpnI-HindIII
(SEQ ID NO: 82)
CAATTAAAGCTTTTAAGTACTTGGTTTGCTGC
CTCTCGATCG

Rv2389 was PCR amplified from genomic template (94° C. for 0.5 min., 58° C. for 0.5 min., 68° C. for 1.5 min.; 35 cycles), digested with NdeI/HindIII, and ligated into pET28.a. ID58-pET28.a vector was digested with KpnI/HindIII to drop out the insert. ID58 was ligated into Rv2389-pET28.a vector (also digested with KpnI/HindIII). The fusion construct has a polynucleotide sequence set forth in SEQ ID NO: 80, encoding the fusion protein set forth in SEQ ID NO: 83. ID69 was expressed in host BL-21plysS (1 L, 2XYT growth media, 37° C.). Cell pellet was suspended in lysis buffer (20 mM Tris pH8, 100 mM NaCl, 2 mM PMSF) and froze. ID69 forms an inclusion body and was purified the same as ID83.

### Example 17

### Cloning and Expression of Recombinant Fusion Protein ID83

The following primers were used in for cloning the fusion construct ID83, which comprises fusion partners from Rv1813, Rv3620 and Rv2608:

5′: Rv1813mat-5NdeI-KpnI
(SEQ ID NO: 85)
CAATTACATATGGGTACCCATCTCGCCAACGGT
TCGATG
3′: Rv1813mat-3SacIgo
(SEQ ID NO: 86)
CAATTAGAGCTCGTTGCACGCCCAGTTGACGAT
5′: Rv3620-5SacI
(SEQ ID NO: 87)
CAATTAGAGCTCATGACCTCGCGTTTTATGACG
3′: Rv3620-3SaIIgo
(SEQ ID NO: 88)
CAATTAGTCGACGCTGCTGAGGATCTGCTGGGA
5′: Rv2608-5SaII
(SEQ ID NO: 89)
CAATTAGTCGACATGAATTTCGCCGTTTTGCCG
3′: Rv2608-3ScaI-HindIII
(SEQ ID NO: 90)
CAATTAAAGCTTTTAAGTACTGAAAAGTCGGGG
TAGCGCCGG

Rv1813 and Rv3620 were PCR amplified from genomic template DNA (94° C. for 0.5 min.; 58° C. for 0.5 min., 58° C. for 1.5 min.; 35 cycles). Rv1813 was digested with NdeI/SacI then cloned into pET28.a vector. Rv3620 was digested with SacI/SaII then ligated into the Rv1813pET construct. Rv2608 was amplified from plasmid template by PCR (94° C. for 0.5 min., 58° C. for 0.5 min., 68° C. for 1.5 min.; 35 cycles). Product was digested with SaII/HindIII and cloned into pET28.a-Rv1813-3620 vector. The fusion construct has a polynucleotide sequence set forth in SEQ ID NO: 84, encoding the fusion protein set forth in SEQ ID NO: 91.

ID83 was expressed in host BL-21plysS (2 L, 2XYT growth media, 37° C.). Induced with 1 mM IPTG at OD 0.77 and harvested at OD 1.93. Cell pellet was suspended in lysis buffer (20 mM Tris pH8, 100 mM NaCl, 2 mM PMSF) and froze. The cell pellet was then thawed, lysed by sonication, and spun at 7,000 rcf for 20 minutes. ID83 is an inclusion body protein. The pellet was washed 2× with 1% Chaps. The pellet was solubilized in 60 mL in binding buffer (8M urea, 20 mM Tris pH 8, 100 mM NaCl) and bound to 16 mL Ni-NTA resin at RT for 1 hour. The resin was washed (50 mL 0.5% DOC for 20 minutes; 80 mL 60% IPA for 30 minutes, 50 mL 0.5% DOC rinse) and then eluted with binding buffer with 300 mM imidazol. The supernatant from the first bind was bound to an additional 8 mL resin and processed as indicated above. The aforementioned purifications removed breakdown products. Another Ni-NTA bind was performed overnight at 4° C. in 160 mL (binding buffer with 50 mM NaCl) with 32 mL resin. The resin was washed and eluted as indicated above. The fractions from this bind were dialyzed in 20 mM Tris pH8.

### Example 18

### Cloning and Expression of Recombinant Fusion Protein ID94

The following primers were used in for cloning the fusion construct ID94, which comprises fusion partners derived from Rv2389, Rv1813, Rv3620 and Rv2608:

5′: Rv2389mat-5NdeI
(SEQ ID NO: 93)
CAATTACATATGGACGACATCGATTGGGACGCC
3′: Rv2389mat-3KpnI-HindIII
(SEQ ID NO: 94)
CAATTAAAGCTTTTAAGTACTTGGTTTGCTGCCT
CTCGATCG

Rv2389 was PCR amplified from genomic template (94° C. for 0.5 min., 58° C. for 0.5 min., 68° C. for 1.5 min., 35 cycles), digested with NdeI/HindIII, and ligated into pET28.a. ID83-pET28.a vector was digested with KpnI/HindIII to drop out the insert. ID83 was ligated into Rv2389-pET28.a vector (also digested with KpnI/HindIII). The fusion construct has a polynucleotide sequence set forth in SEQ ID NO: 92, encoding the fusion protein set forth in SEQ ID NO: 95. ID94 was expressed in host BL-21plysS (1 L, 2XYT growth media, 37° C.). Expression was induced with 1 mM IPTG at OD 0.50 and harvested at OD 1.41. Cell pellet was suspended in lysis buffer (20 mM Tris pH8, 100 mM NaCl, 2 mM PMSF) and froze. ID94 forms an inclusion body and was processed the same as ID83. ID94 did not bind well overnight so the volume was doubled with 8M urea and BME was added to 10 mM. The less concentrated solutions were bound the Ni-NTA resin at RT for 2 hours then overnight at 4° C. The resin was washed and eluted as previously indicated. The fractions from this purification were dialyzed in 20 mM Tris pH8.

### Example 19

### Cloning and Expression of Recombinant Fusion Protein ID95

ID95 is a fusion construct comprising fusion partners derived from Rv2389, Rv3810, Rv1813, Rv3620 and Rv0496. ID58-pET28.a vector was digested with KpnI/HindIII to drop out the insert. The ID58 insert was ligated into previously made 36f.1-pET28.a vector (also digested with KpnI/HindIII). The fusion construct has a polynucleotide sequence set forth in SEQ ID NO: 96, encoding the fusion protein set forth in SEQ ID NO: 97. ID95 was expressed in host BL-21plysS (1 L, 2XYT growth media, 37° C.). Cell pellet was suspended in lysis buffer (20 mM Tris pH8, 100 mM NaCl, 2 mM PMSF) and froze. ID95 forms an inclusion body and was purified the same as ID83.

### Example 20

### Cloning and Expression of Recombinant Fusion Protein Id120

ID120 is a fusion construct comprising fusion partners derived from Rv2389, Rv3810, Rv1813, Rv3620 and Rv2608. ID83-pET28.a vector was digested with KpnI/HindIII to drop out the insert. The ID83 insert was ligated into previously made 36f.1-pET28.a vector (also digested with KpnI/HindIII). The fusion construct has a polynucleotide sequence set forth in SEQ ID NO: 98, encoding the fusion protein set forth in SEQ ID NO: 99. ID120 was expressed in host BL-21plysS (1 L, 2XYT growth media, 37° C.). Expression was induced with 1 mM IPTG at OD 0.50 and cells were harvested at OD 1.41. Cell pellet was suspended in lysis buffer (20 mM Tris pH8, 100 mM NaCl, 2 mM PMSF) and froze. ID120 forms an inclusion body and was processed the same as ID83. ID120 did not bind well overnight so the volume was doubled with 8M urea and BME was added to 10 mM. The less concentrated solutions were bound to Ni-NTA resin at RT for 2 hours then overnight at 4° C. The resin was washed and eluted as previously indicated. The fractions from this purification were dialyzed in 20 mM Tris pH8.

### Example 21

### Recognition of Mtb Antigens by Ppd+ Human Pbmc and Splenocytes from Mtb Infected Mice

This example demonstrates that Mtb antigen of the invention induce memory recall responses in human PBMC from PPD+ healthy donors, and splenocytes isolated from mice infected with Mycobacterium tuberculosis.

### Material & Methods

### Human PBMC In Vitro Stimulation and Cytokine ELISA

PBMC were obtained through apheresis or purified from heparinized blood from 7 PPD−, and 15 PPD+ healthy donors. PBMC were plated in triplicate 96-well tissue culture plates at 2-2.5×10⁵ cells/well and cultured with medium, PHA (10 μg/ml), Mycobacterium tuberculosis (Mtb) lysate (10 μg/ml), or each recombinant protein (50 μg/ml) for 72 h. Supernatants were harvested and analyzed for IFN-γ by a double-sandwich ELISA using specific mAb (eBioscience), and following the manufacturer's protocol.

### Mouse Cytokine ELISPOT

Spleen from Mycobacterium tuberculosis-infected mice were harvested at different times post-infection, and single splenocyte suspensions were obtained by conventional procedures. An ELISPOT assay was used to determine the relative number of IFN-γ or TNF-expressing splenocytes. MultiScreen 96-well filtration plates (Millipore, Bedford, MA) were coated with 10 μg/ml rat anti-mouse IFN-γ, or TNF, capture Ab (eBioscience) and incubated overnight at 4° C. Plates were washed with PBS, blocked with RPMI 1640 and 10% FBS for at least 1 h at room temperature, and washed again. Spleen cells were plated, in duplicate, at 2×10⁵ cells/well, and stimulated with the specific rAg at a 10 μg/ml for 48 h at 37° C. The plates were subsequently washed with PBS and 0.1% Tween and incubated overnight at 4° C. with a biotin-conjugated, rat anti-mouse IFN-γ, or TNF, secondary Ab (eBioscience) at 5 μg/ml in PBS, 0.5% BSA, and 0.1% Tween. The filters were developed using the Vectastain ABC avidin peroxidase conjugate and Vectastain AEC substrate kits (Vector Laboratories, Burlingame, CA) according to the manufacturer's protocol. The reaction was stopped by washing the plates with deionized water, plates were dried in the dark, and spots were counted.

### Results

### Recognition of Mtb Recombinant Proteins by Human PPD+ PBMC

PBMC from PPD+ and PPD− donors were cultured for 72 h with Mtb Rv0164, Rv0455, Rv0496, Rv2608, Rv3020, Rv3478, Rv3619, Rv3620, Rv1738, Rv1813, Rv3810, Rv2389, Rv2866, Rv3876, Rv0054, Rv0410, Rv0655, Rv0831, Rv1009, Rv1099, Rv1240, Rv1288, Rv1410, Rv1569, Rv1789, Rv1818, Rv1860, Rv1886, Rv1908, Rv2220, Rv2032, Rv2623, Rv2875, Rv3044, Rv3310, Rv3881, Rv0577, Rv1626, Rv0733, Rv2520, Rv1253, Rv1980, Rv3628, Rv1884, and Rv1511 recombinant proteins. A description of the production of these recombinant antigens is described elsewhere herein. The concentration of IFN-γ was further analyzed in the cell culture supernatants.

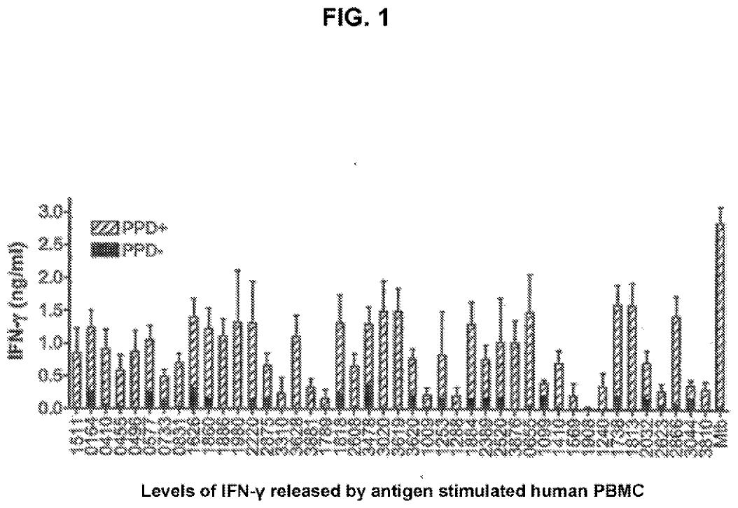

All the recombinant proteins tested, except Rv1908, were presented to and activated T cells from PPD+ donors to produce IFN-γ (FIG. 1).

Only background levels of IFN-γ were detected in response to these antigens using PBMC from PPD− controls. 5- to 70-fold increases in IFN-γ concentration were measured in PBMC cultures from PPD+ donors compared to PPD-controls, indicating antigen specific recognition of these recombinant proteins from donors previously exposed to Mycobacterium tuberculosis or Mycobacterium bovis (vaccinated with BCG).

Recognition of Mtb Recombinant Proteins by Splenocytes from M. tuberculosis-Infected Mice

Mice were infected by low dose aerosol exposure with Mycobacterium tuberculosis H37Rv strain, and spleens were harvested at different time post-infection. An ELISPOT assay was used to determine the relative number of TNF-expressing splenocytes in response to Mtb recombinant Rv0164, Rv0455, Rv0496, Rv2608, Rv3020, Rv3478, Rv3619, Rv3620, Rv1738, Rv1813, Rv3810, Rv2389, Rv2866, Rv0054, Rv0655, Rv0831, Rv1009, Rv1240, Rv1288, Rv1410, Rv1569, Rv1789, Rv1818, Rv1860, Rv1886, Rv1908, Rv2220, Rv2032, Rv2875, Rv3044, Rv3310, Rv3881, Rv0577, Rv1626, Rv0733, Rv1253, Rv1980, Rv3628, Rv1884, Rv3875, Rv1511 and ID83 proteins during a 48 h in vitro culture.

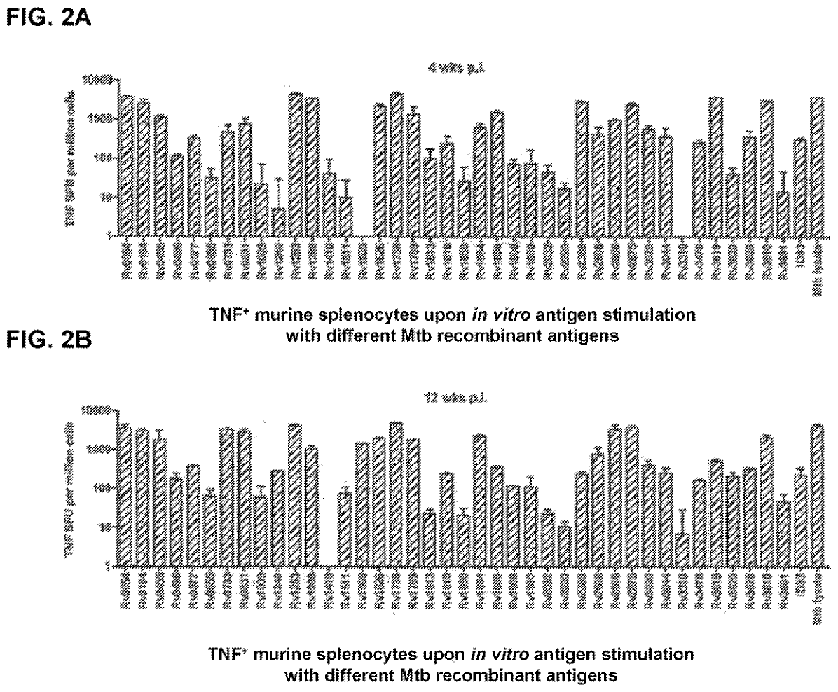

All the recombinant and fusion proteins tested induced an increase in the number of TNF+ splenocytes from Mycobacterium tuberculosis-infected mice 28 days (FIG. 2, upper panel), 60 days (data not shown), and 90 days post-infection (FIG. 2, lower panel).

Together these data indicate that Mycobacterium tuberculosis infection in mice induced immune responses to Mtb proteins, including to Rv0164, Rv0455, Rv0496, Rv2608, Rv3020, Rv3478, Rv3619, Rv3620, Rv1738, Rv1813, Rv3810, Rv2389, Rv2866, Rv0054, Rv0655, Rv0831, Rv1009, Rv1240, Rv1288, Rv1410, Rv1569, Rv1789, Rv1818, Rv1860, Rv1886, Rv1908, Rv2220, Rv2032, Rv2875, Rv3044, Rv3310, Rv3881, Rv0577, Rv1626, Rv0733, Rv1253, Rv1980, Rv3628, Rv1884, Rv1511 and ID83 proteins.

Thus, both humans naturally exposed to, and mice infected by an aerosol challenge with virulent, Mycobacterium tuberculosis-mounted immune responses to bacterial proteins, as evidenced by recall responses to Mtb lysate and PPD. In addition, increase in IFN-y and TNF cytokine responses to Rv0164, Rv0455, Rv0496, Rv2608, Rv3020, Rv3478, Rv3619, Rv3620, Rv1738, Rv1813, Rv3810, Rv2389, Rv2866, Rv3876, Rv0054, Rv0410, Rv0655, Rv0831, Rv1009, Rv1099, Rv1240, Rv1288, Rv1410, Rv1569, Rv1789, Rv1818, Rv1860, Rv1886, Rv1908, Rv2220, Rv2032, Rv2623, Rv2875, Rv3044, Rv3310, Rv3881, Rv0577, Rv1626, Rv0733, Rv2520, Rv1253, Rv1980, Rv3628, Rv1884, Rv1511 and ID83 protein upon in vitro stimulation indicates that these antigens (1) are recognized by previously exposed individuals (presence of memory T cells), (2) could be used as immuno-therapeutics or (3) could be used as diagnostics.

### Example 22

### Immune Responses to Mtb Antigens in C57BL/6 Mice and Protection Against Aerosol Challenge with Mtb

This example demonstrates that immunization of mice with Mtb antigens of the invention is immunogenic and can provide protection against aerosol Mycobacterium tuberculosis challenge.

### Material & Methods:

### Recombinant Antigens and Adjuvant Formulations

Recombinant proteins were produced as described above. CpG 1826 was obtained from Coley Pharmaceuticals (Wellesley, MA).

### Immunization

Female C57/BL6 mice were obtained from Charles River and age-matched (5-7 week) within each experiment. Mice were immunized three times (3 week apart) with 8 μg of recombinant Rv0164 (SEQ ID NO: 1), Rv0496 (SEQ ID NO: 6), Rv2608 (SEQ ID NO: 26), Rv3020 (SEQ ID NO: 36), Rv3478 (SEQ ID NO: 41), Rv3619 (SEQ ID NO: 46), Rv3620 (SEQ ID NO: 51), Rv1738 (SEQ ID NO: 11), Rv1813 (SEQ ID NO: 16), Rv3810 (SEQ ID NO: 56), Rv2389 (SEQ ID NO: 21), Rv2866 (SEQ ID NO: 31), Rv0831 (SEQ ID NO: 115), Rv1288 (SEQ ID NO: 127), Rv1569 (SEQ ID NO: 133), Rv1789 (SEQ ID NO: 136), Rv1818 (SEQ ID NO: 139), Rv1860 (SEQ ID NO: 142), Rv1886 (SEQ ID NO: 145), Rv2220 (SEQ ID NO: 154), Rv2032 (SEQ ID NO: 151), Rv2623 (SEQ ID NO: 160), Rv2875 (SEQ ID NO: 163), Rv3044 (SEQ ID NO: 166), Rv0577 (SEQ ID NO: 184), Rv1626 (SEQ ID NO: 187), Rv0733 (SEQ ID NO: 190), Rv3628 (SEQ ID NO: 202), and Rv1884 (SEQ ID NO: 205) protein formulated with 25 μg of the adjuvant CpG. Mice in the saline, adjuvant only, and BCG control groups received three doses of PBS, three doses of adjuvant alone, or a single dose of 5×10⁴ BCG CFU respectively. Mice were injected with a total volume of 100 μl/mouse via the s.c. route.

### Cytokine ELISA

Three weeks after the last boost, spleen from animals designated for immunogenicity studies were harvested, and splenocytes were obtained by conventional procedures. For cytokine analysis, splenocytes were plated in duplicate 96-well tissue culture plates at 2.5×10⁵ cells/well and cultured with medium, Con A 3 μg/ml, PPD 10 μg/ml, Mtb lysate 10 μg/ml, or each recombinant protein 10 μg/ml for 72 h. Supernatants were harvested and analyzed for IFN-γ by a double-sandwich ELISA using specific mAb (eBioscience), and following the manufacturer's protocol.

### Cytokine ELISPOT

MultiScreen 96-well filtration plates (Millipore, Bedford, MA) were coated with 10 μg/ml rat anti-mouse IFN-y or TNF capture Ab (eBioscience) and incubated overnight at 4° C. Plates were washed with PBS, blocked with RPMI 1640 and 10% FBS for at least 1 h at room temperature, and washed again. Splenocytes were plated in duplicate at 2×10⁵ cells/well, and stimulated with medium, Con A 3 μg/ml, PPD 10 μg/ml, or each recombinant protein 10 μg/ml for 48 h at 37° C. The plates were subsequently washed with PBS and 0.1% Tween-20 and incubated for 2 h with a biotin-conjugated rat anti-mouse IFN-γ or TNF secondary Ab (eBioscience) at 5 μg/ml in PBS, 0.5% BSA, and 0.1% Tween-20. The filters were developed using the Vectastain ABC avidin peroxidase conjugate and Vectastain AEC substrate kits (Vector Laboratories, Burlingame, CA) according to the manufacturer's protocol. The reaction was stopped by washing the plates with deionized water, plates were dried in the dark, and spots were counted on a automated ELISPOT reader (C.T.L. Serie3A Analyzer, Cellular Technology Ltd, Cleveland, OH), and analyzed with Immunospot® (CTL Analyzer LLC).

### IgG Isotype ELISA

Animals were bled 1 wk after the last immunization and serum IgG1 and IgG2c antibody titers were determined. Nunc-Immuno Polysorb plates were coated for 4 h at room temperature with 2 μg/ml of recombinant protein in 0.1 M bicarbonate buffer, blocked overnight at 4° C. with PBS Tween-20 0.05% BSA 1%, washed with PBS Tween-20 0.05%, incubated for 2 h at room temperature with sera at a 1:50 dilution and subsequent 5-fold serial dilutions, washed, and incubated for 1 h with anti-IgG1-HRP or anti-IgG2c-HRP 1:2000 in PBS Tween-20 0.05% BSA 0.1%. Plates were washed and developed using SureBlue TMB substrate (KPL Inc., Gaithersburg, MD). The enzymatic reaction was stopped with 1N H₂SO₄, and plates were read within 30 min at 450 nm with a reference filter set at 650 nm using a microplate ELISA reader (Molecular Devices, Sunnyvale, CA) and SoftMax Pro5. Endpoint titers were determined with GraphPad Prism 4 (GraphPad Software Inc., San Diego, CA) with a cutoff of 0.1.

### Protection Experiment

Mice were immunized s.c., three times, 3 weeks apart, with 8 □g of each recombinant protein from a subset of Mtb antigens, and mixed with the adjuvant CpG. Positive control mice were immunized with BCG (5×10⁴ CFU) in the base of the tail (once), and negative control animals were injected with saline, or adjuvant alone. Thirty days after the last immunization, mice were challenged by low dose aerosol exposure with Mycobacterium tuberculosis H37Rv strain (ATCC 35718; American Type Culture Collection, Manassas, VA) using a UW-Madison aerosol exposure chamber (Madison, WI) calibrated to deliver 50-100 bacteria into the lungs. Four weeks later, mice were euthanized, and lung and spleen homogenates were prepared in PBS/Tween 80 (0.05%). Bacterial counts were determine by plating serial dilutions of individual whole organs on nutrient Middlebrook 7H11 Bacto Agar (BD Biosciences, Cockeysville, MD) and counting bacterial colony formation after 14-day incubation at 37° C. in humidified air and 5% CO₂. Data are expressed as Log 10 of the mean number of bacteria recovered±SD, and Log 10 Reduction in CFU=Log 10 CFU for the vaccinated group−Log 10 CFU for the Saline treated group.

### Results:

Immune Responses to Recombinant Mtb Antigens Adjuvanted with CpG.

C57BL/6 mice were immunized three times, three weeks apart, with recombinant Mtb Rv0164, Rv0455, Rv0496, Rv2608, Rv3020, Rv3478, Rv3619, Rv3620, Rv1738, Rv1813, Rv3810, Rv2389, Rv2866, Rv0831, Rv1818, Rv1886, Rv2032, Rv2623, Rv2875, Rv3044, Rv0577, Rv1626, Rv3628, and Rv1884 proteins formulated with 25 μg of the adjuvant CpG. One week, and three weeks after the last immunization, the presence of antigen specific antibody, and memory T lymphocytes respectively, were assessed.

The specific serum IgG isotype Ab response was measured by conventional ELISA by coating each of the recombinant protein onto a plate and serially diluting the different sera. IgG2c:IgG1 endpoint titer ratios were determined for each vaccine group (Table 1). Saline, CpG adjuvant alone, or BCG immunization did not induce an IgG1 or IgG2c antibody response specific to any or the Mtb recombinant proteins tested (data not shown). Immunization with each of the Mtb recombinant proteins with the adjuvant CpG induced antigen specific IgG1 and IgG2c.

TABLE 1
Immune responses to Mtb antigens

| Antigen | IFN-γ^{a} | TNF^{a} | IrgG^{b} | Antigen | IFN-γ | TNF | IgG |
|---|---|---|---|---|---|---|---|
| Rv0577 | 523(8) | 388(297) | 0.98 | Rv0496 | 68(52) | 24(5) | *1.21 |
| Rv1626 | 20(21) | 268(117) | *1.19 | Rv0831 | 24(12) | 24(8) | *1.19 |
| Rv2875 | 428(172) | 137(60) | *1.05 | Rv1886 | 590(106) | 102(37) | 1.00 |
| Rv2608 | 798(11) | 175(105) | 1.09 | Rv3020 | 48(27) | 20(16) | *1.18 |
| Rv3478 | 453(4) | 149(73) | 1.03 | Rv3619 | 604(184) | 1261(319) | *1.13 |
| Rv3044 | 331(161) | 57(1) | *1.05 | Rv1813 | 388(103) | 32(13) | *1.18 |
| Rv0164 | 163(87) | 94(58) | *1.17 | Rv2380 | 39(49) | 92(31) | 1.02 |
| Rv0455 | 24(12) | 44(24) | 1.06 | Rv2623 | 21(12) | 2(1) | *1.14 |
| Rv1738 | 24(16) | 32(16) | 1.23 | Rv2866 | 104(56) | 32(12) | *1.31 |
| Rv1818 | 155(72) | 10(2) | *0.90 | Rv3620 | 184(44) | 72(33) | *1.13 |
| Rv1684 | 1600(372) | ND^{c} | 1.01 | Rv3628 | 16(8) | ND | 1.09 |
| Rv2032 | 28(16) | ND | *1.14 | Rv3810 | 44(56) | 7(10) | 1.08 |

^{a}Spot-Forming-Unit per million cells (SD). Mice were immunized s.c. three times, three wks aport with Mtb antigens (RV#) + CpG. Cytokine responses to the antigens were determined by ELISPOT 3 wks after the last injection.
^{b}IgG2c:IgG1 ratio,
*P < 0.05, Student′s t Test,
^{c}ND, not done.

Three weeks after the last immunization, splenocytes were prepared and assayed by ELISPOT to determine the relative number of IFN-γ or TNF-expressing splenocytes in response to medium alone, the mitogen ConA, PPD, Mtb lysate, and each of the recombinant Mtb proteins.

Injection with saline, or CpG adjuvant alone did not induce IFN-γ or TNF responses specific to any of the recombinant proteins (data not shown).

Immunization with each of the Mtb recombinant proteins with the adjuvant CpG induced antigen specific IFN-γ and/or TNF recall responses by activated splenocytes (Table 1). Lower levels of IFN-γ in response to Mtb lysate and PPD were also observed (data not shown), suggesting that these proteins are naturally found in mycobacterial lysates and partially purified derivatives.

Together, these results indicate that immunization with the different recombinant Mtb antigens in CpG induced a Th1-type memory response with predominant IgG2c, IFN-γ, and TNF.

Protection afforded by the different Mtb recombinant proteins, adjuvanted with CpG, against an aerosol challenge with Mtb H37Rv.

Number of viable bacilli, expressed as mean Log 10 CFU, in the lung and spleen of mice vaccinated with Mtb recombinant protein Rv0496, Rv2608, Rv3020, Rv3478, Rv3619, Rv3620, Rv1813, Rv1569, Rv1789, Rv1860, Rv1886, Rv2220, Rv2875, Rv3044, Rv0577, Rv1626, and Rv0733, adjuvanted with CpG, were determined 4 weeks post aerosol challenge with ˜50 CFU of virulent Mycobacterium tuberculosis H37Rv. The mean Log 10 CFU in the lung of mice immunized with the different recombinant proteins was compared to the mean Log 10 CFU obtained in mice receiving placebo (saline) or BCG, the current and only vaccine against TB. The difference in mean Log 10 CFU in the saline group vs the vaccinated groups is expressed as Log 10 reduction in CFU.

Immunization of mice with three doses of Rv3478+CpG or Rv2608+CpG resulted in a decrease in viable Mtb bacilli, in lung (0.66, respectively 0.58) close to that afforded by BCG vaccination (0.78) (Table 2). Immunization with each of Rv0496, Rv3020, Rv3619, Rv3620, Rv1813, Rv1569, Rv1789, Rv1860, Rv1886, Rv2220, Rv2875, Rv3044, Rv0577, Rv1626, and Rv0733, adjuvanted with CpG, also afforded some protection against Mtb infection. CpG adjuvant alone did not reduce lung bacterial burden (−0.09).

TABLE 2
Vaccine-induced protection against Mtb^{a}
CFU Reduction (Log₁₀)^{b}

| | | | | | | | |
|---|---|---|---|---|---|---|---|
| Rv0496 | 0.11 | Rv0577 | 0.36 | Rv1886 | 0.20 | Rv3478 | 0.66 |
| Rv0733 | 0.23 | Rv1626 | 0.32 | Rv1569 | 0.12 | Rv3044 | 0.43 |
| Rv0831 | 0.13 | Rv2875 | 0.44 | Rv1789 | 0.15 | Rv2220 | 0.25 |
| Rv1411 | 0.11 | Rv2608 | 0.58 | Rv3020 | 0.17 | BCG | 0.78 |
| Rv1860 | 0.19 | Rv3619 | 0.24 | Rv1813 | 0.14 | CpG | −0.09 |

^{a}Mice were immunized s.c. three times, three wks apart with 8 μg Mtb antigens (Rv#) + 25 μg CpG.
^{b}Reduction of viable bacteria (CFU) in the lungs compared to saline immunized animals 4 wks after a low dose aerosol challenge with M. tuberculosis H37Rv or Erdman strains.

These results are surprising in that levels of protection against Mtb infection were achieved with 3 doses of a single recombinant protein adjuvanted with CpG.

### Example 23

### Immune Responses to a Mixture of Mtb Antigens in C57Bl/6 Mice and Protection Against Aerosol Challenge with Mtb

This example demonstrates that immunization of mice with a mixture of Mtb antigens of the invention is immunogenic and can provide protection against aerosol Mycobacterium tuberculosis challenge.

### Material & Methods:

### Recombinant Antigens and Adjuvant Formulations

Recombinant proteins were produced as described above. CpG 1826 was obtained from Coley Pharmaceuticals (Wellesley, MA).

### Immunization

Female C57/BL6 mice were obtained from Charles River and age-matched (5-7 week) within each experiment. Mice were immunized three times (3 week apart) with 6 or 8 μg of recombinant Rv2608, Rv3620, and Rv1813 protein formulated with 25 μg of the adjuvant CpG. Mice in the adjuvant only, and BCG control groups received three doses of adjuvant alone, or a single dose of 5×10⁴ BCG CFU respectively. Mice were injected with a total volume of 100 μl/mouse via the s.c. route.

### Cytokine ELISA

Three weeks after the last boost, spleen from animals designated for immunogenicity studies were harvested, and splenocytes were obtained by conventional procedures. For cytokine analysis, splenocytes were plated in duplicate 96-well tissue culture plates at 2.5×10⁵ cells/well and cultured with medium, Con A 3 μg/ml, PPD 10 μg/ml, Mtb lysate 10 μg/ml, or each recombinant protein 10 μg/ml for 72 h. Supernatants were harvested and analyzed for IFN-γ by a double-sandwich ELISA using specific mAb (eBioscience), and following the manufacturer's protocol.

### Cytokine ELISPOT

MultiScreen 96-well filtration plates (Millipore, Bedford, MA) were coated with 10 μg/ml rat anti-mouse IFN-γ or TNF capture Ab (eBioscience) and incubated overnight at 4° C. Plates were washed with PBS, blocked with RPMI 1640 and 10% FBS for at least 1 h at room temperature, and washed again. Splenocytes were plated in duplicate at 2×10⁵ cells/well, and stimulated with medium, Con A 3 μg/ml, PPD 10 μg/ml, or each recombinant protein 10 μg/ml for 48 h at 37° C. The plates were subsequently washed with PBS and 0.1% Tween-20 and incubated for 2 h with a biotin-conjugated rat anti-mouse IFN-γ or TNF secondary Ab (eBioscience) at 5 μg/ml in PBS, 0.5% BSA, and 0.1% Tween-20. The filters were developed using the Vectastain ABC avidin peroxidase conjugate and Vectastain AEC substrate kits (Vector Laboratories, Burlingame, CA) according to the manufacturer's protocol. The reaction was stopped by washing the plates with deionized water, plates were dried in the dark, and spots were counted on a automated ELISPOT reader (C.T.L. Serie3A Analyzer, Cellular Technology Ltd, Cleveland, OH), and analyzed with Immunospot® (CTL Analyzer LLC).

### IgG Isotype ELISA

Animals were bled 1 wk after the last immunization and serum IgG1 and IgG2c antibody titers were determined. Nunc-Immuno Polysorb plates were coated for 4 h at room temperature with 2 μg/ml of recombinant protein in 0.1 M bicarbonate buffer, blocked overnight at 4° C. with PBS Tween-20 0.05% BSA 1%, washed with PBS Tween-20 0.05%, incubated for 2 h at room temperature with sera at a 1:50 dilution and subsequent 5-fold serial dilutions, washed, and incubated for 1 h with anti-IgG1-HRP or anti-IgG2c-HRP 1:2000 in PBS Tween-20 0.05% BSA 0.1%. Plates were washed and developed using SureBlue TMB substrate (KPL Inc., Gaithersburg, MD). The enzymatic reaction was stopped with 1N H₂SO₄, and plates were read within 30 min at 450 nm with a reference filter set at 650 nm using a microplate ELISA reader (Molecular Devices, Sunnyvale, CA) and SoftMax Pro5. Endpoint titers were determined with GraphPad Prism 4 (GraphPad Software Inc., San Diego, CA) with a cutoff of 0.1.

### Protection Experiment

Mice were immunized s.c., three times, 3 weeks apart, with 6 or 8 μg of each recombinant protein from a subset of Mtb antigens, and mixed with the adjuvant CpG. Positive control mice were immunized with BCG (5×10⁴ CFU) in the base of the tail (once), and negative control animals were injected with adjuvant alone. Thirty days after the last immunization, mice were challenged by low dose aerosol exposure with Mycobacterium tuberculosis H37Rv strain (ATCC 35718; American Type Culture Collection, Manassas, VA) using a UW-Madison aerosol exposure chamber (Madison, WI) calibrated to deliver 50-100 bacteria into the lungs. Four weeks later, mice were euthanized, and lung and spleen homogenates were prepared in PBS/Tween 80 (0.05%). Bacterial counts were determine by plating serial dilutions of individual whole organs on nutrient Middlebrook 7H11 Bacto Agar (BD Biosciences, Cockeysville, MD) and counting bacterial colony formation after 14-day incubation at 37° C. in humidified air and 5% CO 2. Data are expressed as Log 10 of the mean number of bacteria recovered±SD, and Log 10 Reduction in CFU=Log 10 CFU for the vaccinated group−Log 10 CFU for the Saline treated group.

### Results:

Immune Responses to a Mixture of Recombinant Mtb Antigens Adjuvanted with CpG.

C57BL/6 mice were immunized three times, three weeks apart, with each recombinant Mtb Rv2608, Rv3620, and Rv1813 proteins, separately (8 μg) or in a mixture (6 μg each), formulated with 25 μg of the adjuvant CpG. One week, and three weeks after the last immunization, the presence of antigen specific antibody, and memory T lymphocytes respectively, were assessed.

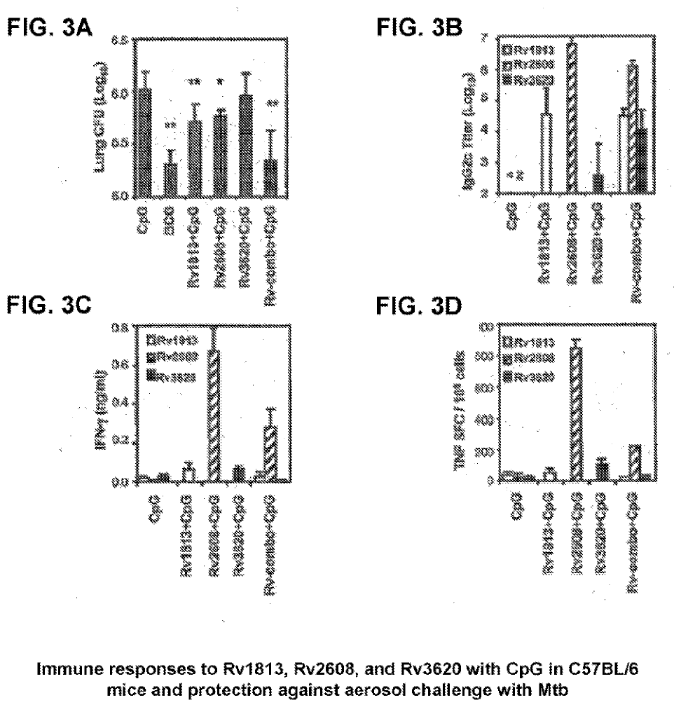

The specific serum IgG isotype Ab response was measured by conventional ELISA by coating each of the recombinant protein onto a plate and serially diluting the different sera. IgG2c endpoint titers were determined for each vaccine group. CpG adjuvant alone or BCG immunization did not induce an IgG1 or IgG2c antibody response specific to any or the Mtb recombinant proteins tested (FIG. 3B, and data not shown). Immunization with each of the Mtb recombinant proteins with the adjuvant CpG induced antigen specific IgG1 (data not shown) and IgG2c (FIG. 3B).

Three weeks after the last immunization, splenocytes were prepared and assayed by ELISA or ELISPOT to determine the relative level of IFN-y or number of TNF-expressing splenocytes in response to medium alone, the mitogen ConA, PPD, Mtb lysate, and each of the recombinant Mtb proteins.

Injection with CpG adjuvant alone did not induce IFN-γ or TNF responses specific to any of the recombinant proteins (FIG. 3C-D).

Immunization with each of the Mtb recombinant proteins with the adjuvant CpG induced antigen specific IFN-γ and TNF recall responses by activated splenocytes (FIG. 3C-D). Lower levels of cytokine responses were observed when the three antigens were used as a mixture.

Together, these results indicate that immunization with the different recombinant Mtb antigens, separately or as a mixture, in CpG induced a Th1-type memory response with predominant IgG2c, IFN-γ, and TNF.

Protection Afforded by a Mixture of Different Mtb Recombinant Proteins, Adjuvanted with CpG, Against an Aerosol Challenge with Mtb H37Rv.

Number of viable bacilli, expressed as mean Log 10 CFU, in the lung of mice vaccinated with Mtb recombinant protein Rv2608, Rv3620, and Rv1813, separately (8 μg) or in a mixture (6 μg each), adjuvanted with CpG, were determined 4 weeks post aerosol challenge with ˜50 CFU of virulent Mycobacterium tuberculosis H37Rv. The mean Log 10 CFU in the lung of mice immunized with the different recombinant proteins was compared to the mean Log 10 CFU obtained in mice receiving adjuvant alone or BCG, the current and only vaccine against TB. The difference in mean Log 10 CFU in the adjuvant group vs the vaccinated groups is expressed as Log 10 reduction in CFU.

Immunization of mice with three doses of Rv2608+Rv3620+Rv1813+CpG resulted in a decrease in viable Mtb bacilli in lung (Log 10 reduction in CFU of 0.67) close to that afforded by BCG vaccination (0.71) (FIG. 3A). Immunization with Rv2608 or Rv1813, adjuvanted with CpG, also afforded some protection against Mtb infection (0.24 and 0.30 respectively). Immunization with Rv3620+CpG or CpG adjuvant alone did not reduce lung bacterial burden. The reduction in CFU achieved by injecting a mixture of three Mtb antigens was higher than adding up individual effects.

These results are surprising in that levels of protection against Mtb infection were increased with 3 doses of a mixture or three recombinant proteins adjuvanted with CpG, compared to 3 doses of individual proteins with CpG.

### Example 24

### Immune Responses to Id83 and Id93 Fusion Proteins in C57Bl/6 Mice and Protection Against Aerosol Challenge with Mtb

This example demonstrates that immunization of mice with fusion proteins of the invention is immunogenic and can provide protection against aerosol Mycobacterium tuberculosis challenge.

### Material & Methods:

### Fusion Proteins and Adjuvant Formulations

Fusion proteins were produced as described above. CpG 1826 was obtained from Coley Pharmaceuticals (Wellesley, MA). Glucopyranosyl lipid A (GLA) was obtained from Avanti (Alabaster, AL) and Gardiquimod (GDQ) was obtained from Invivogen (San Diego, CA). Oil-in-water sable emulsions (−SE) were prepared by standard techniques.

### Immunization

Female C57/BL6 mice were obtained from Charles River and age-matched (5-7 week) within each experiment. Mice were immunized three times (3 week apart) with 8 μg of ID83 and ID93 fusion protein formulated with 20 μg of the adjuvant GLA-SE, or 8 μg ID83 fusion protein formulated with 20-25 μg of the adjuvant GLA-SE, GDQ-SE, CpG-SE, GLA/GDQ-SE, GLA/CpG-SE, CpG/GDQ-SE. Mice in the saline, adjuvant only, and BCG control groups received three doses of PBS, three doses of adjuvant alone, or a single dose of 5×10⁴ BCG CFU respectively. Mice were injected with a total volume of 100 μl/mouse via the s.c. route.

### Cytokine ELISA

Three weeks after the last boost, spleen from animals designated for immunogenicity studies were harvested, and splenocytes were obtained by conventional procedures. For cytokine analysis, splenocytes were plated in duplicate 96-well tissue culture plates at 2.5×10⁵ cells/well and cultured with medium, Con A 3 μg/ml, PPD 10 μg/ml, Mtb lysate 10 μg/ml, or each fusion protein 10 μg/ml for 72 h. Supernatants were harvested and analyzed for IFN-γ by a double-sandwich ELISA using specific mAb (eBioscience), and following the manufacturer's protocol.

### IgG Isotype ELISA

Animals were bled 1 wk after the last immunization and serum IgG1 and IgG2c antibody titers were determined. Nunc-Immuno Polysorb plates were coated for 4 h at room temperature with 2 μg/ml of recombinant protein in 0.1 M bicarbonate buffer, blocked overnight at 4° C. with PBS Tween-20 0.05% BSA 1%, washed with PBS Tween-20 0.05%, incubated for 2 h at room temperature with sera at a 1:50 dilution and subsequent 5-fold serial dilutions, washed, and incubated for 1 h with anti-IgG1-HRP or anti-IgG2c-HRP 1:2000 in PBS Tween-20 0.05% BSA 0.1%. Plates were washed and developed using SureBlue TMB substrate (KPL Inc., Gaithersburg, MD). The enzymatic reaction was stopped with 1N H₂SO₄, and plates were read within 30 min at 450 nm with a reference filter set at 650 nm using a microplate ELISA reader (Molecular Devices, Sunnyvale, CA) and SoftMax Pro5. Endpoint titers were determined with GraphPad Prism 4 (GraphPad Software Inc., San Diego, CA) with a cutoff of 0.1.

### Protection Experiment

Mice were immunized s.c., three times, 3 weeks apart, with 8 μg of the fusion protein, formulated in the indicated adjuvant. Positive control mice were immunized with BCG (5×10⁴ CFU) in the base of the tail (once), and negative control animals were injected with saline, or adjuvant alone. Thirty days after the last immunization, mice were challenged by low dose aerosol exposure with Mycobacterium tuberculosis H37Rv strain (ATCC 35718; American Type Culture Collection, Manassas, VA) using a UW-Madison aerosol exposure chamber (Madison, WI) calibrated to deliver 50-100 bacteria into the lungs. Four weeks later, mice were euthanized, and lung and spleen homogenates were prepared in PBS/Tween 80 (0.05%). Bacterial counts were determine by plating serial dilutions of individual whole organs on nutrient Middlebrook 7H11 Bacto Agar (BD Biosciences, Cockeysville, MD) and counting bacterial colony formation after 14-day incubation at 37° C. in humidified air and 5% CO₂. Data are expressed as Log 10 of the mean number of bacteria recovered±SD, and Log 10 Reduction in CFU=Log 10 CFU for the vaccinated group−Log 10 CFU for the Saline treated group.

### Results:

Immune Responses to ID83 and ID93 Adjuvanted with GLA-SE

C57BL/6 mice were immunized three times, three weeks apart, with ID83 or ID93 fusion proteins formulated with 20 μg of the adjuvant GLA-SE. One week, and three weeks after the last immunization, the presence of antigen specific antibody, and memory T lymphocytes respectively, were assessed.

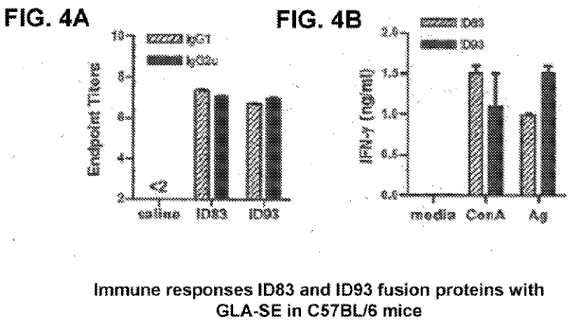

The specific serum IgG isotype Ab response was measured by conventional ELISA by coating each of the recombinant protein onto a plate and serially diluting the different sera. Endpoint titers were determined for each vaccine group. Saline did not induce an IgG1 or IgG2c antibody response specific to ID83 or ID93 fusion proteins (FIG. 4A) nor did GLA-SE adjuvant alone (data not shown). Immunization with ID83 or ID93 fusion protein with the adjuvant GLA-SE induced antigen specific IgG1 and IgG2c.

Three weeks after the last immunization, splenocytes were prepared and assayed by ELISA to determine the relative level of IFN-γ produced by splenocytes in response to medium alone, the mitogen ConA, and each of the fusion proteins.

Injection with saline or GLA-SE adjuvant alone did not induce IFN-y responses specific to ID83 or ID93 fusion proteins (data not shown).

Immunization with ID83 or ID93 fusion protein with the adjuvant GLA-SE induced antigen specific IFN-γ recall responses by activated splenocytes (FIG. 4B).

Together, these results indicate that immunization with the different fusion proteins in GLA-SE induced B and T cell immune responses.

Immunogenicity of ID83 Formulated with Different Adjuvants

C57BL/6 mice were immunized three times, three weeks apart, with ID83 fusion protein formulated with 20-25 μg of the adjuvant GLA-SE, GDQ-SE, CpG-SE, GLA/GDQ-SE, GLA/CpG-SE, CpG/GDQ-SE. One week, and three weeks after the last immunization, the presence of antigen specific antibody, and memory T lymphocytes respectively, were assessed.

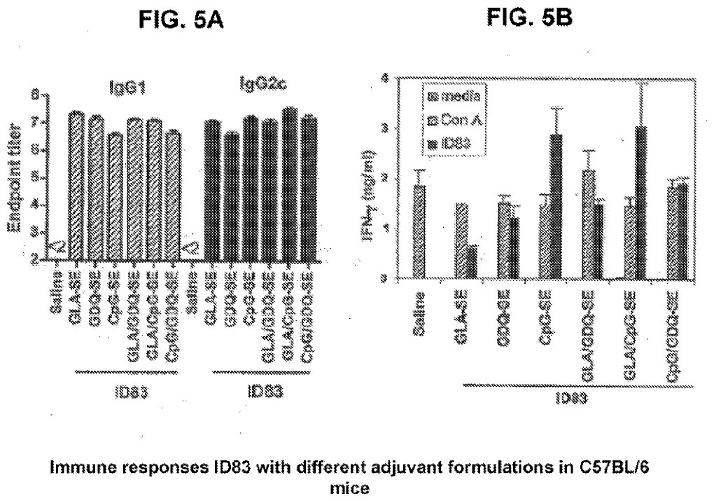

The specific serum IgG isotype Ab response was measured by conventional ELISA by coating each of the recombinant protein onto a plate and serially diluting the different sera. Endpoint titers were determined for each vaccine group. Saline did not induce an IgG1 or IgG2c antibody response specific to ID83 fusion proteins. Immunization with ID83 with the different adjuvants induced antigen specific IgG1 and IgG2c (FIG. 5A).

Three weeks after the last immunization, splenocytes were prepared and assayed by ELISA to determine the relative level of IFN-γ produced by splenocytes in response to medium alone, the mitogen ConA, and ID83 fusion protein.

Injection with saline did not induce IFN-γ responses specific to ID83 fusion protein. Immunization with ID83 fusion protein with the different adjuvants induced antigen specific IFN-γ recall responses by activated splenocytes (FIG. 5B).

Together, these results indicate that immunization with ID83 fusion protein in a variety of adjuvants induced B and T cell immune responses. Protection afforded by ID83 and ID93 fusion proteins, formulated with the adjuvant GLA-SE, against an aerosol challenge with Mtb H37Rv.

Number of viable bacilli, expressed as mean Log 10 CFU, in the lung of mice vaccinated with ID83 or ID93 fusion proteins adjuvanted with GLA-SE, were determined 4 weeks post aerosol challenge with ˜50 CFU of virulent M. tuberculosis H37RV.

The mean Log 10 CFU in the lung of mice immunized with the different fusion proteins was compared to the mean Log 10 CFU obtained in mice receiving placebo (saline) or BCG. The difference in mean Log 10 CFU in the saline group vs the vaccinated groups is expressed as Log 10 reduction in CFU.

Immunization of mice with three doses of ID83+GLA-SE or ID93+GLA-SE resulted in a decrease in viable Mtb bacilli in the lung of Mtb-infected mice of 0.34, respectively 0.48 Log 10 (Table 3). These results demonstrate that protection against Mtb infection was achieved with 3 doses of two different fusion proteins adjuvanted with GLA-SE.

TABLE 3
Number of viable bacilli in the lung of vaccinated mice.

| Groups | CFU^{a} | SD | Diff^{b} | Groups | CFU | SD | Diff. |
|---|---|---|---|---|---|---|---|
| Saline | 5.79 | 0.09 | N/A^{c} | Saline | 5.94 | 0.15 | N/A |
| BCG | 5.06 | 0.18 | 0.73 | BCG | 5.07 | 0.20 | 0.57 |
| ID83 + GLA − SE | 5.45 | 0.23 | 0.34 | ID93 + GLA − SE | 5.46 | 0.21 | 0.48 |

^{a}CFU = colony-forming-units. Values represent the number of viable bacilli in the lungs of infected mice and are expressed as Log₁₀.
^{b}Difference = Log₁₀ CFU for the Saline group-Log₁₀ CFU for the vaccinated treated group.
^{c}N/A = not applicable.

Protection afforded by ID83 formulated with different adjuvants, in C57BL/6 mice, against an aerosol challenge with Mtb H37Rv.

Number of viable bacilli, expressed as mean Log 10 CFU, in the lung of mice vaccinated with ID83 fusion protein formulated with 20-25 μg of the adjuvant GLA-SE, CpG-SE, or GLA/CpG-SE were determined 4 weeks post aerosol challenge with ˜50 CFU of virulent M. tuberculosis H37Rv.

The mean Log 10 CFU in the lung of mice immunized with ID83 in the different adjuvants was compared to the mean Log 10 CFU obtained in mice receiving placebo (saline) or BCG. The difference in mean Log 10 CFU in the saline group vs the vaccinated groups is expressed as Log 10 reduction in CFU.

Immunization of mice with three doses of ID83 with different adjuvants resulted in a decrease in viable Mtb bacilli in the lung of Mtb-infected mice (Table 4). These results are promising in that protection against Mtb infection was achieved with 3 doses of two different fusion proteins adjuvanted with GLA-SE.

TABLE 4
Number of viable bacilli in the lung of vaccinated mice.

| Groups | CFU^{a} | SD^{b} | CFU Reduction^{c} | P value^{d} |
|---|---|---|---|---|
| Saline | 6.28 | 0.22 | | |
| BCG | 5.01 | 0.15 | 1.27 | <0.01 |
| ID83 + GLA-SE | 5.75 | 0.22 | 0.53 | <0.01 |
| ID83 + CpG-SE | 5.79 | 0.12 | 0.49 | <0.01 |
| ID83 + GLA/CpG-SE | 5.62 | 0.22 | 0.66 | <0.01 |

^{a}CFU = colony-forming-units. Values represents the number of viable bacilli in the lungs of infected mice and are expressed as Log₁₀.
^{b}SD, standard deviation
^{c}CFU Reduction = Log₁₀ CFU for the Saline group - Log₁₀ CFU for the vaccinated treated group.
^{d}P value is calculated with one-way ANOVA followed by Dunnett's multiple comparison Test. P values <0.05 are considered statistically significant

Together, these results indicate that vaccination with ID83 fusion protein adjuvanted with CpG-SE, GLA-SE, or CpG/GLA-SE reduced the bacterial burden and partially protected mice from M. tuberculosis infection. ID83+CpG/GLA-SE was the most effective formulation in reducing the number of viable bacteria in the lungs of Mtb-infected mice.

Protection afforded by ID83 formulated with GLA/CpG-SE, in guinea pigs, against an aerosol challenge with Mtb H37Rv.

Survival of guinea pigs vaccinated with ID83 fusion protein formulated with 20/25 μg of the adjuvant GLA/CpG-SE were followed for 200 days post aerosol challenge with ˜50 CFU of virulent M. tuberculosis H37Rv.

The survival of guinea pigs immunized with ID83 in GLA/CpG-SE adjuvant was compared to the survival of guinea pigs receiving placebo (saline) or BCG.

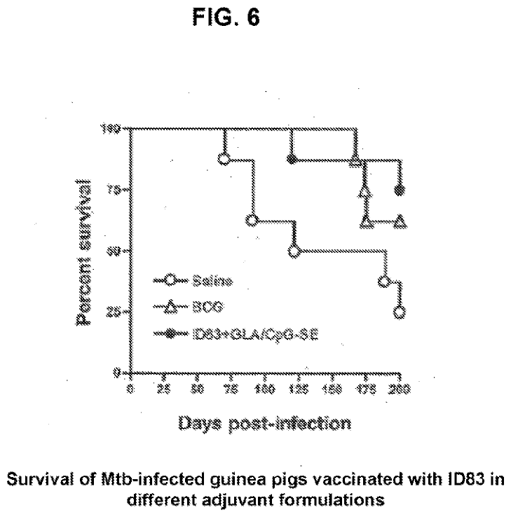

Immunization of guinea pigs with three doses of ID83 with different adjuvants resulted in increased survival of Mtb-infected guinea pig (FIG. 6). At day 200 post-infection, 75% of the animals vaccinated with ID83+GLA/CpG-SE were still alive, compared with 25% of the guinea pigs in the placebo group. 62% of guinea pigs immunized with BCG were alive at day 200 post-infection with Mtb.

These results demonstrate that protection against Mtb infection was achieved with 3 doses of ID83 fusion protein formulated with GLA/CpG-SE. In addition, vaccination with ID83+GLA/CpG-SE protected Mtb-infected guinea pigs longer than BCG.

Together, these results indicate that vaccination with ID83 fusion protein adjuvanted with CpG-SE, GLA-SE, or CpG/GLA-SE reduced the bacterial burden in the lungs of Mtb-infected mice, and partially protected guinea pigs from M. tuberculosis infection. ID83+CpG/GLA-SE was the most effective formulation in reducing the number of viable bacteria in the lungs of Mtb-infected mice and prolonging the survival of Mtb-infected guinea pigs.

Vaccination of mice with three doses of ID83 or ID93 fusion protein, adjuvanted with GLA-SE, induced antibody and Th1 T cell memory responses along with reduction in viable bacilli counts in the lung of mice infected with M. tuberculosis. Furthermore, a combination of CpG and GLA-SE was observed to be most immunogenic and conferred increased protection to M. tuberculosis challenge.

### Example 25

### Immune Responses to AD5-ID83 in C57BL/6 Mice and Protection Against an Aerosol M. Tuberculosis Challenge

This example demonstrates that immunization of mice with an adenovirus vector engineered to express ID83 fusion proteins of the invention is immunogenic in C57BL/6 mice.

### Material & Methods:

### Virus Construction and Purification

Ad5-ID83 was constructed using the AdEasy™ XL AdenoviralVector System (Stratagene #240010). Briefly, ID83 was amplified from plasmid DNA using PCR, digested with HinDIII and EcoRV, and ligated into pShuttle-CMV to make ID83-pShuttleCMV. ID83-pShuttleCMV was linearized by digesting with Pmel and electroporated (2.4 kV, 186Ω, 0.2 cm gap cuvette) into Escherichia coli BJ5183-AD-1 electro-competent cells (Stratagene #200157). Recombinant Ad5-ID83 plasmids were identified by digesting with Pacl. Pacl digested Ad5-ID83 plasmid (4 μg) was transfected into AD-239 cells in 60 mm plates using Polyfect reagent (Invitrogen #301107). After 4 days cells were harvested in 3 mL media and lysed by three cycles of freeze/thaw. Lysate supernatant was used to amplify virus for purification by CsCl gradient centrifugation.

### Immunization

Female C57/BL6 mice were obtained from Charles River and age-matched (5-7 week) within each experiment. Mice were immunized two times (3 week apart) with 5×10⁸ Ad5-ID83 viral particles. Mice in the saline, and BCG control groups received PBS or a single dose of 5×10⁴ BCG CFU respectively. Mice were injected with a total volume of 100 μl/mouse via the i.m. route.

### Cytokine ELISPOT

MultiScreen 96-well filtration plates (Millipore, Bedford, MA) were coated with 10 μg/ml rat anti-mouse IFN-γ or TNF capture Ab (eBioscience) and incubated overnight at 4° C. Plates were washed with PBS, blocked with RPMI 1640 and 10% FBS for at least 1 h at room temperature, and washed again. Splenocytes were plated in duplicate at 2×10⁵ cells/well, and stimulated with medium, Con A 3 μg/ml, PPD 10 μg/ml, or each recombinant protein 10 μg/ml for 48 h at 37° C. The plates were subsequently washed with PBS and 0.1% Tween-20 and incubated for 2 h with a biotin-conjugated rat anti-mouse IFN-γ or TNF secondary Ab (eBioscience) at 5 μg/ml in PBS, 0.5% BSA, and 0.1% Tween-20. The filters were developed using the Vectastain ABC avidin peroxidase conjugate and Vectastain AEC substrate kits (Vector Laboratories, Burlingame, CA) according to the manufacturer's protocol. The reaction was stopped by washing the plates with deionized water, plates were dried in the dark, and spots were counted on a automated ELISPOT reader (C.T.L. Serie3A Analyzer, Cellular Technology Ltd, Cleveland, OH), and analyzed with Immunospot® (CTL Analyzer LLC).

### Protection Experiment

Mice were immunized s.c., three times, 3 weeks apart, with 8 μg of the fusion protein, formulated in the indicated adjuvant. Positive control mice were immunized with BCG (5×10⁴ CFU) in the base of the tail (once), and negative control animals were injected with saline, or adjuvant alone. Thirty days after the last immunization, mice were challenged by low dose aerosol exposure with Mycobacterium tuberculosis H37Rv strain (ATCC 35718; American Type Culture Collection, Manassas, VA) using a UW-Madison aerosol exposure chamber (Madison, WI) calibrated to deliver 50-100 bacteria into the lungs. Four weeks later, mice were euthanized, and lung and spleen homogenates were prepared in PBS/Tween 80 (0.05%). Bacterial counts were determine by plating serial dilutions of individual whole organs on nutrient Middlebrook 7H11 Bacto Agar (BD Biosciences, Cockeysville, MD) and counting bacterial colony formation after 14-day incubation at 37° C. in humidified air and 5% CO₂. Data are expressed as Log 10 of the mean number of bacteria recovered±SD, and Log 10 Reduction in CFU=Log 10 CFU for the vaccinated group−Log 10 CFU for the Saline treated group.

### Results:

### Immune Responses to Ad5-ID83

C57BL/6 mice were immunized two times, three weeks apart, with Ad5-ID83.

Three weeks after the last immunization, splenocytes were prepared and assayed by ELISPOT to determine the relative number of IFN-γ-expressing splenocytes in response to medium alone, the mitogen ConA, and each of the fusion proteins.

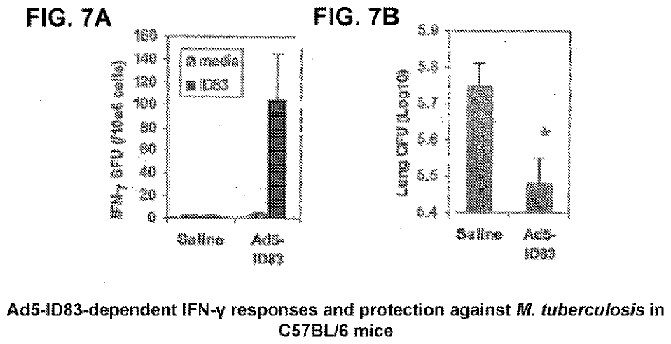

Immunization with Ad5-ID83 induced antigen specific IFN-γ recall responses by activated splenocytes (FIG. 7A). Injection with saline did not induce IFN-γ responses specific to ID83.

Protection Afforded by Ad5-ID83 Against an Aerosol Challenge with Mtb H37Rv.

Number of viable bacilli, expressed as mean Log 10 CFU, in the lung of mice vaccinated with 5×10⁸ Ad5-ID83 viral particles, were determined 4 weeks post aerosol challenge with ˜50 CFU of virulent M. tuberculosis H37RV.

The mean Log 10 CFU in the lung of mice immunized with Ad5-ID83 was compared to the mean Log 10 CFU obtained in mice receiving placebo (saline). The difference in mean Log 10 CFU in the saline group vs the vaccinated groups is expressed as Log 10 reduction in CFU.

Immunization of mice with two doses of Ad5-ID83 resulted in a decrease in viable Mtb bacilli in the lung of Mtb-infected mice of 0.27 (FIG. 7B). These results are promising in that protection against Mtb infection was achieved with only 2 doses of Ad5-ID83.

Together, these results indicate that immunization with Ad5-ID83 induced T cell immune responses and partially protected mice from an aerosol M. tuberculosis challenge.

### Example 26

### Immunotherapy with Mtb Rv1813, Rv2608, and Rv3620 Recombinant Proteins with the Adjuvant Gla-Se

This example demonstrates that immunization of mice with a mixture of recombinant proteins of the invention along with standard antibiotic therapy can prolong the life of M. tuberculosis-infected mice.

### Material & Methods:

### Recombinant Proteins and Adjuvant Formulations

Recombinant proteins were produced as described above. Glucopyranosyl lipid A (GLA) was obtained from Avanti (Alabaster, AL). Stable oil-in-water emulsions (−SE) were prepared.

Aerosol Challenge with M. tuberculosis

Female SWR/J mice were obtained from Jackson Laboratories and age-matched (5-7 week) within each experiment. Mice were challenged by low dose aerosol exposure with M. tuberculosis H37Rv strain (ATCC 35718; American Type Culture Collection, Manassas, VA) using a UW-Madison aerosol exposure chamber (Madison, WI) calibrated to deliver 50-100 bacteria into the lungs. Survival of mice was monitored for 225 days post-infection

### Therapy

Two weeks after an aerosol challenge with M. tuberculosis, standard antibiotic treatment was started. Mice were given 50 mg/l of rifampin and 85 mg/l isoniazide in their drinking water for 60 days. Some mice received additional immunotherapy and were immunized on day76, day97, and day118 post-infection with 6 μg of each Rv1813, Rv2608, and Rv3620 recombinant protein formulated with 20 μg of the adjuvant GLA-SE. Mice were injected with a total volume of 100 μl/mouse via the s.c. route. Mouse survival was monitored for 225 days.

### Results:

Protection Afforded by a Combination of Antibiotics+Rv1813, Rv2608, and Rv3620 with GLA-SE Immunotherapy, in M. tuberculosis-Infected Mice.

Survival of Mtb-infected mice treated with a standard regimen of rifampin+isoiazide antibiotics (Rx) or with a combination of Rx+immunization with Rv1813, Rv2608, and Rv3620 recombinant proteins formulated with 20 μg of the adjuvant GLA-SE (immunotherapy) was followed for 225 days post aerosol challenge with ˜50 CFU of virulent M. tuberculosis H37Rv.

The survival of mice treated with Rx+immunotherapy was compared to the survival of mice receiving Rx alone or placebo (saline).

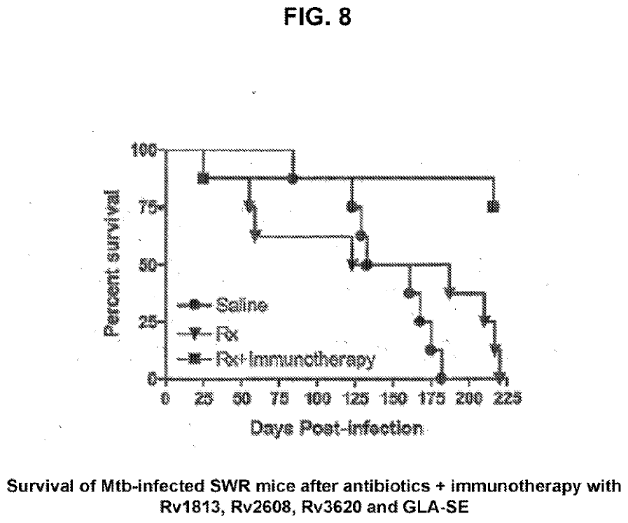

Treatment of mice with three doses of Rv1813, Rv2608, and Rv3620 recombinant proteins with GLA-SE, in addition to Rx, resulted in increased survival of Mtb-infected mice (FIG. 8). At day 225 post-infection, 75% of the animal vaccinated with Rv1813, Rv2608, and Rv3620 with GLA-SE were still alive, compared with 0% of the mice in the antibiotic (Rx) treatment alone group, and 0% in the placebo group.

These results demonstrate that protection against Mtb infection was achieved with antibiotics+3 doses of Rv1813, Rv2608, and Rv3620 with GLA-SE. In addition, treatment with antibiotics+Rv1813, Rv2608, and Rv3620 with GLA-SE protected Mtb-infected mice longer than antibiotics alone.

Together, these results indicate that immunotherapy with Rv1813, Rv2608, and Rv3620 with GLA-SE along with antibiotics induced immune responses that helped mice control an established M. tuberculosis infection.

### Example 27

### Serological Diagnosis of Tuberculosis

This example identifies M. tuberculosis antigens and antigen fusions having increased sensitivity and specificity for serological diagnosis of tuberculosis infection.

Polysorp 96 well plates (Nunc, Rochester, NY) were coated with 2 μg/ml recombinant antigen in bicarbonate buffer overnight at 4° C. and blocked for 2 hours at room temperature with PBST with 1% (w/v) BSA on a plate shaker. Serum were diluted appropriately to 1/200 in PBST with 0.1% BSA, added to each well and plates were incubated at room temperature for 2 hours with shaking. Plates were washed with PBST with 0.1% BSA and then HRP conjugated IgG immunoglobulin (Sigma, St. Louis, MO), diluted 1:10000 in PBST and 0.1% BSA, was added to each well and incubated at room temperature for 60 minutes with shaking. After washing, plates were developed with peroxidase color substrate (KPL, Baltimore MD) with reaction quenched by addition of 1N H₂SO₄ after 10 minutes. The corrected optical density of each well at 450-570 nm was read using a VERSAmax® microplate reader (Molecular Devices, Sunnyvale, CA).

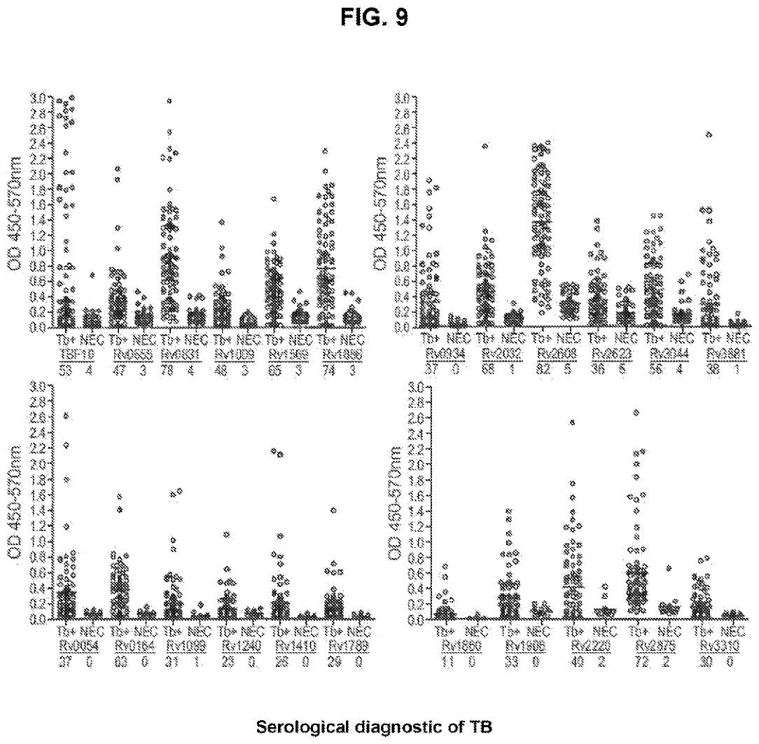

The results of these experiments are summarized in FIG. 9. A panel of sputum positive, tuberculosis confirmed serum samples (TB, N=80-92) and a panel of tuberculosis negative, healthy control serum (NEC, N=40-46) were analyzed for reactivity with selected tuberculosis antigens. A previously characterized antigen, TBF10, was used as a positive control and found to give seropostive responses to 53 of the 92 tuberculosis positive serum samples. The reactivity of individual antigens are shown in FIGS. 9, with all the antigens listed displaying reactivity to 11-82 of the tuberculosis serum samples, with low or no reactivity to the healthy controls. The reactivity of a given antigen varied to the serum panel such that 100% positive responses could be obtained through selection of proper antigen combinations.

### Example 28

### Cloning and Expression of Recombinant Rv0577

Using H37Rv genomic DNA as template, Rv0577 was PCR amplified using the following primers:

5′-Rv0577-NdeI
(SEQ ID NO: 295)
CAATTACATATGAGAGTTTTGTTGCTGGGACCG
3′: Rv0577-HindIII-
(SEQ ID NO: 296)
CAATTAAAGCTTCTACTTTCCAGAGCCCGCAACGC

Amplification was performed under the following conditions: 94° C. 0.5 min., 55° C. 0.5 min., 68° C. 2 min for 30 cycles, to give the product set forth in SEQ ID NO:185. The PCR product was digested with NdeI/HindIII and cloned into pET28.a vector. Rv0733 was expressed by host strain BL-21plysS. The supernatant was bound with Ni resin under denaturing conditions. The Ni-NTA purification was followed by an anion exchange purification. Dialyzed in 20 mM Tris pH 8. The amino acid sequence of the recombinant protein is set forth in SEQ ID NO: 186.

### Example 29

### Cloning and Expression of Recombinant Rv1626

Using H37Rv genomic DNA as template, Rv1626 was PCR amplified using the following primers:

5′-Rv1626-NdeI
(SEQ ID NO: 297)
CAATTACATATGACCGGCCCCACCACCGCGCC
3′-Rv1628-HIndIII
(SEQ ID NO: 298)
CAATTAAAGCTTTCAGGTGTCTTTGGGTGTTCCGAG

Amplification was performed under the following conditions: 94° C. 0.5 min., 55° C. 0.5 min., 68° C. 2 min for 30 cycles, to give the product set forth in SEQ ID NO:188. The PCR product was digested with NdeI/HindIII and cloned into pET28.a vector._Rv1626 was expressed by host strain BL-21plysS. The supernatant was bound with Ni resin under denaturing conditions. The Ni-NTA purification was followed by an anion exchange purification. Dialyzed in 20 mM Tris pH 8. The amino acid sequence of the recombinant protein is set forth in SEQ ID NO: 189.

### Example 30

### Cloning and Expression of Recombinant Rv0733

Using H37Rv genomic DNA as template, Rv0733 was PCR amplified using the following primers:

5′-Rv0733-5NdeI
(SEQ ID NO: 299)
CAATTACATATGAGAGTTTTGTTGCTGGGACCG
3′-Rv0733-HindIII
(SEQ ID NO: 300)
CAATTAAAGCTTCTACTTTCCAGAGCCCGCAACGC

Amplification was performed under the following conditions: 94° C. 0.5 min., 55° C. 0.5 min., 68° C. 2 min for 30 cycles, to give the product set forth in SEQ ID NO: 191. The PCR product was digested with NdeI/HindIII and cloned into pET28.a vector. Rv0733 was expressed by host strain BL-21plysS. The supernatant was bound with Ni resin under denaturing conditions. The Ni-NTA purification was followed by an anion exchange purification. Dialyzed in 20 mM Tris pH 8. The amino acid sequence of the recombinant protein is set forth in SEQ ID NO: 192.

### Example 31

### Cloning and Expression of Recombinant Rv2520

Using H37Rv genomic DNA as template, Rv2520 was PCR amplified using the following primers:

5′-Rv2520-NdeI-6his
(SEQ ID NO: 301)
CAATTACATATGCATCACCATCACCATCACGTG
GTGGACCGCGATCCCAATACC
3′-Rv2520-EcoRI
(SEQ ID NO: 302)
CAATTAGAATTCTCAGCGATTCCTGATCTTGTG

Amplification was performed under the following conditions: 94° C. 0.5 min., 55° C. 0.5 min., 68° C. 2 min for 30 cycles, to give the product set forth in SEQ ID NO:194. The PCR product was digested with NdeI/EcoRI and cloned into a modified pET 28a missing the upstream 6 histidine and the 5′linker sequence. Rv2520 was transformed into expression hosts BL-21pLysS and Rosetta pLysS. Both expressed equally, but proceeded with the BL-21 pLysS cell strain. Following cell lysis, the supernatant fraction was bound with Ni-NTA resin under denaturing conditions. The Ni-NTA purification was followed by an anion exchange purification. Purified fractions were dialyzed into 20 mM Tris pH 8. The amino acid sequence of the recombinant protein is set forth in SEQ ID NO: 195.

### Example 32

### Cloning and Expression of Recombinant Rv1253

Using H37Rv genomic DNA as template, Rv1253 was PCR amplified using the following primers:

5′-Rv1253-NdeI
(SEQ ID NO: 303)
CTGGATCCCATATGGCCTTCCCGGAATATTCGC
3′-Rv1253-EcoRI
(SEQ ID NO: 304)
CTAGCTGAATTCTCATCCGACGTGTTTCCGCCG

Amplification was performed under the following conditions: 94° C. 0.5 min., 55° C. 0.5 min., 68° C. 2 min for 30 cycles, to give the product set forth in SEQ ID NO:197. The PCR product was digested with NdeI/EcoRII and cloned into the pET28.a vector. Rv1511 was transformed into expression host Rosetta plysS. After lysis of a 1 L induction, the recombinant protein was expressed in the inclusion body pellet. Ni-NTA affinity purification was done under denaturing conditions, then dialyzed against 20 mM Tris pH 8.0. The amino acid sequence of the recombinant protein is set forth in SEQ ID NO: 198.

### Example 33

### Cloning and Expression of Recombinant Rv1980

Using H37Rv genomic DNA as template, Rv1980 was PCR amplified using the following primers:

5′-Rv1980-NdeI-24
(SEQ ID NO: 305)
CAATTACATATGGCGCCCAAGACCTACTGCGAG
3′-Rv1980-HindII
(SEQ ID NO: 306)
CAATTAAAGCTTCTAGGCCAGCATCGAGTCGATCGC

Amplification was performed under the following conditions: 94° C. 0.5 min., 55° C. 0.5 min., 68° C. 2 min for 30 cycles, to give the product set forth in SEQ ID NO: 200. The PCR product was digested with NdeI/HindIII and cloned into pET28.a vector. Rv1980 was transformed into expression host Rosetta plysS. After lysis of a 1 L induction, the recombinant protein was expressed in the inclusion body pellet. Ni-NTA affinity purification was done under denaturing conditions, then dialyzed against 20 mM Tris pH 8.0. The amino acid sequence of the recombinant protein is set forth in SEQ ID NO: 201.

### Example 34

### Cloning and Expression of Recombinant Rv3628

Using H37Rv genomic DNA as template, Rv3628 was PCR amplified using the following primers:

5′-Rv3628-Nde-6hisI
(SEQ ID NO: 307)
CAATTACATATGCATCACCATCACCATCACATGCAATTCGACG
TGACCATC
3′-Rv3628-EcoRI
(SEQ ID NO: 308)
CAATTAGAATTCTCAGTGTGTACCGGCCTTGAAGCG

Amplification was performed under the following conditions: 94° C. 0.5 min., 55° C. 0.5 min., 68° C. 2 min for 30 cycles, to give the product set forth in SEQ ID NO: 203. Using H37Rv genomic DNA as template, Rv3628 was PCR′d with conditions 95° C. 1 min., 58° C. 1 min., 72° C. 1.5 min for 35 cycles. The PCR product was digested with NdeI/EcoRI and cloned into pET 17b. Rv3628 was transformed into expression hosts BL-21plysE and Rosetta plysS. Both expressed equally, but proceeded with the plysE construct. After lysis of a 1 L induction, it went into the inclusion body. Ni-NTA was done under denaturing conditions, then dialyzed against 10 mM Tris pH 8.0. The amino acid sequence of the recombinant protein is set forth in SEQ ID NO: 204.

### Example 35

### Cloning and Expression of Recombinant Rv1844

Using H37Rv genomic DNA as template, Rv1844 was PCR amplified using the following primers:

5′-Rv1884-NdeI-6his30
(SEQ ID NO: 309)
CAATTACATATGCATCACCATCACCATCACACTTCCGGCGAT
ATGTCGAGC
3′-Rv1884-EcoRI
(SEQ ID NO: 310)
CAATTAGAATTCTCAGCGCGGAATACTTGCCTG

Amplification was performed under the following conditions: 94° C. 0.5 min., 55° C. 0.5 min., 68° C. 2 min for 30 cycles, to give the product set forth in SEQ ID NO: 206. The PCR product was digested with NdeI/EcoRI and cloned into pET 17b. Plasmid containing the Rv1884 gene was transformed into expression hosts BL-21plysE and plysS. Both expressed equally, but proceeded with the plysE. After lysis of a 1 L induction, it remained in the insoluble inclusion body fraction. Ni-NTA was done under denaturing conditions, then dialyzed against 10 mM Tris pH 8.0. The amino acid sequence of the recombinant protein is set forth in SEQ ID NO: 207.

### Example 36

### Cloning and Expression of Recombinant Rv3872

Using H37Rv genomic DNA as template, Rv3872 was PCR amplified using the following primers:

5′-Rv3872-NdeI
(SEQ ID NO: 311)
GTGCTAGCCATATGGAAAAAATGTCACATGATC
3′-Rv3872-HindIII
(SEQ ID NO: 312)
CTGGATCCAAGCTTCTATTCGGCGAAGACGCCGGC

Amplification was performed under the following conditions: 94° C. 0.5 min., 55° C. 0.5 min., 68° C. 2 min for 30 cycles, to give the product set forth in SEQ ID NO: 209. The PCR product was digested with NdeI/HindIII and cloned into pET28.a vector. Rv3872 was transformed into expression host Rosetta plysS. After lysis of a 1 L induction, the recombinant protein was expressed in the soluble supernatant fraction. Ni-NTA affinity purification was done 2× under native conditions, then dialyzed against 20 mM Tris pH 8.0. The amino acid sequence of the recombinant protein is set forth in SEQ ID NO: 210.

### Example 37

### Cloning and Expression of Recombinant Rv3873

Using H37Rv genomic DNA as template, Rv3873 was PCR amplified using the following primers:

5′-Rv3873-NdeI
(SEQ ID NO: 313)
GTGCTAGCCATATGCTGTGGCACGCAATGCCAC
3′-3873-HindIII
(SEQ ID NO: 314)
CTGGATCCAAGCTTTCACCAGTCGTCCTCTTCGTC

Amplification was performed under the following conditions: 94° C. 0.5 min., 55° C. 0.5 min., 68° C. 2 min for 30 cycles, to give the product set forth in SEQ ID NO: 212. The PCR product was digested with NdeI/HindII and cloned into pET28a vector. Plasmid containing the Rv3873 gene was transformed into expression host Rosetta plysS. After lysis of a 1 L induction, the recombinant protein was expressed in the soluble supernatant fraction. Ni-NTA affinity purification was done 2× under native conditions, then dialyzed against 20 mM Tris pH 8.0. The amino acid sequence of the recombinant protein is set forth in SEQ ID NO: 213.

### Example 38

### Cloning and Expression of Recombinant Rv1511

Using H37Rv genomic DNA as template, Rv1511 was PCR amplified using the following primers:

5′-Rv1511-NdeI
(SEQ ID NO: 315)
CAATTACATATGCATCACCATCACCATCACGTGAAGCGAGCG
CTCATCACC
3′-Rv1511-EcoRI
(SEQ ID NO: 316)
CAATTAGAATTCTCATGTCCGGCCGGCGATCATCG

Amplification was performed under the following conditions: 94° C. 0.5 min., 55° C. 0.5 min., 68° C. 2 min for 30 cycles, to give the product set forth in SEQ ID NO: 214. The PCR product was digested with NdeI/EcoRI and cloned into pET 28a, minus the 5′ linker. Rv1511 was transformed into expression hosts BL-21plysS and Rosetta plysS. Both expressed equally, but proceeded with the BL-21 cells. After lysis of a 1 L induction, the recombinant protein was expressed in the inclusion body pellet. Ni-NTA affinity purification was done under denaturing conditions, then dialyzed against 10 mM Tris pH 9.5. The amino acid sequence of the recombinant protein is set forth in SEQ ID NO: 215.

### Example 39

### Cloning and Expression of Recombinant Fusion Protein Id93

The following primers were used in for cloning the fusion construct ID93, which comprises fusion partners derived from Rv3619, Rv1813, Rv3620 and Rv2608:

5′: Rv1813mat-5NdeI-KpnI
(SEQ ID NO: 218)
CAATTACATATGGGTACCCATCTCGCCAACGGTTCGATG
3′: Rv1813mat-3SacIgo
(SEQ ID NO: 219)
CAATTAGAGCTCGTTGCACGCCCAGTTGACGAT
5′: Rv3620-5SacI
(SEQ ID NO: 220)
CAATTAGAGCTCATGACCTCGCGTTTTATGACG
3′: Rv3620-3SalIgo
(SEQ ID NO: 221)
CAATTAGTCGACGCTGCTGAGGATCTGCTGGGA
5′: Rv2608-5SalI
(SEQ ID NO: 222)
CAATTAGTCGACATGAATTTCGCCGTTTTGCCG
3′: Rv2608-3ScaI-HindIII
(SEQ ID NO: 223)
CAATTAAAGCTTTTAAGTACTGAAAAGTCGGGGTAGCGCCGG
5′: Rv3619-5NdeI
(SEQ ID NO: 224)
CAATTACATATGACCATCAACTATCAATTC
3′: Rv3619-3KpnI
(SEQ ID NO: 225)
CAATTAGGTACCGGCCCAGCTGGAGCCGACGGC

Rv1813 and Rv3620 were PCR amplified from H37Rv genomic template DNA (94° C. for 0:30; 58° C. for 0:30; 58° C. for 1:30; 35 cycles). Rv1813 was digested with NdeI/SacI then cloned into pET28.a vector. Rv3620 was digested with SacI/SaII then ligated into the Rv1813pET construct. The fusion construct has a polynucleotide sequence set forth in SEQ ID NO: 217, encoding the fusion protein set forth in SEQ ID NO: 226. Rv2608 was amplified from plasmid template by PCR (94° C. for 0:30; 58° C. for 0:30; 68° C. for 1:30; 35 cycles). Product was digested with SaII/HindIII and cloned into pET28.a-Rv1813-3620 vector. Rv3619 was amplified same as above and digested with NdeI/KpnI then ligated into the ID83 vector. ID93 was expressed in host BL-21plysS (2 L, 2XYT growth media, 37° C.). Induced with 1 mM IPTG at OD 0.77 and harvested at OD 1.93. Cell pellet was suspended in lysis buffer (20 mM Tris pH8, 100 mM NaCl, 2 mM PMSF) and froze. The cell pellet was then thawed, lysed by sonication, and spun at 7,000 rcf for 20 minutes ID83 is an inclusion body protein. The pellet was washed 2× with 1% Chaps. The pellet was solubilized in 60 mL in binding buffer (8M urea, 20 mM Tris pH 8, 100 mM NaCl) and bound to 16 mL Ni-NTA resin at RT for 1 hour. The resin was washed (50 mL 0.5% DOC for 20 minutes; 80 mL 60% IPA for 30 minutes, 50 mL 0.5% DOC rinse) and then eluted with binding buffer with 300 mM imidazole. The supernatant from the first bind was bound to an additional 8 mL resin and processed as indicated above. The aforementioned purifications removed breakdown products. Another Ni-NTA bind was done overnight at 4° C. in 160 mL (binding buffer with 50 mM NaCl) with 32 mL resin. The resin was washed and eluted as indicated above. The fractions from this bind were dialyzed in 20 mM Tris pH8.

### Example 40

### Cloning and Expression of Recombinant Fusion Protein Id91

The following primers were used in for cloning the fusion construct ID91, which comprises fusion partners derived from Rv3619, Rv2389, Rv3478 and Rv1886:

5′-Rv3619-5NdeI
(SEQ ID NO: 228)
CAATTACATATGACCATCAACTATCAATTC
3′-Rv3619-3KpnI
(SEQ ID NO: 229)
CAATTAGGTACCGGCCCAGCTGGAGCCGACGG
5′-Rv2389-KpnI
(SEQ ID NO: 230)
TGGGCCGGTACCGACGACATCGATTGGGACGCC
3′-Rv2389-BamHI
(SEQ ID NO: 231)
AATCCACCACGGATCCATCGTCCCTGCTCCCCGAAC
5′-Rv3478-BamHI
(SEQ ID NO: 232)
CAGGGACGATGGATCCGTGGTGGATTTCGGGGCGTTAC
3′-Rv3478-EcoRI
(SEQ ID NO: 233)
CCGGGAGAAGAATTCTCCGGCGGCCGGTGTGCGGG
5′-Rv1886-EcoRI
(SEQ ID NO: 234)
GCCGCCGGAGAATTCTTCTCCCGGCCGGGGTGCC
3′-Rv1886matR HindIII
(SEQ ID NO: 235)
GATATCAAGCTTTCAGCCGGCGCCTAACGAAC

The fusion construct has a polynucleotide sequence set forth in SEQ ID NO: 227, encoding the fusion protein set forth in SEQ ID NO: 236.

### Example 41

### Cloning and Expression of Recombinant Fusion Protein Id71

The following primers were used in for cloning the fusion construct ID71, which comprises fusion partners derived from Rv3619, Rv2389, Rv3478 (N180) and Rv1886:

5′-Rv3619-5NdeI
(SEQ ID NO: 238)
CAATTACATATGACCATCAACTATCAATTC
3′-Rv3619-3KpnI
(SEQ ID NO: 239)
CAATTAGGTACCGGCCCAGCTGGAGCCGACGG
5′-Rv2389-KpnI
(SEQ ID NO: 240)
TGGGCCGGTACCGACGACATCGATTGGGACGCC
3′-Rv2389-BamHI
(SEQ ID NO: 241)
AATCCACCACGGATCCATCGTCCCTGCTCCCCGAAC
5′-Rv3478-N180-EcoRI
(SEQ ID NO: 242)
CGGCCGGGAGAAGAATTCCCCGCCGGGGTTGGTGATCAG
5′-Rv1886-EcoRI
(SEQ ID NO: 243)
GCCGCCGGAGAATTCTTCTCCCGGCCGGGGCTGCC
3′-Rv1886matR HindIII
(SEQ ID NO: 244)
GATATCAAGCTTTCAGCCGGCGCCTAACGAAC

The fusion construct has a polynucleotide sequence set forth in SEQ ID NO: 237, encoding the fusion protein set forth in SEQ ID NO: 245.

### Example 42

### Cloning and Expression of Recombinant Fusion Protein Id114

The following primers were used in for cloning the fusion construct ID114, which comprises fusion partners derived from Rv1813, Rv3620, Rv2608 and Rv1886:

5′: Rv2608-5SalI
(SEQ ID NO: 247)
CAATTAGTCGACATGAATTTCGCCGTTTTGCCG
3′: Rv2608-3ScaI-HindIII
(SEQ ID NO: 248)
CAATTAAAGCTTTTAAGTACTGAAAAGTCGGGGTAGCGCCGG
5′-Rv1886-2608-ScaI
(SEQ ID NO: 249)
CGGCGCTACCCCGACTTTTCAGTACTTTCTCCCGGCCGGGG
CTGCCG
3′-Rv1886matR HindIII
(SEQ ID NO: 250)
GATATCAAGCTTTCAGCCGGCGCCTAACGAAC

Rv1813 and Rv3620 were PCR amplified from H37Rv genomic template DNA (94° C. for 0:30; 58° C. for 0:30; 58° C. for 1:30; 35 cycles). The fusion construct has a polynucleotide sequence set forth in SEQ ID NO: 246, encoding the fusion protein set forth in SEQ ID NO: 251.

### Example 43

### Cloning and Expression of Recombinant Fusion Protein Id125

The following primers were used in for cloning the fusion construct ID125, which comprises fusion partners derived from Rv3619, Rv1813, Rv3620, Rv2608 and Rv1886:

5′: Rv2608-5SalI
(SEQ ID NO: 253)
CAATTAGTCGACATGAATTTCGCCGTTTTGCCG
3′: Rv2608-3ScaI-HindIII
(SEQ ID NO: 254)
CAATTAAAGCTTTTAAGTACTGAAAAGTCGGGGTAGCGCCGG
5′-Rv1886-2608-ScaI
(SEQ ID NO: 255)
CGGCGCTACCCCGACTTTTCAGTACTTTCTCCCGGCCGGGG
CTGCCG
3′-Rv1886matR HindIII
(SEQ ID NO: 256)
GATATCAAGCTTTCAGCCGGCGCCTAACGAAC

The fusion construct has a polynucleotide sequence set forth in SEQ ID NO: 252, encoding the fusion protein set forth in SEQ ID NO: 257.

### Example 44

### Cloning and Expression of Recombinant Fusion Protein Did85

The following primers were used in for cloning the fusion construct DID85, which comprises fusion partners derived from Rv2032, Rv2875, and Rv0831:

5′-Rv2032-NdeI-6his
(SEQ ID NO: 259)
GATACACATATGCACCATCACCATCACCACATGCCGGACACC
ATGGTGAC
3′-Rv2032-GGSGGS-BamHI
(SEQ ID NO: 260)
CATGGATCCGCTACCGCCAGAACCACCCCGGTGATCCTTAG
CCCGAAC
5′-Rv2875-BamHI
(SEQ ID NO: 261)
GGTGGTTCTGGCGGTAGCGGATTCATGGGCGATCTGGTGAG
CCCG
3′-Rv2875R-EcoRI
(SEQ ID NO: 262)
CATGAATTCAGAACCGCCGCTTCCGCCCGCCGGAGGCATTA
GCACGC
5′-Rv0831F-EcoRI
(SEQ ID NO: 263)
GGCGGAAGCGGCGGTTCTGAATTCATGCTCCCCGAGACAAA
TCAG
3′-Rv0831R-HindIII
(SEQ ID NO: 264)
TAGAATTCAAGCTTTTACTGGCGAAGCAGCTCATC

The genes for Rv2032, Rv2875, and Rv0831 were PCR amplified from existing Plasmid DNA (94° C. for 0:30; 58° C. for 0:30; 58° C. for 1:30; 30 cycles) using the above primer sequences. The three amplified PCR products were used in a second round of PCR to amplify the full length fusion gene product using the 5′-Rv2032-NdeI-6his and 3′-Rv0831R-HindIII primers. The resulting PCR product was digested with NdeI/HindIII and cloned into pET29a vector. DID85 was expressed by host strain BL-21plysS. The fusion construct has a polynucleotide sequence set forth in SEQ ID NO: 258, encoding the fusion protein set forth in SEQ ID NO: 265. After lysis of a 1 L induction, it went into the inclusion body. Ni-NTA was done under denaturing conditions, followed by anion exchange chromatography. Purified fractions were dialyzed against 10 mM Tris pH 8.0.

### Example 45

### Cloning and Expression of Recombinant Fusion Protein Did92

The following primers were used in for cloning the fusion construct DID92, which comprises fusion partners derived from Rv3044, Rv1009, and Rv0614:

5′-Rv3044-NdeI-6his
(SEQ ID NO: 267)
GATACACATATGCACCATCACCATCACCACATGGGCAGCAGC
CATCATCATC
3′-Rv3044-NcoI
(SEQ ID NO: 268)
CATATCGAGCTCGTTGATCGGCGCGTCGACCC
5′-Rv1009-NcoI-GGSGGS linker
(SEQ ID NO: 269)
ATCAACGAGCTCGGAGGTTCTGGTGGAAGCGCATGCAAAAC
GGTGACGTTGAC
3′-Rv1009-EcoRI
(SEQ ID NO: 270)
CATATCGAATTCGCGCGCACCCGCTCGTGCAGC
5′-Rv0164-EcoRI-GGSGGS linker
(SEQ ID NO. 271)
CATGTCGAATTCGGTGGAAGCGGAGGTTCTATGACGGCAAT
CTCGTGCTCAC
3′-Rv0164-HindIII
(SEQ ID NO: 272)
CATATCAAGCTTTTAGCTGGCCGCCAGCTGCTC

The fusion construct has a polynucleotide sequence set forth in SEQ ID NO: 266, encoding the fusion protein set forth in SEQ ID NO: 273.

### Example 46

### Cloning and Expression of Recombinant Fusion Protein Did108

The following primers were used in for cloning the fusion construct DID108, which comprises fusion partners derived from Rv3872, Rv3873, Rv3875 and Rv3881:

5′-Rv3872-NdeI-6his
(SEQ ID NO: 275)
GATACACATATGCACCATCACCATCACCACATGGAAAAAATG
TCACATGATC
3′-Rv3872-SacI
(SEQ ID NO: 276)
GATACATGAGCTCTTCGGCGAAGACGCCGGCGGC
5′-Rv3873-SacI-GGSGGS linker
(SEQ ID NO: 277)
GATACAGAGCTCGGAGGTTCCGGTGGAAGCATGCTGTGGCA
CGCAATGCC
3′-Rv3873-EcoRI
(SEQ ID NO: 278)
GATACAGAATTCCCAGTCGTCCTCTTCGTCCCAG
5′-Rv3875-EcoRI-GGSGGS linker
(SEQ ID NO: 279)
GACAGAATTCGGTGGCAGTGGAGGATCTATGACAGAGCAGC
AGTGGAAT
3′-Rv3875-NheI
(SEQ ID NO: 280)
CATATCAGCTAGC TGCGAACATCCCAGTGACGTTG
5′-Rv3881-NheI-GGSGGS linker
(SEQ ID NO: 281)
CATATCAGCTAGCGGAGGTTCCGGTGGAAGCATGACGCAGT
CGCAGACCGTG
3′-Rv3881-HindIII
(SEQ ID NO: 282)
CATATCAAAGCTT TCACTTCGACTCCTTACTGTC

The fusion construct has a polynucleotide sequence set forth in SEQ ID NO: 274, encoding the fusion protein set forth in SEQ ID NO: 283.

### Example 47

### Cloning and Expression of Recombinant Fusion Protein DID93

The following primers were used in for cloning the fusion construct DID93, which comprises fusion partners derived from Rv1099, Rv0655, and Rv0054:

5′-Rv1099-NdeI
(SEQ ID NO: 285)
TAGGATCCCATATGGAGCTGGTCCGGGTGACC
3′-Rv1099-EcoRI-GGSGGS linker
(SEQ ID NO: 286)
CACGAATTCGCTTCCACCAGAACCTCCGGGCAATGGGTACA
CGGCGC
5′-Rv0655-EcoRI-GGSGGS Linker
(SEQ ID NO: 287)
GGAGGTTCTGGTGGAAGCGAATTCGTGCGATACAGTGACTC
ATAC
3′-Rv0655-SacI
(SEQ ID NO: 288)
GCCACGAGCTCAGAACCGCCGCTTCCACCCTGGCCGATTTC
GTGCACCGC
5′-Rv0054-SacI-GGSGGS linker
(SEQ ID NO: 289)
GCCAGGGTGGAAGCGGCGGTTCTGAGCTCGTGGCTGGTGA
CACCACCATC
3′Rv0054-HindIII
(SEQ ID NO: 290)
CAATTAAAGCTTTCAGAATGGCGGTTCGTCATCGCC

The fusion construct has a polynucleotide sequence set forth in SEQ ID NO:284, encoding the fusion protein set forth in SEQ ID NO: 291

### Example 48

### Cloning and Expression of Recombinant Fusion Protein Rv3875

Using H37Rv genomic DNA as template, Rv3875 was PCR amplified using the following primers:

5′-Rv3875-6His-NdeI
(SEQ ID NO: 317)
CCATTACATATGCATCACCATCACCATCACATGACAGAGCAG
CAGTGGAA
3′-Rv3875-EcoRI
(SEQ ID NO: 318)
CCATTAGAATTCCTATGCGAACATCCCAGTGAC

The amino acid sequence of the recombinant protein is set forth in SEQ ID NO: 294.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method for stimulating an immune response comprising:
administering to a subject an effective amount of a composition comprising two or more Mycobacterium tuberculosis antigens,
wherein the two or more antigens comprise the antigen Rv3478 comprising the amino acid sequence set forth in SEQ ID NO:41 and the antigen Rv3619 comprising the amino acid sequence set forth in SEQ ID NO:46, or
an antigen comprising an amino acid sequence having at least 90% sequence identity to the amino acid sequence set forth in SEQ ID NO: 41 and an antigen comprising an amino acid sequence having at least 90% sequence identity to the amino acid sequence set forth in SEQ ID NO: 46; and
wherein the two or more antigens are covalently linked in the form of a fusion polypeptide.

2. The method of claim 1, wherein the two or more antigens comprise an antigen comprising an amino acid sequence having at least 99% sequence identity to the amino acid sequence set forth in SEQ ID NO: 41 and an antigen comprising an amino acid sequence having at least 99% sequence identity to the amino acid sequence set forth in SEQ ID NO: 46.

3. The method of claim 1, wherein the two or more antigens are directly linked.

4. The method of claim 1, wherein the two or more antigens are linked via an amino acid linker.

5. The method of claim 1, wherein the composition further comprises an immunostimulant.

6. The method of claim 5, wherein the immunostimulant is selected from the group consisting of glucopyranosyl lipid adjuvant (GLA), adjuvant system-2 (AS-2), monophosphoryl lipid A, 3-de-O-acylated monophosphoryl lipid A, incomplete Freund's adjuvant (IFA), Quillaja saponaria 21 (QS21), cell wall skeleton (CWS), trehalose dicorynomycolate (TDM), aminoalkyl glucosaminide phosphates (AGPs), CpG-containing oligonucleotides, Toll-like receptor agonists, LelF, saponins, saponin mimetics, biological and synthetic lipid A, imiquimod, gardiquimod, resiquimod, polyI:C, and flagellin, or a combination thereof.

7. The method of claim 6, wherein the immunostimulant is GLA.

8. The method of claim 1, wherein the two or more antigens further comprise the antigen Rv2389 comprising the amino acid sequence set forth in SEQ ID NO: 21 or an antigen comprising an amino acid sequence having at least 90% sequence identity to the amino acid sequence set forth in SEQ ID NO: 21.

9. The method of claim 8, wherein the two or more antigens further comprise an antigen comprising an amino acid sequence having at least 99% sequence identity to the amino acid sequence set forth in SEQ ID NO: 21.

10. The method of claim 1, wherein the two or more antigens further comprise the antigen Rv1886 comprising the amino acid sequence set forth in SEQ ID NO: 145 or an antigen comprising an amino acid sequence having at least 90% sequence identity to the amino acid sequence set forth in SEQ ID NO: 145.

11. The method of claim 10, wherein the two or more antigens further comprise an antigen comprising an amino acid sequence having at least 99% sequence identity to the amino acid sequence set forth in SEQ ID NO: 145.

12. The method of claim 1, wherein the two or more antigens further comprise the antigen Rv2389 comprising the amino acid sequence set forth in SEQ ID NO: 21 or an antigen comprising an amino acid sequence having at least 90% sequence identity to the amino acid sequence set forth in SEQ ID NO: 21 and the antigen Rv1886 comprising the amino acid sequence set forth in SEQ ID NO: 145 or an antigen comprising an amino acid sequence having at least 90% sequence identity to the amino acid sequence set forth in SEQ ID NO: 145.

13. The method of claim 12, wherein the two or more antigens further comprise an antigen comprising an amino acid sequence having at least 99% sequence identity to the amino acid sequence set forth in SEQ ID NO: 21 and an amino acid sequence having at least 99% sequence identity to the amino acid sequence set forth in SEQ ID NO: 145.

14. The method of claim 1, wherein the fusion polypeptide is ID91 comprising the amino acid sequence set forth in SEQ ID NO: 236 or an amino acid sequence having at least 90% sequence identity to the amino acid sequence set forth in SEQ ID NO: 236.

15. The method of claim 14, wherein the fusion polypeptide comprises an amino acid sequence having at least 99% sequence identity to the amino acid sequence set forth in SEQ ID NO: 236.


SEQUENCE LISTING (318 sequences)

SEQ ID NO: 1 - protein, 161 aa, Mycobacterium tuberculosis
MTAISCSPRPRYASRMPVLSKTVEVTADAASIMAIVADIERYPEWNEGVKGAWVLARYDDGRPSQVRLDTAVQGIEGTYIHAVYYPGENQIQTVMQQGELFAKQEQLFSVVATGAASLLTVDMDVQVTMPVPEPMVKMLLNNVLEHLAENLKQRAEQLAAS

SEQ ID NO: 2 - DNA, 495 bp, Mycobacterium tuberculosis
  source 1..495: mol_type = genomic DNA
catatgacggcaatctcgtgctcaccgcgacccaggtatgcttcccgaatgccagttttgagcaagaccgtcgaggtcaccgccgacgccgcatcgatcatggccatcgttgccgatatcgagcgctacccagagtggaatgaaggggtcaagggcgcatgggtgctcgctcgctacgatgacgggcgtcccagccaggtgcggctcgacaccgctgttcaaggcatcgagggcacctatatccacgccgtgtactacccaggcgaaaaccagattcaaaccgtcatgcagcagggtgaactgtttgccaagcaggagcagctgttcagtgtggtggcaaccggcgccgcgagcttgctcacggtggacatggacgtccaggtcaccatgccggtgcccgagccgatggtgaagatgctgctcaacaacgtcctggagcatctcgccgaaaatctcaagcagcgcgccgagcagctggcggccagctaaaagctt

SEQ ID NO: 3 - protein, 181 aa, Mycobacterium tuberculosis
MGSSHHHHHHSSGLVPRGSHMTAISCSPRPRYASRMPVLSKTVEVTADAASIMAIVADIERYPEWNEGVKGAWVLARYDDGRPSQVRLDTAVQGIEGTYIHAVYYPGENQIQTVMQQGELFAKQEQLFSVVATGAASLLTVDMDVQVTMPVPEPMVKMLLNNVLEHLAENLKQRAEQLAAS

SEQ ID NO: 4 - DNA, 33 bp, synthetic construct
  misc_feature 1..33: note = Cloning primer
  source 1..33: mol_type = other DNA
taggatcccatatgacggcaatctcgtgctcac

SEQ ID NO: 5 - DNA, 35 bp, synthetic construct
  misc_feature 1..35: note = Cloning primer
  source 1..35: mol_type = other DNA
tagaattcaagcttttagctggccgccagctgctc

SEQ ID NO: 6 - protein, 328 aa, Mycobacterium tuberculosis
VVDAHRGGHPTPMSSTKATLRLAEATDSSGKITKRGADKLISTIDEFAKIAISSGCAELMAFATSAVRDAENSEDVLSRVRKETGVELQALRGEDESRLTFLAVRRWYGWSAGRILNLDIGGGSLEVSSGVDEEPEIALSLPLGAGRLTREWLPDDPPGRRRVAMLRDWLDAELAEPSVTVLEAGSPDLAVATSKTFRSLARLTGAAPSMAGPRVKRTLTANGLRQLIAFISRMTAVDRAELEGVSADRAPQIVAGALVAEASMRALSIEAVEICPWALREGLILRKLDSEADGTALIESSSVHTSVRAVGGQPADRNAANRSRGSKP

SEQ ID NO: 7 - DNA, 996 bp, Mycobacterium tuberculosis
  source 1..996: mol_type = genomic DNA
catatggtcgatgcccaccgcggcggccacccgaccccgatgagctcgacgaaggccacgctgcggctggccgaggccaccgacagctcgggcaagatcaccaagcgcggagccgacaagctgatttccaccatcgacgaattcgccaagattgccatcagctcgggctgtgccgagctgatggccttcgccacgtcggcggtccgcgacgccgagaattccgaggacgtcctgtcccgggtgcgcaaagagaccggtgtcgagttgcaggcgctgcgtggggaggacgagtcacggctgaccttcctggccgtgcgacgatggtacgggtggagcgctgggcgcatcctcaacctcgacatcggcggcggctcgctggaagtgtccagtggcgtggacgaggagcccgagattgcgttatcgctgcccctgggcgccggacggttgacccgagagtggctgcccgacgatccgccgggccggcgccgggtggcgatgctgcgagactggctggatgccgagctggccgagcccagtgtgaccgtcctggaagccggcagccccgacctggcggtcgcaacgtcgaagacgtttcgctcgttggcgcgactaaccggtgcggccccatccatggccgggccgcgggtgaagaggaccctaacggcaaatggtctgcggcaactcatcgcgtttatctctaggatgacggcggttgaccgtgcagaactggaaggggtaagcgccgaccgagcgccgcagattgtggccggcgccctggtggcagaggcgagcatgcgagcactgtcgatagaagcggtggaaatctgcccgtgggcgctgcgggaaggtctcatcttgcgcaaactcgacagcgaagccgacggaaccgccctcatcgagtcttcgtctgtgcacacttcggtgcgtgccgtcggaggtcagccagctgatcggaacgcggccaaccgatcgagaggcagcaaaccatgaaagctt

SEQ ID NO: 8 - protein, 348 aa, Mycobacterium tuberculosis
MGSSHHHHHHSSGLVPRGSHMVDAHRGGHPTPMSSTKATLRLAEATDSSGKITKRGADKLISTIDEFAKIAISSGCAELMAFATSAVRDAENSEDVLSRVRKETGVELQALRGEDESRLTFLAVRRWYGWSAGRILNLDIGGGSLEVSSGVDEEPEIALSLPLGAGRLTREWLPDDPPGRRRVAMLRDWLDAELAEPSVTVLEAGSPDLAVATSKTFRSLARLTGAAPSMAGPRVKRTLTANGLRQLIAFISRMTAVDRAELEGVSADRAPQIVAGALVAEASMRALSIEAVEICPWALREGLILRKLDSEADGTALIESSSVHTSVRAVGGQPADRNAANRSRGSKP

SEQ ID NO: 9 - DNA, 32 bp, synthetic construct
  misc_feature 1..32: note = Cloning primer
  source 1..32: mol_type = other DNA
taggatcccatatggtcgatgcccaccgcggc

SEQ ID NO: 10 - DNA, 35 bp, synthetic construct
  misc_feature 1..35: note = Cloning primer
  source 1..35: mol_type = other DNA
tagaattcaagctttcatggtttgctgcctctcga

SEQ ID NO: 11 - protein, 94 aa, Mycobacterium tuberculosis
MCGDQSDHVLQHWTVDISIDEHEGLTRAKARLRWREKELVGVGLARLNPADRNVPEIGDELSVARALSDLGKRMLKVSTHDIEAVTHQPARLLY

SEQ ID NO: 12 - DNA, 315 bp, Mycobacterium tuberculosis
  source 1..315: mol_type = genomic DNA
catatgcatcaccatcaccatcacatgtgcggcgaccagtcggatcacgtgctgcagcactggaccgtcgacatatcgatcgacgaacacgaaggattgactcgggcgaaggcacggctgcgttggcgggaaaaggaattggtgggtgttggcctggcaaggctcaatccggccgaccgcaacgtccccgagatcggcgatgaactctcggtcgcccgagccttgtccgacttggggaagcgaatgttgaaggtgtcgacccacgacatcgaagctgttacccatcagccggcgcgattgttgtattgagaattc

SEQ ID NO: 13 - protein, 101 aa, Mycobacterium tuberculosis
MHHHHHHMCGDQSDHVLQHWTVDISIDEHEGLTRAKARLRWREKELVGVGLARLNPADRNVPEIGDELSVARALSDLGKRMLKVSTHDIEAVTHQPARLLY

SEQ ID NO: 14 - DNA, 51 bp, synthetic construct
  misc_feature 1..51: note = Cloning primer
  source 1..51: mol_type = other DNA
caattacatatgcatcaccatcaccatcacatgtgcggcgaccagtcggat

SEQ ID NO: 15 - DNA, 33 bp, synthetic construct
  misc_feature 1..33: note = Cloning primer
  source 1..33: mol_type = other DNA
caattagaattctcaatacaacaatcgcgccgg

SEQ ID NO: 16 - protein, 143 aa, Mycobacterium tuberculosis
MITNLRRRTAMAAAGLGAALGLGILLVPTVDAHLANGSMSEVMMSEIAGLPIPPIIHYGAIAYAPSGASGKAWHQRTPARAEQVALEKCGDKTCKVVSRFTRCGAVAYNGSKYQGGTGLTRRAAEDDAVNRLEGGRIVNWACN

SEQ ID NO: 17 - DNA, 363 bp, Mycobacterium tuberculosis
  source 1..363: mol_type = genomic DNA
catatgcatcaccatcaccatcatctcgccaacggttcgatgtcggaagtcatgatgtcggaaattgccgggttgcctatccctccgattatccattacggggcgattgcctatgcccccagcggcgcgtcgggcaaagcgtggcaccagcgcacaccggcgcgagcagagcaagtcgcactagaaaagtgcggtgacaagacttgcaaagtggttagtcgcttcaccaggtgcggcgcggtcgcctacaacggctcgaaataccaaggcggaaccggactcacgcgccgcgcggcagaagacgacgccgtgaaccgactcgaaggcgggcggatcgtcaactgggcgtgcaactaagaattc

SEQ ID NO: 18 - protein, 117 aa, Mycobacterium tuberculosis
MHHHHHHLANGSMSEVMMSEIAGLPIPPIIHYGAIAYAPSGASGKAWHQRTPARAEQVALEKCGDKTCKVVSRFTRCGAVAYNGSKYQGGTGLTRRAAEDDAVNRLEGGRIVNWACN

SEQ ID NO: 19 - DNA, 51 bp, synthetic construct
  misc_feature 1..51: note = Cloning primer
  source 1..51: mol_type = other DNA
caattacatatgcatcaccatcaccatcaccatctcgccaacggttcgatg

SEQ ID NO: 20 - DNA, 33 bp, synthetic construct
  misc_feature 1..33: note = Cloning primer
  source 1..33: mol_type = other DNA
caattagaattcttagttgcacgcccagttgac

SEQ ID NO: 21 - protein, 154 aa, Mycobacterium tuberculosis
MTPGLLTTAGAGRPRDRCARIVCTVFIETAVVATMFVALLGLSTISSKADDIDWDAIAQCESGGNWAANTGNGLYGGLQISQATWDSNGGVGSPAAASPQQQIEVADNIMKTQGPGAWPKCSSCSQGDAPLGSLTHILTFLAAETGGCSGSRDD

SEQ ID NO: 22 - DNA, 347 bp, Mycobacterium tuberculosis
  source 1..347: mol_type = genomic DNA
catatgcatcaccatcaccatcacgacgacatcgattgggacgccatcgcgcaatgcgaatccggcggcaattgggcggccaacaccggtaacgggttatacggtggtctgcagatcagccaggcgacgtgggattccaacggtggtgtcgggtcgccggcggccgcgagtccccagcaacagatcgaggtcgcagacaacattatgaaaacccaaggcccgggtgcgtggccgaaatgtagttcttgtagtcagggagacgcaccgctgggctgctcacccacatcctgacgttcctcgcggccgagactggaggttgttcggggagcagggacgattgagaattc

SEQ ID NO: 23 - protein, 112 aa, Mycobacterium tuberculosis
MHHHHHHDDIDWDAIAQCESGGNWAANTGNGLYGGLQISQATWDSNGGVGSPAAASPQQQIEVADNIMKTQGPGAWPKCSSCSQGDAPLGSLTHILTFLAAETGGCSGSRDD

SEQ ID NO: 24 - DNA, 51 bp, synthetic construct
  misc_feature 1..51: note = Cloning primer
  source 1..51: mol_type = other DNA
caattacatatgcatcaccatcaccatcacgacgacatcgattgggacgcc

SEQ ID NO: 25 - DNA, 33 bp, synthetic construct
  misc_feature 1..33: note = Cloning primer
  source 1..33: mol_type = other DNA
caattagaattctcaatcgtccctgctccccga

SEQ ID NO: 26 - protein, 580 aa, Mycobacterium tuberculosis
MNFAVLPPEVNSARIFAGAGLGPMLAAASAWDGLAEELHAAAGSFASVTTGLAGDAWHGPASLAMTRAASPYVGWLNTAAGQAAQAAGQARLAASAFEATLAATVSPAMVAANRTRLASLVAANLLGQNAPAIAAAEAEYEQIWAQDVAAMFGYHSAASAVATQLAPIQEGLQQQLQNVLAQLASGNLGSGNVGVGNIGNDNIGNANIGFGNRGDANIGIGNIGDRNLGIGNTGNWNIGIGITGNGQIGFGKPANPDVLVVGNGGPGVTALVMGGTDSLLPLPNIPLLEYAARFITPVHPGYTATFLETPSQFFPFTGLNSLTYDVSVAQGVTNLHTAIMAQLAAGNEVVVFGTSQSATIATFEMRYLQSLPAHLRPGLDELSFTLTGNPNRPDGGILTRFGFSIPQLGFTLSGATPADAYPTVDYAFQYDGVNDFPKYPLNVFATANAIAGILFLHSGLIALPPDLASGVVQPVSSPDVLTTYILLPSQDLPLLVPLRAIPLLGNPLADLIQPDLRVLVELGYDRTAHQDVPSPFGLFPDVDWAEVAADLQQGAVQGVNDALSGLGLPPPWQPALPRLF

SEQ ID NO: 27 - DNA, 1752 bp, Mycobacterium tuberculosis
  source 1..1752: mol_type = genomic DNA
catatgaatttcgccgttttgccgccggaggtgaattcggcgcgcatattcgccggtgcgggcctgggcccaatgctggcggcggcgtcggcctgggacgggttggccgaggagttgcatgccgcggcgggctcgttcgcgtcggtgaccaccgggttggcgggcgacgcgtggcatggtccggcgtcgctggcgatgacccgcgcggccagcccgtatgtggggtggttgaacacggcggcgggtcaggccgcgcaggcggccggccaggcgcggctagcggcgagcgcgttcgaggcgacgctggcggccaccgtgtctccagcgatggtcgcggccaaccggacacggctggcgtcgctggtggcagccaacttgctgggccagaacgccccggcgatcgcggccgcggaggctgaatacgagcagatatgggcccaggacgtggccgcgatgttcggctatcactccgccgcgtcggcggtggccacgcagctggcgcctattcaagagggtttgcagcagcagctgcaaaacgtgctggcccagttggctagcgggaacctgggcagcggaaatgtgggcgtcggcaacatcggcaacgacaacattggcaacgcaaacatcggcttcggaaatcgaggcgacgccaacatcggcatcgggaatatcggcgacagaaacctcggcattgggaacaccggcaattggaatatcggcatcggcatcaccggcaacggacaaatcggcttcggcaagcctgccaaccccgacgtcttggtggtgggcaacggcggcccgggagtaaccgcgttggtcatgggcggcaccgacagcctactgccgctgcccaacatccccttactcgagtacgctgcgcggttcatcacccccgtgcatcccggatacaccgctacgttcctggaaacgccatcgcagtttttcccattcaccgggctgaatagcctgacctatgacgtctccgtggcccagggcgtaacgaatctgcacaccgcgatcatggcgcaactcgcggcgggaaacgaagtcgtcgtcttcggcacctcccaaagcgccacgatagccaccttcgaaatgcgctatctgcaatccctgccagcacacctgcgtccgggtctcgacgaattgtcctttacgttgaccggcaatcccaaccggcccgacggtggcattcttacgcgttttggcttctccataccgcagttgggtttcacattgtccggcgcgacgcccgccgacgcctaccccaccgtcgattacgcgttccagtacgacggcgtcaacgacttccccaaatacccgctgaatgtcttcgcgaccgccaacgcgatcgcgggcatccttttcctgcactccgggttgattgcgttgccgcccgatcttgcctcgggcgtggttcaaccggtgtcctcaccggacgtcctgaccacctacatcctgctgcccagccaagatctgccgctgctggtcccgctgcgtgctatccccctgctgggaaacccgcttgccgacctcatccagccggacttgcgggtgctcgtcgagttgggttatgaccgcaccgcccaccaggacgtgcccagcccgttcggactgtttccggacgtcgattgggccgaggtggccgcggacctgcagcaaggcgccgtgcaaggcgtcaacgacgccctgtccggactggggctgccgccgccgtggcagccggcgctaccccgacttttctaaaagctt

SEQ ID NO: 28 - protein, 600 aa, Mycobacterium tuberculosis
MGSSHHHHHHSSGLVPRGSHMNFAVLPPEVNSARIFAGAGLGPMLAAASAWDGLAEELHAAAGSFASVTTGLAGDAWHGPASLAMTRAASPYVGWLNTAAGQAAQAAGQARLAASAFEATLAATVSPAMVAANRTRLASLVAANLLGQNAPAIAAAEAEYEQIWAQDVAAMFGYHSAASAVATQLAPIQEGLQQQLQNVLAQLASGNLGSGNVGVGNIGNDNIGNANIGFGNRGDANIGIGNIGDRNLGIGNTGNWNIGIGITGNGQIGFGKPANPDVLVVGNGGPGVTALVMGGTDSLLPLPNIPLLEYAARFITPVHPGYTATFLETPSQFFPFTGLNSLTYDVSVAQGVTNLHTAIMAQLAAGNEVVVFGTSQSATIATFEMRYLQSLPAHLRPGLDELSFTLTGNPNRPDGGILTRFGFSIPQLGFTLSGATPADAYPTVDYAFQYDGVNDFPKYPLNVFATANAIAGILFLHSGLIALPPDLASGVVQPVSSPDVLTTYILLPSQDLPLLVPLRAIPLLGNPLADLIQPDLRVLVELGYDRTAHQDVPSPFGLFPDVDWAEVAADLQQGAVQGVNDALSGLGLPPPWQPALPRLF

SEQ ID NO: 29 - DNA, 32 bp, synthetic construct
  misc_feature 1..32: note = Cloning primer
  source 1..32: mol_type = other DNA
taggatcccatatgaatttcgccgttttgccg

SEQ ID NO: 30 - DNA, 36 bp, synthetic construct
  misc_feature 1..36: note = Cloning primer
  source 1..36: mol_type = other DNA
tagaattcaagcttttagaaaagtcggggtagcgcc

SEQ ID NO: 31 - protein, 87 aa, Mycobacterium tuberculosis
MPYTVRFTTTARRDLHKLPPRILAAVVEFAFGDLSREPLRVGKPLRRELAGTFSARRGTYRLLYRIDDEHTTVVILRVDHRADIYRR

SEQ ID NO: 32 - DNA, 33 bp, synthetic construct
  misc_feature 1..33: note = Cloning primer
  source 1..33: mol_type = other DNA
caattacatatgccttccaccgtgcccttcacc

SEQ ID NO: 33 - DNA, 36 bp, synthetic construct
  misc_feature 1..36: note = Cloning primer
  source 1..36: mol_type = other DNA
caattaaagcttctatcggcggtagatgtccgcgcg

SEQ ID NO: 34 - DNA, 330 bp, Mycobacterium tuberculosis
  source 1..330: mol_type = genomic DNA
atgggcagcagccatcatcatcatcatcacagcagcggcctggtgccgcgcggcagccatatgccttacaccgtgcggttcaccacaaccgcgcgtcgagacctccacaagctgccaccgcgcatcctcgcggcagtggtcgaattcgcgttcggcgatctgtcgcgcgagcccctgcgggtgggcaagccccttcggcgcgagttggccggcacgttcagcgcgcgtcgcggaacgtaccgcctgctgtaccggattgacgacgagcacacaacggtagtgatcctgcgcgtcgatcaccgcgcggacatctaccgccgatagaagctt

SEQ ID NO: 35 - protein, 107 aa, Mycobacterium tuberculosis
  VARIANT 21: note = Xaa = Any Amino Acid
MGSSHHHHHHSSGLVPRGSHXPYTVRFTTTARRDLHKLPPRILAAVVEFAFGDLSREPLRVGKPLRRELAGTFSARRGTYRLLYRIDDEHTTVVILRVDHRADIYRR

SEQ ID NO: 36 - protein, 97 aa, Mycobacterium tuberculosis
MSLLDAHIPQLIASHTAFAAKAGLMRHTIGQAEQQAMSAQAFHQGESAAAFQGAHARFVAAAAKVNTLLDIAQANLGEAAGTYVAADAAAASSYTGF

SEQ ID NO: 37 - DNA, 303 bp, Mycobacterium tuberculosis
  source 1..303: mol_type = genomic DNA
catatgagtttgttggatgcccatattccgcagttgatcgcttcgcatacggcgtttgccgctaaggcggggttgatgcggcatacgatcggtcaggccgagcagcaggcgatgtcggcgcaggcgtttcatcagggagagtccgcggcggcgtttcagggtgcgcatgcccggtttgtggccgcggccgccaaggtcaataccttgctggatatcgcgcaagccaatttgggtgaggccgcgggcacgtatgtggccgccgatgccgccgccgcgtccagctacaccgggttttaaaagctt

SEQ ID NO: 38 - protein, 117 aa, Mycobacterium tuberculosis
MGSSHHHHHHSSGLVPRGSHMSLLDAHIPQLIASHTAFAAKAGLMRHTIGQAEQQAMSAQAFHQGESAAAFQGAHARFVAAAAKVNTLLDIAQANLGEAAGTYVAADAAAASSYTGF

SEQ ID NO: 39 - DNA, 34 bp, synthetic construct
  misc_feature 1..34: note = Cloning primer
  source 1..34: mol_type = other DNA
taggatcccatatgagtttgttggatgcccatat

SEQ ID NO: 40 - DNA, 36 bp, synthetic construct
  misc_feature 1..36: note = Cloning primer
  source 1..36: mol_type = other DNA
tagaattcaagcttttaaaacccggtgtagctggac

SEQ ID NO: 41 - protein, 393 aa, Mycobacterium tuberculosis
VVDFGALPPEINSARMYAGPGSASLVAAAKMWDSVASDLFSAASAFQSVVWGLTVGSWIGSSAGLMAAAASPYVAWMSVTAGQAQLTAAQVRVAAAAYETAYRLTVPPPVIAENRTELMTLTATNLLGQNTPAIEANQAAYSQMWGQDAEAMYGYAATAATATEALLPFEDAPLITNPGGLLEQAVAVEEAIDTAAANQLMNNVPQALQQLAQPAQGVVPSSKLGGLWTAVSPHLSPLSNVSSIANNHMSMMGTGVSMTNTLHSMLKGLAPAAAQAVETAAENGVWAMSSLGSQLGSSLGSSGLGAGVAANLGRAASVGSLSVPPAWAAANQAVTPAARALPLTSLTSAAQTAPGHMLGGLPLGHSVNAGSGINNALRVPARAYAIPRTPAAG

SEQ ID NO: 42 - DNA, 1191 bp, Mycobacterium tuberculosis
  source 1..1191: mol_type = genomic DNA
catatggtggatttcggggcgttaccaccggagatcaactccgcgaggatgtacgccggcccgggttcggcctcgctggtggccgccgcgaagatgtgggacagcgtggcgagtgacctgttttcggccgcgtcggcgtttcagtcggtggtctggggtctgacggtggggtcgtggataggttcgtcggcgggtctgatggcggcggcggcctcgccgtatgtggcgtggatgagcgtcaccgcggggcaggcccagctgaccgccgcccaggtccgggttgctgcggcggcctacgagacagcgtataggctgacggtgcccccgccggtgatcgccgagaaccgtaccgaactgatgacgctgaccgcgaccaacctcttggggcaaaacacgccggcgatcgaggccaatcaggccgcatacagccagatgtggggccaagacgcggaggcgatgtatggctacgccgccacggcggcgacggcgaccgaggcgttgctgccgttcgaggacgccccactgatcaccaaccccggcgggctccttgagcaggccgtcgcggtcgaggaggccatcgacaccgccgcggcgaaccagttgatgaacaatgtgccccaagcgctgcaacagctggcccagccagcgcagggcgtcgtaccttcttccaagctgggtgggctgtggacggcggtctcgccgcatctgtcgccgctcagcaacgtcagttcgatagccaacaaccacatgtcgatgatgggcacgggtgtgtcgatgaccaacaccttgcactcgatgttgaagggcttagctccggcggcggctcaggccgtggaaaccgcggcggaaaacggggtctgggcgatgagctcgctgggcagccagctgggttcgtcgctgggttcttcgggtctgggcgctggggtggccgccaacttgggtcgggcggcctcggtcggttcgttgtcggtgccgccagcatgggccgcggccaaccaggcggtcaccccggcggcgcgggcgctgccgctgaccagcctgaccagcgccgcccaaaccgcccccggacacatgctgggcgggctaccgctggggcactcggtcaacgccggcagcggtatcaacaatgcgctgcgggtgccggcacgggcctacgcgataccccgcacaccggccgccggatagaagctt

SEQ ID NO: 43 - protein, 413 aa, Mycobacterium tuberculosis
MGSSHHHHHHSSGLVPRGSHMVDFGALPPEINSARMYAGPGSASLVAAAKMWDSVASDLFSAASAFQSVVWGLTVGSWIGSSAGLMAAAASPYVAWMSVTAGQAQLTAAQVRVAAAAYETAYRLTVPPPVIAENRTELMTLTATNLLGQNTPAIEANQAAYSQMWGQDAEAMYGYAATAATATEALLPFEDAPLITNPGGLLEQAVAVEEAIDTAAANQLMNNVPQALQQLAQPAQGVVPSSKLGGLWTAVSPHLSPLSNVSSIANNHMSMMGTGVSMTNTLHSMLKGLAPAAAQAVETAAENGVWAMSSLGSQLGSSLGSSGLGAGVAANLGRAASVGSLSVPPAWAAANQAVTPAARALPLTSLTSAAQTAPGHMLGGLPLGHSVNAGSGINNALRVPARAYAIPRTPAAG

SEQ ID NO: 44 - DNA, 33 bp, synthetic construct
  misc_feature 1..33: note = Cloning primer
  source 1..33: mol_type = other DNA
taggatcccatatggtggatttcggggcgttac

SEQ ID NO: 45 - DNA, 35 bp, synthetic construct
  misc_feature 1..35: note = Cloning primer
  source 1..35: mol_type = other DNA
tagaattcaagcttctatccggcggccggtgtgcg

SEQ ID NO: 46 - protein, 94 aa, Mycobacterium tuberculosis
MTINYQFGDVDAHGAMIRAQAGSLEAEHQAIISDVLTASDFWGGAGSAACQGFITQLGRNFQVIYEQANAHGQKVQAAGNNMAQTDSAVGSSWA

SEQ ID NO: 47 - DNA, 294 bp, Mycobacterium tuberculosis
  source 1..294: mol_type = genomic DNA
catatgaccatcaactatcaattcggggacgtcgacgctcacggcgccatgatccgcgctcaggccgggtcgctggaggccgagcatcaggccatcatttctgatgtgttgaccgcgagtgacttttggggcggcgccggttcggcggcctgccaggggttcattacccagctgggccgtaacttccaggtgatctacgagcaggccaacgcccacgggcagaaggtgcaggctgccggcaacaacatggcacaaaccgacagcgccgtcggctccagctgggcctaaaagctt

SEQ ID NO: 48 - protein, 114 aa, Mycobacterium tuberculosis
MGSSHHHHHHSSGLVPRGSHMTINYQFGDVDAHGAMIRAQAGSLEAEHQAIISDVLTASDFWGGAGSAACQGFITQLGRNFQVIYEQANAHGQKVQAAGNNMAQTDSAVGSSWA

SEQ ID NO: 49 - DNA, 33 bp, synthetic construct
  misc_feature 1..33: note = Cloning primer
  source 1..33: mol_type = other DNA
taggatcccatatgaccatcaactatcaattcg

SEQ ID NO: 50 - DNA, 35 bp, synthetic construct
  misc_feature 1..35: note = Cloning primer
  source 1..35: mol_type = other DNA
tagaattcaagcttttaggcccagctggagccgac

SEQ ID NO: 51 - protein, 98 aa, Mycobacterium tuberculosis
MTSRFMTDPHAMRDMAGRFEVHAQTVEDEARRMWASAQNISGAGWSGMAEATSLDTMTQMNQAFRNIVNMLHGVRDGLVRDANNYEQQEQASQQILSS

SEQ ID NO: 52 - DNA, 306 bp, Mycobacterium tuberculosis
  source 1..306: mol_type = genomic DNA
catatgacctcgcgttttatgacggatccgcacgcgatgcgggacatggcgggccgttttgaggtgcacgcccagacggtggaggacgaggctcgccggatgtgggcgtccgcgcaaaacatttccggcgcgggctggagtggcatggccgaggcgacctcgctagacaccatgacccagatgaatcaggcgtttcgcaacatcgtgaacatgctgcacggggtgcgtgacgggctggttcgcgacgccaacaactacgaacagcaagagcaggcctcccagcagatcctcagcagctgaaagctt

SEQ ID NO: 53 - protein, 118 aa, Mycobacterium tuberculosis
MGSSHHHHHHSSGLVPRGSHMTSRFMTDPHAMRDMAGRFEVHAQTVEDEARRMWASAQNISGAGWSGMAEATSLDTMTQMNQAFRNIVNMLHGVRDGLVRDANNYEQQEQASQQILSS

SEQ ID NO: 54 - DNA, 32 bp, synthetic construct
  misc_feature 1..32: note = Cloning primer
  source 1..32: mol_type = other DNA
taggatcccatatgacctcgcgttttatgacg

SEQ ID NO: 55 - DNA, 35 bp, synthetic construct
  misc_feature 1..35: note = Cloning primer
  source 1..35: mol_type = other DNA
tagaattcaagctttcagctgctgaggatctgctg

SEQ ID NO: 56 - protein, 284 aa, Mycobacterium tuberculosis
VPNRRRRKLSTAMSAVAALAVASPCAYFLVYESTETTERPEHHEFKQAAVLTDLPGELMSALSQGLSQFGINIPPVPSLTGSGDASTGLTGPGLTSPGLTSPGLTSPGLTDPALTSPGLTPTLPGSLAAPGTTLAPTPGVGANPALTNPALTSPTGATPGLTSPTGLDPALGGANEIPITTPVGLDPGADGTYPILGDPTLGTIPSSPATTSTGGGGLVNDVMQVANELGASQAIDLLKGVLMPSIMQAVQNGGAAAPAASPPVPPIPAAAAVPPTDPITVPVA

SEQ ID NO: 57 - DNA, 819 bp, Mycobacterium tuberculosis
  source 1..819: mol_type = genomic DNA
catatgcatcaccatcaccatcacagtccttgtgcatattttcttgtctacgaatcaaccgaaacgaccgagcggcccgagcaccatgaattcaagcaggcggcggtgttgaccgacctgcccggcgagctgatgtccgcgctatcgcaggggttgtcccagttcgggatcaacataccgccggtgcccagcctgaccgggagcggcgatgccagcacgggtctaaccggtcctggcctgactagtccgggattgaccagcccgggattgaccagcccgggcctcaccgaccctgcccttaccagtccgggcctgacgccaaccctgcccggatcactcgccgcgcccggcaccaccctggcgccaacgcccggcgtgggggccaatccggcgctcaccaaccccgcgctgaccagcccgaccggggcgacgccgggattgaccagcccgacgggtttggatcccgcgctgggcggcgccaacgaaatcccgattacgacgccggtcggattggatcccggggctgacggcacctatccgatcctcggtgatccaacactggggaccataccgagcagccccgccaccacctccaccggcggcggcggtctcgtcaacgacgtgatgcaggtggccaacgagttgggcgccagtcaggctatcgacctgctaaaaggtgtgctaatgccgtcgatcatgcaggccgtccagaatggcggcgcggccgcgccggcagccagcccgccggtcccgcccatccccgcggccgcggcggtgccaccgacggacccaatcaccgtgccggtcgcctaactcgag

SEQ ID NO: 58 - protein, 269 aa, Mycobacterium tuberculosis
MHHHHHHSPCAYFLVYESTETTERPEHHEFKQAAVLTDLPGELMSALSQGLSQFGINIPPVPSLTGSGDASTGLTGPGLTSPGLTSPGLTSPGLTDPALTSPGLTPTLPGSLAAPGTTLAPTPGVGANPALTNPALTSPTGATPGLTSPTGLDPALGGANEIPITTPVGLDPGADGTYPILGDPTLGTIPSSPATTSTGGGGLVNDVMQVANELGASQAIDLLKGVLMPSIMQAVQNGGAAAPAASPPVPPIPAAAAVPPTDPITVPVA

SEQ ID NO: 59 - DNA, 54 bp, synthetic construct
  misc_feature 1..54: note = Cloning primer
  source 1..54: mol_type = other DNA
caattacatatgcatcaccatcaccatcacagtccttgtgcatattttcttgtc

SEQ ID NO: 60 - DNA, 36 bp, synthetic construct
  misc_feature 1..36: note = Cloning primer
  source 1..36: mol_type = other DNA
caattactcgagttaggcgaccggcacggtgattgg

SEQ ID NO: 61 - protein, 666 aa, Mycobacterium tuberculosis
MAADYDKLFRPHEGMEAPDDMAAQPFFDPSASFPPAPASANLPKPNGQTPPPTSDDLSERFVSAPPPPPPPPPPPPPTPMPIAAGEPPSPEPAASKPPTPPMPIAGPEPAPPKPPTPPMPIAGPEPAPPKPPTPPMPIAGPAPTPTESQLAPPRPPTPQTPTGAPQQPESPAPHVPSHGPHQPRRTAPAPPWAKMPIGEPPPAPSRPSASPAEPPTRPAPQHSRRARRGHRYRTDTERNVGKVATGPSIQARLRAEEASGAQLAPGTEPSPAPLGQPRSYLAPPTRPAPTEPPPSPSPQRNSGRRAERRVHPDLAAQHAAAQPDSITAATTGGRRRKRAAPDLDATQKSLRPAAKGPKVKKVKPQKPKATKPPKVVSQRGWRHWVHALTRINLGLSPDEKYELDLHARVRRNPRGSYQIAVVGLKGGAGKTTLTAALGSTLAQVRADRILALDADPGAGNLADRVGRQSGATIADVLAEKELSHYNDIRAHTSVNAVNLEVLPAPEYSSAQRALSDADWHFIADPASRFYNLVLADCGAGFFDPLTRGVLSTVSGVVVVASVSIDGAQQASVALDWLRNNGYQDLASRACVVINHIMPGEPNVAVKDLVRHFEQQVQPGRVVVMPWDRHIAAGTEISLDLLDPIYKRKVLELAAALSDDFERAGRR

SEQ ID NO: 62 - DNA, 2010 bp, Mycobacterium tuberculosis
  source 1..2010: mol_type = genomic DNA
catatggcggccgactacgacaagctcttccggccgcacgaaggtatggaagctccggacgatatggcagcgcagccgttcttcgaccccagtgcttcgtttccgccggcgcccgcatcggcaaacctaccgaagcccaacggccagactccgcccccgacgtccgacgacctgtcggagcggttcgtgtcggccccgccgccgccacccccacccccacctccgcctccgccaactccgatgccgatcgccgcaggagagccgccctcgccggaaccggccgcatctaaaccacccacaccccccatgcccatcgccggacccgaaccggccccacccaaaccacccacaccccccatgcccatcgccggacccgaaccggccccacccaaaccacccacacctccgatgcccatcgccggacctgcacccaccccaaccgaatcccagttggcgccccccagaccaccgacaccacaaacgccaaccggagcgccgcagcaaccggaatcaccggcgccccacgtaccctcgcacgggccacatcaaccccggcgcaccgcaccagcaccgccctgggcaaagatgccaatcggcgaacccccgcccgctccgtccagaccgtctgcgtccccggccgaaccaccgacccggcctgccccccaacactcccgacgtgcgcgccggggtcaccgctatcgcacagacaccgaacgaaacgtcgggaaggtagcaactggtccatccatccaggcgcggctgcgggcagaggaagcatccggcgcgcagctcgcccccggaacggagccctcgccagcgccgttgggccaaccgagatcgtatctggctccgcccacccgccccgcgccgacagaacctccccccagcccctcgccgcagcgcaactccggtcggcgtgccgagcgacgcgtccaccccgatttagccgcccaacatgccgcggcgcaacctgattcaattacggccgcaaccactggcggtcgtcgccgcaagcgtgcagcgccggatctcgacgcgacacagaaatccttaaggccggcggccaaggggccgaaggtgaagaaggtgaagccccagaaaccgaaggccacgaagccgcccaaagtggtgtcgcagcgcggctggcgacattgggtgcatgcgttgacgcgaatcaacctgggcctgtcacccgacgagaagtacgagctggacctgcacgctcgagtccgccgcaatccccgcgggtcgtatcagatcgccgtcgtcggtctcaaaggtggggctggcaaaaccacgctgacagcagcgttggggtcgacgttggctcaggtgcgggccgaccggatcctggctctagacgcggatccaggcgccggaaacctcgccgatcgggtagggcgacaatcgggcgcgaccatcgctgatgtgcttgcagaaaaagagctgtcgcactacaacgacatccgcgcacacactagcgtcaatgcggtcaatctggaagtgctgccggcaccggaatacagctcggcgcagcgcgcgctcagcgacgccgactggcatttcatcgccgatcctgcgtcgaggttttacaacctcgtcttggctgattgtggggccggcttcttcgacccgctgacccgcggcgtgctgtccacggtgtccggtgtcgtggtcgtggcaagtgtctcaatcgacggcgcacaacaggcgtcggtcgcgttggactggttgcgcaacaacggttaccaagatttggcgagccgcgcatgcgtggtcatcaatcacatcatgccgggagaacccaatgtcgcagttaaagacctggtgcggcatttcgaacagcaagttcaacccggccgggtcgtggtcatgccgtgggacaggcacattgcggccggaaccgagatttcactcgacttgctcgaccctatctacaagcgcaaggtcctcgaattggccgcagcgctatccgacgatttcgagagggctggacgtcgttgaggattc

SEQ ID NO: 63 - protein, 452 aa, Mycobacterium tuberculosis
HMHHHHHHSRRARRGHRYRTDTERNVGKVATGPSIQARLRAEEASGAQLAPGTEPSPAPLGQPRSYLAPPTRPAPTEPPPSPSPQRNSGRRAERRVHPDLAAQHAAAQPDSITAATTGGRRRKRAAPDLDATQKSLRPAAKGPKVKKVKPQKPKATKPPKVVSQRGWRHWVHALTRINLGLSPDEKYELDLHARVRRNPRGSYQIAVVGLKGGAGKTTLTAALGSTLAQVRADRILALDADPGAGNLADRVGRQSGATIADVLAEKELSHYNDIRAHTSVNAVNLEVLPAPEYSSAQRALSDADWHFIADPASRFYNLVLADCGAGFFDPLTRGVLSTVSGVVVVASVSIDGAQQASVALDWLRNNGYQDLASRACVVINHIMPGEPNVAVKDLVRHFEQQVQPGRVVVMPWDRHIAAGTEISLDLLDPIYKRKVLELAAALSDDFERAGRR

SEQ ID NO: 64 - DNA, 32 bp, synthetic construct
  misc_feature 1..32: note = Cloning primer
  source 1..32: mol_type = other DNA
gatcccatgggcatatggcggccgactacgac

SEQ ID NO: 65 - DNA, 28 bp, synthetic construct
  misc_feature 1..28: note = Cloning primer
  source 1..28: mol_type = other DNA
gtcagaattctcaacgacgtccagccct

SEQ ID NO: 66 - DNA, 1185 bp, synthetic construct
  misc_feature 1..1185: note = Mycobaterium tuberculosi fusion sequence
  source 1..1185: mol_type = other DNA
atgggcagcagccatcatcatcatcatcacagcagcggcctggtgccgcgcggcagccatatggacgacatcgattgggacgccatcgcgcaatgcgaatccggcggcaattgggcggccaacaccggtaacgggttatacggtggtctgcagatcagccaggcgacgtgggattccaacggtggtgtcgggtcgccggcggccgcgagtccccagcaacagatcgaggtcgcagacaacattatgaaaacccaaggcccgggtgcgtggccgaaatgtagttcttgtagtcagggagacgcaccgctgggctcgctcacccacatcctgacgttcctcgcggccgagactggaggttgttcggggagcagggacgatgagctcagtccttgtgcatattttcttgtctacgaatcaaccgaaacgaccgagcggcccgagcaccatgaattcaagcaggcggcggtgttgaccgacctgcccggcgagctgatgtccgcgctatcgcaggggttgtcccagttcgggatcaacataccgccggtgcccagcctgaccgggagcggcgatgccagcacgggtctaaccggtcctggcctgactagtccgggattgaccagcccgggattgaccagcccgggcctcaccgaccctgcccttaccagtccgggcctgacgccaaccctgcccggatcactcgccgcgcccggcaccaccctggcgccaacgcccggcgtgggggccaatccggcgctcaccaaccccgcgctgaccagcccgaccggggcgacgccgggattgaccagcccgacgggtttggatcccgcgctgggcggcgccaacgaaatcccgattacgacgccggtcggattggatcccggggctgacggcacctatccgatcctcggtgatccaacactggggaccataccgagcagccccgccaccacctccaccggcggcggcggtctcgtcaacgacgtgatgcaggtggccaacgagttgggcgccagtcaggctatcgacctgctaaaaggtgtgctaatgccgtcgatcatgcaggccgtccagaatggcggcgcggccgcgccggcagccagcccgccggtcccgcccatccccgcggccgcggcggtgccaccgacggacccaatcaccgtgccggtcgccggtacctaaaagctt

SEQ ID NO: 67 - protein, 392 aa, synthetic construct
  REGION 1..392: note = Mycobaterium tuberculosi fusion sequence
MGSSHHHHHHSSGLVPRGSHMDDIDWDAIAQCESGGNWAANTGNGLYGGLQISQATWDSNGGVGSPAAASPQQQIEVADNIMKTQGPGAWPKCSSCSQGDAPLGSLTHILTFLAAETGGCSGSRDDELSPCAYFLVYESTETTERPEHHEFKQAAVLTDLPGELMSALSQGLSQFGINIPPVPSLTGSGDASTGLTGPGLTSPGLTSPGLTSPGLTDPALTSPGLTPTLPGSLAAPGTTLAPTPGVGANPALTNPALTSPTGATPGLTSPTGLDPALGGANEIPITTPVGLDPGADGTYPILGDPTLGTIPSSPATTSTGGGGLVNDVMQVANELGASQAIDLLKGVLMPSIMQAVQNGGAAAPAASPPVPPIPAAAAVPPTDPITVPVAGT

SEQ ID NO: 68 - DNA, 33 bp, synthetic construct
  misc_feature 1..33: note = Cloning primer
  source 1..33: mol_type = other DNA
caattacatatggacgacatcgattgggacgcc

SEQ ID NO: 69 - DNA, 33 bp, synthetic construct
  misc_feature 1..33: note = Cloning primer
  source 1..33: mol_type = other DNA
caattagagctcatcgtccctgctccccgaaca

SEQ ID NO: 70 - DNA, 34 bp, synthetic construct
  misc_feature 1..34: note = Cloning primer
  source 1..34: mol_type = other DNA
caattagagctcagtccttgtgcatattttcttg

SEQ ID NO: 71 - DNA, 42 bp, synthetic construct
  misc_feature 1..42: note = Cloning primer
  source 1..42: mol_type = other DNA
caattaaagcttttaggtaccggcgaccggcacggtgattgg

SEQ ID NO: 72 - DNA, 1707 bp, synthetic construct
  misc_feature 1..1707: note = Mycobacterium tuberculosis fusion sequence
  source 1..1707: mol_type = other DNA
atgggcagcagccatcatcatcatcatcacagcagcggcctggtgccgcgcggcagccatatgggtacccatctcgccaacggttcgatgtcggaagtcatgatgtcggaaattgccgggttgcctatccctccgattatccattacggggcgattgcctatgcccccagcggcgcgtcgggcaaagcgtggcaccagcgcacaccggcgcgagcagagcaagtcgcactagaaaagtgcggtgacaagacttgcaaagtggttagtcgcttcaccaggtgcggcgcggtcgcctacaacggctcgaaataccaaggcggaaccggactcacgcgccgcgcggcagaagacgacgccgtgaaccgactcgaaggcgggcggatcgtcaactgggcgtgcaacgagctcatgacctcgcgttttatgacggatccgcacgcgatgcgggacatggcgggccgttttgaggtgcacgcccagacggtggaggacgaggctcgccggatgtgggcgtccgcgcaaaacatctcgggcgcgggctggagtggcatggccgaggcgacctcgctagacaccatgacccagatgaatcaggcgtttcgcaacatcgtgaacatgctgcacggggtgcgtgacgggctggttcgcgacgccaacaactacgaacagcaagagcaggcctcccagcagatcctcagcagcgtcgacgtggtcgatgcccaccgcggcggccacccgaccccgatgagctcgacgaaggccacgctgcggctggccgaggccaccgacagctcgggcaagatcaccaagcgcggagccgacaagctgatttccaccatcgacgaattcgccaagattgccatcagctcgggctgtgccgagctgatggccttcgccacgtcggcggtccgcgacgccgagaattccgaggacgtcctgtcccgggtgcgcaaagagaccggtgtcgagttgcaggcgctgcgtggggaggacgagtcacggctgaccttcctggccgtgcgacgatggtacgggtggagcgctgggcgcatcctcaacctcgacatcggcggcggctcgctggaagtgtccagtggcgtggacgaggagcccgagattgcgttatcgctgcccctgggcgccggacggttgacccgagagtggctgcccgacgatccgccgggccggcgccgggtggcgatgctgcgagactggctggatgccgagctggccgagcccagtgtgaccgtcctggaagccggcagccccgacctggcggtcgcaacgtcgaagacgtttcgctcgttggcgcgactaaccggtgcggccccatccatggccgggccgcgggtgaagaggaccctaacggcaaatggtctgcggcaactcatcgcgtttatctctaggatgacggcggttgaccgtgcagaactggaaggggtaagcgccgaccgagcgccgcagattgtggccggcgccctggtggcagaggcgagcatgcgagcactgtcgatagaagcggtggaaatctgcccgtgggcgctgcgggaaggtctcatcttgcgcaaactcgacagcgaagccgacggaaccgccctcatcgagtcttcgtctgtgcacacttcggtgcgtgccgtcggaggtcagccagctgatcggaacgcggccaaccgatcgagaggcagcaaaccaagtacttaaaagctt

SEQ ID NO: 73 - DNA, 39 bp, synthetic construct
  misc_feature 1..39: note = Cloning primer
  source 1..39: mol_type = other DNA
caattacatatgggtacccatctcgccaacggttcgatg

SEQ ID NO: 74 - DNA, 33 bp, synthetic construct
  misc_feature 1..33: note = Cloning primer
  source 1..33: mol_type = other DNA
caattagagctcgttgcacgcccagttgacgat

SEQ ID NO: 75 - DNA, 33 bp, synthetic construct
  misc_feature 1..33: note = Cloning primer
  source 1..33: mol_type = other DNA
caattagagctcatgacctcgcgttttatgacg

SEQ ID NO: 76 - DNA, 33 bp, synthetic construct
  misc_feature 1..33: note = Cloning primer
  source 1..33: mol_type = other DNA
caattagtcgacgctgctgaggatctgctggga

SEQ ID NO: 77 - DNA, 33 bp, synthetic construct
  misc_feature 1..33: note = Cloning primer
  source 1..33: mol_type = other DNA
caattagtcgacatggtcgatgcccaccgcggc

SEQ ID NO: 78 - DNA, 42 bp, synthetic construct
  misc_feature 1..42: note = Cloning primer
  source 1..42: mol_type = other DNA
caattaaagcttttaagtacttggtttgctgcctctcgatcg

SEQ ID NO: 79 - protein, 566 aa, synthetic construct
  REGION 1..566: note = Mycobacterium tuberculosis fusion sequence
MGSSHHHHHHSSGLVPRGSHMGTHLANGSMSEVMMSEIAGLPIPPIIHYGAIAYAPSGASGKAWHQRTPARAEQVALEKCGDKTCKVVSRFTRCGAVAYNGSKYQGGTGLTRRAAEDDAVNRLEGGRIVNWACNELMTSRFMTDPHAMRDMAGRFEVHAQTVEDEARRMWASAQNISGAGWSGMAEATSLDTMTQMNQAFRNIVNMLHGVRDGLVRDANNYEQQEQASQQILSSVDVVDAHRGGHPTPMSSTKATLRLAEATDSSGKITKRGADKLISTIDEFAKIAISSGCAELMAFATSAVRDAENSEDVLSRVRKETGVELQALRGEDESRLTFLAVRRWYGWSAGRILNLDIGGGSLEVSSGVDEEPEIALSLPLGAGRLTREWLPDDPPGRRRVAMLRDWLDAELAEPSVTVLEAGSPDLAVATSKTFRSLARLTGAAPSMAGPRVKRTLTANGLRQLIAFISRMTAVDRAELEGVSADRAPQIVAGALVAEASMRALSIEAVEICPWALREGLILRKLDSEADGTALIESSSVHTSVRAVGGQPADRNAANRSRGSKPST

SEQ ID NO: 80 - DNA, 2022 bp, synthetic construct
  misc_feature 1..2022: note = Mycobacterium tuberculosis fusion sequence
  source 1..2022: mol_type = other DNA
atgggcagcagccatcatcatcatcatcacagcagcggcctggtgccgcgcggcagccatatggacgacatcgattgggacgccatcgcgcaatgcgaatccggcggcaattgggcggccaacaccggtaacgggttatacggtggtctgcagatcagccaggcgacgtgggattccaacggtggtgtcgggtcgccggcggccgcgagtccccagcaacagatcgaggtcgcagacaacattatgaaaacccaaggcccgggtgcgtggccgaaatgtagttcttgtagtcagggagacgcaccgctgggctcgctcacccacatcctgacgttcctcgcggccgagactggaggttgttcggggagcagggacgatggtacccatctcgccaacggttcgatgtcggaagtcatgatgtcggaaattgccgggttgcctatccctccgattatccattacggggcgattgcctatgcccccagcggcgcgtcgggcaaagcgtggcaccagcgcacaccggcgcgagcagagcaagtcgcactagaaaagtgcggtgacaagacttgcaaagtggttagtcgcttcaccaggtgcggcgcggtcgcctacaacggctcgaaataccaaggcggaaccggactcacgcgccgcgcggcagaagacgacgccgtgaaccgactcgaaggcgggcggatcgtcaactgggcgtgcaacgagctcatgacctcgcgttttatgacggatccgcacgcgatgcgggacatggcgggccgttttgaggtgcacgcccagacggtggaggacgaggctcgccggatgtgggcgtccgcgcaaaacatctcgggcgcgggctggagtggcatggccgaggcgacctcgctagacaccatgacccagatgaatcaggcgtttcgcaacatcgtgaacatgctgcacggggtgcgtgacgggctggttcgcgacgccaacaactacgaacagcaagagcaggcctcccagcagatcctcagcagcgtcgacatggtcgatgcccaccgcggcggccacccgaccccgatgagctcgacgaaggccacgctgcggctggccgaggccaccgacagctcgggcaagatcaccaagcgcggagccgacaagctgatttccaccatcgacgaattcgccaagattgccatcagctcgggctgtgccgagctgatggccttcgccacgtcggcggtccgcgacgccgagaattccgaggacgtcctgtcccgggtgcgcaaagagaccggtgtcgagttgcaggcgctgcgtggggaggacgagtcacggctgaccttcctggccgtgcgacgatggtacgggtggagcgctgggcgcatcctcaacctcgacatcggcggcggctcgctggaagtgtccagtggcgtggacgaggagcccgagattgcgttatcgctgcccctgggcgccggacggttgacccgagagtggctgcccgacgatccgccgggccggcgccgggtggcgatgctgcgagactggctggatgccgagctggccgagcccagtgtgaccgtcctggaagccggcagccccgacctggcggtcgcaacgtcgaagacgtttcgctcgttggcgcgactaaccggtgcggccccatccatggccgggccgcgggtgaagaggaccctaacggcaaatggtctgcggcaactcatcgcgtttatctctaggatgacggcggttgaccgtgcagaactggaaggggtaagcgccgaccgagcgccgcagattgtggccggcgccctggtggcagaggcgagcatgcgagcactgtcgatagaagcggtggaaatctgcccgtgggcgctgcgggaaggtctcatcttgcgcaaactcgacagcgaagccgacggaaccgccctcatcgagtcttcgtctgtgcacacttcggtgcgtgccgtcggaggtcagccagctgatcggaacgcggccaaccgatcgagaggcagcaaaccaagtacttaaaagctt

SEQ ID NO: 81 - DNA, 33 bp, synthetic construct; identical to SEQ ID NO: 68
  misc_feature 1..33: note = Cloning primer
  source 1..33: mol_type = other DNA

SEQ ID NO: 82 - DNA, 42 bp, synthetic construct; identical to SEQ ID NO: 78
  misc_feature 1..42: note = Cloning primer
  source 1..42: mol_type = other DNA

SEQ ID NO: 83 - protein, 671 aa, synthetic construct
  REGION 1..671: note = Mycobacterium tuberculosis fusion sequence
MGSSHHHHHHSSGLVPRGSHMDDIDWDAIAQCESGGNWAANTGNGLYGGLQISQATWDSNGGVGSPAAASPQQQIEVADNIMKTQGPGAWPKCSSCSQGDAPLGSLTHILTFLAAETGGCSGSRDDGTHLANGSMSEVMMSEIAGLPIPPIIHYGAIAYAPSGASGKAWHQRTPARAEQVALEKCGDKTCKVVSRFTRCGAVAYNGSKYQGGTGLTRRAAEDDAVNRLEGGRIVNWACNELMTSRFMTDPHAMRDMAGRFEVHAQTVEDEARRMWASAQNISGAGWSGMAEATSLDTMTQMNQAFRNIVNMLHGVRDGLVRDANNYEQQEQASQQILSSVDMVDAHRGGHPTPMSSTKATLRLAEATDSSGKITKRGADKLISTIDEFAKIAISSGCAELMAFATSAVRDAENSEDVLSRVRKETGVELQALRGEDESRLTFLAVRRWYGWSAGRILNLDIGGGSLEVSSGVDEEPEIALSLPLGAGRLTREWLPDDPPGRRRVAMLRDWLDAELAEPSVTVLEAGSPDLAVATSKTFRSLARLTGAAPSMAGPRVKRTLTANGLRQLIAFISRMTAVDRAELEGVSADRAPQIVAGALVAEASMRALSIEAVEICPWALREGLILRKLDSEADGTALIESSSVHTSVRAVGGQPADRNAANRSRGSKPST

SEQ ID NO: 84 - DNA, 2463 bp, synthetic construct
  misc_feature 1..2463: note = Mycobacterium tuberculosis fusion sequence
  source 1..2463: mol_type = other DNA
atgggcagcagccatcatcatcatcatcacagcagcggcctggtgccgcgcggcagccatatgggtacccatctcgccaacggttcgatgtcggaagtcatgatgtcggaaattgccgggttgcctatccctccgattatccattacggggcgattgcctatgcccccagcggcgcgtcgggcaaagcgtggcaccagcgcacaccggcgcgagcagagcaagtcgcactagaaaagtgcggtgacaagacttgcaaagtggttagtcgcttcaccaggtgcggcgcggtcgcctacaacggctcgaaataccaaggcggaaccggactcacgcgccgcgcggcagaagacgacgccgtgaaccgactcgaaggcgggcggatcgtcaactgggcgtgcaacgagctcatgacctcgcgttttatgacggatccgcacgcgatgcgggacatggcgggccgttttgaggtgcacgcccagacggtggaggacgaggctcgccggatgtgggcgtccgcgcaaaacatctcgggcgcgggctggagtggcatggccgaggcgacctcgctagacaccatgacccagatgaatcaggcgtttcgcaacatcgtgaacatgctgcacggggtgcgtgacgggctggttcgcgacgccaacaactacgaacagcaagagcaggcctcccagcagatcctcagcagcgtcgacatcaatttcgccgttttgccgccggaggtgaattcggcgcgcatattcgccggtgcgggcctgggcccaatgctggcggcggcgtcggcctgggacgggttggccgaggagttgcatgccgcggcgggctcgttcgcgtcggtgaccaccgggttggcgggcgacgcgtggcatggtccggcgtcgctggcgatgacccgcgcggccagcccgtatgtggggtggttgaacacggcggcgggtcaggccgcgcaggcggccggccaggcgcggctagcggcgagcgcgttcgaggcgacgctggcggccaccgtgtctccagcgatggtcgcggccaaccggacacggctggcgtcgctggtggcagccaacttgctgggccagaacgccccggcgatcgcggccgcggaggctgaatacgagcagatatgggcccaggacgtggccgcgatgttcggctatcactccgccgcgtcggcggtggccacgcagctggcgcctattcaagagggtttgcagcagcagctgcaaaacgtgctggcccagttggctagcgggaacctgggcagcggaaatgtgggcgtcggcaacatcggcaacgacaacattggcaacgcaaacatcggcttcggaaatcgaggcgacgccaacatcggcatcgggaatatcggcgacagaaacctcggcattgggaacaccggcaattggaatatcggcatcggcatcaccggcaacggacaaatcggcttcggcaagcctgccaaccccgacgtcttggtggtgggcaacggcggcccgggagtaaccgcgttggtcatgggcggcaccgacagcctactgccgctgcccaacatccccttactcgagtacgctgcgcggttcatcacccccgtgcatcccggatacaccgctacgttcctggaaacgccatcgcagtttttcccattcaccgggctgaatagcctgacctatgacgtctccgtggcccagggcgtaacgaatctgcacaccgcgatcatggcgcaactcgcggcgggaaacgaagtcgtcgtcttcggcacctcccaaagcgccacgatagccaccttcgaaatgcgctatctgcaatccctgccagcacacctgcgtccgggtctcgacgaattgtcctttacgttgaccggcaatcccaaccggcccgacggtggcattcttacgcgttttggcttctccataccgcagttgggtttcacattgtccggcgcgacgcccgccgacgcctaccccaccgtcgattacgcgttccagtacgacggcgtcaacgacttccccaaatacccgctgaatgtcttcgcgaccgccaacgcgatcgcgggcatccttttcctgcactccgggttgattgcgttgccgcccgatcttgcctcgggcgtggttcaaccggtgtcctcaccggacgtcctgaccacctacatcctgctgcccagccaagatctgccgctgctggtcccgctgcgtgctatccccctgctgggaaacccgcttgccgacctcatccagccggacttgcgggtgctcgtcgagttgggttatgaccgcaccgcccaccaggacgtgcccagcccgttcggactgtttccggacgtcgattgggccgaggtggccgcggacctgcagcaaggcgccgtgcaaggcgtcaacgacgccctgtccggactggggctgccgccgccgtggcagccggcgctaccccgacttttcagtacttaaaagctt

SEQ ID NO: 85 - DNA, 39 bp, synthetic construct; identical to SEQ ID NO: 73
  misc_feature 1..39: note = Cloning primer
  source 1..39: mol_type = other DNA

SEQ ID NO: 86 - DNA, 33 bp, synthetic construct; identical to SEQ ID NO: 74
  misc_feature 1..33: note = Cloning primer
  source 1..33: mol_type = other DNA

SEQ ID NO: 87 - DNA, 33 bp, synthetic construct; identical to SEQ ID NO: 75
  misc_feature 1..33: note = Cloning primer
  source 1..33: mol_type = other DNA

SEQ ID NO: 88 - DNA, 33 bp, synthetic construct; identical to SEQ ID NO: 76
  misc_feature 1..33: note = Cloning primer
  source 1..33: mol_type = other DNA

SEQ ID NO: 89 - DNA, 33 bp, synthetic construct
  misc_feature 1..33: note = Cloning primer
  source 1..33: mol_type = other DNA
caattagtcgacatgaatttcgccgttttgccg

SEQ ID NO: 90 - DNA, 42 bp, synthetic construct
  misc_feature 1..42: note = Cloning primer
  source 1..42: mol_type = other DNA
caattaaagcttttaagtactgaaaagtcggggtagcgccgg

SEQ ID NO: 91 - protein, 818 aa, synthetic construct
  REGION 1..818: note = Mycobacterium tuberculosis fusion sequence
MGSSHHHHHHSSGLVPRGSHMGTHLANGSMSEVMMSEIAGLPIPPIIHYGAIAYAPSGASGKAWHQRTPARAEQVALEKCGDKTCKVVSRFTRCGAVAYNGSKYQGGTGLTRRAAEDDAVNRLEGGRIVNWACNELMTSRFMTDPHAMRDMAGRFEVHAQTVEDEARRMWASAQNISGAGWSGMAEATSLDTMTQMNQAFRNIVNMLHGVRDGLVRDANNYEQQEQASQQILSSVDINFAVLPPEVNSARIFAGAGLGPMLAAASAWDGLAEELHAAAGSFASVTTGLAGDAWHGPASLAMTRAASPYVGWLNTAAGQAAQAAGQARLAASAFEATLAATVSPAMVAANRTRLASLVAANLLGQNAPAIAAAEAEYEQIWAQDVAAMFGYHSAASAVATQLAPIQEGLQQQLQNVLAQLASGNLGSGNVGVGNIGNDNIGNANIGFGNRGDANIGIGNIGDRNLGIGNTGNWNIGIGITGNGQIGFGKPANPDVLVVGNGGPGVTALVMGGTDSLLPLPNIPLLEYAARFITPVHPGYTATFLETPSQFFPFTGLNSLTYDVSVAQGVTNLHTAIMAQLAAGNEVVVFGTSQSATIATFEMRYLQSLPAHLRPGLDELSFTLTGNPNRPDGGILTRFGFSIPQLGFTLSGATPADAYPTVDYAFQYDGVNDFPKYPLNVFATANAIAGILFLHSGLIALPPDLASGVVQPVSSPDVLTTYILLPSQDLPLLVPLRAIPLLGNPLADLIQPDLRVLVELGYDRTAHQDVPSPFGLFPDVDWAEVAADLQQGAVQGVNDALSGLGLPPPWQPALPRLFST

SEQ ID NO: 92 - DNA, 2778 bp, synthetic construct
  misc_feature 1..2778: note = Mycobacterium tuberculosis fusion sequence
  source 1..2778: mol_type = other DNA
atgggcagcagccatcatcatcatcatcacagcagcggcctggtgccgcgcggcagccatatggacgacatcgattgggacgccatcgcgcaatgcgaatccggcggcaattgggcggccaacaccggtaacgggttatacggtggtctgcagatcagccaggcgacgtgggattccaacggtggtgtcgggtcgccggcggccgcgagtccccagcaacagatcgaggtcgcagacaacattatgaaaacccaaggcccgggtgcgtggccgaaatgtagttcttgtagtcagggagacgcaccgctgggctcgctcacccacatcctgacgttcctcgcggccgagactggaggttgttcggggagcagggacgatggtacccatctcgccaacggttcgatgtcggaagtcatgatgtcggaaattgccgggttgcctatccctccgattatccattacggggcgattgcctatgcccccagcggcgcgtcgggcaaagcgtggcaccagcgcacaccggcgcgagcagagcaagtcgcactagaaaagtgcggtgacaagacttgcaaagtggttagtcgcttcaccaggtgcggcgcggtcgcctacaacggctcgaaataccaaggcggaaccggactcacgcgccgcgcggcagaagacgacgccgtgaaccgactcgaaggcgggcggatcgtcaactgggcgtgcaacgagctcatgacctcgcgttttatgacggatccgcacgcgatgcgggacatggcgggccgttttgaggtgcacgcccagacggtggaggacgaggctcgccggatgtgggcgtccgcgcaaaacatctcgggcgcgggctggagtggcatggccgaggcgacctcgctagacaccatgacccagatgaatcaggcgtttcgcaacatcgtgaacatgctgcacggggtgcgtgacgggctggttcgcgacgccaacaactacgaacagcaagagcaggcctcccagcagatcctcagcagcgtcgacatcaatttcgccgttttgccgccggaggtgaattcggcgcgcatattcgccggtgcgggcctgggcccaatgctggcggcggcgtcggcctgggacgggttggccgaggagttgcatgccgcggcgggctcgttcgcgtcggtgaccaccgggttggcgggcgacgcgtggcatggtccggcgtcgctggcgatgacccgcgcggccagcccgtatgtggggtggttgaacacggcggcgggtcaggccgcgcaggcggccggccaggcgcggctagcggcgagcgcgttcgaggcgacgctggcggccaccgtgtctccagcgatggtcgcggccaaccggacacggctggcgtcgctggtggcagccaacttgctgggccagaacgccccggcgatcgcggccgcggaggctgaatacgagcagatatgggcccaggacgtggccgcgatgttcggctatcactccgccgcgtcggcggtggccacgcagctggcgcctattcaagagggtttgcagcagcagctgcaaaacgtgctggcccagttggctagcgggaacctgggcagcggaaatgtgggcgtcggcaacatcggcaacgacaacattggcaacgcaaacatcggcttcggaaatcgaggcgacgccaacatcggcatcgggaatatcggcgacagaaacctcggcattgggaacaccggcaattggaatatcggcatcggcatcaccggcaacggacaaatcggcttcggcaagcctgccaaccccgacgtcttggtggtgggcaacggcggcccgggagtaaccgcgttggtcatgggcggcaccgacagcctactgccgctgcccaacatccccttactcgagtacgctgcgcggttcatcacccccgtgcatcccggatacaccgctacgttcctggaaacgccatcgcagtttttcccattcaccgggctgaatagcctgacctatgacgtctccgtggcccagggcgtaacgaatctgcacaccgcgatcatggcgcaactcgcggcgggaaacgaagtcgtcgtcttcggcacctcccaaagcgccacgatagccaccttcgaaatgcgctatctgcaatccctgccagcacacctgcgtccgggtctcgacgaattgtcctttacgttgaccggcaatcccaaccggcccgacggtggcattcttacgcgttttggcttctccataccgcagttgggtttcacattgtccggcgcgacgcccgccgacgcctaccccaccgtcgattacgcgttccagtacgacggcgtcaacgacttccccaaatacccgctgaatgtcttcgcgaccgccaacgcgatcgcgggcatccttttcctgcactccgggttgattgcgttgccgcccgatcttgcctcgggcgtggttcaaccggtgtcctcaccggacgtcctgaccacctacatcctgctgcccagccaagatctgccgctgctggtcccgctgcgtgctatccccctgctgggaaacccgcttgccgacctcatccagccggacttgcgggtgctcgtcgagttgggttatgaccgcaccgcccaccaggacgtgcccagcccgttcggactgtttccggacgtcgattgggccgaggtggccgcggacctgcagcaaggcgccgtgcaaggcgtcaacgacgccctgtccggactggggctgccgccgccgtggcagccggcgctaccccgacttttcagtacttaaaagctt

SEQ ID NO: 93 - DNA, 33 bp, synthetic construct; identical to SEQ ID NO: 68
  misc_feature 1..33: note = Cloning primer
  source 1..33: mol_type = other DNA

SEQ ID NO: 94 - DNA, 42 bp, synthetic construct; identical to SEQ ID NO: 78
  misc_feature 1..42: note = Cloning primer
  source 1..42: mol_type = other DNA

SEQ ID NO: 95 - protein, 923 aa, synthetic construct
  REGION 1..923: note = Mycobacterium tuberculosis fusion sequence
MGSSHHHHHHSSGLVPRGSHMDDIDWDAIAQCESGGNWAANTGNGLYGGLQISQATWDSNGGVGSPAAASPQQQIEVADNIMKTQGPGAWPKCSSCSQGDAPLGSLTHILTFLAAETGGCSGSRDDGTHLANGSMSEVMMSEIAGLPIPPIIHYGAIAYAPSGASGKAWHQRTPARAEQVALEKCGDKTCKVVSRFTRCGAVAYNGSKYQGGTGLTRRAAEDDAVNRLEGGRIVNWACNELMTSRFMTDPHAMRDMAGRFEVHAQTVEDEARRMWASAQNISGAGWSGMAEATSLDTMTQMNQAFRNIVNMLHGVRDGLVRDANNYEQQEQASQQILSSVDINFAVLPPEVNSARIFAGAGLGPMLAAASAWDGLAEELHAAAGSFASVTTGLAGDAWHGPASLAMTRAASPYVGWLNTAAGQAAQAAGQARLAASAFEATLAATVSPAMVAANRTRLASLVAANLLGQNAPAIAAAEAEYEQIWAQDVAAMFGYHSAASAVATQLAPIQEGLQQQLQNVLAQLASGNLGSGNVGVGNIGNDNIGNANIGFGNRGDANIGIGNIGDRNLGIGNTGNWNIGIGITGNGQIGFGKPANPDVLVVGNGGPGVTALVMGGTDSLLPLPNIPLLEYAARFITPVHPGYTATFLETPSQFFPFTGLNSLTYDVSVAQGVTNLHTAIMAQLAAGNEVVVFGTSQSATIATFEMRYLQSLPAHLRPGLDELSFTLTGNPNRPDGGILTRFGFSIPQLGFTLSGATPADAYPTVDYAFQYDGVNDFPKYPLNVFATANAIAGILFLHSGLIALPPDLASGVVQPVSSPDVLTTYILLPSQDLPLLVPLRAIPLLGNPLADLIQPDLRVLVELGYDRTAHQDVPSPFGLFPDVDWAEVAADLQQGAVQGVNDALSGLGLPPPWQPALPRLFST

SEQ ID NO: 96 - DNA, 2814 bp, synthetic construct
  misc_feature 1..2814: note = Mycobacterium tuberculosis fusion sequence
  source 1..2814: mol_type = other DNA
atgggcagcagccatcatcatcatcatcacagcagcggcctggtgccgcgcggcagccatatggacgacatcgattgggacgccatcgcgcaatgcgaatccggcggcaattgggcggccaacaccggtaacgggttatacggtggtctgcagatcagccaggcgacgtgggattccaacggtggtgtcgggtcgccggcggccgcgagtccccagcaacagatcgaggtcgcagacaacattatgaaaacccaaggcccgggtgcgtggccgaaatgtagttcttgtagtcagggagacgcaccgctgggctcgctcacccacatcctgacgttcctcgcggccgagactggaggttgttcggggagcagggacgatgagctcagtccttgtgcatattttcttgtctacgaatcaaccgaaacgaccgagcggcccgagcaccatgaattcaagcaggcggcggtgttgaccgacctgcccggcgagctgatgtccgcgctatcgcaggggttgtcccagttcgggatcaacataccgccggtgcccagcctgaccgggagcggcgatgccagcacgggtctaaccggtcctggcctgactagtccgggattgaccagcccgggattgaccagcccgggcctcaccgaccctgcccttaccagtccgggcctgacgccaaccctgcccggatcactcgccgcgcccggcaccaccctggcgccaacgcccggcgtgggggccaatccggcgctcaccaaccccgcgctgaccagcccgaccggggcgacgccgggattgaccagcccgacgggtttggatcccgcgctgggcggcgccaacgaaatcccgattacgacgccggtcggattggatcccggggctgacggcacctatccgatcctcggtgatccaacactggggaccataccgagcagccccgccaccacctccaccggcggcggcggtctcgtcaacgacgtgatgcaggtggccaacgagttgggcgccagtcaggctatcgacctgctaaaaggtgtgctaatgccgtcgatcatgcaggccgtccagaatggcggcgcggccgcgccggcagccagcccgccggtcccgcccatccccgcggccgcggcggtgccaccgacggacccaatcaccgtgccggtcgccggtacccatctcgccaacggttcgatgtcggaagtcatgatgtcggaaattgccgggttgcctatccctccgattatccattacggggcgattgcctatgcccccagcggcgcgtcgggcaaagcgtggcaccagcgcacaccggcgcgagcagagcaagtcgcactagaaaagtgcggtgacaagacttgcaaagtggttagtcgcttcaccaggtgcggcgcggtcgcctacaacggctcgaaataccaaggcggaaccggactcacgcgccgcgcggcagaagacgacgccgtgaaccgactcgaaggcgggcggatcgtcaactgggcgtgcaacgagctcatgacctcgcgttttatgacggatccgcacgcgatgcgggacatggcgggccgttttgaggtgcacgcccagacggtggaggacgaggctcgccggatgtgggcgtccgcgcaaaacatctcgggcgcgggctggagtggcatggccgaggcgacctcgctagacaccatgacccagatgaatcaggcgtttcgcaacatcgtgaacatgctgcacggggtgcgtgacgggctggttcgcgacgccaacaactacgaacagcaagagcaggcctcccagcagatcctcagcagcgtcgacatggtcgatgcccaccgcggcggccacccgaccccgatgagctcgacgaaggccacgctgcggctggccgaggccaccgacagctcgggcaagatcaccaagcgcggagccgacaagctgatttccaccatcgacgaattcgccaagattgccatcagctcgggctgtgccgagctgatggccttcgccacgtcggcggtccgcgacgccgagaattccgaggacgtcctgtcccgggtgcgcaaagagaccggtgtcgagttgcaggcgctgcgtggggaggacgagtcacggctgaccttcctggccgtgcgacgatggtacgggtggagcgctgggcgcatcctcaacctcgacatcggcggcggctcgctggaagtgtccagtggcgtggacgaggagcccgagattgcgttatcgctgcccctgggcgccggacggttgacccgagagtggctgcccgacgatccgccgggccggcgccgggtggcgatgctgcgagactggctggatgccgagctggccgagcccagtgtgaccgtcctggaagccggcagccccgacctggcggtcgcaacgtcgaagacgtttcgctcgttggcgcgactaaccggtgcggccccatccatggccgggccgcgggtgaagaggaccctaacggcaaatggtctgcggcaactcatcgcgtttatctctaggatgacggcggttgaccgtgcagaactggaaggggtaagcgccgaccgagcgccgcagattgtggccggcgccctggtggcagaggcgagcatgcgagcactgtcgatagaagcggtggaaatctgcccgtgggcgctgcgggaaggtctcatcttgcgcaaactcgacagcgaagccgacggaaccgccctcatcgagtcttcgtctgtgcacacttcggtgcgtgccgtcggaggtcagccagctgatcggaacgcggccaaccgatcgagaggcagcaaaccaagtacttaaaagctt

SEQ ID NO: 97 - protein, 935 aa, synthetic construct
  REGION 1..935: note = Mycobacterium tuberculosis fusion sequence
MGSSHHHHHHSSGLVPRGSHMDDIDWDAIAQCESGGNWAANTGNGLYGGLQISQATWDSNGGVGSPAAASPQQQIEVADNIMKTQGPGAWPKCSSCSQGDAPLGSLTHILTFLAAETGGCSGSRDDELSPCAYFLVYESTETTERPEHHEFKQAAVLTDLPGELMSALSQGLSQFGINIPPVPSLTGSGDASTGLTGPGLTSPGLTSPGLTSPGLTDPALTSPGLTPTLPGSLAAPGTTLAPTPGVGANPALTNPALTSPTGATPGLTSPTGLDPALGGANEIPITTPVGLDPGADGTYPILGDPTLGTIPSSPATTSTGGGGLVNDVMQVANELGASQAIDLLKGVLMPSIMQAVQNGGAAAPAASPPVPPIPAAAAVPPTDPITVPVAGTHLANGSMSEVMMSEIAGLPIPPIIHYGAIAYAPSGASGKAWHQRTPARAEQVALEKCGDKTCKVVSRFTRCGAVAYNGSKYQGGTGLTRRAAEDDAVNRLEGGRIVNWACNELMTSRFMTDPHAMRDMAGRFEVHAQTVEDEARRMWASAQNISGAGWSGMAEATSLDTMTQMNQAFRNIVNMLHGVRDGLVRDANNYEQQEQASQQILSSVDMVDAHRGGHPTPMSSTKATLRLAEATDSSGKITKRGADKLISTIDEFAKIAISSGCAELMAFATSAVRDAENSEDVLSRVRKETGVELQALRGEDESRLTFLAVRRWYGWSAGRILNLDIGGGSLEVSSGVDEEPEIALSLPLGAGRLTREWLPDDPPGRRRVAMLRDWLDAELAEPSVTVLEAGSPDLAVATSKTFRSLARLTGAAPSMAGPRVKRTLTANGLRQLIAFISRMTAVDRAELEGVSADRAPQIVAGALVAEASMRALSIEAVEICPWALREGLILRKLDSEADGTALIESSSVHTSVRAVGGQPADRNAANRSRGSKPST

SEQ ID NO: 98 - DNA, 3570 bp, synthetic construct
  misc_feature 1..3570: note = Mycobacterium tuberculosis fusion sequence
  source 1..3570: mol_type = other DNA
atgggcagcagccatcatcatcatcatcacagcagcggcctggtgccgcgcggcagccatatggacgacatcgattgggacgccatcgcgcaatgcgaatccggcggcaattgggcggccaacaccggtaacgggttatacggtggtctgcagatcagccaggcgacgtgggattccaacggtggtgtcgggtcgccggcggccgcgagtccccagcaacagatcgaggtcgcagacaacattatgaaaacccaaggcccgggtgcgtggccgaaatgtagttcttgtagtcagggagacgcaccgctgggctcgctcacccacatcctgacgttcctcgcggccgagactggaggttgttcggggagcagggacgatgagctcagtccttgtgcatattttcttgtctacgaatcaaccgaaacgaccgagcggcccgagcaccatgaattcaagcaggcggcggtgttgaccgacctgcccggcgagctgatgtccgcgctatcgcaggggttgtcccagttcgggatcaacataccgccggtgcccagcctgaccgggagcggcgatgccagcacgggtctaaccggtcctggcctgactagtccgggattgaccagcccgggattgaccagcccgggcctcaccgaccctgcccttaccagtccgggcctgacgccaaccctgcccggatcactcgccgcgcccggcaccaccctggcgccaacgcccggcgtgggggccaatccggcgctcaccaaccccgcgctgaccagcccgaccggggcgacgccgggattgaccagcccgacgggtttggatcccgcgctgggcggcgccaacgaaatcccgattacgacgccggtcggattggatcccggggctgacggcacctatccgatcctcggtgatccaacactggggaccataccgagcagccccgccaccacctccaccggcggcggcggtctcgtcaacgacgtgatgcaggtggccaacgagttgggcgccagtcaggctatcgacctgctaaaaggtgtgctaatgccgtcgatcatgcaggccgtccagaatggcggcgcggccgcgccggcagccagcccgccggtcccgcccatccccgcggccgcggcggtgccaccgacggacccaatcaccgtgccggtcgccggtacccatctcgccaacggttcgatgtcggaagtcatgatgtcggaaattgccgggttgcctatccctccgattatccattacggggcgattgcctatgcccccagcggcgcgtcgggcaaagcgtggcaccagcgcacaccggcgcgagcagagcaagtcgcactagaaaagtgcggtgacaagacttgcaaagtggttagtcgcttcaccaggtgcggcgcggtcgcctacaacggctcgaaataccaaggcggaaccggactcacgcgccgcgcggcagaagacgacgccgtgaaccgactcgaaggcgggcggatcgtcaactgggcgtgcaacgagctcatgacctcgcgttttatgacggatccgcacgcgatgcgggacatggcgggccgttttgaggtgcacgcccagacggtggaggacgaggctcgccggatgtgggcgtccgcgcaaaacatctcgggcgcgggctggagtggcatggccgaggcgacctcgctagacaccatgacccagatgaatcaggcgtttcgcaacatcgtgaacatgctgcacggggtgcgtgacgggctggttcgcgacgccaacaactacgaacagcaagagcaggcctcccagcagatcctcagcagcgtcgacatcaatttcgccgttttgccgccggaggtgaattcggcgcgcatattcgccggtgcgggcctgggcccaatgctggcggcggcgtcggcctgggacgggttggccgaggagttgcatgccgcggcgggctcgttcgcgtcggtgaccaccgggttggcgggcgacgcgtggcatggtccggcgtcgctggcgatgacccgcgcggccagcccgtatgtggggtggttgaacacggcggcgggtcaggccgcgcaggcggccggccaggcgcggctagcggcgagcgcgttcgaggcgacgctggcggccaccgtgtctccagcgatggtcgcggccaaccggacacggctggcgtcgctggtggcagccaacttgctgggccagaacgccccggcgatcgcggccgcggaggctgaatacgagcagatatgggcccaggacgtggccgcgatgttcggctatcactccgccgcgtcggcggtggccacgcagctggcgcctattcaagagggtttgcagcagcagctgcaaaacgtgctggcccagttggctagcgggaacctgggcagcggaaatgtgggcgtcggcaacatcggcaacgacaacattggcaacgcaaacatcggcttcggaaatcgaggcgacgccaacatcggcatcgggaatatcggcgacagaaacctcggcattgggaacaccggcaattggaatatcggcatcggcatcaccggcaacggacaaatcggcttcggcaagcctgccaaccccgacgtcttggtggtgggcaacggcggcccgggagtaaccgcgttggtcatgggcggcaccgacagcctactgccgctgcccaacatccccttactcgagtacgctgcgcggttcatcacccccgtgcatcccggatacaccgctacgttcctggaaacgccatcgcagtttttcccattcaccgggctgaatagcctgacctatgacgtctccgtggcccagggcgtaacgaatctgcacaccgcgatcatggcgcaactcgcggcgggaaacgaagtcgtcgtcttcggcacctcccaaagcgccacgatagccaccttcgaaatgcgctatctgcaatccctgccagcacacctgcgtccgggtctcgacgaattgtcctttacgttgaccggcaatcccaaccggcccgacggtggcattcttacgcgttttggcttctccataccgcagttgggtttcacattgtccggcgcgacgcccgccgacgcctaccccaccgtcgattacgcgttccagtacgacggcgtcaacgacttccccaaatacccgctgaatgtcttcgcgaccgccaacgcgatcgcgggcatccttttcctgcactccgggttgattgcgttgccgcccgatcttgcctcgggcgtggttcaaccggtgtcctcaccggacgtcctgaccacctacatcctgctgcccagccaagatctgccgctgctggtcccgctgcgtgctatccccctgctgggaaacccgcttgccgacctcatccagccggacttgcgggtgctcgtcgagttgggttatgaccgcaccgcccaccaggacgtgcccagcccgttcggactgtttccggacgtcgattgggccgaggtggccgcggacctgcagcaaggcgccgtgcaaggcgtcaacgacgccctgtccggactggggctgccgccgccgtggcagccggcgctaccccgacttttcagtacttaaaagctt

SEQ ID NO: 99 - protein, 1187 aa, synthetic construct
  REGION 1..1187: note = Mycobacterium tuberculosis fusion sequence
MGSSHHHHHHSSGLVPRGSHMDDIDWDAIAQCESGGNWAANTGNGLYGGLQISQATWDSNGGVGSPAAASPQQQIEVADNIMKTQGPGAWPKCSSCSQGDAPLGSLTHILTFLAAETGGCSGSRDDELSPCAYFLVYESTETTERPEHHEFKQAAVLTDLPGELMSALSQGLSQFGINIPPVPSLTGSGDASTGLTGPGLTSPGLTSPGLTSPGLTDPALTSPGLTPTLPGSLAAPGTTLAPTPGVGANPALTNPALTSPTGATPGLTSPTGLDPALGGANEIPITTPVGLDPGADGTYPILGDPTLGTIPSSPATTSTGGGGLVNDVMQVANELGASQAIDLLKGVLMPSIMQAVQNGGAAAPAASPPVPPIPAAAAVPPTDPITVPVAGTHLANGSMSEVMMSEIAGLPIPPIIHYGAIAYAPSGASGKAWHQRTPARAEQVALEKCGDKTCKVVSRFTRCGAVAYNGSKYQGGTGLTRRAAEDDAVNRLEGGRIVNWACNELMTSRFMTDPHAMRDMAGRFEVHAQTVEDEARRMWASAQNISGAGWSGMAEATSLDTMTQMNQAFRNIVNMLHGVRDGLVRDANNYEQQEQASQQILSSVDINFAVLPPEVNSARIFAGAGLGPMLAAASAWDGLAEELHAAAGSFASVTTGLAGDAWHGPASLAMTRAASPYVGWLNTAAGQAAQAAGQARLAASAFEATLAATVSPAMVAANRTRLASLVAANLLGQNAPAIAAAEAEYEQIWAQDVAAMFGYHSAASAVATQLAPIQEGLQQQLQNVLAQLASGNLGSGNVGVGNIGNDNIGNANIGFGNRGDANIGIGNIGDRNLGIGNTGNWNIGIGITGNGQIGFGKPANPDVLVVGNGGPGVTALVMGGTDSLLPLPNIPLLEYAARFITPVHPGYTATFLETPSQFFPFTGLNSLTYDVSVAQGVTNLHTAIMAQLAAGNEVVVFGTSQSATIATFEMRYLQSLPAHLRPGLDELSFTLTGNPNRPDGGILTRFGFSIPQLGFTLSGATPADAYPTVDYAFQYDGVNDFPKYPLNVFATANAIAGILFLHSGLIALPPDLASGVVQPVSSPDVLTTYILLPSQDLPLLVPLRAIPLLGNPLADLIQPDLRVLVELGYDRTAHQDVPSPFGLFPDVDWAEVAADLQQGAVQGVNDALSGLGLPPPWQPALPRLFST

SEQ ID NO: 100 - protein, 164 aa, Mycobacterium tuberculosis
VAGDTTITIVGNLTADPELRFTPSGAAVANFTVASTPRIYDRQTGEWKDGEALFLRCNIWREAAENVAESLTRGARVIVSGRLKQRSFETREGEKRTVIEVEVDEIGPSLRYATAKVNKASRSGGFGSGSRPAPAQTSSASGDDPWGSAPASGSFGGGDDEPPF

SEQ ID NO: 101 - DNA, 507 bp, Mycobacterium tuberculosis
  source 1..507: mol_type = genomic DNA
catatggtggctggtgacaccaccatcaccatcgtcggaaatctgaccgctgaccccgagctgcggttcaccccgtccggtgcggccgtggcgaatttcaccgtggcgtcaacgccccggatctatgaccgtcagaccggcgaatggaaagacggcgaagcgctgttcctccggtgcaatatctggcgggaggcggccgagaacgtggccgagagcctcacccggggggcacgagtcatcgttagcgggcggcttaagcagcggtcgtttgaaacccgtgagggcgagaagcgcaccgtcatcgaggtcgaggtcgatgagattgggccttcgcttcggtacgccaccgccaaggtcaacaaggccagccgcagcggcgggtttggcagcggatcccgtccggcgccggcgcagaccagcagcgcctcgggagatgacccgtggggcagcgcaccggcgtcgggttcgttcggcggcggcgatgacgaaccgccattctgaaagctt

SEQ ID NO: 102 - protein, 185 aa, Mycobacterium tuberculosis
MGSSHHHHHHSSGLVPRGSHMVAGDTTITIVGNLTADPELRFTPSGAAVANFTVASTPRIYDRQTGEWKDGEALFLRCNIWREAAENVAESLTRGARVIVSGRLKQRSFETREGEKRTVIEVEVDEIGPSLRYATAKVNKASRSGGFGSGSRPAPAQTSSASGDDPWGSAPASGSFGGGDDEPPF

SEQ ID NO: 103 - protein, 161 aa, Mycobacterium tuberculosis; identical to SEQ ID NO: 1

SEQ ID NO: 104 - DNA, 495 bp, Mycobacterium tuberculosis; identical to SEQ ID NO: 2
  source 1..495: mol_type = genomic DNA

SEQ ID NO: 105 - protein, 181 aa, Mycobacterium tuberculosis; identical to SEQ ID NO: 3

SEQ ID NO: 106 - protein, 750 aa, Mycobacterium tuberculosis
MAKASETERSGPGTQPADAQTATSATVRPLSTQAVFRPDFGDEDNFPHPTLGPDTEPQDRMATTSRVRPPVRRLGGGLVEIPRAPDIDPLEALMTNPVVPESKRFCWNCGRPVGRSDSETKGASEGWCPYCGSPYSFLPQLNPGDIVAGQYEVKGCIAHGGLGWIYLALDRNVNGRPVVLKGLVHSGDAEAQAMAMAERQFLAEVVHPSIVQIFNFVEHTDRHGDPVGYIVMEYVGGQSLKRSKGQKLPVAEAIAYLLEILPALSYLHSIGLVYNDLKPENIMLTEEQLKLIDLGAVSRINSFGYLYGTPGFQAPEIVRTGPTVATDIYTVGRTLAALTLDLPTRNGRYVDGLPEDDPVLKTYDSYGRLLRRAIDPDPRQRFTTAEEMSAQLTGVLREVVAQDTGVPRPGLSTIFSPSRSTFGVDLLVAHTDVYLDGQVHAEKLTANEIVTALSVPLVDPTDVAASVLQATVLSQPVQTLDSLRAARHGALDADGVDFSESVELPLMEVRALLDLGDVAKATRKLDDLAERVGWRWRLVWYRAVAELLTGDYDSATKHFTEVLDTFPGELAPKLALAATAELAGNTDEHKFYQTVWSTNDGVISAAFGLARARSAEGDRVGAVRTLDEVPPTSRHFTTARLTSAVTLLSGRSTSEVTEEQIRDAARRVEALPPTEPRVLQIRALVLGGALDWLKDNKASTNHILGFPFTSHGLRLGVEASLRSLARVAPTQRHRYTLVDMANKVRPTSTF

SEQ ID NO: 107 - DNA, 2289 bp, Mycobacterium tuberculosis
  source 1..2289: mol_type = genomic DNA
catatgcatatgcatcaccatcaccatcacatggccaaagcgtcagagaccgaacgttcgggccccggcacccaaccggcggacgcccagaccgcgacgtccgcgacggttcgacccctgagcacccaggcggtgttccgccccgatttcggcgatgaggacaacttcccccatccgacgctcggcccggacaccgagccgcaagaccggatggccaccaccagccgggtgcgcccgccggtcagacggctgggcggcggcctggtggaaatcccgcgggcgcccgatatcgatccgcttgaggccctgatgaccaacccggtggtgccggagtccaagcggttctgctggaactgtggacgtcccgtcggccggtccgactcggagaccaagggagcttcagagggctggtgtccctattgcggcagcccgtattcgttcctgccgcagctaaatcccggggacatcgtcgccggccagtacgaggtcaaaggctgcatcgcgcacggcggactgggctggatctacctcgctctcgaccgcaatgtcaacggccgtccggtggtgctcaagggcctggtgcattccggtgatgccgaagcgcaggcaatggcgatggccgaacgccagttcctggccgaggtggtgcacccgtcgatcgtgcagatcttcaactttgtcgagcacaccgacaggcacggggatccggtcggctacatcgtgatggaatacgtcggcgggcaatcgctcaaacgcagcaagggtcagaaactgcccgtcgcggaggccatcgcctacctgctggagatcctgccggcgctgagctacctgcattccatcggcttggtctacaacgacctgaagccggaaaacatcatgctgaccgaggaacagctcaagctgatcgacctgggcgcggtatcgcggatcaactcgttcggctacctctacgggaccccaggcttccaggcgcccgagatcgtgcggaccggtccgacggtggccaccgacatctacaccgtgggacgcacgctcgcggcgctcacgctggacctgcccacccgcaatggccgttatgtggatgggctacccgaagacgacccggtgctgaaaacctacgactcttacggccggttgctgcgcagggccatcgaccccgatccgcggcaacggttcaccaccgccgaagagatgtccgcgcaattgacgggcgtgttgcgggaggtggtcgcccaggacaccggggtgccgcggccagggctatcaacgatcttcagtcccagtcggtcgacatttggagtggacctgctggtggcgcacaccgacgtgtatctggacgggcaggtgcacgcggagaagctgaccgccaacgagatcgtgaccgcgctgtcggtgccgctggtcgatccgaccgacgtcgcagcttcggtcctgcaggccacggtgctctcccagccggtgcagaccctagactcgctgcgcgcggcccgccacggtgcgctggacgccgacggcgtcgacttctccgagtcagtggagctgccgctaatggaagtccgcgcgctgctggatctcggcgatgtggccaaggccacccgaaaactcgacgatctggccgaacgcgttggctggcgatggcgattggtctggtaccgggccgtcgccgagctgctcaccggcgactatgactcggccaccaaacatttcaccgaggtgctggatacctttcccggcgagctggcgcccaagctcgccctggccgccaccgccgaactagccggcaacaccgacgaacacaagttctatcagacggtgtggagcaccaacgacggcgtgatctcggcggctttcggactggccagagcccggtcggccgaaggtgatcgggtcggcgccgtgcgcacgctcgacgaggtaccgcccacttctcggcatttcaccacggcacggctgaccagcgcggtgactctgttgtccggccggtcaacgagtgaagtcaccgaggaacagatccgcgacgccgcccgaagagtggaggcgctgcccccgaccgaaccacgcgtgctgcagatccgcgccctggtgctgggtggcgcgctggactggctgaaggacaacaaggccagcaccaaccacatcctcggtttcccgttcaccagtcacgggctgcggctgggtgtcgaggcgtcactgcgcagcctggcccgggtagctcccactcaacggcatcgctacacgctggtggacatggccaacaaggtccggcccaccagcacgttctaagaattc

SEQ ID NO: 108 - protein, 758 aa, Mycobacterium tuberculosis
HMHHHHHHMAKASETERSGPGTQPADAQTATSATVRPLSTQAVFRPDFGDEDNFPHPTLGPDTEPQDRMATTSRVRPPVRRLGGGLVEIPRAPDIDPLEALMTNPVVPESKRFCWNCGRPVGRSDSETKGASEGWCPYCGSPYSFLPQLNPGDIVAGQYEVKGCIAHGGLGWIYLALDRNVNGRPVVLKGLVHSGDAEAQAMAMAERQFLAEVVHPSIVQIFNFVEHTDRHGDPVGYIVMEYVGGQSLKRSKGQKLPVAEAIAYLLEILPALSYLHSIGLVYNDLKPENIMLTEEQLKLIDLGAVSRINSFGYLYGTPGFQAPEIVRTGPTVATDIYTVGRTLAALTLDLPTRNGRYVDGLPEDDPVLKTYDSYGRLLRRAIDPDPRQRFTTAEEMSAQLTGVLREVVAQDTGVPRPGLSTIFSPSRSTFGVDLLVAHTDVYLDGQVHAEKLTANEIVTALSVPLVDPTDVAASVLQATVLSQPVQTLDSLRAARHGALDADGVDFSESVELPLMEVRALLDLGDVAKATRKLDDLAERVGWRWRLVWYRAVAELLTGDYDSATKHFTEVLDTFPGELAPKLALAATAELAGNTDEHKFYQTVWSTNDGVISAAFGLARARSAEGDRVGAVRTLDEVPPTSRHFTTARLTSAVTLLSGRSTSEVTEEQIRDAARRVEALPPTEPRVLQIRALVLGGALDWLKDNKASTNHILGFPFTSHGLRLGVEASLRSLARVAPTQRHRYTLVDMANKVRPTSTF

SEQ ID NO: 109 - protein, 328 aa, Mycobacterium tuberculosis; identical to SEQ ID NO: 6

SEQ ID NO: 110 - DNA, 996 bp, Mycobacterium tuberculosis; identical to SEQ ID NO: 7
  source 1..996: mol_type = genomic DNA

SEQ ID NO: 111 - protein, 348 aa, Mycobacterium tuberculosis; identical to SEQ ID NO: 8

SEQ ID NO: 112 - protein, 359 aa, Mycobacterium tuberculosis
VRYSDSYHTTGRWQPRASTEGFPMGVSIEVNGLTKSFGSSRIWEDVTLTIPAGEVSVLLGPSGTGKSVFLKSLIGLLRPERGSIIIDGTDIIECSAKELYEIRTLFGVLFQDGALFGSMNLYDNTAFPLREHTKKKESEIRDIVMEKLALVGLGGDEKKFPGEISGGMRKRAGLARALVLDPQIILCDEPDSGLDPVRTAYLSQLIMDINAQIDATILIVTHNINIARTVPDNMGMLFRKHLVMFGPREVLLTSDEPVVRQFLNGRRIGPIGMSEEKDEATMAEEQALLDAGHHAGGVEEIEGVPPQISATPGMPERKAVARRQARVREMLHTLPKKAQAAILDDLEGTHKYAVHEIGQ

SEQ ID NO: 113 - DNA, 1088 bp, Mycobacterium tuberculosis
  source 1..1088: mol_type = genomic DNA
catatgcgatacagtgactcataccacacaacgggccggtggcagccacgagcgtcgacagagggtttcccatgggcgtcagcatcgaggtcaacggactaacgaagtccttcgggtcctcgaggatctgggaagatgtcacgctaacgatccccgccggggaggtcagcgtgctgctgggcccatcgggtaccggcaaatcggtgtttctgaaatctctgatcggcctcctgcggccggagcgcggctcgatcatcatcgacggcaccgacatcatcgaatgctcggccaaggagctttacgagatccgcacattgttcggcgtgctgtttcaggacggtgccctgttcgggtcgatgaacctctacgacaacaccgcgttccccctgcgtgagcacaccaagaaaaaggaaagcgagatccgtgacatcgtcatggagaagctggccctagtcggcctgggtggggacgagaagaagttccccggcgagatctccggcgggatgcgtaagcgtgccggcctagcgcgtgccctggtccttgacccgcagatcattctctgcgacgagcccgactcgggtctggacccggttcgtaccgcctacctgagccagctgatcatggacatcaacgcccagatcgacgccaccatcctgatcgtgacgcacaacatcaacatcgcccgcaccgtgccggacaacatgggcatgttgttccgcaagcatttggtgatgttcgggccgcgggaggtgctactcaccagcgacgagccggtggtgcggcagttcctcaacggccggcgcatcggcccgatcggcatgtccgaggagaaggacgaggccaccatggccgaagagcaggccctgctcgatgccggccaccacgcgggcggtgtcgaggaaatcgagggcgtgccgccgcagatcagcgcgacaccgggcatgccggagcgcaaagcggtcgcccggcgtcaggctcgggttcgcgagatgttgcacacgctgcccaaaaaggcccaggcggcgatcctcgacgatctcgagggcacgcacaagtacgcggtgcacgaaatcggccagtaaaagctt

SEQ ID NO: 114 - protein, 379 aa, Mycobacterium tuberculosis
MGSSHHHHHHSSGLVPRGSHMRYSDSYHTTGRWQPRASTEGFPMGVSIEVNGLTKSFGSSRIWEDVTLTIPAGEVSVLLGPSGTGKSVFLKSLIGLLRPERGSIIIDGTDIIECSAKELYEIRTLFGVLFQDGALFGSMNLYDNTAFPLREHTKKKESEIRDIVMEKLALVGLGGDEKKFPGEISGGMRKRAGLARALVLDPQIILCDEPDSGLDPVRTAYLSQLIMDINAQIDATILIVTHNINIARTVPDNMGMLFRKHLVMFGPREVLLTSDEPVVRQFLNGRRIGPIGMSEEKDEATMAEEQALLDAGHHAGGVEEIEGVPPQISATPGMPERKAVARRQARVREMLHTLPKKAQAAILDDLEGTHKYAVHEIGQ

SEQ ID NO: 115 - protein, 270 aa, Mycobacterium tuberculosis
MLPETNQDEVQPNAPVALVTVEIRHPTTDSLTESANRELKHLLINDLPIERQAQDVSWGMTAPGGAPTPVADRFVRYVNRDNTTAASLKNQAIVVETTAYRSFEAFTDVVMRVVDARAQVSSIVGLERILRFVLEIRVPAGVDGRITWSNWIDEQLLGPQRFTPGGLVLTEWQGAAVYRELQPGKSLIVRYGPGMGQALDPNYHLRRITPAQTGPFFLLDIDSFWTPSGGSIPEYNRDALVSTFQDLYGPAQVVFQEMITSRLKDELLRQ

SEQ ID NO: 116 - DNA, 825 bp, Mycobacterium tuberculosis
  source 1..825: mol_type = genomic DNA
catatgctccccgagacaaatcaggatgaggtccagcccaacgcacccgttgccctggtgacggtggaaatccgtcacccgacaacggattcgctcaccgaatcagcgaaccgggagctcaaacacctgcttatcaatgatctaccgatcgaacgccaggcgcaggacgtcagctgggggatgacggcgcccggtggagcccccaccccggtcgcggatcgtttcgttcgttatgtcaatcgcgataacaccaccgccgcttcactgaagaaccaggcgatagtcgtggagaccaccgcctaccgcagctttgaggcctttaccgacgttgtgatgcgggtcgtggatgctcgcgcgcaggtctcgtcaatcgttgggttggagcgtatcggtcttcgctttgttctggagatccgcgtccccgcgggtgtcgacggccggatcacgtggagcaactggatcgacgagcagctgctcgggccgcagcgtttcactcccggcggcctggtcctgaccgagtggcagggtgccgcagtctaccgtgagctacaaccaggcaaatcgctcatcgtgcgctacggcccgggtatgggccaagcgcttgatcccaattaccatctgcgccgaataacacccgcccaaaccggaccattcttcctgctggacatcgatagcttttggactcccagtggcggctccattcccgagtacaacagggacgccttagtgtcgacattccaggacctgtacggtccggcccaggtcgtgtttcaggagatgatcaccagtcgcctgaaagatgagctgcttcgccagtaaaagctt

SEQ ID NO: 117 - protein, 291 aa, Mycobacterium tuberculosis
MGSSHHHHHHSSGLVPRGSHMMLPETNQDEVQPNAPVALVTVEIRHPTTDSLTESANRELKHLLINDLPIERQAQDVSWGMTAPGGAPTPVADRFVRYVNRDNTTAASLKNQAIVVETTAYRSFEAFTDVVMRVVDARAQVSSIVGLERILRFVLEIRVPAGVDGRITWSNWIDEQLLGPQRFTPGGLVLTEWQGAAVYRELQPGKSLIVRYGPGMGQALDPNYHLRRITPAQTGPFFLLDIDSFWTPSGGSIPEYNRDALVSTFQDLYGPAQVVFQEMITSRLKDELLRQ

SEQ ID NO: 118 - protein, 362 aa, Mycobacterium tuberculosis
MLRLVVGALLLVLAFAGGYAVAACKTVTLTVDGTAMRVTTMKSRVIDIVEENGFSVDDRDDLYPAAGVQVHDADTIVLRRSRPLQISLDGHDAKQVWTTASTVDEALAQLAMTDTAPAAASRASRVPLSGMALPVVSAKTVQLNDGGLVRTVHLPAPNVAGLLSAAGVPLLQSDHVVPAATAPIVEGMQIQVTRNRIKKVTERLPLPPNARRVEDPEMNMSREVVEDPGVPGTQDVTFAVAEVNGVETGRLPVANVVVTPAHEAVVRVGTKPGTEVPPVIDGSIWDAIAGCEAGGNWAINTGNGYYGGVQFDQGTWEANGGLRYAPRADLATREEQIAVAEVTRLRQGWGAWPVCAARAGAR

SEQ ID NO: 119 - DNA, 1053 bp, Mycobacterium tuberculosis
  source 1..1053: mol_type = genomic DNA
catatgcatcaccatcaccatcacgcatgcaaaacggtgacgttgaccgtcgacggaaccgcgatgcgggtgaccacgatgaaatcgcgggtgatcgacatcgtcgaagagaacgggttctcagtcgacgaccgcgacgacctgtatcccgcggccggcgtgcaggtccatgacgccgacaccatcgtgctgcggcgtagccgtccgctgcagatctcgctggatggtcacgacgctaagcaggtgtggacgaccgcgtcgacggtggacgaggcgctggcccaactcgcgatgaccgacacggcgccggccgcggcttctcgcgccagccgcgtcccgctgtccgggatggcgctaccggtcgtcagcgccaagacggtgcagctcaacgacggcgggttggtgcgcacggtgcacttgccggcccccaatgtcgcggggctgctgagtgcggccggcgtgccgctgttgcaaagcgaccacgtggtgcccgccgcgacggccccgatcgtcgaaggcatgcagatccaggtgacccgcaatcggatcaagaaggtcaccgagcggctgccgctgccgccgaacgcgcgtcgtgtcgaggacccggagatgaacatgagccgggaggtcgtcgaagacccgggggttccggggacccaggatgtgacgttcgcggtagctgaggtcaacggcgtcgagaccggccgtttgcccgtcgccaacgtcgtggtgaccccggcccacgaagccgtggtgcgggtgggcaccaagcccggtaccgaggtgcccccggtgatcgacggaagcatctgggacgcgatcgccggctgtgaggccggtggcaactgggcgatcaacaccggcaacgggtattacggtggtgtgcagtttgaccagggcacctgggaggccaacggcgggctgcggtatgcaccccgcgctgacctcgccacccgcgaagagcagatcgccgttgccgaggtgacccgactgcgtcaaggttggggcgcctggccggtatgtgctgcacgagcgggtgcgcgctgagaattc

SEQ ID NO: 120 - protein, 348 aa, Mycobacterium tuberculosis
HMHHHHHHACKTVTLTVDGTAMRVTTMKSRVIDIVEENGFSVDDRDDLYPAAGVQVHDADTIVLRRSRPLQISLDGHDAKQVWTTASTVDEALAQLAMTDTAPAAASRASRVPLSGMALPVVSAKTVQLNDGGLVRTVHLPAPNVAGLLSAAGVPLLQSDHVVPAATAPIVEGMQIQVTRNRIKKVTERLPLPPNARRVEDPEMNMSREVVEDPGVPGTQDVTFAVAEVNGVETGRLPVANVVVTPAHEAVVRVGTKPGTEVPPVIDGSIWDAIAGCEAGGNWAINTGNGYYGGVQFDQGTWEANGGLRYAPRADLATREEQIAVAEVTRLRQGWGAWPVCAARAGAR

SEQ ID NO: 121 - protein, 328 aa, Mycobacterium tuberculosis
MELVRVTEAGAMAAGRWVGRGDKEGGDGAAVDAMRELVNSVSMRGVVVIGEGEKDHAPMLYNGEEVGNGDGPECDFAVDPIDGTTLMSKGMTNAISVLAVADRGTMFDPSAVFYMNKIAVGPDAAHVLDITAPISENIRAVAKVKDLSVRDMTVCILDRPRHAQLIHDVRATGARIRLITDGDVAGAISACRPHSGTDLLAGIGGTPEGIIAAAAIRCMGGAIQAQLAPRDDAERRKALEAGYDLNQVLTTEDLVSGENVFFCATGVTDGDLLKGVRYYPGGCTTHSIVMRSKSGTVRMIEAYHRLSKLNEYSAIDFTGDSSAVYPLP

SEQ ID NO: 122 - DNA, 999 bp, Mycobacterium tuberculosis
  source 1..999: mol_type = genomic DNA
catatgatggagctggtccgggtgaccgaggccggagccatggccgcgggccgctgggtaggccgcggcgacaaggagggcggcgacggcgcggcggtcgacgcgatgcgcgaactggtcaactcggtttccatgcgcggggtggtggtcatcggcgaaggcgaaaaggaccacgcaccaatgctctacaacggcgaagaagtgggcaacggcgacggaccggaatgcgactttgccgtcgaccccattgacggcaccacgctgatgagcaagggcatgaccaacgccatctcggtgctggcggtagccgatcgcggcaccatgttcgacccgtcggcggtgttctacatgaacaaaatcgccgtcggccccgatgccgcacacgtgctggatatcaccgcgccgatctcggaaaacatccgagcggtcgccaaggtcaaggacctgtcggtgcgagacatgacggtgtgcatcctggacaggccgcggcacgcgcaactcatccacgacgtccgcgccaccggggcccggatccggctgatcaccgatggcgacgtcgccggcgcgatctcggcgtgccgaccgcactccggcaccgacctgctagctgggatcggcggcaccccggagggaatcatcgccgccgcggcgatccgctgcatgggcggggcgatccaggcgcagctcgccccgcgcgacgacgcggaacgccgcaaggccctagaagccggttacgacctgaaccaggtcttgaccaccgaagatctggtgtccggggaaaacgtcttcttctgcgccactggggtcaccgacggcgacctgctcaagggagtgcgttactaccccggcggctgcaccacccattcgatcgtgatgcgctcgaagtccggcaccgtccggatgatcgaggcctaccaccggctttcaaagctcaacgaatactccgcgatcgacttcaccggcgacagcagcgccgtgtacccattgccctaaaagctt

SEQ ID NO: 123 - protein, 348 aa, Mycobacterium tuberculosis
MGSSHHHHHHSSGLVPRGSHMELVRVTEAGAMAAGRWVGRGDKEGGDGAAVDAMRELVNSVSMRGVVVIGEGEKDHAPMLYNGEEVGNGDGPECDFAVDPIDGTTLMSKGMTNAISVLAVADRGTMFDPSAVFYMNKIAVGPDAAHVLDITAPISENIRAVAKVKDLSVRDMTVCILDRPRHAQLIHDVRATGARIRLITDGDVAGAISACRPHSGTDLLAGIGGTPEGIIAAAAIRCMGGAIQAQLAPRDDAERRKALEAGYDLNQVLTTEDLVSGENVFFCATGVTDGDLLKGVRYYPGGCTTHSIVMRSKSGTVRMIEAYHRLSKLNEYSAIDFTGDSSAVYPLP

SEQ ID NO: 124 - protein, 329 aa, Mycobacterium tuberculosis
VSASPLKVAVTGAAGQIGYSLLFRLASGSLLGPDRPIELRLLEIEPALQALEGVVMELDDCAFPLLSGVEIGSDPQKIFDGVSLALLVGARPRGAGMERSDLLEANGAIFTAQGKALNAVAADDVRVGVTGNPANTNALIAMTNAPDIPRERFSALTRLDHNRAISQLAAKTGAAVTDIKKMTIWGNHSATQYPDLFHAEVAGKNAAEVVNDQAWIEDEFIPTVAKRGAAIIDARGASSAASAASATIDAARDWLLGTPADDWVSMAVVSDGSYGVPEGLISSFPVTTKGGNWTIVSGLEIDEFSRGRIDKSTAELADERSAVTELGLI

SEQ ID NO: 125 - DNA, 999 bp, Mycobacterium tuberculosis
  source 1..999: mol_type = genomic DNA
catatgagcgctagtcctctcaaggtcgccgttaccggcgccgccggccaaatcggctacagcctgttgttccgcctggccagcggctctttgctgggccctgaccgtccgatcgagctgcggctgctcgagatcgagccggcactgcaggcgctcgagggtgtggtgatggaactcgacgactgcgctttcccgctgttgtccggggtggagatcggttcagatccccagaagatcttcgatggcgtgagcctggccctgctggtcggagcccgcccccggggcgcgggcatggagcgaagtgacctgctggaggccaacggcgcgatcttcaccgctcagggcaaagccctcaacgctgtcgccgcggatgacgttcgcgtcggggtgaccggcaaccccgccaacaccaacgcgctgatcgcgatgaccaatgcgcccgacattccccgcgagcggttctcggcgctcacccggctggaccacaatcgggcgatctcgcagctggccgccaagaccggcgcggcggtcaccgacatcaagaagatgacgatctggggcaatcactcggccacccagtaccccgacctgttccacgcggaggtcgccggaaagaacgcggccgaagtggtcaacgaccaggcctggatcgaggatgaattcatcccgacggtcgccaagcgcggtgcggcgatcatcgatgcgcgcggcgcgtcgtcggccgcctcggccgcgtcggcaaccatcgacgctgcccgggactggttgctggggacgccggcggacgattgggtctcgatggccgtcgtctccgacgggtcctacggggtgccggagggcttgatctcctcgtttccggtcaccaccaagggcggcaactggacgatcgtgagcggcttggagatcgacgagttctcccgcggccggatcgacaagtcaaccgccgagttggctgacgagcgcagcgcggtcaccgagctcggcctgatctgaaagctt

SEQ ID NO: 126 - protein, 349 aa, Mycobacterium tuberculosis
MGSSHHHHHHSSGLVPRGSHMSASPLKVAVTGAAGQIGYSLLFRLASGSLLGPDRPIELRLLEIEPALQALEGVVMELDDCAFPLLSGVEIGSDPQKIFDGVSLALLVGARPRGAGMERSDLLEANGAIFTAQGKALNAVAADDVRVGVTGNPANTNALIAMTNAPDIPRERFSALTRLDHNRAISQLAAKTGAAVTDIKKMTIWGNHSATQYPDLFHAEVAGKNAAEVVNDQAWIEDEFIPTVAKRGAAIIDARGASSAASAASATIDAARDWLLGTPADDWVSMAVVSDGSYGVPEGLISSFPVTTKGGNWTIVSGLEIDEFSRGRIDKSTAELADERSAVTELGLI

SEQ ID NO: 127 - protein, 456 aa, Mycobacterium tuberculosis
MVSTHAVVAGETLSALALRFYGDAELYRLIAAASGIADPDVVNVGQRLIMPDFTRYTVVAGDTLSALALRFYGDAELNWLIAAASGIADPDVVNVGQRLIMPDFTRYTVVAGDTLSALAARFYGDASLYPLIAAVNGIADPGVIDVGQVLVIFIGRSDGFGLRIVDRNENDPRLWYYRFQTSAIGWNPGVNVLLPDDYRTSGRTYPVLYLFHGGGTDQDFRTFDFLGIRDLTAGKPIIIVMPDGGHAGWYSNPVSSFVGPRNWETFHIAQLLPWIEANFRTYAEYDGRAVAGFSMGGFGALKYAAKYYGHFASASSHSGPASLRRDFGLVVHWANLSSAVLDLGGGTVYGAPLWDQARVSADNPVERIDSYRNKRIFLVAGTSPDPANWFDSVNETQVLAGQREFRERLSNAGIPHESHEVPGGHVFRPDMFRLDLDGIVARLRPASIGAAAERAD

SEQ ID NO: 128 - DNA, 1380 bp, Mycobacterium tuberculosis
  source 1..1380: mol_type = genomic DNA
catatggtcagcacacatgcggttgtcgcgggggagacgctgtcggcgttggcgttgcgcttctatggcgacgcggaactgtatcggctgatcgccgccgccagcgggatcgccgatcccgacgtcgtcaatgtggggcagcggctgattatgcctgacttcacgcgatacaccgttgttgccggggacacgctgtcggcgttggcgttgcgcttctatggcgacgcggaattgaattggctgatcgccgccgccagcgggatcgccgatcccgacgtcgtcaatgtggggcagcggctgattatgcctgacttcacgcgatacaccgttgttgccggggacacgctgtcggcattggctgcgcgcttctatggcgacgcctccctatatccgcttatcgccgccgtcaatggcatcgccgatcctggcgtcatcgacgtcgggcaggtactggtcatattcatcgggcgtagcgacgggttcggcctaaggatcgtggaccgcaacgagaacgatccccgcctgtggtactaccggttccagacctccgcgatcggctggaaccccggagtcaacgtcctgcttcccgatgactaccgcaccagcggacgcacctatcccgtcctctacctgttccacggcggcggcaccgaccaggatttccgcacgttcgactttctgggcatccgcgacctgaccgccggaaagccgatcatcatcgtgatgcccgacggcgggcacgcgggctggtattccaacccggtcagctcgttcgtcggcccacggaactgggagacattccacatcgcccagctgctcccctggatcgaggcgaacttccgaacctacgccgaatacgacggccgcgcggtcgccgggttttcgatgggtggcttcggcgcgctgaagtacgcagcaaagtactacggccacttcgcgtcggcgagcagccactccggaccggcaagtctgcgccgcgacttcggcctggtagtgcattgggcaaacctgtcctcggcggtgctggatctaggcggcggcacggtttacggcgcgccgctctgggaccaagctagggtcagcgccgacaacccggtcgagcgtatcgacagctaccgcaacaagcggatcttcctggtcgccggcaccagtccggacccggccaactggttcgacagcgtgaacgagacccaggtgctagccgggcagagggagttccgcgaacgcctcagcaacgccggcatcccgcatgaatcgcacgaggtgcctggcggtcacgtcttccggcccgacatgttccgtctcgacctcgacggcatcgtcgcccggctgcgccccgcgagcatcggggcggccgcagaacgcgccgattagaagctt

SEQ ID NO: 129 - protein, 476 aa, Mycobacterium tuberculosis
MGSSHHHHHHSSGLVPRGSHMVSTHAVVAGETLSALALRFYGDAELYRLIAAASGIADPDVVNVGQRLIMPDFTRYTVVAGDTLSALALRFYGDAELNWLIAAASGIADPDVVNVGQRLIMPDFTRYTVVAGDTLSALAARFYGDASLYPLIAAVNGIADPGVIDVGQVLVIFIGRSDGFGLRIVDRNENDPRLWYYRFQTSAIGWNPGVNVLLPDDYRTSGRTYPVLYLFHGGGTDQDFRTFDFLGIRDLTAGKPIIIVMPDGGHAGWYSNPVSSFVGPRNWETFHIAQLLPWIEANFRTYAEYDGRAVAGFSMGGFGALKYAAKYYGHFASASSHSGPASLRRDFGLVVHWANLSSAVLDLGGGTVYGAPLWDQARVSADNPVERIDSYRNKRIFLVAGTSPDPANWFDSVNETQVLAGQREFRERLSNAGIPHESHEVPGGHVFRPDMFRLDLDGIVARLRPASIGAAAERAD

SEQ ID NO: 130 - protein, 518 aa, Mycobacterium tuberculosis
MRAGRRVAISAGSLAVLLGALDTYVVVTIMRDIMNSVGIPINQLHRITWIVTMYLLGYIAAMPLLGRASDRFGRKLMLQVSLAGFIIGSVVTALAGHFGDFHMLIAGRTIQGVASGALLPITLALGADLWSQRNRAGVLGGIGAAQELGSVLGPLYGIFIVWLLHDWRDVFWINVPLTAIAMVMIHFSLPSHDRSTEPERVDLVGGLLLALALGLAVIGLYNPNPDGKHVLPDYGAPLLVGALVAAVAFFGWERFARTRLIDPAGVHFRPFLSALGASVAAGAALMVTLVDVELFGQGVLQMDQAQAAGMLLWFLIALPIGAVTGGWIATRAGDRAVAFAGLLIAAYGYWLISHWPVDLLADRHNILGLFTVPAMHTDLVVAGLGLGLVIGPLSSATLRVVPSAQHGIASAAVVVARMTGMLIGVAALSAWGLYRFNQILAGLSAAIPPNASLLERAAAIGARYQQAFALMYGEIFTITAIVCVFGAVLGLLISGRKEHADEPEVQEQPTLAPQVEPL

SEQ ID NO: 131 - DNA, 996 bp, Mycobacterium tuberculosis
  source 1..996: mol_type = genomic DNA
catatggagctggtccgggtgaccgaggccggagccatggccgcgggccgctgggtaggccgcggcgacaaggagggcggcgacggcgcggcggtcgacgcgatgcgcgaactggtcaactcggtttccatgcgcggggtggtggtcatcggcgaaggcaaaaaggaccacgcaccaatgctctacaacggcgaagaagtgggcaacggcgacggaccggaatgcgactttgccgtcgaccccattgacggcaccacgctgatgagcaagggcatgaccaacgccatctcggtgctggcggtagccgatcgcggcaccatgttcgacccgtcggcggtgttctacatgaacaaaatcgccgtcggccccgatgccgcacacgtgctggatatcaccgcgccgatctcggaaaacatccgagcggtcgccaaggtcaaggacctgtcggtgcgagacatgacggtgtgcatcctggacaggccgcggcacgcgcaactcatccacgacgtccgcgccaccggggcccggatccggctgatcaccgatggcgacgtcgccggcgcgatctcggcgtgccgaccgcactccggcaccgacctgctagctgggatcggcggcaccccggagggaatcatcgccgccgcggcgatccgctgcatgggcggggcgatccaggcgcagctcgccccgcgcgacgacgcggaacgccgcaaggccctagaagccggttacgacctgaaccaggtcttgaccaccgaagatctggtgtccggggaaaacgtcttcttctgcgccactggggtcaccgacggcgacctgctcaagggagtgcgttactaccccggcggctgcaccacccattcgatcgtgatgcgctcgaagtccggcaccgtccggatgatcgaggcctaccaccggctttcaaagctcaacgaatactccgcgatcgacttcaccggcgacagcagcgccgtgtacccattgccctaaaagctt

SEQ ID NO: 132 - protein, 349 aa, Mycobacterium tuberculosis
MGSSHHHHHHSSGLVPRGSHHMELVRVTEAGAMAAGRWVGRGDKEGGDGAAVDAMRELVNSVSMRGVVVIGEGEKDHAPMLYNGEEVGNGDGPECDFAVDPIDGTTLMSKGMTNAISVLAVADRGTMFDPSAVFYMNKIAVGPDAAHVLDITAPISENIRAVAKVKDLSVRDMTVCILDRPRHAQLIHDVRATGARIRLITDGDVAGAISACRPHSGTDLLAGIGGTPEGIIAAAAIRCMGGAIQAQLAPRDDAERRKALEAGYDLNQVLTTEDLVSGENVFFCATGVTDGDLLKGVRYYPGGCTTHSIVMRSKSGTVRMIEAYHRLSKLNEYSAIDFTGDSSAVYPLP

SEQ ID NO: 133 - protein, 386 aa, Mycobacterium tuberculosis
MKAATQARIDDSPLAWLDAVQRQRHEAGLRRCLRPRPAVATELDLASNDYLGLSRHPAVIDGGVQALRIWGAGATGSRLVTGDTKLHQQFEAELAEFVGAAAGLLFSSGYTANLGAVVGLSGPGSLLVSDARSHASLVDACRLSRARVVVTPHRDVDAVDAALRSRDEQRAVVVTDSVFSADGSLAPVRELLEVCRRHGALLLVDEAHGLGVRGGGRGLLYELGLAGAPDVVMTTTLSKALGSQGGVVLGPTPVRAHLIDAARPFIFDTGLAPAAVGAARAALRVLQAEPWRPQAVLNHAGELARMCGVAAVPDSAMVSVILGEPESAVAAAAACLDAGVKVGCFRPPTVPAGTSRLRLTARASLNAGELELARRVLTDVLAVARR

SEQ ID NO: 134 - DNA, 1170 bp, Mycobacterium tuberculosis
  source 1..1170: mol_type = genomic DNA
catatgaaagccgccacgcaggcacggatcgacgattcaccgttggcctggttggacgcggtgcagcggcagcgccacgaggccggactgcggcgctgcctgcggccgcgtcccgcggtcgccaccgagctggacttggcctccaacgactatctcggtctgtcccgacatcccgccgtcatcgacggcggcgtccaggcgctgcggatctggggcgccggcgccaccgggtcgcgcctggttaccggcgacaccaagctgcaccagcaattcgaggccgagctcgccgagttygtcggcgctgccgcgggattgctgttctcctctggctacacggccaacctgggcgccgtggtcggcctgtccggcccgggttccctgctggtgtccgacgcccgttcgcatgcgtcgttggtggatgcctgtcggctgtcgcgggcgcgggttgtggtgacgccgcaccgcgacgtcgacgccgtggacgccgcgctgcgatcgcgcgacgagcagcgcgccgtcgtcgtcaccgactcggtgttcagcgccgacggctcgctggcgccggttcgggagttgcttgaggtctgccggcgtcatggtgcgctgcttctggtggacgaggcgcacggcctgggtgtgcgtggcggcggacgcgggctgctctacgagttaggtctagcgggtgcgcccgacgtggtgatgaccaccacgctgtccaaggcgctgggcagccagggtggtgtggtgctcgggccgacgccggtgcgggcccatctgatcgatgctgcccggccgttcatcttcgacaccggtctggcgccggcggcggtgggtgccgcacgggccgcgctgcgcgtcttgcaggccgagccgtggcgaccgcaggcggtgctcaaccacgctggtgaacttgcgcggatgtgcggtgtggctgcggtgccggactcggcgatggtgtcggtgatcctgggcgagccggagtcggcagtggccgccgcggcggcctgcctggacgccggggtcaaggtgggctgcttccggccgccgacggtgcccgcgggtacgtcgcggctgcggctgaccgcgcgcgcatcgctgaacgccggcgagctcgagctggcccggcgggtgctgacggatgttctcgccgtggcgcgccgttgaaagctt

SEQ ID NO: 135 - protein, 406 aa, Mycobacterium tuberculosis
MGSSHHHHHHSSGLVPRGSHMKAATQARIDDSPLAWLDAVQRQRHEAGLRRCLRPRPAVATELDLASNDYLGLSRHPAVIDGGVQALRIWGAGATGSRLVTGDTKLHQQFEAELAEFVGAAAGLLFSSGYTANLGAVVGLSGPGSLLVSDARSHASLVDACRLSRARVVVTPHRDVDAVDAALRSRDEQRAVVVTDSVFSADGSLAPVRELLEVCRRHGALLLVDEAHGLGVRGGGRGLLYELGLAGAPDVVMTTTLSKALGSQGGVVLGPTPVRAHLIDAARPFIFDTGLAPAAVGAARAALRVLQAEPWRPQAVLNHAGELARMCGVAAVPDSAMVSVILGEPESAVAAAAACLDAGVKVGCFRPPTVPAGTSRLRLTARASLNAGELELARRVLTDVLAVARR

SEQ ID NO: 136 - protein, 393 aa, Mycobacterium tuberculosis
MDFGALPPEVNSVRMYAGPGSAPMVAAASAWNGLAAELSSAATGYETVITQLSSEGWLGPASAAMAEAVAPYVAWMSAAAAQAEQAATQARAAAAAFEAAFAATVPPPLIAANRASLMQLISTNVFGQNTSAIAAAEAQYGEMWAQDSAAMYAYAGSSASASAVTPFSTPPQIANPTAQGTQAAAVATAAGTAQSTLTEMITGLPNALQSLTSPLLQSSNGPLSWLWQILFGTPNFPTSISALLTDLQPYASFFYNTEGLPYFSIGMGNNFIQSAKTLGLIGSAAPAAVAAAGDAAKGLPGLGGMLGGGPVAAGLGNAASVGKLSVPPVWSGPLPGSVTPGAAPLPVSTVSAAPEAAPGSLLGGLPLAGAGGAGAGPRYGFRPTVMARPPFAG

SEQ ID NO: 137 - DNA, 1191 bp, Mycobacterium tuberculosis
  source 1..1191: mol_type = genomic DNA
catatggattttggggcgttgccgccggaggtcaattcggtgcggatgtatgccggtcctggctcggcaccaatggtcgctgcggcgtcggcctggaacgggttggccgcggagctgagttcggcggccaccggttatgagacggtgatcactcagctcagcagtgaggggtggctaggtccggcgtcagcggcgatggccgaggcagttgcgccgtatgtggcgtggatgagtgccgctgcggcgcaagccgagcaggcggccacacaggccagggccgccgcggccgcttttgaggcggcgtttgccgcgacggtgcctccgccgttgatcgcggccaaccgggcttcgttgatgcagctgatctcgacgaatgtctttggtcagaacacctcggcgatcgcggccgccgaagctcagtacggcgagatgtgggcccaagactccgcggcgatgtatgcctacgcgggcagttcggcgagcgcctcggcggtcacgccgtttagcacgccgccgcagattgccaacccgaccgctcagggtacgcaggccgcggccgtggccaccgccgccggtaccgcccagtcgacgctgacggagatgatcaccgggctacccaacgcgctgcaaagcctcacctcacctctgttgcagtcgtctaacggtccgctgtcgtggctgtggcagatcttgttcggcacgcccaatttccccacctcaatttcggcactgctgaccgacctgcagccctacgcgagcttcttctataacaccgagggcctgccgtacttcagcatcggcatgggcaacaacttcattcagtcggccaagaccctgggattgatcggctcggcggcaccggctgcggtcgcggctgctggggatgccgccaagggcttgcctggactgggcgggatgctcggtggcgggccggtggcggcgggtctgggcaatgcggcttcggttggcaagctgtcggtgccgccggtgtggagtggaccgttgcccgggtcggtgactccgggggctgctccgctaccggtgagtacggtcagtgccgccccggaggcggcgcccggaagcctgttgggcggcctgccgctagctggtgcgggcggggccggcgcgggtccacgctacggattccgtcccaccgtcatggctcgcccacccttcgccggatagaagctt

SEQ ID NO: 138 - protein, 413 aa, Mycobacterium tuberculosis
MGSSHHHHHHSSGLVPRGSHMDFGALPPEVNSVRMYAGPGSAPMVAAASAWNGLAAELSSAATGYETVITQLSSEGWLGPASAAMAEAVAPYVAWMSAAAAQAEQAATQARAAAAAFEAAFAATVPPPLIAANRASLMQLISTNVFGQNTSAIAAAEAQYGEMWAQDSAAMYAYAGSSASASAVTPFSTPPQIANPTAQGTQAAAVATAAGTAQSTLTEMITGLPNALQSLTSPLLQSSNGPLSWLWQILFGTPNFPTSISALLTDLQPYASFFYNTEGLPYFSIGMGNNFIQSAKTLGLIGSAAPAAVAAAGDAAKGLPGLGGMLGGGPVAAGLGNAASVGKLSVPPVWSGPLPGSVTPGAAPLPVSTVSAAPEAAPGSLLGGLPLAGAGGAGAGPRYGFRPTVMARPPFAG

SEQ ID NO: 139 - protein, 498 aa, Mycobacterium tuberculosis
MSFVVTIPEALAAVATDLAGIGSTIGTANAAAAVPTTTVLAAAADEVSAAMAALFSGHAQAYQALSAQAALFHEQFVRALTAGAGSYAAAEAASAAPLEGVLDVINAPALALLGRPLIGNGANGAPGTGANGGDGGILIGNGGAGGSGAAGMPGGNGGAAGLFGNGGAGGAGGNVASGTAGFGGAGGAGGLLYGAGGAGGAGGRAGGGVGGIGGAGGAGGNGGLLFGAGGAGGVGGLAADAGDGGAGGDGGLFFGVGGAGGAGGTGTNVTGGAGGAGGNGGLLFGAGGVGGVGGDGVAFLGTAPGGPGGAGGAGGLFGVGGAGGAGGIGLVGNGGAGGSGGSALLWGDGGAGGAGGVGSTTGGAGGAGGNAGLLVGAGGAGGAGALGGGATGVGGAGGNGGTAGLLFGAGGAGGFGFGGAGGAGGLGGKAGLIGDGGDGGAGGNGTGAKGGDGGAGGGAILVGNGGNGGNAGSGTPNGSAGTGGAGGLLGKNGMNGLP

SEQ ID NO: 140 - DNA, 1506 bp, Mycobacterium tuberculosis
  source 1..1506: mol_type = genomic DNA
catatgtcatttgtggtcacgatcccggaggcgctagcggcggtggcgaccgatttggcgggtatcgggtcgacgatcggcaccgccaacgcggccgccgcggtcccgaccacgacggtgttggccgccgccgccgatgaggtgtcggcggcgatggcggcattgttctccggacacgcccaggcctatcaggcgctgagcgcccaggcggcgctgtttcacgagcagttcgtgcgggcgctcaccgccggggcgggctcgtatgcggccgccgaggccgccagcgcggccccgctagagggtgtgctcgacgtgatcaacgcccccgccctggcgctgttggggcgcccactgatcggtaacggagccaacggggccccggggaccggggcaaacggcggcgacggcggaatcttgatcggcaacggcggggccggcggctccggcgcggccggcatgcccgggggcaacggcggagccgctggcctgttcggcaacggcggggccggcggcgccggggggaacgtagcgtccggcaccgcagggttcggcggggccggcggggccggcgggctgctctacggcgccggcggggccggcggcgccggcggacgcgccggtggtggggtgggcggtattggtggggccggcggggccggcggcaatggcgggctgctgttcggcgccggcggggccggcggcgtcggcggactcgcggctgacgccggtgacggcggggccggcggagacggcgggttgttcttcggcgtgggcggtgccggcggggccggcggcaccggcactaatgtcaccggcggtgccggcggggccggcggcaatggcgggctcctgttcggcgccggcggggtgggcggtgttggcggtgacggtgtggcattcctgggcaccgcccccggcgggcccggtggtgccggcggggccggtgggctgttcggcgtcggtggggccggcggcgccggcggaatcggattggtcgggaacggcggtgccggggggtccggcgggtccgccctgctctggggcgacggcggtgccggcggcgcgggtggggtcgggtccactaccggcggtgccggcggggcgggcggcaacgccggcctgctggtaggcgccggcggggccggcggcgccggcgcactcggcggtggcgctaccggggtgggcggcgccggcggaaacggcggcactgcgggcctgctgtttggtgccggcggcgccggcggattcggcttcggcggtgccgggggcgccggtgggctcggcggcaaagccgggctgatcggcgacggcggtgacggcggcgccggaggaaacggcaccggtgccaagggcggtgacggcggcgctggcggcggtgccatcctggtcggcaacggcggcaacggcggcaacgccgggagtggcacacctaacggcagcgcgggcaccggcggtgccggcgggctgttgggtaagaacgggatgaacgggttaccgtagaagctt

SEQ ID NO: 141 - protein, 518 aa, Mycobacterium tuberculosis
MGSSHHHHHHSSGLVPRGSHMSFVVTIPEALAAVATDLAGIGSTIGTANAAAAVPTTTVLAAAADEVSAAMAALFSGHAQAYQALSAQAALFHEQFVRALTAGAGSYAAAEAASAAPLEGVLDVINAPALALLGRPLIGNGANGAPGTGANGGDGGILIGNGGAGGSGAAGMPGGNGGAAGLFGNGGAGGAGGNVASGTAGFGGAGGAGGLLYGAGGAGGAGGRAGGGVGGIGGAGGAGGNGGLLFGAGGAGGVGGLAADAGDGGAGGDGGLFFGVGGAGGAGGTGTNVTGGAGGAGGNGGLLFGAGGVGGVGGDGVAFLGTAPGGPGGAGGAGGLFGVGGAGGAGGIGLVGNGGAGGSGGSALLWGDGGAGGAGGVGSTTGGAGGAGGNAGLLVGAGGAGGAGALGGGATGVGGAGGNGGTAGLLFGAGGAGGFGFGGAGGAGGLGGKAGLIGDGGDGGAGGNGTGAKGGDGGAGGGAILVGNGGNGGNAGSGTPNGSAGTGGAGGLLGKNGMNGLP

SEQ ID NO: 142 - protein, 325 aa, Mycobacterium tuberculosis
MHQVDPNLTRRKGRLAALAIAAMASASLVTVAVPATANADPEPAPPVPTTAASPPSTAAAPPAPATPVAPPPPAAANTPNAQPGDPNAAPPPADPNAPPPPVIAPNAPQPVRIDNPVGGFSFALPAGWVESDAAHFDYGSALLSKTTGDPPFPGQPPPVANDTRIVLGRLDQKLYASAEATDSKAAARLGSDMGEFYMPYPGTRINQETVSLDANGVSGSASYYEVKFSDPSKPNGQIWTGVIGSPAANAPDAGPPQRWFVVWLGTANNPVDKGAAKALAESIRPLVAPPPAPAPAPAEPAPAPAPAGEVAPTPTTPTPQRTLPA

SEQ ID NO: 143 - DNA, 873 bp, Mycobacterium tuberculosis
  source 1..873: mol_type = genomic DNA
catatggatccggagccagcgcccccggtacccacaacggccgcctcgccgccgtcgaccgctgcagcgccacccgcaccggcgacacctgttgcccccccaccaccggccgccgccaacacgccgaatgcccagccgggcgatcccaacgcagcacctccgccggccgacccgaacgcaccgccgccacctgtcattgccccaaacgcaccccaacctgtccggatcgacaacccggttggaggattcagcttcgcgctgcctgctggctgggtggagtctgacgccgcccacttcgactacggttcagcactcctcagcaaaaccaccggggacccgccatttcccggacagccgccgccggtggccaatgacacccgtatcgtgctcggccggctagaccaaaagctttacgccagcgccgaagccaccgactccaaggccgcggcccggttgggctcggacatgggtgagttctatatgccctacccgggcacccggatcaaccaggaaaccgtctcgctcgacgccaacggggtgtctggaagcgcgtcgtattacgaagtcaagttcagcgatccgagtaagccgaacggccagatctggacgggcgtaatcggctcgcccgcggcgaacgcaccggacgccgggccccctcagcgctggtttgtggtatggctcgggaccgccaacaacccggtggacaagggcgcggccaaggcgctggccgaatcgatccggcctttggtcgccccgccgccggcgccggcaccggctcctgcagagcccgctccggcgccggcgccggccggggaagtcgctcctaccccgacgacaccgacaccgcagcggaccttaccggcctgagaattc

SEQ ID NO: 144 - protein, 307 aa, Mycobacterium tuberculosis
MGSSHHHHHHSSGLVPRGSHMDPEPAPPVPTTAASPPSTAAAPPAPATPVAPPPPAAANTPNAQPGDPNAAPPPADPNAPPPPVIAPNAPQPVRIDNPVGGFSFALPAGWVESDAAHFDYGSALLSKTTGDPPFPGQPPPVANDTRIVLGRLDQKLYASAEATDSKAAARLGSDMGEFYMPYPGTRINQETVSLDANGVSGSASYYEVKFSDPSKPNGQIWTGVIGSPAANAPDAGPPQRWFVVWLGTANNPVDKGAAKALAESIRPLVAPPPAPAPAPAEPAPAPAPAGEVAPTPTTPTPQRTLPA

SEQ ID NO: 145 - protein, 325 aa, Mycobacterium tuberculosis
MTDVSRKIRAWGRRLMIGTAAAVVLPGLVGLAGGAATAGAFSRPGLPVEYLQVPSPSMGRDIKVQFQSGGNNSPAVYLLDGLRAQDDYNGWDINTPAFEWYYQSGLSIVMPVGGQSSFYSDWYSPACGKAGCQTYKWETFLTSELPQWLSANRAVKPTGSAAIGLSMAGSSAMILAAYHPQQFIYAGSLSALLDPSQGMGPSLIGLAMGDAGGYKAADMWGPSSDPAWERNDPTQQIPKLVANNTRLWVYCGNGTPNELGGANIPAEFLENFVRSSNLKFQDAYNAAGGHNAVFNFPPNGTHSWEYWGAQLNAMKGDLQSSLGAG

SEQ ID NO: 146 - DNA, 904 bp, Mycobacterium tuberculosis
  source 1..904: mol_type = genomic DNA
catatgcatcaccatcaccatcacttctcccggccggggctgccggtcgagtacctgcaggtgccgtcgccgtcgatgggccgcgacatcaaggttcagttccagagcggtgggaacaactcacctgcggtttatctgctcgacggcctgcgcgcccaagacgactacaacggctgggatatcaacaccccggcgttcgagtggtactaccagtcgggactgtcgatagtcatgccggtcggcgggcagtccagcttctacagcgactggtacagcccggcctgcggtaaggctggctgccagacttacaagtgggaaaccttcctgaccagcgagctgccgcaatggttgtccgccaacagggccgtgaagcccaccggcagcgctgcaatcggcttgtcgatggccggctcgtcggcaatgatcttggccgcctaccacccccagcagttcatctacgccggctcgctgtcggccctgctggacccctctcaggggatggggcctagcctgatcggcctcgcgatgggtgacgccggcggttacaaggccgcagacatgtggggtccctcgagtgacccggcatgggagcgcaacgaccctacgcagcagatccccaagctggtcgcaaacaacacccggctatgggtttattgcgggaacggcaccccgaacgagttgggcggtgccaacatacccgccgagttcttggagaacttcgttcgtagcagcaacctgaagttccaggatgcgtacaacgccgcgggcgggcacaacgccgtgttcaacttcccgcccaacggcacgcacagctgggagtactggggcgctcagctcaacgccatgaagggtgacctgcagagttcgttaggcgccggctgacgggatcaaccgaagggaattc

SEQ ID NO: 147 - protein, 293 aa, Mycobacterium tuberculosis
HMHHHHHHFSRPGLPVEYLQVPSPSMGRDIKVQFQSGGNNSPAVYLLDGLRAQDDYNGWDINTPAFEWYYQSGLSIVMPVGGQSSFYSDWYSPACGKAGCQTYKWETFLTSELPQWLSANRAVKPTGSAAIGLSMAGSSAMILAAYHPQQFIYAGSLSALLDPSQGMGPSLIGLAMGDAGGYKAADMWGPSSDPAWERNDPTQQIPKLVANNTRLWVYCGNGTPNELGGANIPAEFLENFVRSSNLKFQDAYNAAGGHNAVFNFPPNGTHSWEYWGAQLNAMKGDLQSSLGAG

SEQ ID NO: 148 - protein, 740 aa, Mycobacterium tuberculosis
VPEQHPPITETTTGAASNGCPVVGHMKYPVEGGGNQDWWPNRLNLKVLHQNPAVADPMGAAFDYAAEVATIDVDALTRDIEEVMTTSQPWWPADYGHYGPLFIRMAWHAAGTYRIHDGRGGAGGGMQRFAPLNSWPDNASLDKARRLLWPVKKKYGKKLSWADLIVFAGNCALESMGFKTFGFGFGRVDQWEPDEVYWGKEATWLGDERYSGKRDLENPLAAVQMGLIYVNPEGPNGNPDPMAAAVDIRETFRRMAMNDVETAALIVGGHTFGKTHGAGPADLVGPEPEAAPLEQMGLGWKSSYGTGTGKDAITSGIEVVWTNTPTKWDNSFLEILYGYEWELTKSPAGAWQYTAKDGAGAGTIPDPFGGPGRSPTMLATDLSLRVDPIYERITRRWLEHPEELADEFAKAWYKLIHRDMGPVARYLGPLVPKQTLLWQDPVPAVSHDLVGEAEIASLKSQIRASGLTVSQLVSTAWAAASSFRGSDKRGGANGGRIRLQPQVGWEVNDPDGDLRKVIRTLEEIQESFNSAAPGNIKVSFADLVVLGGCAAIEKAAKAAGHNITVPFTPGRTDASQEQTDVESFAVLEPKADGFRNYLGKGNPLPAEYMLLDKANLLTLSAPEMTVLVGGLRVLGANYKRLPLGVFTEASESLTNDFFVNLLDMGITWEPSPADDGTYQGKDGSGKVKWTGSRVDLVFGSNSELRALVEVYGADDAQPKFVQDFVAAWDKVMNLDRFDVR

SEQ ID NO: 149 - DNA, 2157 bp, Mycobacterium tuberculosis
  source 1..2157: mol_type = genomic DNA
catatgaaataccccgtcgagggcggcggaaaccaggactggtggcccaaccggctcaatctgaaggtactgcaccaaaacccggccgtcgctgacccgatgggtgcggcgttcgactatgccgcggaggtcgcgaccatcgacgttgacgccctgacgcgggacatcgaggaagtgatgaccacctcgcagccgtggtggcccgccgactacggccactacgggccgctgtttatccggatggcgtggcacgctgccggcacctaccgcatccacgacggccgcggcggcgccgggggcggcatgcagcggttcgcgccgcttaacagctggcccgacaacgccagcttggacaaggcgcgccggctgctgtggccggtcaagaagaagtacggcaagaagctctcatgggcggacctgattgttttcgccggcaactgcgcgctggaatcgatgggcttcaagacgttcgggttcggcttcggccgggtcgaccagtgggagcccgatgaggtctattggggcaaggaagccacctggctcggcgatgagcgttacagcggtaagcgggatctggagaacccgctggccgcggtgcagatggggctgatctacgtgaacccggaggggccgaacggcaacccggaccccatggccgcggcggtcgacattcgcgagacgtttcggcgcatggccatgaacgacgtcgaaacagcggcgctgatcgtcggcggtcacactttcggtaagacccatggcgccggcccggccgatctggtcggccccgaacccgaggctgctccgctggagcagatgggcttgggctggaagagctcgtatggcaccggaaccggtaaggacgcgatcaccagcggcatcgaggtcgtatggacgaacaccccgacgaaatgggacaacagtttcctcgagatcctgtacggctacgagtgggagctgacgaagagccctgctggcgcttggcaatacaccgccaaggacggcgccggtgccggcaccatcccggacccgttcggcgggccagggcgctccccgacgatgctggccactgacctctcgctgcgggtggatccgatctatgagcggatcacgcgtcgctggctggaacaccccgaggaattggccgacgagttcgccaaggcctggtacaagctgatccaccgagacatgggtcccgttgcgagataccttgggccgctggtccccaagcagaccctgctgtggcaggatccggtccctgcggtcagccacgacctcgtcggcgaagccgagattgccagccttaagagccagatccgggcatcgggattgactgtctcacagctagtttcgaccgcatgggcggcggcgtcgtcgttccgtggtagcgacaagcgcggcggcgccaacggtggtcgcatccgcctgcagccacaagtcgggtgggaggtcaacgaccccgacggggatctgcgcaaggtcattcgcaccctggaagagatccaggagtcattcaactccgcggcgccggggaacatcaaagtgtccttcgccgacctcgtcgtgctcggtggctgtgccgccatagagaaagcagcaaaggcggctggccacaacatcacggtgcccttcaccccgggccgcacggatgcgtcgcaggaacaaaccgacgtggaatcctttgccgtgctggagcccaaggcagatggcttccgaaactacctcggaaagggcaacccgttgccggccgagtacatgctgctcgacaaggcgaacctgcttacgctcagtgcccctgagatgacggtgctggtaggtggcctgcgcgtcctcggcgcaaactacaagcgcttaccgctgggcgtgttcaccgaggcctccgagtcactgaccaacgacttcttcgtgaacctgctcgacatgggtatcacctgggagccctcgccagcagatgacgggacctaccagggcaaggatggcagtggcaaggtgaagtggaccggcagccgcgtggacctggtcttcgggtccaactcggagttgcgggcgcttgtcgaggtctatggcgccgatgacgcgcagccgaagttcgtgcaggacttcgtcgctgcctgggacaaggtgatgaacctcgacaggttcgacgtgcgctgaaagctt

SEQ ID NO: 150 - protein, 735 aa, Mycobacterium tuberculosis
MGSSHHHHHHSSGLVPRGSHMKYPVEGGGNQDWWPNRLNLKVLHQNPAVADPMGAAFDYAAEVATIDVDALTRDIEEVMTTSQPWWPADYGHYGPLFIRMAWHAAGTYRIHDGRGGAGGGMQRFAPLNSWPDNASLDKARRLLWPVKKKYGKKLSWADLIVFAGNCALESMGFKTFGFGFGRVDQWEPDEVYWGKEATWLGDERYSGKRDLENPLAAVQMGLIYVNPEGPNGNPDPMAAAVDIRETFRRMAMNDVETAALIVGGHTFGKTHGAGPADLVGPEPEAAPLEQMGLGWKSSYGTGTGKDAITSGIEVVWTNTPTKWDNSFLEILYGYEWELTKSPAGAWQYTAKDGAGAGTIPDPFGGPGRSPTMLATDLSLRVDPIYERITRRWLEHPEELADEFAKAWYKLIHRDMGPVARYLGPLVPKQTLLWQDPVPAVSHDLVGEAEIASLKSQIRASGLTVSQLVSTAWAAASSFRGSDKRGGANGGRIRLQPQVGWEVNDPDGDLRKVIRTLEEIQESFNSAAPGNIKVSFADLVVLGGCAAIEKAAKAAGHNITVPFTPGRTDASQEQTDVESFAVLEPKADGFRNYLGKGNPLPAEYMLLDKANLLTLSAPEMTVLVGGLRVLGANYKRLPLGVFTEASESLTNDFFVNLLDMGITWEPSPADDGTYQGKDGSGKVKWTGSRVDLVFGSNSELRALVEVYGADDAQPKFVQDFVAAWDKVMNLDRFDVR

SEQ ID NO: 151 - protein, 331 aa, Mycobacterium tuberculosis
MPDTMVTTDVIKSAVQLACRAPSLHNSQPWRWIAEDHTVALFLDKDRVLYATDHSGREALLGCGAVLDHFRVAMAAAGTTANVERFPNPNDPLHLASIDFSPADFVTEGHRLRADAILLRRTDRLPFAEPPDWDLVESQLRTTVTADTVRIDVIADDMRPELAAASKLTESLRLYDSSYHAELFWWTGAFETSEGIPHSSLVSAAESDRVTFGRDFPVVANTDRRPEFGHDRSKVLVLSTYDNERASLLRCGEMLSAVLLDATMAGLATCTLTHITELHASRDLVAALIGQPATPQALVRVGLAPEMEEPPPATPRRPIDEVFHVRAKDHR

SEQ ID NO: 152 - DNA, 1026 bp, Mycobacterium tuberculosis
  source 1..1026: mol_type = genomic DNA
catatgcatcaccatcaccatcacatgccggacaccatggtgaccaccgatgtcatcaagagcgcggtgcagttggcctgccgcgcaccgtcgctccacaacagccagccctggcgctggatagccgaggaccacacggttgcgctgttcctcgacaaggatcgggtgctttacgcgaccgaccactccggccgggaagcgctgctggggtgcggcgccgtactcgaccactttcgggtggcgatggcggccgcgggtaccaccgccaatgtggaacggtttcccaaccccaacgatcctttgcatctggcgtcaattgacttcagcccggccgatttcgtcaccgagggccaccgtctaagggcggatgcgatcctactgcgccgtaccgaccggctgcctttcgccgagccgccggattgggacttggtggagtcgcagttgcgcacgaccgtcaccgccgacacggtgcgcatcgacgtcatcgccgacgatatgcgtcccgaactggcggcggcgtccaaactcaccgaatcgctgcggctctacgattcgtcgtatcatgccgaactcttttggtggacaggggcttttgagacttctgagggcataccgcacagttcattggtatcggcggccgaaagtgaccgggtcaccttcggacgcgacttcccggtcgtcgccaacaccgataggcgcccggagtttggccacgaccgctctaaggtcctggtgctctccacctacgacaacgaacgcgccagcctactgcgctgcggcgagatgctttccgccgtattgcttgacgccaccatggctgggcttgccacctgcacgctgacccacatcaccgaactgcacgccagccgagacctggtcgcagcgctgattgggcagcccgcaactccgcaagccttggttcgcgtcggtctggccccggagatggaagagccgccaccggcaacgcctcggcgaccaatcgatgaagtgtttcacgttcgggctaaggatcaccggtaggaattc

SEQ ID NO: 153 - protein, 338 aa, Mycobacterium tuberculosis
MHHHHHHMPDTMVTTDVIKSAVQLACRAPSLHNSQPWRWIAEDHTVALFLDKDRVLYATDHSGREALLGCGAVLDHFRVAMAAAGTTANVERFPNPNDPLHLASIDFSPADFVTEGHRLRADAILLRRTDRLPFAEPPDWDLVESQLRTTVTADTVRIDVIADDMRPELAAASKLTESLRLYDSSYHAELFWWTGAFETSEGIPHSSLVSAAESDRVTFGRDFPVVANTDRRPEFGHDRSKVLVLSTYDNERASLLRCGEMLSAVLLDATMAGLATCTLTHITELHASRDLVAALIGQPATPQALVRVGLAPEMEEPPPATPRRPIDEVFHVRAKDHR

SEQ ID NO: 154 - protein, 478 aa, Mycobacterium tuberculosis
VTEKTPDDVFKLAKDEKVEYVDVRFCDLPGIMQHFTIPASAFDKSVFDDGLAFDGSSIRGFQSIHESDMLLLPDPETARIDPFRAAKTLNINFFVHDPFTLEPYSRDPRNIARKAENYLISTGIADTAYFGAEAEFYIFDSVSFDSRANGSFYEVDAISGWWNTGAATEADGSPNRGYKVRHKGGYFPVAPNDQYVDLRDKMLTNLINSGFILEKGHHEVGSGGQAEINYQFNSLLHAADDMQLYKYIIKNTAWQNGKTVTFMPKPLFGDNGSGMHCHQSLWKDGAPLMYDETGYAGLSDTARHYIGGLLHHAPSLLAFTNPTVNSYKRLVPGYEAPINLVYSQRNRSACVRIPITGSNPKAKRLEFRSPDSSGNPYLAFSAMLMAGLDGIKNKIEPQAPVDKDLYELPPEEAASIPQTPTQLSDVIDRLEADHEYLTEGGVFTNDLIETWISFKRENEIEPVNIRPHPYEFALYYDV

SEQ ID NO: 155 - DNA, 1445 bp, Mycobacterium tuberculosis
  source 1..1445: mol_type = genomic DNA
catatgacggaaaagacgcccgacgacgtcttcaaacttgccaaggacgagaaggtcgaatatgcgacgtccggttctgtgacctgcctggcatcatgcagcacttcacgattccggcttcggcctttgacaagagcgtgtttgacgacggcttggcctttgacggctcgtcgattcgcgggttccagtcgatccacgaatccgacatgttgcttcttcccgatcccgagacggcgcgcatcgacccgttccgcgcggccaagacgctgaatatcaacttctttgtgcacgacccgttcaccctggagccgtactcccgcgacccgcgcaacatcgcccgcaaggccgagaactacctgatcagcactggcatcgccgacaccgcatacttcggcgccgaggccgagttctacattttcgattcggtgagcttcgactcgcgcgccaacggctccttctacgaggtggacgccatctcggggtggtggaacaccggcgcggcgaccgaggccgacggcagtcccaaccggggctacaaggtccgccacaagggcgggtatttcccagtggcccccaacgaccaatacgtcgacctgcgcgacaagatgctgaccaacctgatcaactccggcttcatcctggagaagggccaccacgaggtgggcagcggcggacaggccgagatcaactaccagttcaattcgctgctgcacgccgccgacgacatgcagttgtacaagtacatcatcaagaacaccgcctggcagaacggcaaaacggtcacgttcatgcccaagccgctgttcggcgacaacgggtccggcatgcactgtcatcagtcgctgtggaaggacggggccccgctgatgtacgacgagacgggttatgccggtctgtcggacacggcccgtcattacatcggcggcctgttacaccacgcgccgtcgctgctggccttcaccaacccgacggtgaactcctacaagcggctggttcccggttacgaggccccgatcaacctggtctatagccagcgcaaccggtcggcatgcgtgcgcatcccgatcaccggcagcaacccgaaggccaagcggctggagttccgaagccccgactcgtcgggcaacccgtatctggcgttctcggccatgctgatggcaggcctggacggtatcaagaacaagatcgagccgcaggcgcccgtcgacaaggatctctacgagctgccgccggaagaggccgcgagtatcccgcagactccgacccagctgtcagatgtgatcgaccgtctcgaggccgaccacgaatacctcaccgaaggaggggtgttcacaaacgacctgatcgagacgtggatcagtttcaagcgcgaaaacgagatcgagccggtcaacatccggccgcatccctacgaattcgcgctgtactacgacgtttaaaagctt

SEQ ID NO: 156 - protein, 498 aa, Mycobacterium tuberculosis
MGSSHHHHHHSSGLVPRGSHMTEKTPDDVFKLAKDEKVEYVDVRFCDLPGIMQHFTIPASAFDKSVFDDGLAFDGSSIRGFQSIHESDMLLLPDPETARIDPFRAAKTLNINFFVHDPFTLEPYSRDPRNIARKAENYLISTGIADTAYFGAEAEFYIFDSVSFDSRANGSFYEVDAISGWWNTGAATEADGSPNRGYKVRHKGGYFPVAPNDQYVDLRDKMLTNLINSGFILEKGHHEVGSGGQAEINYQFNSLLHAADDMQLYKYIIKNTAWQNGKTVTFMPKPLFGDNGSGMHCHQSLWKDGAPLMYDETGYAGLSDTARHYIGGLLHHAPSLLAFTNPTVNSYKRLVPGYEAPINLVYSQRNRSACVRIPITGSNPKAKRLEFRSPDSSGNPYLAFSAMLMAGLDGIKNKIEPQAPVDKDLYELPPEEAASIPQTPTQLSDVIDRLEADHEYLTEGGVFTNDLIETWISFKRENEIEPVNIRPHPYEFALYYDV

SEQ ID NO: 157 - protein, 580 aa, Mycobacterium tuberculosis; identical to SEQ ID NO: 26

SEQ ID NO: 158 - DNA, 1752 bp, Mycobacterium tuberculosis; identical to SEQ ID NO: 27
  source 1..1752: mol_type = genomic DNA

SEQ ID NO: 159 - protein, 600 aa, Mycobacterium tuberculosis; identical to SEQ ID NO: 28

SEQ ID NO: 160 - protein, 297 aa, Mycobacterium tuberculosis
MSSGNSSLGIIVGIDDSPAAQVAVRWAARDAELRKIPLTLVHAVSPEVATWLEVPLPPGVLRWQQDHGRHLIDDALKVVEQASLRAGPPTVHSEIVPAAAVPTLVDMSKDAVLMVVGCLGSGRWPGRLLGSVSSGLLRHAHCPVVIIHDEDSVMPHPQQAPVLVGVDGSSASELATAIAFDEASRRNVDLVALHAWSDVDVSEWPGIDWPATQSMAEQVLAERLAGWQERYPNVAITRVVVRDQPARQLVQRSEEAQLVVVGSRGRGGYAGMLVGSVGETVAQLARTPVIVARESLT

SEQ ID NO: 161 - DNA, 903 bp, Mycobacterium tuberculosis
  source 1..903: mol_type = genomic DNA
catatgtcatcgggcaattcatctctgggaattatcgtcgggatcgacgattcaccggccgcacaggttgcggtgcggtgggcagctcgggatgcggagttgcgaaaaatccctctgacgctcgtgcacgcggtgtcgccggaagtagccacctggctggaggtgccactgccgccgggcgtgctgcgatggcagcaggatcacgggcgccacctgatcgacgacgcactcaaggtggttgaacaggcttcgctgcgcgctggtccccccacggtccacagtgaaatcgttccggcggcagccgttcccacattggtcgacatgtccaaagacgcagtgctgatggtcgtgggttgtctcggaagtgggcggtggccgggccggctgctcggttcggtcagttccggcctgctccgccacgcgcactgtccggtcgtgatcatccacgacgaagattcggtgatgccgcatccccagcaagcgccggtgctagttggcgttgacggctcgtcggcctccgagctggcgaccgcaatcgcattcgacgaagcgtcgcggcgaaacgtggacctggtggcgctgcacgcatggagcgacgtcgatgtgtcggagtggcccggaatcgattggccggcaactcagtcgatggccgagcaggtgctggccgagcggttggcgggttggcaggagcggtatcccaacgtagccataacccgcgtggtggtgcgcgatcagccggcccgccagctcgtccaacgctccgaggaagcccagctggtcgtggtcggcagccggggccgcggcggctacgccggaatgctggtggggtcggtaggcgaaaccgttgctcagctggcgcggacgccggtcatcgtggcacgcgagtcgctgacttagaagctt

SEQ ID NO: 162 - protein, 317 aa, Mycobacterium tuberculosis
MGSSHHHHHHSSGLVPRGSHMSSGNSSLGIIVGIDDSPAAQVAVRWAARDAELRKIPLTLVHAVSPEVATWLEVPLPPGVLRWQQDHGRHLIDDALKVVEQASLRAGPPTVHSEIVPAAAVPTLVDMSKDAVLMVVGCLGSGRWPGRLLGSVSSGLLRHAHCPVVIIHDEDSVMPHPQQAPVLVGVDGSSASELATAIAFDEASRRNVDLVALHAWSDVDVSEWPGIDWPATQSMAEQVLAERLAGWQERYPNVAITRVVVRDQPARQLVQRSEEAQLVVVGSRGRGGYAGMLVGSVGETVAQLARTPVIVARESLT

SEQ ID NO: 163 - protein, 193 aa, Mycobacterium tuberculosis
MKVKNTIAATSFAAAGLAALAVAVSPPAAAGDLVGPGCAEYAAANPTGPASVQGMSQDPVAVAASNNPELTTLTAALSGQLNPQVNLVDTLNSGQYTVFAPTNAAFSKLPASTIDELKTNSSLLTSILTYHVVAGQTSPANVVGTRQTLQGASVTVTGQGNSLKVGNADVVCGGVSTANATVYMIDSVLMPPA

SEQ ID NO: 164 - DNA, 503 bp, Mycobacterium tuberculosis
  source 1..503: mol_type = genomic DNA
catatgggcgatctggtgagcccgggctgcgcggaatacgcggcagccaatcccactgggccggcctcggtgcagggaatgtcgcaggacccggtcgcggtggcggcctcgaacaatccggagttgacaacgctgacggctgcactgtcgggccagctcaatccgcaagtaaacctggtggacaccctcaacagcggtcagtacacggtgttcgcaccgaccaacgcggcatttagcaagctgccggcatccacgatcgacgagctcaagaccaattcgtcactgctgaccagcatcctgacctaccacgtagtggccggccaaaccagcccggccaacgtcgtcggcacccgtcagaccctccagggcgccagcgtgacggtgaccggtcagggtaacagcctcaaggtcggtaacgccgacgtcgtctgtggtggggtgtctaccgccaacgcgacggtgtacatgattgacagcgtgctaatgcctccggcgtaaaagct

SEQ ID NO: 165 - protein, 185 aa, Mycobacterium tuberculosis
MGSSHHHHHHSSGLVPRGSHMMGDLVSPGCAEYAAANPTGPASVQGMSQDPVAVAASNNPELTTLTAALSGQLNPQVNLVDTLNSGQYTVFAPTNAAFSKLPASTIDELKTNSSLLTSILTYHVVAGQTSPANVVGTRQTLQGASVTVTGQGNSLKVGNADVVCGGVSTANATVYMIDSVLMPPA

SEQ ID NO: 166 - protein, 359 aa, Mycobacterium tuberculosis
MRSTVAVAVAAAVIAASSGCGSDQPAHKASQSMITPTTQIAGAGVLGNDRKPDESCARAAAAADPGPPTRPAHNAAGVSPEMVQVPAEAQRIVVLSGDQLDALCALGLQSRIVAAALPNSSSSQPSYLGTTVHDLPGVGTRSAPDLRAIAAAHPDLILGSQGLTPQLYPQLAAIAPTVFTAAPGADWENNLRGVGAATARIAAVDALITGFAEHATQVGTKHDATHFQASIVQLTANTMRVYGANNFPASVLSAVGVDRPPSQRFTDKAYIEIGTTAADLAKSPDFSAADADIVYLSCASEAAAERAAVILDSDPWRKLSANRDNRVFVVNDQVWQTGEGMVAARGIVDDLRWVDAPIN

SEQ ID NO: 167 - DNA, 1071 bp, Mycobacterium tuberculosis
  source 1..1071: mol_type = genomic DNA
atgggcagcagccatcatcatcatcatcacagcagcggcctggtgccgcgcggcagccatatgcataaggcgtcacaatcgatgatcacgcccaccacccagatcgccggcgccggggtgctgggaaacgacagaaagccggatgagtcgtgcgcgcgtgcggcggccgcggccgatccggggccaccgacccgaccagcgcacaatgcggcgggagtcagcccggagatggtgcaggtgccggcggaggcgcagcgcatcgtggtgctctccggtgaccagctcgacgcgctgtgcgcgctgggcctgcaatcgcggatcgtcgccgccgcgttgccgaacagctcctcaagtcaaccttcctatctgggcacgaccgtgcatgatctgcccggtgtcggtactcgcagcgcccccgacctgcgcgccattgcggcggctcacccggatctgatcctgggttcgcagggtttgacgccgcagttgtatccgcagctggcggcgatcgccccgacggtgtttaccgcggcaccgggcgcggactgggaaaataacctgcgtggtgtcggtgccgccacggcccgtatcgccgcggtggacgcgctgatcaccgggttcgccgaacacgccacccaggtcgggaccaagcatgacgcgacccacttccaagcgtcgatcgtgcagctgaccgccaacaccatgcgggtatacggcgccaacaacttcccggccagcgtgctgagcgcggtcggcgtcgaccgaccgccgtctcaacggttcaccgacaaggcctacatcgagatcggcaccacggccgccgacctggcgaaatcaccggacttctcggcggccgacgccgatatcgtctacctgtcgtgcgcgtcggaagcagccgcggaacgcgcggccgtcatcctggatagcgacccatggcgcaagctgtccgccaaccgtgacaaccgggtcttcgtcgtcaacgaccaggtatggcagaccggcgagggtatggtcgctgcccgcggcattgtcgatgatctgcgctgggtcgacgcgccgatcaactagaagctt

SEQ ID NO: 168 - protein, 354 aa, Mycobacterium tuberculosis
MGSSHHHHHHSSGLVPRGSHMHKASQSMITPTTQIAGAGVLGNDRKPDESCARAAAAADPGPPTRPAHNAAGVSPEMVQVPAEAQRIVVLSGDQLDALCALGLQSRIVAAALPNSSSSQPSYLGTTVHDLPGVGTRSAPDLRAIAAAHPDLILGSQGLTPQLYPQLAAIAPTVFTAAPGADWENNLRGVGAATARIAAVDALITGFAEHATQVGTKHDATHFQASIVQLTANTMRVYGANNFPASVLSAVGVDRPPSQRFTDKAYIEIGTTAADLAKSPDFSAADADIVYLSCASEAAAERAAVILDSDPWRKLSANRDNRVFVVNDQVWQTGEGMVAARGIVDDLRWVDAPIN

SEQ ID NO: 169 - protein, 299 aa, Mycobacterium tuberculosis
MLRGIQALSRPLTRVYRALAVIGVLAASLLASWVGAVPQVGLAASALPTFAHVVIVVEENRSQAAIIGNKSAPFINSLAANGAMMAQAFAETHPSEPNYLALFAGNTFGLTKNTCPVNGGALPNLGSELLSAGYTFMGFAEDLPAVGSTVCSAGKYARKHVPWVNFSNVPTTLSVPFSAFPKPQNYPGLPTVSFVIPNADNDMHDGSIAQGDAWLNRHLSAYANWAKTNNSLLVVTWDEDDGSSRNQIPTVFYGAHVRPGTYNETISHYNVLSTLEQIYGLPKTGYATNAPPITDIWGD

SEQ ID NO: 170 - DNA, 789 bp, Mycobacterium tuberculosis
  source 1..789: mol_type = genomic DNA
catatggctagcatgagtgccctgccgaccttcgcgcacgtggtcatcgtggtggaggagaaccgctcgcaggccgccatcatcggtaacaagtcggctcccttcatcaattcgctggccgccaacggcgcgatgatggcccaggcgttcgccgaaacacacccgagcgaaccgaactacctggcactgttcgctggcaacacattcgggttgacgaagaacacctgccccgtcaacggcggcgcgctgcccaacctgggttctgagttgctcagcgccggttacacattcatggggttcgccgaagacttgcctgcggtcggctccacggtgtgcagtgcgggcaaatacgcacgcaaacacgtgccgtgggtcaacttcagtaacgtgccgacgacactgtcggtgccgttttcggcatttccgaagccgcagaattaccccggcctgccgacggtgtcgtttgtcatccctaacgccgacaacgacatgcacgacggctcgatcgcccaaggcgacgcctggctgaaccgccacctgtcggcatatgccaactgggccaagacaaacaacagcctgctcgttgtgacctgggacgaagacgacggcagcagccgcaatcagatcccgacggtgttctacggcgcgcacgtgcggcccggaacttacaacgagaccatcagccactacaacgtgctgtccacattggagcagatctacggactgcccaagacgggttatgcgaccaatgctccgccaataaccgatatttggggcgactagaagctt

SEQ ID NO: 171 - protein, 279 aa, Mycobacterium tuberculosis
MGSSHHHHHHSSGLVPRGSHMASMSALPTFAHVVIVVEENRSQAAIIGNKSAPFINSLAANGAMMAQAFAETHPSEPNYLALFAGNTFGLTKNTCPVNGGALPNLGSELLSAGYTFMGFAEDLPAVGSTVCSAGKYARKHVPWVNFSNVPTTLSVPFSAFPKPQNYPGLPTVSFVIPNADNDMHDGSIAQGDAWLNRHLSAYANWAKTNNSLLVVTWDEDDGSSRNQIPTVFYGAHVRPGTYNETISHYNVLSTLEQIYGLPKTGYATNAPPITDIWGD

SEQ ID NO: 172 - protein, 94 aa, Mycobacterium tuberculosis; identical to SEQ ID NO: 46

SEQ ID NO: 173 - DNA, 294 bp, Mycobacterium tuberculosis; identical to SEQ ID NO: 47
  source 1..294: mol_type = genomic DNA

SEQ ID NO: 174 - protein, 114 aa, Mycobacterium tuberculosis; identical to SEQ ID NO: 48

SEQ ID NO: 175 - protein, 284 aa, Mycobacterium tuberculosis; identical to SEQ ID NO: 56

SEQ ID NO: 176 - DNA, 858 bp, Mycobacterium tuberculosis
  source 1..858: mol_type = genomic DNA
atgggcagcagccatcatcatcatcatcacagcagcggcctggtgccgcgcggcagccatatgagtccttgtgcatattttcttgtctacgaatcaaccgaaacgaccgagcggcccgagcaccatgaattcaagcaggcggcggtgttgaccgacctgcccggcgagctgatgtccgcgctatcgcaggggttgtcccagttcgggatcaacataccgccggtgcccagcctgaccgggagcggcgatgccagcacgggtctaaccggtcctggcctgactagtccgggattgaccagcccgggattgaccagcccgggcctcaccgaccctgcccttaccagtccgggcctgacgccaaccctgcccggatcactcgccgcgcccggcaccaccctggcgccaacgcccggcgtgggggccaatccggcgctcaccaaccccgcgctgaccagcccgaccggggcgacgccgggattgaccagcccgacgggtttggatcccgcgctgggcggcgccaacgaaatcccgattacgacgccggtcggattggatcccggggctgacggcacctatccgatcctcggtgatccaacactggggaccataccgagcagccccgccaccacctccaccggcggcggcggtctcgtcaacgacgtgatgcaggtggccaacgagttgggcgccagtcaggctatcgacctgctaaaaggtgtgctaatgccgtcgatcatgcaggccgtccagaatggcggcgcggccgcgccggcagccagcccgccggtcccgcccatccccgcggccgcggcggtgccaccgacggacccaatcaccgtgccggtcgcctaaaagctt

SEQ ID NO: 177 - protein, 283 aa, Mycobacterium tuberculosis
MGSSHHHHHHSSGLVPRGSHMSPCAYFLVYESTETTERPEHHEFKQAAVLTDLPGELMSALSQGLSQFGINIPPVPSLTGSGDASTGLTGPGLTSPGLTSPGLTSPGLTDPALTSPGLTPTLPGSLAAPGTTLAPTPGVGANPALTNPALTSPTGATPGLTSPTGLDPALGGANEIPITTPVGLDPGADGTYPILGDPTLGTIPSSPATTSTGGGGLVNDVMQVANELGASQAIDLLKGVLMPSIMQAVQNGGAAAPAASPPVPPIPAAAAVPPTDPITVPVA

SEQ ID NO: 178 - protein, 460 aa, Mycobacterium tuberculosis
MTQSQTVTVDQQEILNRANEVEAPMADPPTDVPITPCELTAAKNAAQQLVLSADNMREYLAAGAKERQRLATSLRNAAKAYGEVDEEAATALDNDGEGTVQAESAGAVGGDSSAELTDTPRVATAGEPNFMDLKEAARKLETGDQGASLAHFADGWNTFNLTLQGDVKRFRGFDNWEGDAATACEASLDQQRQWILHMAKLSAAMAKQAQYVAQLHVWARREHPTYEDIVGLERLYAENPSARDQILPVYAEYQQRSEKVLTEYNNKAALEPVNPPKPPPAIKIDPPPPPQEQGLIPGFLMPPSDGSGVTPGTGMPAAPMVPPTGSPGGGLPADTAAQLTSAGREAAALSGDVAVKAASLGGGGGGGVPSAPLGSAIGGAESVRPAGAGDIAGLGQGRAGGGAALGGGGMGMPMGAAHQGQGGAKSKGSQQEDEALYTEDRAWTEAVIGNRRRQDSKESK

SEQ ID NO: 179 - DNA, 1449 bp, Mycobacterium tuberculosis
  source 1..1449: mol_type = genomic DNA
atgggcagcagccatcatcatcatcatcacagcagcggcctggtgccgcgcggcagccatatgacgcagtcgcagaccgtgacggtggatcagcaagagattttgaacagggccaacgaggtggaggccccgatggcggacccaccgactgatgtccccatcacaccgtgcgaactcacggcggctaaaaacgccgcccaacagctggtattgtccgccgacaacatgcgggaatacctggcggccggtgccaaagagcggcagcgtctggcgacctcgctgcgcaacgcggccaaggcgtatggcgaggttgatgaggaggctgcgaccgcgctggacaacgacggcgaaggaactgtgcaggcagaatcggccggggccgtcggaggggacagttcggccgaactaaccgatacgccgagggtggccacggccggtgaacccaacttcatggatctcaaagaagcggcaaggaagctcgaaacgggcgaccaaggcgcatcgctcgcgcactttgcggatgggtggaacactttcaacctgacgctgcaaggcgacgtcaagcggttccgggggtttgacaactgggaaggcgatgcggctaccgcttgcgaggcttcgctcgatcaacaacggcaatggatactccacatggccaaattgagcgctgcgatggccaagcaggctcaatatgtcgcgcagctgcacgtgtgggctaggcgggaacatccgacttatgaagacatagtcgggctcgaacggctttacgcggaaaacccttcggcccgcgaccaaattctcccggtgtacgcggagtatcagcagaggtcggagaaggtgctgaccgaatacaacaacaaggcagccctggaaccggtaaacccgccgaagcctccccccgccatcaagatcgacccgcccccgcctccgcaagagcagggattgatccctggcttcctgatgccgccgtctgacggctccggtgtgactcccggtaccgggatgccagccgcaccgatggttccgcctaccggatcgccgggtggtggcctcccggctgacacggcggcacagctgacgtcggctgggcgggaagccgcagcgctgtcgggcgacgtggcggtcaaagcggcatcgctcggtggcggtggaggcggcggggtgccgtcggcgccgttgggatccgcgatcgggggcgccgaatcggtgcggcccgctggcgctggtgacattgccggcttaggccagggaagggccggcggcggcgccgcgctgggcggcggtggcatgggaatgccgatgggtgccgcgcatcagggacaagggggcgccaagtccaagggttctcagcaggaagacgaggcgctctacaccgaggatcgggcatggaccgaggccgtcattggtaaccgtcggcgccaggacagtaaggagtcgaagtgaaagctt

SEQ ID NO: 180 - protein, 480 aa, Mycobacterium tuberculosis
MGSSHHHHHHSSGLVPRGSHMTQSQTVTVDQQEILNRANEVEAPMADPPTDVPITPCELTAAKNAAQQLVLSADNMREYLAAGAKERQRLATSLRNAAKAYGEVDEEAATALDNDGEGTVQAESAGAVGGDSSAELTDTPRVATAGEPNFMDLKEAARKLETGDQGASLAHFADGWNTFNLTLQGDVKRFRGFDNWEGDAATACEASLDQQRQWILHMAKLSAAMAKQAQYVAQLHVWARREHPTYEDIVGLERLYAENPSARDQILPVYAEYQQRSEKVLTEYNNKAALEPVNPPKPPPAIKIDPPPPPQEQGLIPGFLMPPSDGSGVTPGTGMPAAPMVPPTGSPGGGLPADTAAQLTSAGREAAALSGDVAVKAASLGGGGGGGVPSAPLGSAIGGAESVRPAGAGDIAGLGQGRAGGGAALGGGGMGMPMGAAHQGQGGAKSKGSQQEDEALYTEDRAWTEAVIGNRRRQDSKESK

SEQ ID NO: 181 - protein, 148 aa, Mycobacterium tuberculosis
MSRLSSILRAGAAFLVLGIAAATFPQSAAADSTEDFPIPRRMIATTCDAEQYLAAVRDTSPVYYQRYMIDFNNHANLQQATINKAHWFFSLSPAERRDYSEHFYNGDPLTFAWVNHMKIFFNNKGVVAKGTEVCNGYPAGDMSVWNWA

SEQ ID NO: 182 - DNA, 372 bp, Mycobacterium tuberculosis
  source 1..372: mol_type = genomic DNA
catatggccgactccacggaagactttccaatacctcgccggatgatcgcaaccacctgcgacgccgaacaatatctggcggcggtgcgggataccagtccggtgtactaccagcggtacatgatcgacttcaacaaccatgcaaaccttcagcaagcgacgatcaacaaggcgcactggttcttctcgctgtcaccggcggagcgccgagactactccgaacacttttacaatggcgatccgctgacgtttgcctgggtcaatcacatgaaaatcttcttcaacaacaagggcgtcgtcgctaaagggaccgaggtgtgcaatggatacccagccggcgacatgtcggtgtggaactgggcctaaaagctt

SEQ ID NO: 183 - protein, 140 aa, Mycobacterium tuberculosis
MGSSHHHHHHSSGLVPRGSHMADSTEDFPIPRRMIATTCDAEQYLAAVRDTSPVYYQRYMIDFNNHANLQQATINKAHWFFSLSPAERRDYSEHFYNGDPLTFAWVNHMKIFFNNKGVVAKGTEVCNGYPAGDMSVWNWA

SEQ ID NO: 184 - protein, 261 aa, Mycobacterium tuberculosis
MPKRSEYRQGTPNWVDLQTTDQSAAKKFYTSLFGWGYDDNPVPGGGGVYSMATLNGEAVAAIAPMPPGAPEGMPPIWNTYIAVDDVDAVVDKVVPGGGQVMMPAFDIGDAGRMSFITDPTGAAVGLWQANRHIGATLVNETGTLIWNELLTDKPDLALAFYEAVVGLTHSSMEIAAGQNYRVLKAGDAEVGGCMEPPMPGVPNHWHVYFAVDDADATAAKAAAAGGQVIAEPADIPSVGRFAVLSDPQGAIFSVLKPAPQQ

SEQ ID NO: 185 - DNA, 853 bp, Mycobacterium tuberculosis
  source 1..853: mol_type = genomic DNA
atgggcagcagccatcatcatcatcatcacagcagcggcctggtgccgcgcggcagccatatgcccaagagaagcgaatacaggcaaggcacgccgaactgggtcgaccttcagaccaccgatcagtccgccgccaaaaagttctacacatcgttgttcggctggggttacgacgacaacccggtccccggaggcggtggggtctattccatggccacgctgaacggcgaagccgtggccgccatcgcaccgatgcccccgggtgcaccggaggggatgccgccgatctggaacacctatatcgcggtggacgacgtcgatgcggtggtggacaaggtggtgcccgggggcgggcaggtgatgatgccggccttcgacatcggcgatgccggccggatgtcgttcatcaccgatccgaccggcgctgccgtgggcctatggcaggccaatcggcacatcggagcgacgttggtcaacgagacgggcacgctcatctggaacgaactgctcacggacaagccggatttggcgctagcgttctacgaggctgtggttggcctcacccactcgagcatggagatagctgcgggccagaactatcgggtgctcaaggccggcgacgcggaagtcggcggctgtatggaaccgccgatgcccggcgtgccgaatcattggcacgtctactttgcggtggatgacgccgacgccacggcggccaaagccgccgcagcgggcggccaggtcattgcggaaccggctgacattccgtcggtgggccggttcgccgtgttgtccgatccgcagggcgcgatcttcagtgtgttgaagcccgcaccgcagcaataggaagctt

SEQ ID NO: 186 - protein, 281 aa, Mycobacterium tuberculosis
MGSSHHHHHHSSGLVPRGSHMPKRSEYRQGTPNWVDLQTTDQSAAKKFYTSLFGWGYDDNPVPGGGGVYSMATLNGEAVAAIAPMPPGAPEGMPPIWNTYIAVDDVDAVVDKVVPGGGQVMMPAFDIGDAGRMSFITDPTGAAVGLWQANRHIGATLVNETGTLIWNELLTDKPDLALAFYEAVVGLTHSSMEIAAGQNYRVLKAGDAEVGGCMEPPMPGVPNHWHVYFAVDDADATAAKAAAAGGQVIAEPADIPSVGRFAVLSDPQGAIFSVLKPAPQQ

SEQ ID NO: 187 - protein, 205 aa, Mycobacterium tuberculosis
MTGPTTDADAAVPRRVLIAEDEALIRMDLAEMLREEGYEIVGEAGDGQEAVELAELHKPDLVIMDVKMPRRDGIDAASEIASKRIAPIVVLTAFSQRDLVERARDAGAMAYLVKPFSISDLIPAIELAVSRFREITALEGEVATLSERLETRKLVERAKGLLQTKHGMTEPDAFKWIQRAAMDRRTTMKRVAEVVLETLGTPKDT

SEQ ID NO: 188 - DNA, 684 bp, Mycobacterium tuberculosis
  source 1..684: mol_type = genomic DNA
atgggcagcagccatcatcatcatcatcacagcagcggcctggtgccgcgcggcagccatatgaccggccccaccaccgacgccgatgccgctgtcccacgtcgggtcttgatcgcggaagatgaagcgctcatccgcatggacctggccgagatgttgcgagaggagggatatgaaattgtcggcgaggccggcgacggccaggaagccgtcgagctggccgagctgcacaagcccgacctggtgatcatggacgtgaagatgccgcgccgggacgggatcgacgccgcatccgaaatcgccagcaaacgtattgccccgatcgtggtgctgaccgcgttcagccagcgtgatctggtcgaacgtgcgcgtgatgccggggcgatggcatacctggtaaagcctttcagcatcagcgacctgattccagcgattgaattggcggtcagccggttcagggagatcaccgcgttggaaggcgaggtggcgacgctatctgaacggttggaaacccgcaagctggtggaacgagcaaaaggcctgctgcagaccaaacatgggatgaccgagccggacgctttcaagtggattcaacgtgccgccatggatcggcgcaccaccatgaagcgggtggccgaagtcgtgctggaaaccctcggaacacccaaagacacctgaaagctt

SEQ ID NO: 189 - protein, 225 aa, Mycobacterium tuberculosis
MGSSHHHHHHSSGLVPRGSHMTGPTTDADAAVPRRVLIAEDEALIRMDLAEMLREEGYEIVGEAGDGQEAVELAELHKPDLVIMDVKMPRRDGIDAASEIASKRIAPIVVLTAFSQRDLVERARDAGAMAYLVKPFSISDLIPAIELAVSRFREITALEGEVATLSERLETRKLVERAKGLLQTKHGMTEPDAFKWIQRAAMDRRTTMKRVAEVVLETLGTPKDT

SEQ ID NO: 190 - protein, 181 aa, Mycobacterium tuberculosis
MRVLLLGPPGAGKGTQAVKLAEKLGIPQISTGELFRRNIEEGTKLGVEAKRYLDAGDLVPSDLTNELVDDRLNNPDAANGFILDGYPRSVEQAKALHEMLERRGTDIDAVLEFRVSEEVLLERLKGRGRADDTDDVILNRMKVYRDETAPLLEYYRDQLKTVDAVGTMDEVFARALRALGK

SEQ ID NO: 191 - DNA, 613 bp, Mycobacterium tuberculosis
  source 1..613: mol_type = genomic DNA
atgggcagcagccatcatcatcatcatcacagcagcggcctggtgccgcgcggcagccatatgagagttttgttgctgggaccgcccggggcgggcaaggggacgcaggcggtgaagctggccgagaagctcgggatcccgcagatctccaccggcgaactcttccggcgcaacatcgaagagggcaccaagctcggcgtggaagccaaacgctacttggatgccggtgacttggtgccgtccgacttgaccaatgaactcgtcgacgaccggctgaacaatccggacgcggccaacggattcatcttggatggctatccacgctcggtcgagcaggccaaggcgcttcacgagatgctcgaacgccgggggaccgacatcgacgcggtgctggagtttcgtgtgtccgaggaggtgttgttggagcgactcaaggggcgtggccgcgccgacgacaccgacgacgtcatcctcaaccggatgaaggtctaccgcgacgagaccgcgccgctgctggagtactaccgcgaccaattgaagaccgtcgacgccgtcggcaccatggacgaggtgttcgcccgtgcgttgcgggctctgggaaagttagaagctt

SEQ ID NO: 192 - protein, 201 aa, Mycobacterium tuberculosis
MGSSHHHHHHSSGLVPRGSHMRVLLLGPPGAGKGTQAVKLAEKLGIPQISTGELFRRNIEEGTKLGVEAKRYLDAGDLVPSDLTNELVDDRLNNPDAANGFILDGYPRSVEQAKALHEMLERRGTDIDAVLEFRVSEEVLLERLKGRGRADDTDDVILNRMKVYRDETAPLLEYYRDQLKTVDAVGTMDEVFARALRALGK

SEQ ID NO: 193 - protein, 75 aa, Mycobacterium tuberculosis
MVDRDPNTIKQEIDQTRDQLAATIDSLAERANPRRLADDAKTRVIAFLRKPIVTVSLVGIGSVVVVVVIHKIRNR

SEQ ID NO: 194 - DNA, 258 bp, Mycobacterium tuberculosis
  source 1..258: mol_type = genomic DNA
catatgcatcaccatcaccatcacgtggtggaccgcgatcccaataccatcaagcaggagatcgaccaaacccgcgaccaactggcggcgaccatcgattccctcgccgagcgcgccaacccccgccgcctcgccgacgacgcaaaaactcgggtgatcgccttcctcaggaagcccatcgtgaccgtgtcactggtcgggatcgggtctgtggtcgtcgtcgtggtcatccacaagatcaggaatcgctgagaattc

SEQ ID NO: 195 - protein, 82 aa, Mycobacterium tuberculosis
MHHHHHHVVDRDPNTIKQEIDQTRDQLAATIDSLAERANPRRLADDAKTRVIAFLRKPIVTVSLVGIGSVVVVVVIHKIRNR

SEQ ID NO: 196 - protein, 563 aa, Mycobacterium tuberculosis
MAFPEYSPAASAATFADLQIHPRVLRAIGDVGYESPTAIQAATIPALMAGSDVVGLAQTGTGKTAAFAIPMLSKIDITSKVPQALVLVPTRELALQVAEAFGRYGAYLSQLNVLPIYGGSSYAVQLAGLRRGAQVVVGTPGRMIDHLERATLDLSRVDFLVLDEADEMLTMGFADDVERILSETPEYKQVALFSATMPPAIRKLSAKYLHDPFEVTCKAKTAVAENISQSYIQVARKMDALTRVLEVEPFEAMIVFVRTKQATEEIAEKLRARGFSAAAISGDVPQAQRERTITALRDGDIDILVATDVAARGLDVERISHVLNYDIPHDTESYVHRIGRTGRAGRSGAALIFVSPRELHLLKAIEKATRQTLTEAQLPTVEDVNTQRVAKFADSITNALGGPGIELFRRLVEEYEREHDVPMADIAAALAVQCRGGEAFLMAPDPPLSRRNRDQRRDRPQRPKRRPDLTTYRVAVGKRHKIGPGAIVGAIANEGGLHRSDFGQIRIGPDFSLVELPAKLPRATLKKLAQTRISGVLIDLRPYRPPDAARRHNGGKPRRKHVG

SEQ ID NO: 197 - DNA, 1758 bp, Mycobacterium tuberculosis
  source 1..1758: mol_type = genomic DNA
atgggcagcagccatcatcatcatcatcacagcagcggcctggtgccgcgcggcagccatatggccttcccggaatattcgcctgcggcgtccgctgcgacgtttgctgacctgcagattcatccccgcgtcttgcgggcgatcggcgacgtcggttacgagtcaccgacggctatccaggcggctacgatcccggcgttgatggcaggctccgacgtggtggggctggcgcagaccggcaccggcaagacggcggcatttgcgattccgatgctgtccaagatcgacatcaccagcaaggtgccccaggcgctggtgctggtgcccacccgggagctggctctgcaggtggccgaggcgttcggccgctacggtgcctatctgtcgcaactcaacgtgctgccgatctacggcggatcgtcgtatgccgtgcaactggccggattgagacgcggcgcgcaggtggtggttggcacccccggtcgtatgatagaccatctcgaacgggcgaccttggacctgtcgcgggtggactttctagtgctcgatgaggccgatgagatgctgaccatgggtttcgccgacgacgttgagcgcattctgtccgagacccccgaatacaagcaggtcgccctgttttccgcgaccatgccgccggcgatccgcaaactcagcgccaagtatctgcacgatccgttcgaagtcacttgtaaggcgaaaaccgctgtggccgagaatatttcgcagagctacattcaggtagcacggaagatggacgcgctcaccagagtgctcgaagtcgagccgttcgaggcgatgatcgtctttgtccgcaccaagcaggcgaccgaggagattgccgaaaagctgcgtgcccgagggttttccgcggctgccatcagcggtgacgtcccgcaggcgcagcgggagcggaccatcacggcgctgcgggacggcgacatcgatatcctggtcgccaccgatgtggcggcgcgcggactcgacgtggagcggatatcacacgtgcttaactacgacatcccgcacgacaccgagtcctacgtacaccggatcgggcgcaccggcagggccgggcgttcgggagccgcgctgatattcgtctcgccacgggagcttcacctgctcaaggcgatcgaaaaggctacgcggcaaacgcttaccgaggcgcaattgcccaccgtcgaggatgtcaacacccagcgggtggccaagttcgccgattccatcaccaatgcgctgggcggtccgggaatcgagctgttccgccgactggtcgaggagtatgaacgcgagcatgatgtcccgatggctgacatcgccgcggcactggccgtgcagtgccgcggcggtgaggcattcctgatggcacccgacccgccgctttcgcggcgcaaccgcgaccagcgtcgggaccgtccgcaaaggcccaagcgtagaccggacttgaccacctaccgcgtcgccgtcggcaagcggcacaagatcggtccaggcgccatcgtcggcgccatcgccaatgagggtgggctgcaccgcagcgacttcggtcagatccgtatcgggccagacttctcgctagtagaattgccggcgaagctgccccgcgcgacgctcaaaaagcttgcacagacccgtatctcgggtgtgctgatcgaccttcggccataccggccgcccgacgcggcgcgccggcataatggcggcaaaccacggcggaaacacgtcggatgagaattc

SEQ ID NO: 198 - protein, 583 aa, Mycobacterium tuberculosis
MGSSHHHHHHSSGLVPRGSHMAFPEYSPAASAATFADLQIHPRVLRAIGDVGYESPTAIQAATIPALMAGSDVVGLAQTGTGKTAAFAIPMLSKIDITSKVPQALVLVPTRELALQVAEAFGRYGAYLSQLNVLPIYGGSSYAVQLAGLRRGAQVVVGTPGRMIDHLERATLDLSRVDFLVLDEADEMLTMGFADDVERILSETPEYKQVALFSATMPPAIRKLSAKYLHDPFEVTCKAKTAVAENISQSYIQVARKMDALTRVLEVEPFEAMIVFVRTKQATEEIAEKLRARGFSAAAISGDVPQAQRERTITALRDGDIDILVATDVAARGLDVERISHVLNYDIPHDTESYVHRIGRTGRAGRSGAALIFVSPRELHLLKAIEKATRQTLTEAQLPTVEDVNTQRVAKFADSITNALGGPGIELFRRLVEEYEREHDVPMADIAAALAVQCRGGEAFLMAPDPPLSRRNRDQRRDRPQRPKRRPDLTTYRVAVGKRHKIGPGAIVGAIANEGGLHRSDFGQIRIGPDFSLVELPAKLPRATLKKLAQTRISGVLIDLRPYRPPDAARRHNGGKPRRKHVG

SEQ ID NO: 199 - protein, 228 aa, Mycobacterium tuberculosis
MRIKIFMLVTAVVLLCCSGVATAAPKTYCEELKGTDTGQACQIQMSDPAYNINISLPSYYPDQKSLENYIAQTRDKFLSAATSSTPREAPYELNITSATYQSAIPPRGTQAVVLKVYQNAGGTHPTTTYKAFDWDQAYRKPITYDTLWQADTDPLPVVFPIVQGELSKQTGQQVSIAPNAGLDPVNYQNFAVTNDGVIFFFNPGELLPEAAGPTQVLVPRSAIDSMLA

SEQ ID NO: 200 - DNA, 687 bp, Mycobacterium tuberculosis
  source 1..687: mol_type = genomic DNA
atgggcagcagccatcatcatcatcatcacagcagcggcctggtgccgcgcggcagccatatggcgcccaagacctactgcgaggagttgaaaggcaccgataccggccaggcgtgccagattcaaatgtccgacccggcctacaacatcaacatcagcctgcccagttactaccccgaccagaagtcgctggaaaattacatcgcccagacgcgcgacaagttcctcagcgcggccacatcgtccactccacgcgaagccccctacgaattgaatatcacctcggccacataccagtccgcgataccgccgcgtggtacgcaggccgtggtgctcaaggtctaccagaacgccggcggcacgcacccaacgaccacgtacaaggccttcgattgggaccaggcctatcgcaagccaatcacctatgacacgctgtggcaggctgacaccgatccgctgccagtcgtcttccccattgtgcaaggtgaactgagcaagcagaccggacaacaggtatcgatagcgccgaatgccggcttggacccggtgaattatcagaacttcgcagtcacgaacgacggggtgattttcttcttcaacccgggggagttgctgcccgaagcagccggcccaacccaggtattggtcccacgttccgcgatcgactcgatgctggcctagaagctt

SEQ ID NO: 201 - protein, 226 aa, Mycobacterium tuberculosis
MGSSHHHHHHSSGLVPRGSHMAPKTYCEELKGTDTGQACQIQMSDPAYNINISLPSYYPDQKSLENYIAQTRDKFLSAATSSTPREAPYELNITSATYQSAIPPRGTQAVVLKVYQNAGGTHPTTTYKAFDWDQAYRKPITYDTLWQADTDPLPVVFPIVQGELSKQTGQQVSIAPNAGLDPVNYQNFAVTNDGVIFFFNPGELLPEAAGPTQVLVPRSAIDSMLA

SEQ ID NO: 202 - protein, 162 aa, Mycobacterium tuberculosis
MQFDVTIEIPKGQRNKYEVDHETGRVRLDRYLYTPMAYPTDYGFIEDTLGDDGDPLDALVLLPQPVFPGVLVAARPVGMFRMVDEHGGDDKVLCVPAGDPRWDHVQDIGDVPAFELDAIKHFFVHYKDLEPGKFVKAADWVDRAEAEAEVQRSVERFKAGTH

SEQ ID NO: 203 - DNA, 519 bp, Mycobacterium tuberculosis
  source 1..519: mol_type = genomic DNA
catatgcatcaccatcaccatcacatgcaattcgacgtgaccatcgaaattcccaagggccagcgcaacaaatacgaggtcgaccatgagacggggcgggttcgtctggaccggtacctgtacaccccgatggcctacccgaccgactacggcttcatcgaggacaccctaggtgacgatggcgacccgctggacgcgctggtgctgctaccgcagccggtcttccccggggtgctggtggcggcgcggccggtggggatgttccggatggtcgacgagcacggcggcgacgacaaagtgctgtgcgtcccagccggtgacccccggtgggaccacgtccaagacatcggggacgttccggctttcgagctggatgcgatcaagcatttctttgtgcactacaaggacctggaaccaggtaagttcgtcaaggcggccgactgggtcgaccgcgccgaagccgaggcagaggtgcagcgttcagtggagcgcttcaaggccggtacacactgagaattc

SEQ ID NO: 204 - protein, 169 aa, Mycobacterium tuberculosis
MHHHHHHMQFDVTIEIPKGQRNKYEVDHETGRVRLDRYLYTPMAYPTDYGFIEDTLGDDGDPLDALVLLPQPVFPGVLVAARPVGMFRMVDEHGGDDKVLCVPAGDPRWDHVQDIGDVPAFELDAIKHFFVHYKDLEPGKFVKAADWVDRAEAEAEVQRSVERFKAGTH

SEQ ID NO: 205 - protein, 176 aa, Mycobacterium tuberculosis
MHPLPADHGRSRCNRHPISPLSLIGNASATSGDMSSMTRIAKPLIKSAMAAGLVTASMSLSTAVAHAGPSPNWDAVAQCESGGNWAANTGNGKYGGLQFKPATWAAFGGVGNPAAASREQQIAVANRVLAEQGLDAWPTCGAASGLPIALWSKPAQGIKQIINEIIWAGIQASIPR

SEQ ID NO: 206 - DNA, 474 bp, Mycobacterium tuberculosis
  source 1..474: mol_type = genomic DNA
catatgcatcaccatcaccatcacacttccggcgatatgtcgagcatgacaagaatcgccaagccgctcatcaagtccgccatggccgcaggactcgtcacggcatccatgtcgctctccaccgccgttgcccacgccggtcccagcccgaactgggacgccgtcgcgcagtgcgaatccgggggcaactgggcggccaacaccggaaacggcaaatacggcggactgcagttcaagccggccacctgggccgcattcggcggtgtcggcaacccagcagctgcctctcgggaacaacaaatcgcagttgccaatcgggttctcgccgaacagggattggacgcgtggccgacgtgcggcgccgcctctggccttccgatcgcactgtggtcgaaacccgcgcagggcatcaagcaaatcatcaacgagatcatttgggcaggcattcaggcaagtattccgcgctgagaattc

SEQ ID NO: 207 - protein, 154 aa, Mycobacterium tuberculosis
MHHHHHHTSGDMSSMTRIAKPLIKSAMAAGLVTASMSLSTAVAHAGPSPNWDAVAQCESGGNWAANTGNGKYGGLQFKPATWAAFGGVGNPAAASREQQIAVANRVLAEQGLDAWPTCGAASGLPIALWSKPAQGIKQIINEIIWAGIQASIPR

SEQ ID NO: 208 - protein, 99 aa, Mycobacterium tuberculosis
MEKMSHDPIAADIGTQVSDNALHGVTAGSTALTSVTGLVPAGADEVSAQAATAFTSEGIQLLASNASAQDQLHRAGEAVQDVARTYSQIDDGAAGVFAE

SEQ ID NO: 209 - DNA, 366 bp, Mycobacterium tuberculosis
  source 1..366: mol_type = genomic DNA
atgggcagcagccatcatcatcatcatcacagcagcggcctggtgccgcgcggcagccatatggaaaaaatgtcacatgatccgatcgctgccgacattggcacgcaagtgagcgacaacgctctgcacggcgtgacggccggctcgacggcgctgacgtcggtgaccgggctggttcccgcgggggccgatgaggtctccgcccaagcggcgacggcgttcacatcggagggcatccaattgctggcttccaatgcatcggcccaagaccagctccaccgtgcgggcgaagcggtccaggacgtcgcccgcacctattcgcaaatcgacgacggcgccgccggcgtcttcgccgaatagaagctt

SEQ ID NO: 210 - protein, 119 aa, Mycobacterium tuberculosis
MGSSHHHHHHSSGLVPRGSHMEKMSHDPIAADIGTQVSDNALHGVTAGSTALTSVTGLVPAGADEVSAQAATAFTSEGIQLLASNASAQDQLHRAGEAVQDVARTYSQIDDGAAGVFAE

SEQ ID NO: 211 - protein, 367 aa, Mycobacterium tuberculosis
MLWHAMPPELNTARLMAGAGPAPMLAAAAGWQTLSAALDAQAVELTARLNSLGEAWTGGGSDKALAAATPMVVWLQTASTQAKTRAMQATAQAAAYTQAMATTPSLPEIAANHITQAVLTATNFFGINTIPIALTEMDYFIRMWNQAALAMEVYQAETAVNTLFEKLEPMASILDPGASQSTTNPIFGMPSPGSSTPVGQLPPAATQTLGQLGEMSGPMQQLTQPLQQVTSLFSQVGGTGGGNPADEEAAQMGLLGTSPLSNHPLAGGSGPSAGAGLRAESLPGAGGSLTRTPLMSQLIEKPVAPSVMPAAAAGSSATGGAAPVGAGAMGQGAQSGGSTRPGLVAPAPLAQEREEDDEDDWDEEDDW

SEQ ID NO: 212 - DNA, 1173 bp, Mycobacterium tuberculosis
  source 1..1173: mol_type = genomic DNA
atgggcagcagccatcatcatcatcatcacagcagcggcctggtgccgcgcggcagccatatgctgtggcacgcaatgccaccggagctaaataccgcacggctgatggccggcgcgggtccggctccaatgcttgcggcggccgcgggatggcagacgctttcggcggctctggacgctcaggccgtcgagttgaccgcgcgcctgaactctctgggagaagcctggactggaggtggcagcgacaaggcgcttgcggctgcaacgccgatggtggtctggctacaaaccgcgtcaacacaggccaagacccgtgcgatgcaggcgacggcgcaagccgcggcatacacccaggccatggccacgacgccgtcgctgccggagatcgccgccaaccacatcacccaggccgtccttacggccaccaacttcttcggtatcaacacgatcccgatcgcgttgaccgagatggattatttcatccgtatgtggaaccaggcagccctggcaatggaggtctaccaggccgagaccgcggttaacacgcttttcgagaagctcgagccgatggcgtcgatccttgatcccggcgcgagccagagcacgacgaacccgatcttcggaatgccctcccctggcagctcaacaccggttggccagttgccgccggcggctacccagaccctcggccaactgggtgagatgagcggcccgatgcagcagctgacccagccgctgcagcaggtgacgtcgttgttcagccaggtgggcggcaccggcggcggcaacccagccgacgaggaagccgcgcagatgggcctgctcggcaccagtccgctgtcgaaccatccgctggctggtggatcaggccccagcgcgggcgcgggcctgctgcgcgcggagtcgctacctggcgcaggtgggtcgttgacccgcacgccgctgatgtctcagctgatcgaaaagccggttgccccctcggtgatgccggcggctgctgccggatcgtcggcgacgggtggcgccgctccggtgggtgcgggagcgatgggccagggtgcgcaatccggcggctccaccaggccgggtctggtcgcgccggcaccgctcgcgcaggagcgtgaagaagacgacgaggacgactgggacgaagaggacgactggtgaaagctt

SEQ ID NO: 213 - protein, 388 aa, Mycobacterium tuberculosis
MGSSHHHHHHSSGLVPRGSHMLWHAMPPELNTARLMAGAGPAPMLAAAAGWQTLSAALDAQAVELTARLNSLGEAWTGGGSDKALAAATPMVVWLQTASTQAKTRAMQATAQAAAYTQAMATTPSLPEIAANHITQAVLTATNFFGINTIPIALTEMDYFIRMWNQAALAMEVYQAETAVNTLFEKLEPMASILDPGASQSTTNPIFGMPSPGSSTPVGQLPPAATQTLGQLGEMSGPMQQLTQPLQQVTSLFSQVGGTGGGNPADEEAAQMGLLGTSPLSNHPLAGGSGPSAGAGLLRAESLPGAGGSLTRTPLMSQLIEKPVAPSVMPAAAAGSSATGGAAPVGAGAMGQGAQSGGSTRPGLVAPAPLAQEREEDDEDDWDEEDDW

SEQ ID NO: 214 - protein, 340 aa, Mycobacterium tuberculosis
MKRALITGITGQDGSYLAELLLAKGYEVHGLIRRASTFNTSRIDHLYVDPHQPGARLFLHYGDLIDGTRLVTLLSTIEPDEVYNLAAQSHVRVSFDEPVHTGDTTGMGSMRLLEAVRLSRVHCRFYQASSSEMFGASPPPQNELTPFYPRSPYGAAKVYSYWATRNYREAYGLFAVNGILFNHESPRRGETFVTRKITRAVARIKAGIQSEVYMGNLDAVRDWGYAPEYVEGMWRMLQTDEPDDFVLATGRGFTVREFARAAFEHAGLDWQQYVKFDQRYLRPTEVDSLIGDATKAAELLGWRASVHTDELARIMVDADMAALECEGKPWIDKPMIAGRT

SEQ ID NO: 215 - DNA, 1053 bp, Mycobacterium tuberculosis
  source 1..1053: mol_type = genomic DNA
catatgcatcaccatcaccatcacgtgaagcgagcgctcatcaccggaatcaccggccaggacggctcgtatctcgccgaactgctgctggccaaggggtatgaggttcacgggctcatccggcgcgcttcgacgttcaacacctcgcggatcgatcacctctacgtcgacccgcaccaaccgggcgcgcggctgtttctgcactatggtgacctgatcgacggaacccggttggtgaccctgctgagcaccatcgaacccgacgaggtgtacaacctggcggcgcagtcacacgtgcgggtgagcttcgacgaacccgtgcacaccggtgacaccaccggcatgggatccatgcgactgctggaagccgttcggctctctcgggtgcactgccgcttctatcaggcgtcctcgtcggagatgttcggcgcctcgccgccaccgcagaacgagctgacgccgttctacccgcggtcaccgtatggcgccgccaaggtctattcgtactgggcgacccgcaattatcgcgaagcgtacggattgttcgccgttaacggcatcttgttcaatcacgaatcaccgcggcgcggtgagacgttcgtgacccgaaagatcaccagggccgtggcacgcatcaaggccggtatccagtccgaggtctatatgggcaatctggatgcggtccgcgactgggggtacgcgcccgaatacgtcgaaggcatgtggcggatgctgcagaccgacgagcccgacgacttcgttttggcgaccgggcgcggtttcaccgtgcgtgagttcgcgcgggccgcgttcgagcatgccggtttggactggcagcagtacgtgaaattcgaccaacgctatctgcggcccaccgaggtggattcgctgatcggcgacgcgaccaaggctgccgaattgctgggctggagggcttcggtgcacactgacgagttggctcggatcatggtcgacgcggacatggcggcgctggagtgcgaaggcaagccgtggatcgacaagccgatgatcgccggccggacatgagaattc

SEQ ID NO: 216 - protein, 347 aa, Mycobacterium tuberculosis
MHHHHHHVKRALITGITGQDGSYLAELLLAKGYEVHGLIRRASTFNTSRIDHLYVDPHQPGARLFLHYGDLIDGTRLVTLLSTIEPDEVYNLAAQSHVRVSFDEPVHTGDTTGMGSMRLLEAVRLSRVHCRFYQASSSEMFGASPPPQNELTPFYPRSPYGAAKVYSYWATRNYREAYGLFAVNGILFNHESPRRGETFVTRKITRAVARIKAGIQSEVYMGNLDAVRDWGYAPEYVEGMWRMLQTDEPDDFVLATGRGFTVREFARAAFEHAGLDWQQYVKFDQRYLRPTEVDSLIGDATKAAELLGWRASVHTDELARIMVDADMAALECEGKPWIDKPMIAGRT

SEQ ID NO: 217 - DNA, 2742 bp, Mycobacterium tuberculosis
  source 1..2742: mol_type = genomic DNA
atgggcagcagccatcatcatcatcatcacagcagcggcctggtgccgcgcggcagccatatgaccatcaactatcaattcggggacgtcgacgctcacggcgccatgatccgcgctcaggccgggtcgctggaggccgagcatcaggccatcatttctgatgtgttgaccgcgagtgacttttggggcggcgccggttcggcggcctgccaggggttcattacccagctgggccgtaacttccaggtgatctacgagcaggccaacgcccacgggcagaaggtgcaggctgccggcaacaacatggcacaaaccgacagcgccgtcggctccagctgggccggtacccatctcgccaacggttcgatgtcggaagtcatgatgtcggaaattgccgggttgcctatccctccgattatccattacggggcgattgcctatgcccccagcggcgcgtcgggcaaagcgtggcaccagcgcacaccggcgcgagcagagcaagtcgcactagaaaagtgcggtgacaagacttgcaaagtggttagtcgcttcaccaggtgcggcgcggtcgcctacaacggctcgaaataccaaggcggaaccggactcacgcgccgcgcggcagaagacgacgccgtgaaccgactcgaaggcgggcggatcgtcaactgggcgtgcaacgagctcatgacctcgcgttttatgacggatccgcacgcgatgcgggacatggcgggccgttttgaggtgcacgcccagacggtggaggacgaggctcgccggatgtgggcgtccgcgcaaaacatctcgggcgcgggctggagtggcatggccgaggcgacctcgctagacaccatgacccagatgaatcaggcgtttcgcaacatcgtgaacatgctgcacggggtgcgtgacgggctggttcgcgacgccaacaactacgaacagcaagagcaggcctcccagcagatcctcagcagcgtcgacatcaatttcgccgttttgccgccggaggtgaattcggcgcgcatattcgccggtgcgggcctgggcccaatgctggcggcggcgtcggcctgggacgggttggccgaggagttgcatgccgcggcgggctcgttcgcgtcggtgaccaccgggttggcgggcgacgcgtggcatggtccggcgtcgctggcgatgacccgcgcggccagcccgtatgtggggtggttgaacacggcggcgggtcaggccgcgcaggcggccggccaggcgcggctagcggcgagcgcgttcgaggcgacgctggcggccaccgtgtctccagcgatggtcgcggccaaccggacacggctggcgtcgctggtggcagccaacttgctgggccagaacgccccggcgatcgcggccgcggaggctgaatacgagcagatatgggcccaggacgtggccgcgatgttcggctatcactccgccgcgtcggcggtggccacgcagctggcgcctattcaagagggtttgcagcagcagctgcaaaacgtgctggcccagttggctagcgggaacctgggcagcggaaatgtgggcgtcggcaacatcggcaacgacaacattggcaacgcaaacatcggcttcggaaatcgaggcgacgccaacatcggcatcgggaatatcggcgacagaaacctcggcattgggaacaccggcaattggaatatcggcatcggcatcaccggcaacggacaaatcggcttcggcaagcctgccaaccccgacgtcttggtggtgggcaacggcggcccgggagtaaccgcgttggtcatgggcggcaccgacagcctactgccgctgcccaacatccccttactcgagtacgctgcgcggttcatcacccccgtgcatcccggatacaccgctacgttcctggaaacgccatcgcagtttttcccattcaccgggctgaatagcctgacctatgacgtctccgtggcccagggcgtaacgaatctgcacaccgcgatcatggcgcaactcgcggcgggaaacgaagtcgtcgtcttcggcacctcccaaagcgccacgatagccaccttcgaaatgcgctatctgcaatccctgccagcacacctgcgtccgggtctcgacgaattgtcctttacgttgaccggcaatcccaaccggcccgacggtggcattcttacgcgttttggcttctccataccgcagttgggtttcacattgtccggcgcgacgcccgccgacgcctaccccaccgtcgattacgcgttccagtacgacggcgtcaacgacttccccaaatacccgctgaatgtcttcgcgaccgccaacgcgatcgcgggcatccttttcctgcactccgggttgattgcgttgccgcccgatcttgcctcgggcgtggttcaaccggtgtcctcaccggacgtcctgaccacctacatcctgctgcccagccaagatctgccgctgctggtcccgctgcgtgctatccccctgctgggaaacccgcttgccgacctcatccagccggacttgcgggtgctcgtcgagttgggttatgaccgcaccgcccaccaggacgtgcccagcccgttcggactgtttccggacgtcgattgggccgaggtggccgcggacctgcagcaaggcgccgtgcaaggcgtcaacgacgccctgtccggactggggctgccgccgccgtggcagccggcgctaccccgacttttcagtacttaaaagctt

SEQ ID NO: 218 - DNA, 39 bp, synthetic construct; identical to SEQ ID NO: 73
  misc_feature 1..39: note = Primer
  source 1..39: mol_type = other DNA

SEQ ID NO: 219 - DNA, 33 bp, synthetic construct; identical to SEQ ID NO: 74
  misc_feature 1..33: note = Primer
  source 1..33: mol_type = other DNA

SEQ ID NO: 220 - DNA, 33 bp, synthetic construct; identical to SEQ ID NO: 75
  misc_feature 1..33: note = Primer
  source 1..33: mol_type = other DNA

SEQ ID NO: 221 - DNA, 33 bp, synthetic construct; identical to SEQ ID NO: 76
  misc_feature 1..33: note = Primer
  source 1..33: mol_type = other DNA

SEQ ID NO: 222 - DNA, 33 bp, synthetic construct; identical to SEQ ID NO: 89
  misc_feature 1..33: note = Primer
  source 1..33: mol_type = other DNA

SEQ ID NO: 223 - DNA, 42 bp, synthetic construct; identical to SEQ ID NO: 90
  misc_feature 1..42: note = Primer
  source 1..42: mol_type = other DNA

SEQ ID NO: 224 - DNA, 30 bp, synthetic construct
  misc_feature 1..30: note = Primer
  source 1..30: mol_type = other DNA
caattacatatgaccatcaactatcaattc

SEQ ID NO: 225 - DNA, 33 bp, synthetic construct
  misc_feature 1..33: note = Primer
  source 1..33: mol_type = other DNA
caattaggtaccggcccagctggagccgacggc

SEQ ID NO: 226 - protein, 911 aa, Mycobacterium tuberculosis
MGSSHHHHHHSSGLVPRGSHMTINYQFGDVDAHGAMIRAQAGSLEAEHQAIISDVLTASDFWGGAGSAACQGFITQLGRNFQVIYEQANAHGQKVQAAGNNMAQTDSAVGSSWAGTHLANGSMSEVMMSEIAGLPIPPIIHYGAIAYAPSGASGKAWHQRTPARAEQVALEKCGDKTCKVVSRFTRCGAVAYNGSKYQGGTGLTRRAAEDDAVNRLEGGRIVNWACNELMTSRFMTDPHAMRDMAGRFEVHAQTVEDEARRMWASAQNISGAGWSGMAEATSLDTMTQMNQAFRNIVNMLHGVRDGLVRDANNYEQQEQASQQILSSVDINFAVLPPEVNSARIFAGAGLGPMLAAASAWDGLAEELHAAAGSFASVTTGLAGDAWHGPASLAMTRAASPYVGWLNTAAGQAAQAAGQARLAASAFEATLAATVSPAMVAANRTRLASLVAANLLGQNAPAIAAAEAEYEQIWAQDVAAMFGYHSAASAVATQLAPIQEGLQQQLQNVLAQLASGNLGSGNVGVGNIGNDNIGNANIGFGNRGDANIGIGNIGDRNLGIGNTGNWNIGIGITGNGQIGFGKPANPDVLVVGNGGPGVTALVMGGTDSLLPLPNIPLLEYAARFITPVHPGYTATFLETPSQFFPFTGLNSLTYDVSVAQGVTNLHTAIMAQLAAGNEVVVFGTSQSATIATFEMRYLQSLPAHLRPGLDELSFTLTGNPNRPDGGILTRFGFSIPQLGFTLSGATPADAYPTVDYAFQYDGVNDFPKYPLNVFATANAIAGILFLHSGLIALPPDLASGVVQPVSSPDVLTTYILLPSQDLPLLVPLRAIPLLGNPLADLIQPDLRVLVELGYDRTAHQDVPSPFGLFPDVDWAEVAADLQQGAVQGVNDALSGLGLPPPWQPALPRLFST

SEQ ID NO: 227 - DNA, 2664 bp, Mycobacterium tuberculosis
  source 1..2664: mol_type = genomic DNA
catatgatgaccatcaactatcaattcggggacgtcgacgctcacggcgccatgatccgcgctcaggccgggtcgctggaggccgagcatcaggccatcatttctgatgtgttgaccgcgagtgacttttggggcggcgccggttcggcggcctgccaggggttcattacccagctgggccgtaacttccaggtgatctacgagcaggccaacgcccacgggcagaaggtgcaggctgccggcaacaacatggcacaaaccgacagcgccgtcggctccagctgggccggtaccgacgacatcgattgggacgccatcgcgcaatgcgaatccggcggcaattgggcggccaacaccggtaacgggttatacggtggtctgcagatcagccaggcgacgtgggattccaacggtggtgtcgggtcgccggcggccgcgagtccccagcaacagatcgaggtcgcagacaacattatgaaaacccaaggcccgggtgcgtggccgaaatgtagttcttgtagtcagggagacgcaccgctgggctcgctcacccacatcctgacgttcctcgcggccgagactggaggttgttcggggagcagggacgatggatccgtggtggatttcggggcgttaccaccggagatcaactccgcgaggatgtacgccggcccgggttcggcctcgctggtggccgccgcgaagatgtgggacagcgtggcgagtgacctgttttcggccgcgtcggcgtttcagtcggtggtctggggtctgacggtggggtcgtggataggttcgtcggcgggtctgatggcggcggcggcctcgccgtatgtggcgtggatgagcgtcaccgcggggcaggcccagctgaccgccgcccaggtccgggttgctgcggcggcctacgagacagcgtataggctgacggtgcccccgccggtgatcgccgagaaccgtaccgaactgatgacgctgaccgcgaccaacctcttggggcaaaacacgccggcgatcgaggccaatcaggccgcatacagccagatgtggggccaagacgcggaggcgatgtatggctacgccgccacggcggcgacggcgaccgaggcgttgctgccgttcgaggacgccccactgatcaccaaccccggcgggctccttgagcaggccgtcgcggtcgaggaggccatcgacaccgccgcggcgaaccagttgatgaacaatgtgccccaagcgctgcaacagctggcccagccagcgcagggcgtcgtaccttcttccaagctgggtgggctgtggacggcggtctcgccgcatctgtcgccgctcagcaacgtcagttcgatagccaacaaccacatgtcgatgatgggcacgggtgtgtcgatgaccaacaccttgcactcgatgttgaagggcttagctccggcggcggctcaggccgtggaaaccgcggcggaaaacggggtctgggcgatgagctcgctgggcagccagctgggttcgtcgctgggttcttcgggtctgggcgctggggtggccgccaacttgggtcgggcggcctcggtcggttcgttgtcggtgccgccagcatgggccgcggccaaccaggcggtcaccccggcggcgcgggcgctgccgctgaccagcctgaccagcgccgcccaaaccgcccccggacacatgctgggcgggctaccgctggggcactcggtcaacgccggcagcggtatcaacaatgcgctgcgggtgccggcacgggcctacgcgataccccgcacaccggccgccggagaattcttctcccggccggggctgccggtcgagtacctgcaggtgccgtcgccgtcgatgggccgcgacatcaaggttcagttccagagcggtgggaacaactcacctgcggtttatctgctcgacggcctgcgcgcccaagacgactacaacggctgggatatcaacaccccggcgttcgagtggtactaccagtcgggactgtcgatagtcatgccggtcggcgggcagtccagcttctacagcgactggtacagcccggcctgcggtaaggctggctgccagacttacaagtgggaaaccttcctgaccagcgagctgccgcaatggttgtccgccaacagggccgtgaagcccaccggcagcgctgcaatcggcttgtcgatggccggctcgtcggcaatgatcttggccgcctaccacccccagcagttcatctacgccggctcgctgtcggccctgctggacccctctcaggggatggggcctagcctgatcggcctcgcgatgggtgacgccggcggttacaaggccgcagacatgtggggtccctcgagtgacccggcatgggagcgcaacgaccctacgcagcagatccccaagctggtcgcaaacaacacccggctatgggtttattgcgggaacggcaccccgaacgagttgggcggtgccaacatacccgccgagttcttggagaacttcgttcgtagcagcaacctgaagttccaggatgcgtacaacgccgcgggcgggcacaacgccgtgttcaacttcccgcccaacggcacgcacagctgggagtactggggcgctcagctcaacgccatgaagggtgacctgcagagttcgttaggcgccggctgaaagctt

SEQ ID NO: 228 - DNA, 30 bp, synthetic construct; identical to SEQ ID NO: 224
  misc_feature 1..30: note = Primer
  source 1..30: mol_type = other DNA

SEQ ID NO: 229 - DNA, 32 bp, synthetic construct
  misc_feature 1..32: note = Primer
  source 1..32: mol_type = other DNA
caattaggtaccggcccagctggagccgacgg

SEQ ID NO: 230 - DNA, 33 bp, synthetic construct
  misc_feature 1..33: note = Primer
  source 1..33: mol_type = other DNA
tgggccggtaccgacgacatcgattgggacgcc

SEQ ID NO: 231 - DNA, 36 bp, synthetic construct
  misc_feature 1..36: note = Primer
  source 1..36: mol_type = other DNA
aatccaccacggatccatcgtccctgctccccgaac

SEQ ID NO: 232 - DNA, 38 bp, synthetic construct
  misc_feature 1..38: note = Primer
  source 1..38: mol_type = other DNA
cagggacgatggatccgtggtggatttcggggcgttac

SEQ ID NO: 233 - DNA, 35 bp, synthetic construct
  misc_feature 1..35: note = Primer
  source 1..35: mol_type = other DNA
ccgggagaagaattctccggcggccggtgtgcggg

SEQ ID NO: 234 - DNA, 35 bp, synthetic construct
  misc_feature 1..35: note = Primer
  source 1..35: mol_type = other DNA
gccgccggagaattcttctcccggccggggctgcc

SEQ ID NO: 235 - DNA, 32 bp, synthetic construct
  misc_feature 1..32: note = Primer
  source 1..32: mol_type = other DNA
gatatcaagctttcagccggcgcctaacgaac

SEQ ID NO: 236 - protein, 905 aa, Mycobacterium tuberculosis
MGSSHHHHHHSSGLVPRGSHHMMTINYQFGDVDAHGAMIRAQAGSLEAEHQAIISDVLTASDFWGGAGSAACQGFITQLGRNFQVIYEQANAHGQKVQAAGNNMAQTDSAVGSSWAGTDDIDWDAIAQCESGGNWAANTGNGLYGGLQISQATWDSNGGVGSPAAASPQQQIEVADNIMKTQGPGAWPKCSSCSQGDAPLGSLTHILTFLAAETGGCSGSRDDGSVVDFGALPPEINSARMYAGPGSASLVAAAKMWDSVASDLFSAASAFQSVVWGLTVGSWIGSSAGLMAAAASPYVAWMSVTAGQAQLTAAQVRVAAAAYETAYRLTVPPPVIAENRTELMTLTATNLLGQNTPAIEANQAAYSQMWGQDAEAMYGYAATAATATEALLPFEDAPLITNPGGLLEQAVAVEEAIDTAAANQLMNNVPQALQQLAQPAQGVVPSSKLGGLWTAVSPHLSPLSNVSSIANNHMSMMGTGVSMTNTLHSMLKGLAPAAAQAVETAAENGVWAMSSLGSQLGSSLGSSGLGAGVAANLGRAASVGSLSVPPAWAAANQAVTPAARALPLTSLTSAAQTAPGHMLGGLPLGHSVNAGSGINNALRVPARAYAIPRTPAAGEFFSRPGLPVEYLQVPSPSMGRDIKVQFQSGGNNSPAVYLLDGLRAQDDYNGWDINTPAFEWYYQSGLSIVMPVGGQSSFYSDWYSPACGKAGCQTYKWETFLTSELPQWLSANRAVKPTGSAAIGLSMAGSSAMILAAYHPQQFIYAGSLSALLDPSQGMGPSLIGLAMGDAGGYKAADMWGPSSDPAWERNDPTQQIPKLVANNTRLWVYCGNGTPNELGGANIPAEFLENFVRSSNLKFQDAYNAAGGHNAVFNFPPNGTHSWEYWGAQLNAMKGDLQSSLGAG

SEQ ID NO: 237 - DNA, 2025 bp, Mycobacterium tuberculosis
  source 1..2025: mol_type = genomic DNA
catatgatgaccatcaactatcaattcggggacgtcgacgctcacggcgccatgatccgcgctcaggccgggtcgctggaggccgagcatcaggccatcatttctgatgtgttgaccgcgagtgacttttggggcggcgccggttcggcggcctgccaggggttcattacccagctgggccgtaacttccaggtgatctacgagcaggccaacgcccacgggcagaaggtgcaggctgccggcaacaacatggcacaaaccgacagcgccgtcggctccagctgggccggtaccgacgacatcgattgggacgccatcgcgcaatgcgaatccggcggcaattgggcggccaacaccggtaacgggttatacggtggtctgcagatcagccaggcgacgtgggattccaacggtggtgtcgggtcgccggcggccgcgagtccccagcaacagatcgaggtcgcagacaacattatgaaaacccaaggcccgggtgcgtggccgaaatgtagttcttgtagtcagggagacgcaccgctgggctcgctcacccacatcctgacgttcctcgcggccgagactggaggttgttcggggagcagggacgatggatccgtggtggatttcggggcgttaccaccggagatcaactccgcgaggatgtacgccggcccgggttcggcctcgctggtggccgccgcgaagatgtgggacagcgtggcgagtgacctgttttcggccgcgtcggcgtttcagtcggtggtctggggtctgacggtggggtcgtggataggttcgtcggcgggtctgatggcggcggcggcctcgccgtatgtggcgtggatgagcgtcaccgcggggcaggcccagctgaccgccgcccaggtccgggttgctgcggcggcctacgagacagcgtataggctgacggtgcccccgccggtgatcgccgagaaccgtaccgaactgatgacgctgaccgcgaccaacctcttggggcaaaacacgccggcgatcgaggccaatcaggccgcatacagccagatgtggggccaagacgcggaggcgatgtatggctacgccgccacggcggcgacggcgaccgaggcgttgctgccgttcgaggacgccccactgatcaccaaccccggcggggaattcttctcccggccggggctgccggtcgagtacctgcaggtgccgtcgccgtcgatgggccgcgacatcaaggttcagttccagagcggtgggaacaactcacctgcggtttatctgctcgacggcctgcgcgcccaagacgactacaacggctgggatatcaacaccccggcgttcgagtggtactaccagtcgggactgtcgatagtcatgccggtcggcgggcagtccagcttctacagcgactggtacagcccggcctgcggtaaggctggctgccagacttacaagtgggaaaccttcctgaccagcgagctgccgcaatggttgtccgccaacagggccgtgaagcccaccggcagcgctgcaatcggcttgtcgatggccggctcgtcggcaatgatcttggccgcctaccacccccagcagttcatctacgccggctcgctgtcggccctgctggacccctctcaggggatggggcctagcctgatcggcctcgcgatgggtgacgccggcggttacaaggccgcagacatgtggggtccctcgagtgacccggcatgggagcgcaacgaccctacgcagcagatccccaagctggtcgcaaacaacacccggctatgggtttattgcgggaacggcaccccgaacgagttgggcggtgccaacatacccgccgagttcttggagaacttcgttcgtagcagcaacctgaagttccaggatgcgtacaacgccgcgggcgggcacaacgccgtgttcaacttcccgcccaacggcacgcacagctgggagtactggggcgctcagctcaacgccatgaagggtgacctgcagagttcgttaggcgccggctgaaagctt

SEQ ID NO: 238 - DNA, 30 bp, synthetic construct; identical to SEQ ID NO: 224
  misc_feature 1..30: note = Primer
  source 1..30: mol_type = other DNA

SEQ ID NO: 239 - DNA, 32 bp, synthetic construct; identical to SEQ ID NO: 229
  misc_feature 1..32: note = Primer
  source 1..32: mol_type = other DNA

SEQ ID NO: 240 - DNA, 33 bp, synthetic construct; identical to SEQ ID NO: 230
  misc_feature 1..33: note = Primer
  source 1..33: mol_type = other DNA

SEQ ID NO: 241 - DNA, 36 bp, synthetic construct; identical to SEQ ID NO: 231
  misc_feature 1..36: note = Primer
  source 1..36: mol_type = other DNA

SEQ ID NO: 242 - DNA, 39 bp, synthetic construct
  misc_feature 1..39: note = Primer
  source 1..39: mol_type = other DNA
cggccgggagaagaattccccgccggggttggtgatcag

SEQ ID NO: 243 - DNA, 35 bp, synthetic construct; identical to SEQ ID NO: 234
  misc_feature 1..35: note = Primer
  source 1..35: mol_type = other DNA

SEQ ID NO: 244 - DNA, 32 bp, synthetic construct; identical to SEQ ID NO: 235
  misc_feature 1..32: note = Primer
  source 1..32: mol_type = other DNA

SEQ ID NO: 245 - protein, 672 aa, Mycobacterium tuberculosis
HMMTINYQFGDVDAHGAMIRAQAGSLEAEHQAIISDVLTASDFWGGAGSAACQGFITQLGRNFQVIYEQANAHGQKVQAAGNNMAQTDSAVGSSWAGTDDIDWDAIAQCESGGNWAANTGNGLYGGLQISQATWDSNGGVGSPAAASPQQQIEVADNIMKTQGPGAWPKCSSCSQGDAPLGSLTHILTFLAAETGGCSGSRDDGSVVDFGALPPEINSARMYAGPGSASLVAAAKMWDSVASDLFSAASAFQSVVWGLTVGSWIGSSAGLMAAAASPYVAWMSVTAGQAQLTAAQVRVAAAAYETAYRLTVPPPVIAENRTELMTLTATNLLGQNTPAIEANQAAYSQMWGQDAEAMYGYAATAATATEALLPFEDAPLITNPGGEFFSRPGLPVEYLQVPSPSMGRDIKVQFQSGGNNSPAVYLLDGLRAQDDYNGWDINTPAFEWYYQSGLSIVMPVGGQSSFYSDWYSPACGKAGCQTYKWETFLTSELPQWLSANRAVKPTGSAAIGLSMAGSSAMILAAYHPQQFIYAGSLSALLDPSQGMGPSLIGLAMGDAGGYKAADMWGPSSDPAWERNDPTQQIPKLVANNTRLWVYCGNGTPNELGGANIPAEFLENFVRSSNLKFQDAYNAAGGHNAVFNFPPNGTHSWEYWGAQLNAMKGDLQSSLGAG

SEQ ID NO: 246 - DNA, 3318 bp, Mycobacterium tuberculosis
  source 1..3318: mol_type = genomic DNA
atgggcagcagccatcatcatcatcatcacagcagcggcctggtgccgcgcggcagccatatgggtacccatctcgccaacggttcgatgtcggaagtcatgatgtcggaaattgccgggttgcctatccctccgattatccattacggggcgattgcctatgcccccagcggcgcgtcgggcaaagcgtggcaccagcgcacaccggcgcgagcagagcaagtcgcactagaaaagtgcggtgacaagacttgcaaagtggttagtcgcttcaccaggtgcggcgcggtcgcctacaacggctcgaaataccaaggcggaaccggactcacgcgccgcgcggcagaagacgacgccgtgaaccgactcgaaggcgggcggatcgtcaactgggcgtgcaacgagctcatgacctcgcgttttatgacggatccgcacgcgatgcgggacatggcgggccgttttgaggtgcacgcccagacggtggaggacgaggctcgccggatgtgggcgtccgcgcaaaacatctcgggcgcgggctggagtggcatggccgaggcgacctcgctagacaccatgacccagatgaatcaggcgtttcgcaacatcgtgaacatgctgcacggggtgcgtgacgggctggttcgcgacgccaacaactacgaacagcaagagcaggcctcccagcagatcctcagcagcgtcgacatcaatttcgccgttttgccgccggaggtgaattcggcgcgcatattcgccggtgcgggcctgggcccaatgctggcggcggcgtcggcctgggacgggttggccgaggagttgcatgccgcggcgggctcgttcgcgtcggtgaccaccgggttggcgggcgacgcgtggcatggtccggcgtcgctggcgatgacccgcgcggccagcccgtatgtggggtggttgaacacggcggcgggtcaggccgcgcaggcggccggccaggcgcggctagcggcgagcgcgttcgaggcgacgctggcggccaccgtgtctccagcgatggtcgcggccaaccggacacggctggcgtcgctggtggcagccaacttgctgggccagaacgccccggcgatcgcggccgcggaggctgaatacgagcagatatgggcccaggacgtggccgcgatgttcggctatcactccgccgcgtcggcggtggccacgcagctggcgcctattcaagagggtttgcagcagcagctgcaaaacgtgctggcccagttggctagcgggaacctgggcagcggaaatgtgggcgtcggcaacatcggcaacgacaacattggcaacgcaaacatcggcttcggaaatcgaggcgacgccaacatcggcatcgggaatatcggcgacagaaacctcggcattgggaacaccggcaattggaatatcggcatcggcatcaccggcaacggacaaatcggcttcggcaagcctgccaaccccgacgtcttggtggtgggcaacggcggcccgggagtaaccgcgttggtcatgggcggcaccgacagcctactgccgctgcccaacatccccttactcgagtacgctgcgcggttcatcacccccgtgcatcccggatacaccgctacgttcctggaaacgccatcgcagtttttcccattcaccgggctgaatagcctgacctatgacgtctccgtggcccagggcgtaacgaatctgcacaccgcgatcatggcgcaactcgcggcgggaaacgaagtcgtcgtcttcggcacctcccaaagcgccacgatagccaccttcgaaatgcgctatctgcaatccctgccagcacacctgcgtccgggtctcgacgaattgtcctttacgttgaccggcaatcccaaccggcccgacggtggcattcttacgcgttttggcttctccataccgcagttgggtttcacattgtccggcgcgacgcccgccgacgcctaccccaccgtcgattacgcgttccagtacgacggcgtcaacgacttccccaaatacccgctgaatgtcttcgcgaccgccaacgcgatcgcgggcatccttttcctgcactccgggttgattgcgttgccgcccgatcttgcctcgggcgtggttcaaccggtgtcctcaccggacgtcctgaccacctacatcctgctgcccagccaagatctgccgctgctggtcccgctgcgtgctatccccctgctgggaaacccgcttgccgacctcatccagccggacttgcgggtgctcgtcgagttgggttatgaccgcaccgcccaccaggacgtgcccagcccgttcggactgtttccggacgtcgattgggccgaggtggccgcggacctgcagcaaggcgccgtgcaaggcgtcaacgacgccctgtccggactggggctgccgccgccgtggcagccggcgctaccccgacttttcagtactttctcccggccggggctgccggtcgagtacctgcaggtgccgtcgccgtcgatgggccgcgacatcaaggttcagttccagagcggtgggaacaactcacctgcggtttatctgctcgacggcctgcgcgcccaagacgactacaacggctgggatatcaacaccccggcgttcgagtggtactaccagtcgggactgtcgatagtcatgccggtcggcgggcagtccagcttctacagcgactggtacagcccggcctgcggtaaggctggctgccagacttacaagtgggaaaccttcctgaccagcgagctgccgcaatggttgtccgccaacagggccgtgaagcccaccggcagcgctgcaatcggcttgtcgatggccggctcgtcggcaatgatcttggccgcctaccacccccagcagttcatctacgccggctcgctgtcggccctgctggacccctctcaggggatggggcctagcctgatcggcctcgcgatgggtgacgccggcggttacaaggccgcagacatgtggggtccctcgagtgacccggcatgggagcgcaacgaccctacgcagcagatccccaagctggtcgcaaacaacacccggctatgggtttattgcgggaacggcaccccgaacgagttgggcggtgccaacatacccgccgagttcttggagaacttcgttcgtagcagcaacctgaagttccaggatgcgtacaacgccgcgggcgggcacaacgccgtgttcaacttcccgcccaacggcacgcacagctgggagtactggggcgctcagctcaacgccatgaagggtgacctgcagagttcgttaggcgccggctgaaagctt

SEQ ID NO: 247 - DNA, 33 bp, synthetic construct; identical to SEQ ID NO: 89
  misc_feature 1..33: note = Primer
  source 1..33: mol_type = other DNA

SEQ ID NO: 248 - DNA, 42 bp, synthetic construct; identical to SEQ ID NO: 90
  misc_feature 1..42: note = Primer
  source 1..42: mol_type = other DNA

SEQ ID NO: 249 - DNA, 47 bp, synthetic construct
  misc_feature 1..47: note = Primer
  source 1..47: mol_type = other DNA
cggcgctaccccgacttttcagtactttctcccggccggggctgccg

SEQ ID NO: 250 - DNA, 32 bp, synthetic construct; identical to SEQ ID NO: 235
  misc_feature 1..32: note = Primer
  source 1..32: mol_type = other DNA

SEQ ID NO: 251 - protein, 1103 aa, Mycobacterium tuberculosis
MGSSHHHHHHSSGLVPRGSHMGTHLANGSMSEVMMSEIAGLPIPPIIHYGAIAYAPSGASGKAWHQRTPARAEQVALEKCGDKTCKVVSRFTRCGAVAYNGSKYQGGTGLTRRAAEDDAVNRLEGGRIVNWACNELMTSRFMTDPHAMRDMAGRFEVHAQTVEDEARRMWASAQNISGAGWSGMAEATSLDTMTQMNQAFRNIVNMLHGVRDGLVRDANNYEQQEQASQQILSSVDMNFAVLPPEVNSARIFAGAGLGPMLAAASAWDGLAEELHAAAGSFASVTTGLAGDAWHGPASLAMTRAASPYVGWLNTAAGQAAQAAGQARLAASAFEATLAATVSPAMVAANRTRLASLVAANLLGQNAPAIAAAEAEYEQIWAQDVAAMFGYHSAASAVATQLAPIQEGLQQQLQNVLAQLASGNLGSGNVGVGNIGNDNIGNANIGFGNRGDANIGIGNIGDRNLGIGNTGNWNIGIGITGNGQIGFGKPANPDVLVVGNGGPGVTALVMGGTDSLLPLPNIPLLEYAARFITPVHPGYTATFLETPSQFFPFTGLNSLTYDVSVAQGVTNLHTAIMAQLAAGNEVVVFGTSQSATIATFEMRYLQSLPAHLRPGLDELSFTLTGNPNRPDGGILTRFGFSIPQLGFTLSGATPADAYPTVDYAFQYDGVNDFPKYPLNVFATANAIAGILFLHSGLIALPPDLASGVVQPVSSPDVLTTYILLPSQDLPLLVPLRAIPLLGNPLADLIQPDLRVLVELGYDRTAHQDVPSPFGLFPDVDWAEVAADLQQGAVQGVNDALSGLGLPPPWQPALPRLFSTFSRPGLPVEYLQVPSPSMGRDIKVQFQSGGNNSPAVYLLDGLRAQDDYNGWDINTPAFEWYYQSGLSIVMPVGGQSSFYSDWYSPACGKAGCQTYKWETFLTSELPQWLSANRAVKPTGSAAIGLSMAGSSAMILAAYHPQQFIYAGSLSALLDPSQGMGPSLIGLAMGDAGGYKAADMWGPSSDPAWERNDPTQQIPKLVANNTRLWVYCGNGTPNELGGANIPAEFLENFVRSSNLKFQDAYNAAGGHNAVFNFPPNGTHSWEYWGAQLNAMKGDLQSSLGAG

SEQ ID NO: 252 - DNA, 3597 bp, Mycobacterium tuberculosis
  source 1..3597: mol_type = genomic DNA
atgggcagcagccatcatcatcatcatcacagcagcggcctggtgccgcgcggcagccatatgaccatcaactatcaattcggggacgtcgacgctcacggcgccatgatccgcgctcaggccgggtcgctggaggccgagcatcaggccatcatttctgatgtgttgaccgcgagtgacttttggggcggcgccggttcggcggcctgccaggggttcattacccagctgggccgtaacttccaggtgatctacgagcaggccaacgcccacgggcagaaggtgcaggctgccggcaacaacatggcacaaaccgacagcgccgtcggctccagctgggccggtacccatctcgccaacggttcgatgtcggaagtcatgatgtcggaaattgccgggttgcctatccctccgattatccattacggggcgattgcctatgcccccagcggcgcgtcgggcaaagcgtggcaccagcgcacaccggcgcgagcagagcaagtcgcactagaaaagtgcggtgacaagacttgcaaagtggttagtcgcttcaccaggtgcggcgcggtcgcctacaacggctcgaaataccaaggcggaaccggactcacgcgccgcgcggcagaagacgacgccgtgaaccgactcgaaggcgggcggatcgtcaactgggcgtgcaacgagctcatgacctcgcgttttatgacggatccgcacgcgatgcgggacatggcgggccgttttgaggtgcacgcccagacggtggaggacgaggctcgccggatgtgggcgtccgcgcaaaacatctcgggcgcgggctggagtggcatggccgaggcgacctcgctagacaccatgacccagatgaatcaggcgtttcgcaacatcgtgaacatgctgcacggggtgcgtgacgggctggttcgcgacgccaacaactacgaacagcaagagcaggcctcccagcagatcctcagcagcgtcgacatcaatttcgccgttttgccgccggaggtgaattcggcgcgcatattcgccggtgcgggcctgggcccaatgctggcggcggcgtcggcctgggacgggttggccgaggagttgcatgccgcggcgggctcgttcgcgtcggtgaccaccgggttggcgggcgacgcgtggcatggtccggcgtcgctggcgatgacccgcgcggccagcccgtatgtggggtggttgaacacggcggcgggtcaggccgcgcaggcggccggccaggcgcggctagcggcgagcgcgttcgaggcgacgctggcggccaccgtgtctccagcgatggtcgcggccaaccggacacggctggcgtcgctggtggcagccaacttgctgggccagaacgccccggcgatcgcggccgcggaggctgaatacgagcagatatgggcccaggacgtggccgcgatgttcggctatcactccgccgcgtcggcggtggccacgcagctggcgcctattcaagagggtttgcagcagcagctgcaaaacgtgctggcccagttggctagcgggaacctgggcagcggaaatgtgggcgtcggcaacatcggcaacgacaacattggcaacgcaaacatcggcttcggaaatcgaggcgacgccaacatcggcatcgggaatatcggcgacagaaacctcggcattgggaacaccggcaattggaatatcggcatcggcatcaccggcaacggacaaatcggcttcggcaagcctgccaaccccgacgtcttggtggtgggcaacggcggcccgggagtaaccgcgttggtcatgggcggcaccgacagcctactgccgctgcccaacatccccttactcgagtacgctgcgcggttcatcacccccgtgcatcccggatacaccgctacgttcctggaaacgccatcgcagtttttcccattcaccgggctgaatagcctgacctatgacgtctccgtggcccagggcgtaacgaatctgcacaccgcgatcatggcgcaactcgcggcgggaaacgaagtcgtcgtcttcggcacctcccaaagcgccacgatagccaccttcgaaatgcgctatctgcaatccctgccagcacacctgcgtccgggtctcgacgaattgtcctttacgttgaccggcaatcccaaccggcccgacggtggcattcttacgcgttttggcttctccataccgcagttgggtttcacattgtccggcgcgacgcccgccgacgcctaccccaccgtcgattacgcgttccagtacgacggcgtcaacgacttccccaaatacccgctgaatgtcttcgcgaccgccaacgcgatcgcgggcatccttttcctgcactccgggttgattgcgttgccgcccgatcttgcctcgggcgtggttcaaccggtgtcctcaccggacgtcctgaccacctacatcctgctgcccagccaagatctgccgctgctggtcccgctgcgtgctatccccctgctgggaaacccgcttgccgacctcatccagccggacttgcgggtgctcgtcgagttgggttatgaccgcaccgcccaccaggacgtgcccagcccgttcggactgtttccggacgtcgattgggccgaggtggccgcggacctgcagcaaggcgccgtgcaaggcgtcaacgacgccctgtccggactggggctgccgccgccgtggcagccggcgctaccccgacttttcagtactttctcccggccggggctgccggtcgagtacctgcaggtgccgtcgccgtcgatgggccgcgacatcaaggttcagttccagagcggtgggaacaactcacctgcggtttatctgctcgacggcctgcgcgcccaagacgactacaacggctgggatatcaacaccccggcgttcgagtggtactaccagtcgggactgtcgatagtcatgccggtcggcgggcagtccagcttctacagcgactggtacagcccggcctgcggtaaggctggctgccagacttacaagtgggaaaccttcctgaccagcgagctgccgcaatggttgtccgccaacagggccgtgaagcccaccggcagcgctgcaatcggcttgtcgatggccggctcgtcggcaatgatcttggccgcctaccacccccagcagttcatctacgccggctcgctgtcggccctgctggacccctctcaggggatggggcctagcctgatcggcctcgcgatgggtgacgccggcggttacaaggccgcagacatgtggggtccctcgagtgacccggcatgggagcgcaacgaccctacgcagcagatccccaagctggtcgcaaacaacacccggctatgggtttattgcgggaacggcaccccgaacgagttgggcggtgccaacatacccgccgagttcttggagaacttcgttcgtagcagcaacctgaagttccaggatgcgtacaacgccgcgggcgggcacaacgccgtgttcaacttcccgcccaacggcacgcacagctgggagtactggggcgctcagctcaacgccatgaagggtgacctgcagagttcgttaggcgccggctgaaagctt

SEQ ID NO: 253 - DNA, 33 bp, synthetic construct; identical to SEQ ID NO: 89
  misc_feature 1..33: note = Primer
  source 1..33: mol_type = other DNA

SEQ ID NO: 254 - DNA, 42 bp, synthetic construct; identical to SEQ ID NO: 90
  misc_feature 1..42: note = Primer
  source 1..42: mol_type = other DNA

SEQ ID NO: 255 - DNA, 47 bp, synthetic construct; identical to SEQ ID NO: 249
  misc_feature 1..47: note = Primer
  source 1..47: mol_type = other DNA

SEQ ID NO: 256 - DNA, 32 bp, synthetic construct; identical to SEQ ID NO: 235
  misc_feature 1..32: note = Primer
  source 1..32: mol_type = other DNA

SEQ ID NO: 257 - protein, 1196 aa, Mycobacterium tuberculosis
MGSSHHHHHHSSGLVPRGSHMTINYQFGDVDAHGAMIRAQAGSLEAEHQAIISDVLTASDFWGGAGSAACQGFITQLGRNFQVIYEQANAHGQKVQAAGNNMAQTDSAVGSSWAGTHLANGSMSEVMMSEIAGLPIPPIIHYGAIAYAPSGASGKAWHQRTPARAEQVALEKCGDKTCKVVSRFTRCGAVAYNGSKYQGGTGLTRRAAEDDAVNRLEGGRIVNWACNELMTSRFMTDPHAMRDMAGRFEVHAQTVEDEARRMWASAQNISGAGWSGMAEATSLDTMTQMNQAFRNIVNMLHGVRDGLVRDANNYEQQEQASQQILSSVDINFAVLPPEVNSARIFAGAGLGPMLAAASAWDGLAEELHAAAGSFASVTTGLAGDAWHGPASLAMTRAASPYVGWLNTAAGQAAQAAGQARLAASAFEATLAATVSPAMVAANRTRLASLVAANLLGQNAPAIAAAEAEYEQIWAQDVAAMFGYHSAASAVATQLAPIQEGLQQQLQNVLAQLASGNLGSGNVGVGNIGNDNIGNANIGFGNRGDANIGIGNIGDRNLGIGNTGNWNIGIGITGNGQIGFGKPANPDVLVVGNGGPGVTALVMGGTDSLLPLPNIPLLEYAARFITPVHPGYTATFLETPSQFFPFTGLNSLTYDVSVAQGVTNLHTAIMAQLAAGNEVVVFGTSQSATIATFEMRYLQSLPAHLRPGLDELSFTLTGNPNRPDGGILTRFGFSIPQLGFTLSGATPADAYPTVDYAFQYDGVNDFPKYPLNVFATANAIAGILFLHSGLIALPPDLASGVVQPVSSPDVLTTYILLPSQDLPLLVPLRAIPLLGNPLADLIQPDLRVLVELGYDRTAHQDVPSPFGLFPDVDWAEVAADLQQGAVQGVNDALSGLGLPPPWQPALPRLFSTFSRPGLPVEYLQVPSPSMGRDIKVQFQSGGNNSPAVYLLDGLRAQDDYNGWDINTPAFEWYYQSGLSIVMPVGGQSSFYSDWYSPACGKAGCQTYKWETFLTSELPQWLSANRAVKPTGSAAIGLSMAGSSAMILAAYHPQQFIYAGSLSALLDPSQGMGPSLIGLAMGDAGGYKAADMWGPSSDPAWERNDPTQQIPKLVANNTRLWVYCGNGTPNELGGANIPAEFLENFVRSSNLKFQDAYNAAGGHNAVFNFPPNGTHSWEYWGAQLNAMKGDLQSSLGAG

SEQ ID NO: 258 - DNA, 2379 bp, Mycobacterium tuberculosis
  source 1..2379: mol_type = genomic DNA
catatgcatcaccatcaccatcacatgccggacaccatggtgaccaccgatgtcatcaagagcgcggtgcagttggcctgccgcgcaccgtcgctccacaacagccagccctggcgctggatagccgaggaccacacggttgcgctgttcctcgacaaggatcgggtgctttacgcgaccgaccactccggccgggaagcgctgctggggtgcggcgccgtactcgaccactttcgggtggcgatggcggccgcgggtaccaccgccaatgtggaacggtttcccaaccccaacgatcctttgcatctggcgtcaattgacttcagcccggccgatttcgtcaccgagggccaccgtctaagggcggatgcgatcctactgcgccgtaccgaccggctgcctttcgccgagccgccggattgggacttggtggagtcgcagttgcgcacgaccgtcaccgccgacacggtgcgcatcgacgtcatcgccgacgatatgcgtcccgaactggcggcggcgtccaaactcaccgaatcgctgcggctctacgattcgtcgtatcatgccgaactcttttggtggacaggggcttttgagacttctgagggcataccgcacagttcattggtatcggcggccgaaagtgaccgggtcaccttcggacgcgacttcccggtcgtcgccaacaccgataggcgcccggagtttggccacgaccgctctaaggtcctggtgctctccacctacgacaacgaacgcgccagcctactgcgctgcggcgagatgctttccgccgtattgcttgacgccaccatggctgggcttgccacctgcacgctgacccacatcaccgaactgcacgccagccgagacctggtcgcagcgctgattgggcagcccgcaactccgcaagccttggttcgcgtcggtctggccccggagatggaagagccgccaccggcaacgcctcggcgaccaatcgatgaagtgtttcacgttcgggctaaggatcaccggggtggttctggcggtagcggattcatgggcgatctggtgggcccgggctgcgcggaatacgcggcagccaatcccactgggccggcctcggtgcagggaatgtcgcaggacccggtcgcggtggcggcctcgaacaatccggagttgacaacgctgacggctgcactgtcgggccagctcaatccgcaagtaaacctggtggacaccctcaacagcggtcagtacacggtgttcgcaccgaccaacgcggcatttagcaagctgccggcatccacgatcgacgagctcaagaccaattcgtcactgctgaccagcatcctgacctaccacgtagtggccggccaaaccagcccggccaacgtcgtcggcacccgtcagaccctccagggcgccagcgtgacggtgaccggtcagggtaacagcctcaaggtcggtaacgccgacgtcgtctgtggtggggtgtctaccgccaacgcgacggtgtacatgattgacagcgtgctaatgcctccggcgggcggaagcggcggttctgaattcatgctccccgagacaaatcaggatgaggtccagcccaacgcacccgttgccctggtgacggtggaaatccgtcacccgacaacggattcgctcaccgaatcagcgaaccgggagctcaaacacctgcttatcaatgatctaccgatcgaacgccaggcgcaggacgtcagctgggggatgacggcgcccggtggagcccccaccccggtcgcggatcgtttcgttcgttatgtcaatcgcgataacaccaccgccgcttcactgaagaaccaggcgatagtcgtggagaccaccgcctaccgcagctttgaggcctttaccgacgttgtgatgcgggtcgtggatgctcgcgcgcaggtctcgtcaatcgttgggttggagcgtatcggtcttcgctttgttctggagatccgcgtccccgcgggtgtcgacggccggatcacgtggagcaactggatcgacgagcagctgctcgggccgcagcgtttcactcccggcggcctggtcctgaccgagtggcagggtgccgcagtctaccgtgagctacaaccaggcaaatcgctcatcgtgcgctacggcccgggtatgggccaagcgcttgatcccaattaccatctgcgccgaataacacccgcccaaaccggaccattcttcctgctggacatcgatagcttttggactcccagtggcggctccattcccgagtacaacagggacgccttagtgtcgacattccaggacctgtacggtccggcccaggtcgtgtttcaggagatgatcaccagtcgcctgaaagatgagctgcttcgccagtaaaagctt

SEQ ID NO: 259 - DNA, 50 bp, synthetic construct
  misc_feature 1..50: note = Primer
  source 1..50: mol_type = other DNA
gatacacatatgcaccatcaccatcaccacatgccggacaccatggtgac

SEQ ID NO: 260 - DNA, 48 bp, synthetic construct
  misc_feature 1..48: note = Primer
  source 1..48: mol_type = other DNA
catggatccgctaccgccagaaccaccccggtgatccttagcccgaac

SEQ ID NO: 261 - DNA, 45 bp, synthetic construct
  misc_feature 1..45: note = Primer
  source 1..45: mol_type = other DNA
ggtggttctggcggtagcggattcatgggcgatctggtgagcccg

SEQ ID NO: 262 - DNA, 47 bp, synthetic construct
  misc_feature 1..47: note = Primer
  source 1..47: mol_type = other DNA
catgaattcagaaccgccgcttccgcccgccggaggcattagcacgc

SEQ ID NO: 263 - DNA, 45 bp, synthetic construct
  misc_feature 1..45: note = Primer
  source 1..45: mol_type = other DNA
ggcggaagcggcggttctgaattcatgctccccgagacaaatcag

SEQ ID NO: 264 - DNA, 35 bp, synthetic construct
  misc_feature 1..35: note = Primer
  source 1..35: mol_type = other DNA
tagaattcaagcttttactggcgaagcagctcatc

SEQ ID NO: 265 - protein, 790 aa, Mycobacterium tuberculosis
HMHHHHHHMPDTMVTTDVIKSAVQLACRAPSLHNSQPWRWIAEDHTVALFLDKDRVLYATDHSGREALLGCGAVLDHFRVAMAAAGTTANVERFPNPNDPLHLASIDFSPADFVTEGHRLRADAILLRRTDRLPFAEPPDWDLVESQLRTTVTADTVRIDVIADDMRPELAAASKLTESLRLYDSSYHAELFWWTGAFETSEGIPHSSLVSAAESDRVTFGRDFPVVANTDRRPEFGHDRSKVLVLSTYDNERASLLRCGEMLSAVLLDATMAGLATCTLTHITELHASRDLVAALIGQPATPQALVRVGLAPEMEEPPPATPRRPIDEVFHVRAKDHRGGSGGSGFMGDLVGPGCAEYAAANPTGPASVQGMSQDPVAVAASNNPELTTLTAALSGQLNPQVNLVDTLNSGQYTVFAPTNAAFSKLPASTIDELKTNSSLLTSILTYHVVAGQTSPANVVGTRQTLQGASVTVTGQGNSLKVGNADVVCGGVSTANATVYMIDSVLMPPAGGSGGSEFMLPETNQDEVQPNAPVALVTVEIRHPTTDSLTESANRELKHLLINDLPIERQAQDVSWGMTAPGGAPTPVADRFVRYVNRDNTTAASLKNQAIVVETTAYRSFEAFTDVVMRVVDARAQVSSIVGLERIGLRFVLEIRVPAGVDGRITWSNWIDEQLLGPQRFTPGGLVLTEWQGAAVYRELQPGKSLIVRYGPGMGQALDPNYHLRRITPAQTGPFFLLDIDSFWTPSGGSIPEYNRDALVSTFQDLYGPAQVVFQEMITSRLKDELLRQ

SEQ ID NO: 266 - DNA, 2622 bp, Mycobacterium tuberculosis
  source 1..2622: mol_type = genomic DNA
atgggcagcagccatcatcatcatcatcacagcagcggcctggtgccgcgcggcagccatatgcataaggcgtcacaatcgatgatcacgcccaccacccagatcgccggcgccggggtgctgggaaacgacagaaagccggatgagtcgtgcgcgcgtgcggcggccgcggccgatccggggccaccgacccgaccagcgcacaatgcggcgggagtcagcccggagatggtgcaggtgccggcggaggcgcagcgcatcgtggtgctctccggtgaccagctcgacgcgctgtgcgcgctgggcctgcaatcgcggatcgtcgccgccgcgttgccgaacagctcctcaagtcaaccttcctatctgggcacgaccgtgcatgatctgcccggtgtcggtactcgcagcgcccccgacctgcgcgccattgcggcggctcacccggatctgatcctgggttcgcagggtttgacgccgcagttgtatccgcagctggcggcgatcgccccgacggtgtttaccgcggcaccgggcgcggactgggaaaataacctgcgtggtgtcggtgccgccacggcccgtatcgccgcggtggacgcgctgatcaccgggttcgccgaacacgccacccaggtcgggaccaagcatgacgcgacccacttccaagcgtcgatcgtgcagctgaccgccaacaccatgcgggtatacggcgccaacaacttcccggccagcgtgctgagcgcggtcggcgtcgaccgaccgccgtctcaacggttcaccgacaaggcctacatcgagatcggcaccacggccgccgacctggcgaaatcaccggacttctcggcggccgacgccgatatcgtctacctgtcgtgcgcgtcggaagcagccgcggaacgcgcggccgtcatcctggatagcgacccatggcgcaagctgtccgccaaccgtgacaaccgggtcttcgtcgtcaacgaccaggtatggcagaccggcgagggtatggtcgctgcccgcggcattgtcgatgatctgcgctgggtcgacgcgccgatcaacgagctcggaggttctggtggaagcgcatgcaaaacggtgacgttgaccgtcgacggaaccgcgatgcgggtgaccacgatgaaatcgcgggtgatcgacatcgtcgaagagaacgggttctcagtcgacgaccgcgacgacctgtatcccgcggccggcgtgcaggtccatgacgccgacaccatcgtgctgcggcgtagccgtccgctgcagatctcgctggatggtcacgacgctaagcaggtgtggacgaccgcgtcgacggtggacgaggcgctggcccaactcgcgatgaccgacacggcgccggccgcggcttctcgcgccagccgcgtcccgctgtccgggatggcgctaccggtcgtcagcgccaagacggtgcagctcaacgacggcgggttggtgcgcacggtgcacttgccggcccccaatgtcgcggggctgctgagtgcggccggcgtgccgctgttgcaaagcgaccacgtggtgcccgccgcgacggccccgatcgtcgaaggcatgcagatccaggtgacccgcaatcggatcaagaaggtcaccgagcggctgccgctgccgccgaacgcgcgtcgtgtcgaggacccggagatgaacatgagccgggaggtcgtcgaagacccgggggttccggggacccaggatgtgacgttcgcggtagctgaggtcaacggcgtcgagaccggccgtttgcccgtcgccaacgtcgtggtgaccccggcccacgaagccgtggtgcgggtgggcaccaagcccggtaccgaggtgcccccggtgatcgacggaagcatctgggacgcgatcgccggctgtgaggccggtggcaactgggcgatcaacaccggcaacgggtattacggtggtgtgcagtttgaccagggcacctgggaggccaacggcgggctgcggtatgcaccccgcgctgacctcgccacccgcgaagagcagatcgccgttgccgaggtgacccgactgcgtcaaggttggggcgcctggccggtatgtgctgcacgagcgggtgcgcgcgaattcggtggaagcggaggttctatgacggcaatctcgtgctcaccgcgacccaggtatgcttcccgaatgccagttttgagcaagaccgtcgaggtcaccgccgacgccgcatcgatcatggccatcgttgccgatatcgagcgctacccagagtggaatgaaggggtcaagggcgcatgggtgctcgctcgctacgatgacgggcgtcccagccaggtgcggctcgacaccgctgttcaaggcatcgagggcacctatatccacgccgtgtactacccaggcgaaaaccagattcaaaccgtcatgcagcagggtgaactgtttgccaagcaggagcagctgttcagtgtggtggcaaccggcgccgcgagcttgctcacggtggacatggacgtccaggtcaccatgccggtgcccgagccgatggtgaagatgctgctcaacaacgtcctggagcatctcgccgaaaatctcaagcagcgcgccgagcagctggcggccagctaaaagctt

SEQ ID NO: 267 - DNA, 52 bp, synthetic construct
  misc_feature 1..52: note = Primer
  source 1..52: mol_type = other DNA
gatacacatatgcaccatcaccatcaccacatgggcagcagccatcatcatc

SEQ ID NO: 268 - DNA, 32 bp, synthetic construct
  misc_feature 1..32: note = Primer
  source 1..32: mol_type = other DNA
catatcgagctcgttgatcggcgcgtcgaccc

SEQ ID NO: 269 - DNA, 53 bp, synthetic construct
  misc_feature 1..53: note = Primer
  source 1..53: mol_type = other DNA
atcaacgagctcggaggttctggtggaagcgcatgcaaaacggtgacgttgac

SEQ ID NO: 270 - DNA, 33 bp, synthetic construct
  misc_feature 1..33: note = Primer
  source 1..33: mol_type = other DNA
catatcgaattcgcgcgcacccgctcgtgcagc

SEQ ID NO: 271 - DNA, 52 bp, synthetic construct
  misc_feature 1..52: note = Primer
  source 1..52: mol_type = other DNA
catgtcgaattcggtggaagcggaggttctatgacggcaatctcgtgctcac

SEQ ID NO: 272 - DNA, 33 bp, synthetic construct
  misc_feature 1..33: note = Primer
  source 1..33: mol_type = other DNA
catatcaagcttttagctggccgccagctgctc

SEQ ID NO: 273 - protein, 871 aa, Mycobacterium tuberculosis
MGSSHHHHHHSSGLVPRGSHMHKASQSMITPTTQIAGAGVLGNDRKPDESCARAAAAADPGPPTRPAHNAAGVSPEMVQVPAEAQRIVVLSGDQLDALCALGLQSRIVAAALPNSSSSQPSYLGTTVHDLPGVGTRSAPDLRAIAAAHPDLILGSQGLTPQLYPQLAAIAPTVFTAAPGADWENNLRGVGAATARIAAVDALITGFAEHATQVGTKHDATHFQASIVQLTANTMRVYGANNFPASVLSAVGVDRPPSQRFTDKAYIEIGTTAADLAKSPDFSAADADIVYLSCASEAAAERAAVILDSDPWRKLSANRDNRVFVVNDQVWQTGEGMVAARGIVDDLRWVDAPINELGGSGGSACKTVTLTVDGTAMRVTTMKSRVIDIVEENGFSVDDRDDLYPAAGVQVHDADTIVLRRSRPLQISLDGHDAKQVWTTASTVDEALAQLAMTDTAPAAASRASRVPLSGMALPVVSAKTVQLNDGGLVRTVHLPAPNVAGLLSAAGVPLLQSDHVVPAATAPIVEGMQIQVTRNRIKKVTERLPLPPNARRVEDPEMNMSREVVEDPGVPGTQDVTFAVAEVNGVETGRLPVANVVVTPAHEAVVRVGTKPGTEVPPVIDGSIWDAIAGCEAGGNWAINTGNGYYGGVQFDQGTWEANGGLRYAPRADLATREEQIAVAEVTRLRQGWGAWPVCAARAGAREFGGSGGSMTAISCSPRPRYASRMPVLSKTVEVTADAASIMAIVADIERYPEWNEGVKGAWVLARYDDGRPSQVRLDTAVQGIEGTYIHAVYYPGENQIQTVMQQGELFAKQEQLFSVVATGAASLLTVDMDVQVTMPVPEPMVKMLLNNVLEHLAENLKQRAEQLAAS

SEQ ID NO: 274 - DNA, 3171 bp, Mycobacterium tuberculosis
  source 1..3171: mol_type = genomic DNA
catatgcaccatcaccatcaccacatggaaaaaatgtcacatgatccgatcgctgccgacattggcacgcaagtgagcgacaacgctctgcacggcgtgacggccggctcgacggcgctgacgtcggtgaccgggctggttcccgcgggggccgatgaggtctccgcccaagcggcgacggcgttcacatcggagggcatccaattgctggcttccaatgcatcggcccaagaccagctccaccgtgcgggcgaagcggtccaggacgtcgcccgcacctattcgcaaatcgacgacggcgccgccggcgtcttcgccgaagagctcggaggttccggtggaagcatgctgtggcacgcaatgccaccggagctaaataccgcacggctgatggccggcgcgggtccggctccaatgcttgcggcggccgcgggatggcagacgctttcggcggctctggacgctcaggccgtcgagttgaccgcgcgcctgaactctctgggagaagcctggactggaggtggcagcgacaaggcgcttgcggctgcaacgccgatggtggtctggctacaaaccgcgtcaacacaggccaagacccgtgcgatgcaggcgacggcgcaagccgcggcatacacccaggccatggccacgacgccgtcgctgccggagatcgccgccaaccacatcacccaggccgtccttacggccaccaacttcttcggtatcaacacgatcccgatcgcgttgaccgagatggattatttcatccgtatgtggaaccaggcagccctggcaatggaggtctaccaggccgagaccgcggttaacacgcttttcgagaagctcgagccgatggcgtcgatccttgatcccggcgcgagccagagcacgacgaacccgatcttcggaatgccctcccctggcagctcaacaccggttggccagttgccgccggcggctacccagaccctcggccaactgggtgagatgagcggcccgatgcagcagctgacccagccgctgcagcaggtgacgtcgttgttcagccaggtgggcggcaccggcggcggcaacccagccgacgaggaagccgcgcagatgggcctgctcggcaccagtccgctgtcgaaccatccgctggctggtggatcaggccccagcgcgggcgcgggcctgctgcgcgcggagtcgctacctggcgcaggtgggtcgttgacccgcacgccgctgatgtctcagctgatcgaaaagccggttgccccctcggtgatgccggcggctgctgccggatcgtcggcgacgggtggcgccgctccggtgggtgcgggagcgatgggccagggtgcgcaatccggcggctccaccaggccgggtctggtcgcgccggcaccgctcgcgcaggagcgtgaagaagacgacgaggacgactgggacgaagaggacgactgggaattcggtggcagtggaggatctatgacagagcagcagtggaatttcgcgggtatcgaggccgcggcaagcgcaatccagggaaatgtcacgtccattcattccctccttgacgaggggaagcagtccctgaccaagctcgcagcggcctggggcggtagcggttcggaggcgtaccagggtgtccagcaaaaatgggacgccacggctaccgagctgaacaacgcgctgcagaacctggcgcggacgatcagcgaagccggtcaggcaatggcttcgaccgaaggcaacgtcactgggatgttcgcagctagcggaggttccggtggaagcatgacgcagtcgcagaccgtgacggtggatcagcaagagattttgaacagggccaacgaggtggaggccccgatggcggacccaccgactgatgtccccatcacaccgtgcgaactcacggcggctaaaaacgccgcccaacagctggtattgtccgccgacaacatgcgggaatacctggcggccggtgccaaagagcggcagcgtctggcgacctcgctgcgcaacgcggccaaggcgtatggcgaggttgatgaggaggctgcgaccgcgctggacaacgacggcgaaggaactgtgcaggcagaatcggccggggccgtcggaggggacagttcggccgaactaaccgatacgccgagggtggccacggccggtgaacccaacttcatggatctcaaagaagcggcaaggaagctcgaaacgggcgaccaaggcgcatcgctcgcgcactttgcggatgggtggaacactttcaacctgacgctgcaaggcgacgtcaagcggttccgggggtttgacaactgggaaggcgatgcggctaccgcttgcgaggcttcgctcgatcaacaacggcaatggatactccacatggccaaattgagcgctgcgatggccaagcaggctcaatatgtcgcgcagctgcacgtgtgggctaggcgggaacatccgacttatgaagacatagtcgggctcgaacggctttacgcggaaaacccttcggcccgcgaccaaattctcccggtgtacgcggagtatcagcagaggtcggagaaggtgctgaccgaatacaacaacaaggcagccctggaaccggtaaacccgccgaagcctccccccgccatcaagatcgacccgcccccgcctccgcaagagcagggattgatccctggcttcctgatgccgccgtctgacggctccggtgtgactcccggtaccgggatgccagccgcaccgatggttccgcctaccggatcgccgggtggtggcctcccggctgacacggcggcacagctgacgtcggctgggcgggaagccgcagcgctgtcgggygacgtggcggtcaaagcggcatcgctcggtggyggtggaggcggcggggtgccgtcggcgccgttgggatccgcgatcgggggcgccgaatcggtgcggcccgctggcgctggtgacattgccggcttaggccagggaagggccggcggcggcgccgcgctgggcggcggtggcatgggaatgccgatgggtgccgcgcatcagggacaagggggcgccaagtccaagggttctcagcaggaagacgaggcgctctacaccgaggatcgggcatggaccgaggccgtcattggtaaccgtcggcgccaggacagtaaggagtcgaagtgaaagctt

SEQ ID NO: 275 - DNA, 52 bp, synthetic construct
  misc_feature 1..52: note = Primer
  source 1..52: mol_type = other DNA
gatacacatatgcaccatcaccatcaccacatggaaaaaatgtcacatgatc

SEQ ID NO: 276 - DNA, 34 bp, synthetic construct
  misc_feature 1..34: note = Primer
  source 1..34: mol_type = other DNA
gatacatgagctcttcggcgaagacgccggcggc

SEQ ID NO: 277 - DNA, 50 bp, synthetic construct
  misc_feature 1..50: note = Primer
  source 1..50: mol_type = other DNA
gatacagagctcggaggttccggtggaagcatgctgtggcacgcaatgcc

SEQ ID NO: 278 - DNA, 34 bp, synthetic construct
  misc_feature 1..34: note = Primer
  source 1..34: mol_type = other DNA
gatacagaattcccagtcgtcctcttcgtcccag

SEQ ID NO: 279 - DNA, 49 bp, synthetic construct
  misc_feature 1..49: note = Primer
  source 1..49: mol_type = other DNA
gacagaattcggtggcagtggaggatctatgacagagcagcagtggaat

SEQ ID NO: 280 - DNA, 35 bp, synthetic construct
  misc_feature 1..35: note = Primer
  source 1..35: mol_type = other DNA
catatcagctagctgcgaacatcccagtgacgttg

SEQ ID NO: 281 - DNA, 52 bp, synthetic construct
  misc_feature 1..52: note = Primer
  source 1..52: mol_type = other DNA
catatcagctagcggaggttccggtggaagcatgacgcagtcgcagaccgtg

SEQ ID NO: 282 - DNA, 34 bp, synthetic construct
  misc_feature 1..34: note = Primer
  source 1..34: mol_type = other DNA
catatcaaagctttcacttcgactccttactgtc

SEQ ID NO: 283 - protein, 1054 aa, Mycobacterium tuberculosis
HMHHHHHHMEKMSHDPIAADIGTQVSDNALHGVTAGSTALTSVTGLVPAGADEVSAQAATAFTSEGIQLLASNASAQDQLHRAGEAVQDVARTYSQIDDGAAGVFAEELGGSGGSMLWHAMPPELNTARLMAGAGPAPMLAAAAGWQTLSAALDAQAVELTARLNSLGEAWTGGGSDKALAAATPMVVWLQTASTQAKTRAMQATAQAAAYTQAMATTPSLPEIAANHITQAVLTATNFFGINTIPIALTEMDYFIRMWNQAALAMEVYQAETAVNTLFEKLEPMASILDPGASQSTTNPIFGMPSPGSSTPVGQLPPAATQTLGQLGEMSGPMQQLTQPLQQVTSLFSQVGGTGGGNPADEEAAQMGLLGTSPLSNHPLAGGSGPSAGAGLLRAESLPGAGGSLTRTPLMSQLIEKPVAPSVMPAAAAGSSATGGAAPVGAGAMGQGAQSGGSTRPGLVAPAPLAQEREEDDEDDWDEEDDWEFGGSGGSMTEQQWNFAGIEAAASAIQGNVTSIHSLLDEGKQSLTKLAAAWGGSGSEAYQGVQQKWDATATELNNALQNLARTISEAGQAMASTEGNVTGMFAASGGSGGSMTQSQTVTVDQQEILNRANEVEAPMADPPTDVPITPCELTAAKNAAQQLVLSADNMREYLAAGAKERQRLATSLRNAAKAYGEVDEEAATALDNDGEGTVQAESAGAVGGDSSAELTDTPRVATAGEPNFMDLKEAARKLETGDQGASLAHFADGWNTFNLTLQGDVKRFRGFDNWEGDAATACEASLDQQRQWILHMAKLSAAMAKQAQYVAQLHVWARREHPTYEDIVGLERLYAENPSARDQILPVYAEYQQRSEKVLTEYNNKAALEPVNPPKPPPAIKIDPPPPPQEQGLIPGFLMPPSDGSGVTPGTGMPAAPMVPPTGSPGGGLPADTAAQLTSAGREAAALSGDVAVKAASLGGGGGGGVPSAPLGSAIGGAESVRPAGAGDIAGLGQGRAGGGAALGGGGMGMPMGAAHQGQGGAKSKGSQQEDEALYTEDRAWTEAVIGNRRRQDSKESK

SEQ ID NO: 284 - DNA, 2613 bp, Mycobacterium tuberculosis
  source 1..2613: mol_type = genomic DNA
catatggagctggtccgggtgaccgaggccggagccatggccgcgggccgctgggtaggccgcggcgacaaggagggcggcgacggcgcggcggtcgacgcgatgcgcgaactggtcaactcggtttccatgcgcggggtggtggtcatcggcgaaggcgaaaaggaccacgcaccaatgctctacaacggcgaagaagtgggcaacggcgacggaccggaatgcgactttgccgtcgaccccattgacggcaccacgctgatgagcaagggcatgaccaacgccatctcggtgctggcggtagccgatcgcggcaccatgttcgacccgtcggcggtgttctacatgaacaaaatcgccgtcggccccgatgccgcacacgtgctggatatcaccgcgccgatctcggaaaacatccgagcggtcgccaaggtcaaggacctgtcggtgcgagacatgacggtgtgcatcctggacaggccgcggcacgcgcaactcatccacgacgtccgcgccaccggggcccggatccggctgatcaccgatggcgacgtcgccggcgcgatctcggcgtgccgaccgcactccggcaccgacctgctagctgggatcggcggcaccccggagggaatcatcgccgccgcggcgatccgctgcatgggcggggcgatccaggcgcagctcgccccgcgcgacgacgcggaacgccgcaaggccctagaagccggttacgacctgaaccaggtcttgaccaccgaagatctggtgtccggggaaaacgtcttcttctgcgccactggggtcaccgacggcgacctgctcaagggagtgcgttactaccccggcggctgcaccacccattcgatcgtgatgcgctcgaagtccggcaccgtccggatgatcgaggcctaccaccggctttcaaagctcaacgaatactccgcgatcgacttcaccggcgacagcagcgccgtgtacccattgcccggaggttctggtggaagcgaattcgtgcgatacagtgactcataccacacaacgggccggtggcagccacgagcgtcgacagaagggtttcccatgggcgtcagcatcgaggtcaacggactaacgaagtccttcgggtcctcgaggatctgggaagatgtcacgctaacgatccccgccggggaggtcagcgtgctgctgggcccatcgggtaccggcaaatcggtgtttctgaaatctctgatcggcctcctgcggccggagcgcggctcgatcatcatcgacggcaccgacatcatcgaatgctcggccaaggagctttacgagatccgcacattgttcggcgtgctgtttcaggacggtgccctgttcgggtcgatgaacctctacgacaacaccgcgttccccctgcgtgagcacaccaagaaaaaggaaagcgagatccgtgacatcgtcatggagaagctggccctagtcggcctgggtggggacgagaagaagttccccggcgagatctccggcgggatgcgtaagcgtgccggcctagcgcgtgccctggtccttgacccgcagatcattctctgcgacgagcccgactcgggtctggacccggttcgtaccgcctacctgagccagctgatcatggacatcaacgcccagatcgacgccaccatcctgatcgtgacgcacaacatcaacatcgcccgcaccgtgccggacaacatgggcatgttgttccgcaagcatttggtgatgttcgggccgcgggaggtgctactcaccagcgacgagccggtggtgcggcagttcctcaacggccggcgcatcggcccgatcggcatgtccgaggagaaggacgaggccaccatggccgaagagcaggccctgctcgatgccggccaccacgcgggcggtgtcgaggaaatcgagggcgtgccgccgcagatcagcgcgacaccgggcatgccggagcgcaaagcggtcgcccggcgtcaggctcgggttcgcgagatgttgcacacgctgcccaaaaaggcccaggcggcgatcctcgacgatctcgagggcacgcacaagtacgcggtgcacgaaatcggccagggtggaagcggcggttctgagctcgtggctggtgacaccaccatcaccatcgtcggaaatctgaccgctgaccccgagctgcggttcaccccgtccggtgcggccgtggcgaatttcaccgtggcgtcaacgccccggatctatgaccgtcagaccggcgaatggaaagacggcgaagcgctgttcctccggtgcaatatctggcgggaggcggccgagaacgtggccgagagcctcacccggggggcacgagtcatcgttagcgggcggcttaagcagcggtcgtttgaaacccgtgagggcgagaagcgcaccgtcatcgaggtcgaggtcgatgagattgggccttcgcttcggtacgccaccgccaaggtcaacaaggccagccgcagcggcgggtttggcagcggatcccgtccggcgccggcgcagaccagcagcgcctcgggagatgacccgtggggcagcgcaccggcgtcgggttcgttcggcggcggcgatgacgaaccgccattctgaaagctt

SEQ ID NO: 285 - DNA, 32 bp, synthetic construct
  misc_feature 1..32: note = Primer
  source 1..32: mol_type = other DNA
taggatcccatatggagctggtccgggtgacc

SEQ ID NO: 286 - DNA, 47 bp, synthetic construct
  misc_feature 1..47: note = Primer
  source 1..47: mol_type = other DNA
cacgaattcgcttccaccagaacctccgggcaatgggtacacggcgc

SEQ ID NO: 287 - DNA, 45 bp, synthetic construct
  misc_feature 1..45: note = Primer
  source 1..45: mol_type = other DNA
ggaggttctggtggaagcgaattcgtgcgatacagtgactcatac

SEQ ID NO: 288 - DNA, 50 bp, synthetic construct
  misc_feature 1..50: note = Primer
  source 1..50: mol_type = other DNA
gccacgagctcagaaccgccgcttccaccctggccgatttcgtgcaccgc

SEQ ID NO: 289 - DNA, 50 bp, synthetic construct
  misc_feature 1..50: note = Primer
  source 1..50: mol_type = other DNA
gccagggtggaagcggcggttctgagctcgtggctggtgacaccaccatc

SEQ ID NO: 290 - DNA, 36 bp, synthetic construct
  misc_feature 1..36: note = Primer
  source 1..36: mol_type = other DNA
caattaaagctttcagaatggcggttcgtcatcgcc

SEQ ID NO: 291 - protein, 887 aa, Mycobacterium tuberculosis
MGSSHHHHHHSSGLVPRGSHMELVRVTEAGAMAAGRWVGRGDKEGGDGAAVDAMRELVNSVSMRGVVVIGEGEKDHAPMLYNGEEVGNGDGPECDFAVDPIDGTTLMSKGMTNAISVLAVADRGTMFDPSAVFYMNKIAVGPDAAHVLDITAPISENIRAVAKVKDLSVRDMTVCILDRPRHAQLIHDVRATGARIRLITDGDVAGAISACRPHSGTDLLAGIGGTPEGIIAAAAIRCMGGAIQAQLAPRDDAERRKALEAGYDLNQVLTTEDLVSGENVFFCATGVTDGDLLKGVRYYPGGCTTHSIVMRSKSGTVRMIEAYHRLSKLNEYSAIDFTGDSSAVYPLPGGSGGSEFVRYSDSYHTTGRWQPRASTEGFPMGVSIEVNGLTKSFGSSRIWEDVTLTIPAGEVSVLLGPSGTGKSVFLKSLIGLLRPERGSIIIDGTDIIECSAKELYEIRTLFGVLFQDGALFGSMNLYDNTAFPLREHTKKKESEIRDIVMEKLALVGLGGDEKKFPGEISGGMRKRAGLARALVLDPQIILCDEPDSGLDPVRTAYLSQLIMDINAQIDATILIVTHNINIARTVPDNMGMLFRKHLVMFGPREVLLTSDEPVVRQFLNGRRIGPIGMSEEKDEATMAEEQALLDAGHHAGGVEEIEGVPPQISATPGMPERKAVARRQARVREMLHTLPKKAQAAILDDLEGTHKYAVHEIGQGGSGGSELVAGDTTITIVGNLTADPELRFTPSGAAVANFTVASTPRIYDRQTGEWKDGEALFLRCNIWREAAENVAESLTRGARVIVSGRLKQRSFETREGEKRTVIEVEVDEIGPSLRYATAKVNKASRSGGFGSGSRPAPAQTSSASGDDPWGSAPASGSFGGGDDEPPF

SEQ ID NO: 292 - protein, 95 aa, Mycobacterium tuberculosis
MTEQQWNFAGIEAAASAIQGNVTSIHSLLDEGKQSLTKLAAAWGGSGSEAYQGVQQKWDATATELNNALQNLARTISEAGQAMASTEGNVTGMFA

SEQ ID NO: 293 - DNA, 318 bp, Mycobacterium tuberculosis
  source 1..318: mol_type = genomic DNA
catatgcatcaccatcaccatcacatgacagagcagcagtggaatttcgcgggtatcgaggccgcggcaagcgcaatccagggaaatgtcacgtccattcattccctccttgacgaggggaagcagtccctgaccaagctcgcagcggcctggggcggtagcggttcggaggcgtaccagggtgtccagcaaaaatgggacgccacggctaccgagctgaacaacgcgctgcagaacctggcgcggacgatcagcgaagccggtcaggcaatggcttcgaccgaaggcaacgtcactgggatgttcgcataggaattc

SEQ ID NO: 294 - protein, 102 aa, Mycobacterium tuberculosis
MHHHHHHMTEQQWNFAGIEAAASAIQGNVTSIHSLLDEGKQSLTKLAAAWGGSGSEAYQGVQQKWDATATELNNALQNLARTISEAGQAMASTEGNVTGMFA

SEQ ID NO: 295 - DNA, 33 bp, synthetic construct
  misc_feature 1..33: note = Primer
  source 1..33: mol_type = other DNA
caattacatatgagagttttgttgctgggaccg

SEQ ID NO: 296 - DNA, 35 bp, synthetic construct
  misc_feature 1..35: note = Primer
  source 1..35: mol_type = other DNA
caattaaagcttctactttccagagcccgcaacgc

SEQ ID NO: 297 - DNA, 32 bp, synthetic construct
  misc_feature 1..32: note = Primer
  source 1..32: mol_type = other DNA
caattacatatgaccggccccaccaccgcgcc

SEQ ID NO: 298 - DNA, 36 bp, synthetic construct
  misc_feature 1..36: note = Primer
  source 1..36: mol_type = other DNA
caattaaagctttcaggtgtctttgggtgttccgag

SEQ ID NO: 299 - DNA, 33 bp, synthetic construct; identical to SEQ ID NO: 295
  misc_feature 1..33: note = Primer
  source 1..33: mol_type = other DNA

SEQ ID NO: 300 - DNA, 35 bp, synthetic construct; identical to SEQ ID NO: 296
  misc_feature 1..35: note = Primer
  source 1..35: mol_type = other DNA

SEQ ID NO: 301 - DNA, 54 bp, synthetic construct
  misc_feature 1..54: note = Primer
  source 1..54: mol_type = other DNA
caattacatatgcatcaccatcaccatcacgtggtggaccgcgatcccaatacc

SEQ ID NO: 302 - DNA, 33 bp, synthetic construct
  misc_feature 1..33: note = Primer
  source 1..33: mol_type = other DNA
caattagaattctcagcgattcctgatcttgtg

SEQ ID NO: 303 - DNA, 33 bp, synthetic construct
  misc_feature 1..33: note = Primer
  source 1..33: mol_type = other DNA
ctggatcccatatggccttcccggaatattcgc

SEQ ID NO: 304 - DNA, 33 bp, synthetic construct
  misc_feature 1..33: note = Primer
  source 1..33: mol_type = other DNA
ctagctgaattctcatccgacgtgtttccgccg

SEQ ID NO: 305 - DNA, 33 bp, synthetic construct
  misc_feature 1..33: note = Primer
  source 1..33: mol_type = other DNA
caattacatatggcgcccaagacctactgcgag

SEQ ID NO: 306 - DNA, 36 bp, synthetic construct
  misc_feature 1..36: note = Primer
  source 1..36: mol_type = other DNA
caattaaagcttctaggccagcatcgagtcgatcgc

SEQ ID NO: 307 - DNA, 51 bp, synthetic construct
  misc_feature 1..51: note = Primer
  source 1..51: mol_type = other DNA
caattacatatgcatcaccatcaccatcacatgcaattcgacgtgaccatc

SEQ ID NO: 308 - DNA, 36 bp, synthetic construct
  misc_feature 1..36: note = Primer
  source 1..36: mol_type = other DNA
caattagaattctcagtgtgtaccggccttgaagcg

SEQ ID NO: 309 - DNA, 51 bp, synthetic construct
  misc_feature 1..51: note = Primer
  source 1..51: mol_type = other DNA
caattacatatgcatcaccatcaccatcacacttccggcgatatgtcgagc

SEQ ID NO: 310 - DNA, 33 bp, synthetic construct
  misc_feature 1..33: note = Primer
  source 1..33: mol_type = other DNA
caattagaattctcagcgcggaatacttgcctg

SEQ ID NO: 311 - DNA, 33 bp, synthetic construct
  misc_feature 1..33: note = Primer
  source 1..33: mol_type = other DNA
gtgctagccatatggaaaaaatgtcacatgatc

SEQ ID NO: 312 - DNA, 35 bp, synthetic construct
  misc_feature 1..35: note = Primer
  source 1..35: mol_type = other DNA
ctggatccaagcttctattcggcgaagacgccggc

SEQ ID NO: 313 - DNA, 33 bp, synthetic construct
  misc_feature 1..33: note = Primer
  source 1..33: mol_type = other DNA
gtgctagccatatgctgtggcacgcaatgccac

SEQ ID NO: 314 - DNA, 35 bp, synthetic construct
  misc_feature 1..35: note = Primer
  source 1..35: mol_type = other DNA
ctggatccaagctttcaccagtcgtcctcttcgtc

SEQ ID NO: 315 - DNA, 51 bp, synthetic construct
  misc_feature 1..51: note = Primer
  source 1..51: mol_type = other DNA
caattacatatgcatcaccatcaccatcacgtgaagcgagcgctcatcacc

SEQ ID NO: 316 - DNA, 35 bp, synthetic construct
  misc_feature 1..35: note = Primer
  source 1..35: mol_type = other DNA
caattagaattctcatgtccggccggcgatcatcg

SEQ ID NO: 317 - DNA, 50 bp, synthetic construct
  misc_feature 1..50: note = Primer
  source 1..50: mol_type = other DNA
ccattacatatgcatcaccatcaccatcacatgacagagcagcagtggaa

SEQ ID NO: 318 - DNA, 33 bp, synthetic construct
  misc_feature 1..33: note = Primer
  source 1..33: mol_type = other DNA
ccattagaattcctatgcgaacatcccagtgac